(19) United States
(12) Patent Application Publication
(10) Pub. No.: US 2026/0286005 A1
(43) Pub. Date: Sep. 24, 2026

(54) IMPROVED GLYCAN-DEPENDENT IMMUNOTHERAPEUTIC BI-SPECIFIC FUSION PROTEINS AND CHIMERIC ANTIGEN RECEPTORS

(71) Applicant: THE REGENTS OF THE UNIVERSITY OF CALIFORNIA, Oakland, CA (US)

(72) Inventors: Michael DEMETRIOU, Oakland, CA (US); Raymond Wenhou ZHOU, Oakland, CA (US)

(73) Assignee: THE REGENTS OF THE UNIVERSITY OF CALIFORNIA, Oakland, CA (US)

(21) Appl. No.: 18/874,298

(22) PCT Filed: Jun. 9, 2023
(86) PCT No.: PCT/US2023/024898

§ 371 Date: Dec. 12, 2024

Related U.S. Application Data
(60) Provisional application No. 63/351,596, filed on Jun. 13, 2022.

(51) Int. Cl. C07K 16/30; A61K 38/00; A61K 39/00; A61P 35/00; C07K 14/705; C07K 16/28

(52) U.S. Cl. CPC C07K 16/3076; A61P 35/00; C07K 14/7051; C07K 16/2803; C07K 16/2806; C07K 16/2809; C07K 16/2818; C07K 16/2833; C07K 16/2851; C07K 16/2866; C07K 16/2896; A61K 38/00; A61K 2039/505; C07K 2317/31; C07K 2317/622; C07K 2319/03

(57) ABSTRACT

Provided are compositions and methods for treating diseases associated with aberrant glycosylation of cell surface molecules and expression of tumor-associated carbohydrate antigens (TACA). Also provided are bi-specific fusion proteins and chimeric antigen receptors (CARs) specific to tumor-associated carbohydrate antigens (TACA-CARs), vectors encoding the TACA-bi-specific fusion proteins and CARs, and recombinant cells comprising the TACA-bi-specific fusion proteins and CARs.

### CROSS-REFERENCE TO RELATED APPLICATIONS

This application claims the benefit of priority to U.S. Provisional Patent Application No. 63/351,596, filed Jun. 13, 2022, which is hereby incorporated by reference in its entirety for any and all purposes.

### STATEMENT REGARDING FEDERALLY-SPONSORED RESEARCH OR DEVELOPMENT

This invention was made with government support under Grant Number U01CA233078, R41CA233111 and R41CA261408, awarded by National Institutes of Health/National Cancer Institute. The government has certain rights in the invention.

### FIELD OF THE DISCLOSURE

The present disclosure relates generally to the field of pharmacology and immunology and specifically to bi-specific fusion proteins and chimeric antigen receptors that target tumor-associated carbohydrate antigens (TACA bi-specific fusion proteins; TACA-CARs) and the use of immune cells expressing the TACA bi-specific fusion proteins and TACA-CARS to treat a disease associated with aberrant glycosylation of cell surface molecules.

### BACKGROUND OF THE DISCLOSURE

Antigen-targeting cancer immunotherapies such as bi-specific antibodies (e.g., Bi-specific T cell engager) or Chimeric Antigen Receptor T cells (engineered immune cells expressing e.g., a Chimeric Antigen Receptor (CAR)) are the most potent immunotherapies known. Both trigger T cell mediated killing of cancer cells, with complete response rates for CAR T cells as high as ~90% in relapsed/refractory B cell malignancies. Both utilize a single-chain variable fragment (scFv) derived from the variable heavy and light chains of a monoclonal antibody to target antigens expressed in cancer. In bi-specific antibodies, the antigen-specific scFv is fused to a second scFv specific to CD3, while in CARs the antigen-specific scFv is fused to a transmembrane and one or more cytoplasmic signaling domains derived from an immune cell receptor. Both types of chimeric molecules are genetically expressed in T cells. Both therapies are currently approved to treat CD19⁺ B-cell malignancies.

To apply bi-specific proteins and/or CAR T cells to a wide variety of cancer types, a cell surface cancer antigen that can be safely targeted must first be identified. This is a major challenge, particularly for solid cancers. The lack of safe cell-surface protein antigens available for targeting leaves the vast majority of cancer patients without the potential of these emerging immunotherapies. Moreover, even if new safe cell-surface antigens are identified, different bi-specific and/or CAR T cells will likely need to be developed for each different antigen or cancer. This greatly increases development time and costs. Accordingly, there is a need for a better immunotherapeutic approach for targeting an antigen present in multiple common cancers that has limited or no expression in normal tissue. The present disclosure satisfies this unmet need.

### SUMMARY OF THE INVENTION

The present disclosure provides a novel class of immunotherapeutic bi-specific fusion proteins and Chimeric Antigen receptors that effectively target Tumor Associated Carbohydrate Antigens (TACA) for immunotherapy. Specifically, an antigen-binding domain derived from a lectin rather than a monoclonal antibody or fragment thereof was used to engineer bi-specific fusion proteins and CARs to target TACAs irrespective of the carrier protein.

Accordingly, one aspect of the present disclosure provides an isolated nucleic acid molecule encoding a bi-specific fusion protein comprising: (a) an antigen binding domain that selectively binds a tumor-associated carbohydrate antigen (TACA), and (b) an immune cell recognition domain that specifically binds a receptor on an immune effector cell. In some embodiments, the antigen binding domain comprises a TACA-binding domain derived from a lectin; and the antigen binding domain comprises more than one TACA binding domains.

Another aspect of the present disclosure provides an isolated nucleic acid molecule encoding a chimeric antigen receptor (CAR) comprising: (a) an antigen binding domain that selectively binds a tumor-associated carbohydrate antigen (TACA); (b) a transmembrane domain; (c) a costimulatory signaling region; and (d) an intracellular signaling domain. In some embodiments, the antigen binding domain comprises a TACA-binding domain derived from a lectin; and the antigen binding domain comprises more than one TACA binding domains.

In some embodiments, the antigen binding domain of the bi-specific fusion protein or CAR disclosed herein comprises two, three, four, five, six, seven, eight, nine, ten, or more TACA binding domains.

In some embodiments, the more than one TACA binding domains are operably linked by a linker. In some embodiments, the linker is selected from the group consisting of a peptide linker, a non-peptide linker, a chemical unit, a hindered cross-linker, a non-hindered cross-linker. In some embodiments, the linker is a peptide linker. In some embodiments, the peptide linker is at least about 4, at least about 6, at least about 8, at least about 10, at least about 12, at least about 14, or at least about 15 amino acids in length. In some embodiments, the peptide linker is a glycine-serine linker. In some embodiments, the linker comprises the amino acid sequence selected from the group consisting of SEQ ID NO: 128, SEQ ID NO: 129, SEQ ID NO: 127, SEQ ID NO: 130, SEQ ID NO: 131, and SEQ ID NO: 132. In some embodiments, the linker comprises the amino acid sequence of SEQ ID NO: 127. In some embodiments, the linker comprises the amino acid sequence of SEQ ID NO: 131.

In some embodiments, the antigen binding domain of the bi-specific fusion protein or CAR disclosed herein comprises a TACA-binding domain derived from a lectin selected from a galectin, a siglec, a selectin; a C-type lectin; CD301, a polypeptide N-acetylgalactosarninyltransferase (ppGalNAc-T), L-PHA (Phaseolus vulgaris leukoagglutinin); E-PHA (Phaseolus vulgaris erythroagglutinen); tomato lectin (Lycopersicon esculentum lectin; LEA); peanut lectin (Arachis hypogaea Agglutinin; PNA); potato lectin (Solanum tuberosum lectin), pokeweed mitogen (Phytolacca American lectin), wheat germ agglutinin (Triticum Vulgaris lectin); Artocarpus polyphemus lectin (Jacalin letin); Vicia villosa Agglutinin (VVA); Helix pomatia Agglutinin (HPA); Wisteria floribunda Agglutinin (WFA); Sambucus nigra Agglutinin (SNA), BC2L-CNt (lectin from the gram negative bacteria Burkholderia cenocepacia), Maackia amurensis leukoagglutinin (MAL), Psathyrella velutina (PVL), Sclerotium rolfsii lectin (SRL), Eucheuma serra agglutinin (ESA), CLEC17A (Prolectin), Aleuria aurantia lectin, Sambucus sieboldiana lectin (SSA), Glechoma hederacea lectin (Gleheda), Morus nigra agglutinin (Morniga G), Salvia sclarea lectin, Salvia bogotensis lectin, Salvia horminum lectin, Clerodendrum trichotomum lectin, Moluccella laevis lectin, Griffonia simplicifolia (GsLA4), Psophocarpus tetragonolobus (acidic WBAI), Abrus precatorius lectin, Amaranthus caudatus lectin, Amaranthus leucocarpus lectin, Laelia autumnalis lectin, Artocarpus integrifolia lectin, Maclura pomifera lectin, Artocarpus lakoocha lectin, Dolichos biflorus agglutinin, Dolichos biflorus lectin, Glycine max lectin, and Agaricus bisporus lectin.

In some embodiments, the galectin is selected from the group consisting of galectin-1, galectin-2, galectin-3, galectin-4, galectin-5, galectin-6, galectin-7, galectin-8, galectin-9, galectin-10, galectin-11, galectin-12, galectin-13, galectin-14 and galectin-15. In some embodiments, the siglec is selected from the group consisting of siglec-1 (sialoadhesion), siglec-2 (CD22), siglec-3 (CD33), siglec-4 (myelin associated glycoprotein), siglec-5, siglec-6, siglec-7, siglec-8, siglec-9, siglec-10, siglec-11, siglec-12, siglec-13, siglec-14, siglec-15, siglec-16, siglec-17, Siglec E, Siglec F, siglec G and siglec H.

In some embodiments, the polypeptide N-acetylgalactosaminyltransferase (ppGalNAc-T) is selected from the group consisting of ppGalNAc-T1 (GALNT1), ppGalNAc-T2 (GALNT2), ppGalNAc-T3 (GALNT3), ppGalNAc-T4 (GALNT4), ppGalNAc-T5 (GALNT5), ppGalNAc-T6 (GALNT6), ppGalNAc-T7 (GALNT7), ppGalNAc-T8 (GALNT8), ppGalNAc-T9 (GALNT9), ppGalNAc-T10 (GALNT10), ppGalNAc-T12 (GALNT12), ppGalNAc-T13 (GALNT13), ppGalNAc-T14 (GALNT14), ppGalNAc-T15 (GALNT15), ppGalNAc-T16 (GALNT16), ppGalNAc-T17 (GALNT17), ppGalNAc-T18 (GALNT18), ppGalNAc-T Like 5 (GALNTL5), and ppGalNAc-T Like 6 (GALNTL6).

In some embodiments, the antigen binding domain of the bi-specific fusion protein or CAR disclosed herein selectively targets a TACA selected from the group consisting of β1, 6 branching, β1,6GlcNAc-branched N-glycans, T antigen, Tn antigen, sialyl-T epitopes, Thomsen-nouveau (Tn) epitopes (Tn antigen), sialyl-Tn epitopes (sialyl-Tn antigen), α2, 6 sialylation, Sialylation, sialyl-Lewis^{x/a}, di-sialyl-Lewis^{x/a}, sialyl 6-sulfo Lexis^{x}, Lewis-y (Le^{y}), Lewis Y, Globo H, GD2, GD3, GM3, and Fucosyl GM1. In some embodiments, the antigen binding domain selectively targets β1,6GlcNAc-branched N-glycans, Tn epitopes (Tn antigen), sialyl-Tn epitopes (sialyl-Tn antigen), GalNAcα-Serine, GalNAcα-Threonine, GalNAc, or GalNAcβ1.

In some embodiments, the antigen binding domain of the bi-specific fusion protein or CAR disclosed herein comprises the amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152.

In some embodiments, the antigen binding domain comprises an amino acid sequence having at least 90% homology to SEQ ID NO: 100, 101, 102, 103, 104, 105, 106, 107, 108, 109, 133, 134, 135, 136, 136, 137, 139, 140, 141, 142, 143, 144, 145, 146, or 152.

In some embodiments, the isolated nucleic acid comprises an expression vector; and/or an in vitro transcribed RNA.

In some embodiments of the isolated nucleic acid molecule disclosed herein, the isolated nucleic acid molecule encodes a bi-specific fusion protein comprising an amino acid sequence selected from SEQ ID NOs: 1-5, 10-34, 39-42, 47-50, 55-58, and 63-66; or an amino acid sequence having at least 90% sequence identity to an amino acid sequence selected from SEQ ID NOs: 1-5, 10-34, 39-42, 47-50, 55-58, and 63-66. In some embodiments, the isolated nucleic acid molecule encodes a bi-specific fusion protein comprising the amino acid sequence selected from SEQ ID NO: 3-5, 11-13, 19-21, 28-30, 32-34, 40-42, 48-50, 56-58, or 64-66. In some embodiments, the isolated nucleic acid molecule encodes a bi-specific fusion protein comprising the amino acid sequence of SEQ ID NOs: 32-34, 40-42, 48-50, 56-58, or 64.

In some embodiments of the isolated nucleic acid molecule disclosed herein, (a) the bi-specific fusion protein exhibits enhanced binding to Thomsen-nouveau (Tn) antigen expressing tumor cells when compared to a bi-specific fusion protein comprising a flexible linker in the antigen binding domain. In some embodiments, the bi-specific fusion protein exhibits enhanced binding to β1,6GlcNAc-branched N-glycans expressing tumor cells when compared to a bi-specific fusion protein comprising a flexible linker in the antigen binding domain. In those embodiments, the flexible linker is a glycine-serine linker or a linker comprising an amino acid sequence selected from SEQ ID NO: 124, SEQ ID NO: 128, SEQ ID NO: 129, or SEQ ID NO: 127; or an amino acid sequence having at least 90% sequence identity to an amino acid sequence selected from SEQ ID NO: 124, SEQ ID NO: 128, SEQ ID NO: 129, or SEQ ID NO: 127.

In some embodiment of the isolated nucleic acid molecule disclosed herein, the immune effector cell targeted by the bi-specific fusion protein is selected from the group consisting of a T cell, a natural killer (NK) cell, a natural killer T (NKT) cell, a macrophage, a monocyte, a dendritic cell, and a neutrophil. In one embodiment, the immune effector cell is a T cell. In one embodiment, the immune effector cell is an NK cell. In some embodiments, the receptor on the immune effector cell is selected from the group consisting of T-cell receptor (TCR) alpha, TCR beta, TCR gamma, TCR delta, invariant TCR of NKT cells, CD3, CD2, CD28, CD25, CD16, NKG2D, NKG2A, CD138, KIR3DL, NKp46, MICA, and CEACAM1. In some embodiments, the receptor on the immune effector cell is: (i) a T cell receptor selected from the group consisting of CD3, CD2, CD28, and CD25; or (ii) an NK cell receptor selected from the group consisting of NKG2D, NKG2A, CD138, KIR3DL, NKp46, MICA, and CEACAM1.

In some embodiments, the immune cell recognition domain of the bi-specific fusion protein disclosed herein comprises: (i) a peptide, a protein, an antibody, a single domain antibody, a nanobody, an antibody fragment, or single-chain variable fragment (scFv) that selectively binds to a receptor on the immune effector cell; (ii) an antibody Fc domain, optionally an Fc region of an IgG molecule; or (iii) the constant region domains CH2 and/or CH3 of an antibody, preferably CH2 and CH3, optionally with or without a hinge region.

In some embodiments, the immune cell recognition domain comprises: (i) an scFv that selectively binds CD3, CD2, CD28, CD25, CD16, NKG2D, NKG2A, CD138, KIR3DL, NKp46, MICA, and CEACAM1; (ii) the amino acid sequence of SEQ ID NOs: 149, 150 or 151; or (iii) an amino acid sequence having at least 90% sequence identity to the amino acid sequence of SEQ ID NO: 149, 150, or 151.

In some embodiments, the bi-specific fusion protein is an Fc fusion protein comprising the antigen binding domain that selectively binds a tumor-associated carbohydrate antigen (TACA) and the Fc domain.

In some embodiment of the isolated nucleic acid molecule disclosed herein, the transmembrane domain of the CAR comprises a transmembrane region of a molecule selected from the group consisting of T-cell receptor (TCR)-alpha, TCR-beta, CD3-zeta, CD3-epsilon, CD4, CD5, CD8, CD9, CD16, CD22, CD28, CD33, CD37, CD45, CD64, CD80, CD86, CD134 (Ox40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In some embodiments, the transmembrane domain comprises a CD8 transmembrane domain. In some embodiments, the transmembrane domain comprises the amino acid sequence of SEQ ID NO: 148.

In some embodiment of the isolated nucleic acid molecule disclosed herein, the costimulatory domain of the CAR is a costimulatory domain of a molecule selected from the group consisting of CD27, CD28, 4-IBB (CD137), OX40, CD30, CD40, PD-1, ICOS, lymphocyte function-associated antigen-1 (LFA-1), CD2, CD7, CD8, LIGHT, NKG2C, B7-H3, a ligand that specifically binds with CD83, DAP10, DAP12, Lck, Fas, and a combination thereof. In some embodiments, the costimulatory domain comprises: (i) a 4-1BB costimulatory domain; (ii) the amino acid sequence of SEQ ID NO: 114; (iii) a CD28 costimulatory domain; (iv) the amino acid sequence of SEQ ID NO: 113; or (v) a 4-1BB and a CD28 costimulatory domains.

In some embodiment of the isolated nucleic acid molecule disclosed herein, the intracellular domain of the CAR comprises the intracellular signalling domain of a molecule selected from the group consisting of T cell receptor (TCR) zeta, FcR-gamma, FcR-beta, CD3-gamma, CD3-delta, CD3-epsilon, CD3-zeta, CDS, CD5, CD22, CD79a, CD79b, and CD66d. In some embodiments, the intracellular signalling domain comprises a CD3zeta signalling domain; or the amino acid sequence of SEQ ID NO: 115.

In some embodiment of the isolated nucleic acid molecule disclosed herein, the CAR further comprises a hinge domain. In some embodiment, the hinge domain is a protein selected from the group consisting of a CD8a, an Fc fragment of an antibody, a hinge region of an antibody, a CH2 region of an antibody, a CH3 region of an antibody, and an artificial spacer sequence. In some embodiments, the hinge domain is a CD8a hinge domain or wherein the hinge domain comprises the amino acid sequence of SEQ ID NO: 147. In some embodiments, the hinge domain comprises an amino acid sequence selected from the group consisting of SEQ ID NO: 119, 124, 127, 128, 129, 130, 131, 132, and 147.

One aspect of the present disclosure provides an isolated nucleic acid molecule encoding a chimeric antigen receptor (CAR). In some embodiments, the CAR encoded by the isolated nucleic acid molecule comprises: (i) an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99; or (ii) an amino acid sequence having at least 90%, at least 91%, at least 92%, at least 93%, at least 94%, at least 95%, at least 96%, at least 97%, at least 98%, at least 99% sequence identity to an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99.

One aspect of the present disclosure provides a bi-specific fusion protein that selectively binds a tumor-associated carbohydrate antigen (TACA) disclosed herein. In some embodiments, the bi-specific fusion protein is encoded by the isolated nucleic acid described herein.

One aspect of the present disclosure provides a bi-specific fusion protein that selectively binds a tumor-associated carbohydrate antigen (TACA) comprising: (i) an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 146, or 152; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152; and (ii) an immune cell recognition domain that specifically binds a receptor on an immune effector cell.

In some embodiments, the immune cell recognition domain comprises: (i) an antibody Fc domain; (ii) an Fc region of an IgG molecule; (iii) a peptide, a protein, an antibody, a single domain antibody, an antibody fragment, or single-chain variable fragment (scFv) that selectively binds to a receptor on the immune effector cell; or (iv) the constant region domains CH2 and/or CH3 of an antibody, preferably CH2 and CH3, optionally with or without a hinge region.

In some embodiments, the receptor on the immune effector cell is selected from the group consisting of T-cell receptor (TCR) alpha, TCR beta, TCR gamma, TCR delta, invariant TCR from NKT cells, CD3, CD2, CD28, CD25, CD16, NKG2D, NKG2A, CD138, KIR3DL, NKp46, MICA, and CEACAM1.

In some embodiments, the bi-specific fusion protein comprises: (a) the amino acid sequence selected from SEQ ID NO: SEQ ID NOs: 1-5, 10-34, 39-42, 47-50, 55-58, and 63-66; or (b) an amino acid sequence having at least 90% sequence identity to an amino acid sequence selected from SEQ ID NOs: SEQ ID NOs: 1-5, 10-34, 39-42, 47-50, 55-58, and 63-66; or (c) the amino acid sequence selected from SEQ ID NO: 3-5, 11-13, 19-21, 28-30, 32-34, 40-42, 48-50, 56-58, or 64-66; or (d) an amino acid sequence having at least 90% sequence identity to an amino acid sequence selected from SEQ ID NO: 3-5, 11-13, 19-21, 28-30, 32-34, 40-42, 48-50, 56-58, or 64-66.

In some embodiments, the bi-specific fusion protein selectively targets a TACA selected from the group consisting of β1, 6 branching, β1,6GlcNAc-branched N-glycans, T antigen, sialyl-T epitopes, Thomsen-nouveau (Tn) epitopes (Tn antigen), sialyl-Tn epitopes (sialyl-Tn antigen), α2, 6 sialylation, Sialylation, sialyl-Lewis^{x/a}, di-sialyl-Lewis^{x/a}, sialyl 6-sulfo Lexis^{x}, Lewis-y (Le^{y}), Lewis Y, Globo H, GD2, GD3, GM3, and Fucosyl GM1. In some embodiments, the bi-specific fusion protein selectively targets a Tn antigen or a β1,6GlcNAc-branched N-glycan.

In some embodiments, the bi-specific fusion protein that selectively targets a Tn antigen comprises an antigen binding domain having the amino acid sequence selected from SEQ ID NO: 103-109, 142-146, or 152; or an amino acid sequence having at least 90% sequence identity to an amino acid sequence selected from SEQ ID NO: 103-109, 142-146, or 152. In some embodiments, the bi-specific fusion protein that selectively targets a Tn antigen comprises: (a) the amino acid sequence selected from SEQ ID NOs: 26-34, 39-42, 47-50, 55-58, or 63-66; or (b) an amino acid sequence having at least 90% sequence identity to an amino acid sequence selected from SEQ ID NOs: 26-34, 39-42, 47-50, 55-58, or 63-66; (c) the amino acid sequence selected from SEQ ID NOs: 28-30, 32-34, 40-42, 48-50, 56-58, or 64-66; or (d) an amino acid sequence having at least 90% sequence identity to an amino acid sequence selected from SEQ ID NOs: 28-30, 32-34, 40-42, 48-50, 56-58, or 64-66.

In some embodiments, the bi-specific fusion protein that selectively targets a β1,6GlcNAc-branched N-glycan comprises an antigen binding domain having the amino acid sequence selected from SEQ ID NO: 100-102, or 133-141; or an amino acid sequence having at least 90% sequence identity to an amino acid sequence selected from SEQ ID NO: 100-102, or 133-141.

In some embodiments, the bi-specific fusion protein that selectively targets a β1,6GlcNAc-branched N-glycan comprises: (a) the amino acid sequence selected from SEQ ID NOs: 1-5 and 10-25; or (b) an amino acid sequence having at least 90% sequence identity to an amino acid sequence selected from SEQ ID NOs: 1-5 and 10-25; (c) the amino acid sequence selected from SEQ ID NOs: 3-5, 11-13, or 19-21; or (d) an amino acid sequence having at least 90% sequence identity to an amino acid sequence selected from SEQ ID NOs: 3-5, 11-13, or 19-21.

One aspect of the present disclosure provides a bi-specific fusion protein that selectively binds a tumor-associated carbohydrate antigen (TACA) comprising: (i) an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 146, or 152; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152; and (ii) an immune cell recognition domain that specifically binds CD3 on an immune effector cell.

Another aspect of the present disclosure provides a bi-specific fusion protein that selectively binds a tumor-associated carbohydrate antigen (TACA) comprising: (i) a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 146, or 152; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152; and (ii) a peptide, a protein, an antibody, a single domain antibody, an antibody fragment, or single-chain variable fragment (scFv) that selectively binds to a receptor on the immune effector cell; and/or (iii) an Fc domain of antibody, optionally an Fc region of an IgG molecule; or (iv) the constant region domains CH2 and/or CH3 of an antibody, preferably CH2 and CH3, optionally with or without a hinge region.

One aspect of the present disclosure provides a chimeric antigen receptor that selectively binds a tumor-associated carbohydrate antigen (TACA). In some embodiments, the CAR is encoded by the isolated nucleic acid disclosed herein.

One aspect of the present disclosure provides a chimeric antigen receptor that selectively binds a tumor-associated carbohydrate antigen (TACA) comprising: (i) an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 146, or 152; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152; (ii) a CD8 a hinge domain; (iii) a CD8 transmembrane domain; (iv) a CD28 costimulatory and/or a 4-1BB costimulatory domain; and (v) a CD3 zeta intracellular signalling domain.

In some embodiments, the CAR comprises the amino acid sequence of SEQ ID NO: 72, 88, 89, 91, 92, or 93. In some embodiments, the CAR comprises the amino acid sequence of SEQ ID NO: 93, 94, 95, 96, 97, 98, or 99.

One aspect of the present disclosure provides an expression construct comprising the isolated nucleic acid described herein.

In some embodiments, the expression construct further comprises a promoter. In that embodiments, the promoter is selected from an EF-la promoter, a T cell Receptor alpha (TRAC) promoter, interleukin 2 (IL-2) promoter, or cytomegalovirus (CMV) promoter, a simian virus 40 (SV40) early promoter, a mouse mammary tumor virus (MMTV) promoter, a human immunodeficiency virus (HIV) long terminal repeat (LTR) promoter, a Moloney Murine Leukemia Virus (MoMuLV) promoter, an avian leukemia virus promoter, an Epstein-Barr virus immediate early promoter, or a Rous sarcoma virus promoter.

In some embodiments, the expression construct is a viral vector selected from the group consisting of a retroviral vector, a lentiviral vector, an adenoviral vector, and an adeno-associated viral vector. In one embodiment, the expression construct is a lentiviral vector. In one embodiments, the expression construct is a self-inactivating lentiviral vector.

In some embodiments, the expression construct comprises an isolated nucleic acid molecule encoding a bi-specific fusion protein described herein, and an isolated nucleic acid molecule encoding a CAR described herein. In some embodiments, the isolated nucleic acid molecule encoding a bi-specific fusion protein described herein, and the isolated nucleic acid molecule encoding a CAR described herein are operably linked by a nucleic acid molecule encoding a self-cleaving 2A peptide selected from P2A, T2A, E2A, or F2A.

One aspect of the present disclosure provides a modified cell comprising the isolated nucleic acid described herein; the bi-specific fusion protein described herein; the CAR odescribed herein; or the expression vector described herein. In some embodiments, the cell is selected from the group consisting of a T cell, a CD4⁺ T cell, a CD8⁺ T cell, a Natural Killer (NK) cell, a cytotoxic T lymphocyte (CTL), and a regulatory T cell.

In some embodiments, the modified cell is a T cell. In some embodiments, the modified cell is an autologous cell, a xenogeneic cell, or an allogeneic cell.

In some embodiments, the modified cell comprises: (a) a bi-specific fusion protein of disclosed herein, and the CAR disclosed herein or a CAR that targets a tumor antigen; or (b) an isolated nucleic acid molecule encoding a bi-specific fusion protein disclosed herein and an isolated nucleic acid molecule encoding a CAR disclosed herein or a CAR that targets a tumor antigen; or (c) the expression construct disclosed herein.

One aspect of the present disclosure provides a composition comprising: (i) the isolated nucleic acid disclosed herein; (ii) the bi-specific fusion protein disclosed herein; (iii) the CAR disclosed herein; (iv) the expression vector disclosed herein; or (v) the modified cell disclosed herein. In some embodiments, the composition further comprising a pharmaceutically acceptable carrier.

One aspect of the present disclosure provides a method for generating the modified cell disclosed herein, the method comprising introducing into a cell the isolated nucleic acid for generating the modified cell, the bi-specific fusion protein for generating the modified cell; the CAR for generating the modified cell; or the expression vector for generating the modified cell.

One aspect of the present disclosure provides a method of treating cancer in a subject in need thereof, the method comprising administering to the subject a composition described herein.

In some embodiments, the composition comprises: (a) the isolated nucleic acid disclosed herein; (b) the bi-specific fusion protein disclosed herein; (c) the CAR disclosed herein; (d) the bi-specific fusion protein disclosed herein, and the CAR disclosed herein; (e) the expression vector disclosed herein; or (f) the modified cell odisclosed herein.

In some embodiments, the cancer is selected from the group consisting of a hematological malignancy, a solid tumor, a primary or a metastasizing tumor, a leukemia, a carcinoma, a blastoma, a sarcoma, a leukemia, lymphoid malignancies, a melanoma, and a lymphoma.

One aspect of the present disclosure provides a method of treating a cancer in a subject in need thereof, the comprising administering to the subject a therapeutically effective composition comprising a modified cell, wherein the modified cell comprises a bi-specific fusion protein and/or a CAR that selectively binds a tumor-associated carbohydrate antigen (TACA), and wherein the bi-specific fusion protein or the CAR comprises an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 146, and 152; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 100, 101, 102, 103, 104, 105, 106, 107, 108, 109, 110, 133, 134, 135, 136, 136, 137, 139, 140, 141, 142, 143, 144, 145, 146 or 152.

In some embodiments, the bi-specific fusion protein further comprises an immune cell recognition domain that specifically binds a receptor on an immune effector cell. In some embodiments, the immune cell recognition domain specifically binds CD3. In some embodiments, the immune cell recognition domain is an antibody Fc domain and a domain that specifically binds CD3.

In some embodiments of the method of treating a cancer in a subject in need thereof described herein, the bi-specific fusion protein comprises: (a) the amino acid sequence selected from SEQ ID NOs: 1-5, 10-34, 39-42, 47-50, 55-58, and 63-66; or (b) an amino acid sequence having at least 90% sequence identity to an amino acid sequence selected from SEQ ID NOs: 1-5, 10-34, 39-42, 47-50, 55-58, and 63-66; (c) the amino acid sequence selected from SEQ ID NO: 3-5, 11-13, 19-21, 28-30, 32-34, 40-42, 48-50, 56-58, or 64-66; or (d) an amino acid sequence having at least 90% sequence identity to an amino acid sequence selected from SEQ ID NO: 3-5, 11-13, 19-21, 28-30, 32-34, 40-42, 48-50, 56-58, or 64-66.

In some embodiments of the method of treating a cancer in a subject in need thereof described herein, the CAR further comprises a transmembrane domain; a costimulatory domain; and an intracellular signalling domain. In some embodiments, the CAR comprises a CD8 transmembrane domain; a CD28 costimulatory and/or a 4-1BB costimulatory domain; and a CD3 zeta intracellular signalling domain. In some embodiments, the CAR further comprises a hinge domain.

One aspect of the present disclosure provides a method of providing an anti-tumor immunity in a mammal, the method comprising administering to the mammal an effective amount of a population of modified cells disclosed herein.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIGS. 1A-D show a schematic illustration and characterization of Glycan-dependent T cell Recruiter (GlyTR1) bi-specific proteins targeting β1,6GlcNAc-branched Nglycans.

FIG. 1A shows that GlyTR1 proteins are chimeric single polypeptide comprising the variable heavy and light chain domains of an anti-CD3 monoclonal antibody (CD3 scFv, OKT3 clone) linked to one or more L-PHA carbohydrate binding domains (CRDs). L-PHA means Phytohemagglutinin-L; H(6) means 6×-Histidine tag.

FIG. 1B shows a size exclusion chromatography (SEC) analysis of a GlyTR1^{L-PHAxCD3} and protein standards, Sigma (Cat #MWGF1000-1KT). SEC analysis was conducted using a GE Superdex 200 Increase3.2/300 columns. Molecular weights were calculated from trendlines generated from retention times.

FIG. 1C shows a flow cytometric analysis of cell surface binding on Jurkat T cells of GlyTR1^{L-PHAxCD3} and GlyTR1^{L-PHAΔ1-5xCD3}.

(FIG. 1D shows flow cytometric analysis of co-culture assay where Carboxyfluorescein succinimidyl ester (CFSE)-labelled cancer cells were co-cultured with PBMC with E:T at 20:1 using indicated GlyTR1 molecules for 1 day. Live cancer cells were gated for analysis. Cell death (%)=(100−live cancer cell number treated with GlyTR1/live cancer cell number not treated with GlyTR1×100), where live cells are defined as CFSE⁺7AAD⁻. 7-aminoactinomycin D (7-AAD) is a fluorescent intercalator that undergoes a spectral shift upon association with DNA. thermofisher.com/order/catalog/product/A1310.

FIGS. 2A-D show the improved activity of GlyTR1 bi-specific protein targeting β1,6GlcNAc-branched N-glycans and comprising more than one L-PHA carbohydrate binding domains (CRDs) when compared to GlyTR1 comprising one L-PHA carbohydrate binding domains (CRDs).

FIG. 2A shows a SEC analysis of GlyTR1^{LPHA(2)xCD3} and protein standards, GE (Cat #GE28-4038-42), using a GE HiLoad 16/600 Superdex 200 pg columns. Molecular weights were calculated from trendlines generated from volumes.

FIG. 2B shows flow cytometric analysis of cell surface binding of GlyTR1^{L-PHA(2)xCD3}monomers and dimers on Jurkat T cells illustrating that dimeric GlyTR1^{L-PHA(2)xCD3} showed enhanced binding compared to monomeric GlyTR1^{LPHA(2)xCD3}.

FIG. 2B shows flow cytometric analysis of cell surface binding of monomeric GlyTR1L-^{PHAxCD3} and dimeric GlyTR1^{L-PHA(2)xCD3} illustrating that the dimeric GlyTR1^{L-PHA(2)xCD3} showed enhanced binding compared to monomeric GlyTR1L-^{PHAxCD3}.

FIG. 2D shows a line graph illustrating the flow cytometric analysis of co-culture assay where CFSE-labelled cancer cells were co-cultured with PBMC with E:T at 20:1 using indicated GlyTR1 molecules for 1 day; and demonstrating that dimeric GlyTR1^{L-PHA(2)xCD3} enhanced cell death when compared to monomeric GlyTR1L-^{PHAxCD3}. Live cancer cells were gated for analysis. Cell death (%)=(100−live cancer cell number treated with GlyTR1/live cancer cell number not treated with GlyTR1×100), where live cells are defined as CFSE⁺7AAD⁻.

FIGS. 3A-I show T cell dependent cancer killing by dimeric GlyTR1^{L-PHA(2)xCD3} in various cancer cell lines. Carboxyfluorescein succinimidyl ester (CFSE)-labeled cancer cells (as indicated) were co-cultured with/without PBMC (FIGS. 3A-H) or CD8 T cells (FIG. 3I) for 1 day (FIGS. 3A-B) or 3 days (FIGS. 3C-I) with E:T at 1:1 (FIG. 3A), 10:1 (FIG. 3B or 311) and 20:1 (FIGS. 3C-G, 3I), followed by flow cytometry analysis for cell death. Cell Death %=100−(live cells treated with GlyTR/live cells not treated with GlyTR)×100, where live cancer cells are defined as CFSE⁺7AAD⁻ (a, b) or CFSE⁺ FVD-eFluor780⁻ (c-i). Fixable Viable Dye -eFluor™-780 is a fixable viability dye that can be used to irreversibly label dead cells prior to cryopreservation, fixation and/or permeabilization procedures.thermofisher.com/order/catalog/product/65-2860-40. Data are mean±SEM of triplicate incubations.

FIGS. 4A-B show reduced non-cancer-induced T cell activation with dimeric GlyTR1^{L-PHA(2)xCD3}. FIGS. 4A-B show the quantification of flow cytometric analysis of CD69 expression on gated T cells after overnight stimulation of 3-day resting or Kifunensine-treated PBMC with GlyTR1^{L-PHAxCD3} (FIG. 4A) or PBMC (10⁵ cells/ml) with and without MDA-MB-231F cancer cells (10⁵ cells/ml) using dimeric GlyTR1^{L-PHA(2)xCD3} (FIG. 4B). Kifunensine is an inhibitor of class I α-mannosidases that inhibits glycoprotein processing in the endoplasmic reticulum and the golgi.

FIGS. 5A-F show in vivo activity of GlyTR1^{L-PHA(2)xCD3}. FIGS. 5A-B show that GlyTR1^{L-PHA(2)xCD3} induces tumor regression in vivo in NSG mice that were injected i.p. with the indicated cancer cell lines on day 0, then starting on day 6 treated with i.p. 1×10⁷ CD8⁺ T cells every 3-4 days for 2 or 3 injections (as indicated in the graphs) as well as with/without i.p, GlyTR1^{LPHA(2)xCD3} injected twice daily. Tumor burden quantiatated by luciferase activity (photons/second (p/s)) is shown in FIGS. 5C and D. These data illustrate that GlyTR1^{LPHA(2)xCD3} induced marked tumor regression when compared to PBS treatment in both breast cancer and ovarian cancer models. FIG. 5E shows accumulation of GlyTR1^{LPHA(2)xCD3} in lungs with cancer. NSG mice with/without lung metastasis (MDA-MB-231-Fluc) were injected i.v. with/without fluorophore (VivoTag© 680 XL (Perkin Elmer LLC) labelled GlyTR1^{LPHA(2)xCD3} and extracted lungs were imaged for luminescence (tumor) and fluorescence (GlyTR1).

FIG. 5F shows that GlyTR1^{LPHA(2)xCD3} did not induce human T cell activation in non-tumor bearing humanized NSG MI/II^{-/-}. PBMC humanized NSG-MI/II^{-/-} mice were injected s.c. with GlyTR1^{LPHA(2)xCD3} and analyzed 24 hrs later for T cell activation (CD69⁺) in blood by flow cytometry.

FIGS. 6A-E show in vivo half-life and distribution of dimeric GlyTR1^{L-PHA(2)xCD3} FIG. 6B shows that the serum half-life of dimeric GlyTR^{LPHA(2)xCD3} was about 2.7 hrs. C57BL/6 mice (n=2) were injected i.v. with GlyTR1^{LPHA(2)xCD3}, bled at the indicated times and serum analyzed by sandwich ELISA for GlyTR1^{L-PHA(2)xCD3}. FIGS. 6A, and 6C-6E show that GlyTR1^{L-PHA(2)xCD3} was stable in human plasma for up to 21 hours and showed little loss of intact protein and tagged dimeric GlyTR1^{L-PHA(2)xCD3} showed marked accumulation in the liver (FIG. 6E). GlyTR1^{L-PHA(2)xCD3} was fluorescently labelled with VivoTag® 680 XL (Perkin Elmer LLC) and injected i.v. into two C57BL/6 mice. These mice and a mock injected mouse were imaged at various times with the IVIS® Lumina Imager (2s exposure). Fluorescence in the liver and bladder regions were quantified at each time point and plotted as a percentage of total fluorescence after subtraction of background in the mock injected mouse (FIG. 6C). After 8 hrs, the mice were sacrificed, and the indicated organs were extracted and imaged (FIG. 6D). Fluorescence of each organ was quantified and after background subtraction from mock injected, was plotted as a percent of total fluorescence from all imaged organs (FIG. 6E).

FIG. 7 shows immunohistochemistry stainings and schematics of the stainings demonstrating the expression of L-PHA in normal human tissues. The ‘FDA999u’ (BioMAx) normal human tissue microarray containing 32 different tissues with replicates from 3 different individuals were stained with L-PHA-biotin (0.25 ug/ml) for 1 h, with detection by streptavidin-HRP (0.5 hr). The four highest staining tissues are highlighted in a box and shown at higher resolution. Staining of metastatic colon cancer under the same conditions is shown as a positive control. Adr-Adrenal gland, Bon—Bone marrow, Bre—Breast, Ceb—Cerebellum tissue, Cer—Cervix, Col—Colon, Dia—Diaphragm, Eso—Esophagus, Eye—Eye, Hea—Heart, Hyp—Hypophysis, Kid—Kidney, Lar—Larynx, Liv—Liver, Lun—Lung, Lym—Lymph node, Ner—Nerve, Ova—Ovary, Pan—Pancreas, Ple—Pleura, Pro—Prostate, Sal—Salivary gland, Ski—Skin, Sma—Small intestine, Spl—Spleen, Sto—Stomach, Str—Striatedmuscle, Tes—Testis, Thy—Thyroid or Thymus gland,Ton—Tonsil, Ute—Uterus.

FIGS. 8A-B show immunohistochemistry stainings (FIG. 8B) and schematics of the stainings (FIG. 8A) demonstrating the expression of GlyTR1^{LPHA(2)xCD3} in normal human tissue. Specifically, GlyTR1^{LPHA(2)xCD3} showed low but variable expression in the brush border of the small bowel, surface epithelial cells of the stomach, exocrine pancreas (acinus, intracellular), kidney cortex (glomerulus, proximal tubules), prostate and the molecular layer of the cerebellum. However Higher concentrations revealed lower and variable staining in adrenal, parotid duct, thyroid colloid, testis, uterus, spleen, and CNS white matter. The ‘FDA999w’ (BioMax) normal human tissue microarray, containing 32 different tissues with replicates from 3 different individuals, were stained with/without GlyTR1^{LPHA(2)xCD3} (0.5 ug/ml) for 1 h. GlyTR1^{LPHA(2)xCD3} was detected with an anti-HIS-HRP antibody at 1 μg/ml (0.5 hr). Adr—Adrenal gland, Bon—Bone marrow, Bre—Breast, Ceb—Cerebellum tissue, Cer Cervix, Col—Colon, Dia—Diaphragm, Eso—Esophagus, Eye—Eye, Hea—Heart, Hyp—Hypophysis, Kid—Kidney, Lar—Larynx, Liv—Liver, Lun—Lung, Lym—Lymph node, Ner—Nerve, Ova—Ovary, Pan—Pancreas, Per—Pericardium, Pro—Prostate, Sal—Salivary gland, Ske—Skeletal muscle, Ski—Skin, Sma—Small intestine, Spl—Spleen, Sto—Stomach, Tes—Testis, Thy—Thymus gland, Ton—Tonsil, and Ute—Uterus. Scale bar at higher resolution is 2 mm.

FIGS. 9A-B show immunohistochemistry stainings (FIG. 9B) and schematics of the stainings (FIG. 9A) demonstrating the expression of GlyTR^{LPHA(2)xCD3} in normal human tissue as in FIGS. 8A-B. The ‘FDA999—1’ (BioLabs) normal human tissue microarray, containing 32 different tissues with replicates from 3 different individuals, along with prostate cancer and matched normal prostate, were co-stained with GlyTR1^{LPHA(2)xCD3} (0.67 ug/ml) for 1 h, and detected with an nti-HIS-HRP at 1 μg/ml (0.5 hr). AD—Adrenal gland, BO—Bone marrow, BRN—Brain (cerebrum), BRT—Breast, CB—Cerebellum tissue, CR—Cervix, CO—Colon, ES—Esophagus, EY—Eye, HE—Heart, HY—Hypophysis, KI—Kidney, LA—Larynx, LI—Liver, LU—Lung, ME—mesothelium, NE—Nerve, OV—Ovary, PA—Pancreas, PT—Parathyroid, PR—Prostate, SA—Salivary gland, SK—Skin, SM—Small intestine, SP—Spleen, ST—Stomach, SM—Skeletal muscle, TE—Testis, TO—tonsil, TH—Thyroid, THM—Thymus gland, TO—Tonsil, and UT—Uterus. Scale bar at higher resolution is M.

FIGS. 10A-C show the quantification of the binding of GlyTR1^{LPHA(2)xCD3} in normal primary human renal epithelial cells and hepatocytes and GlyTR1^{LPHA(2)xCD3}—induced cytotoxicity demonstrating that normal primary human renal epithelial cells and hepatocytes were insensitive to T cell dependent killing by GlyTR1^{LPHA(2)xCD3} when compared to the robust GlyTR1^{LPHA(2)xCD3}—induced killing of MM1R multiple myeloma cells. FIGS. 10A-C show the quantification of flow cytometric analysis for GlyTR1^{LPHA(2)xCD3} cell surface binding (FIG. 10A) and/or GlyTR1^{LPHA(2)xCD3}—induced CD8⁺ T cell mediated killing (FIGS. 10B-C) (E:T=20:1, 2 days) of normal primary human renal epithelial cells (SciencCell), normal primary human hepatocytes (ScienCell), and MDA-MB-231 breast cancer cells (MHC-I deficient) and/or MM1R multiple myeloma cells (MHC-I deficient) by GlyTR1^{LPHA(2)xCD3}. Data are mean±SEM of triplicate incubations.

FIG. 11 shows L-PHA immunohistochemistry staining of normal human versus mouse tissue demonstrating L-PHA positive staining in mouse surface epithelial cells of the stomach, brush border of the small intestine and kidney (tubules>glomerulus). The ‘FDA999u’ (BioMAx) normal human tissue microarray and the mouse (C57BL6, AMS545 (Pantomics)) tissue microarray containing 32 and 22 normal tissues, respectively were stained with L-PHA-biotin (0.5 ug/ml) for 1 h, with detection by streptavidin-HRP (0.5 hr). The four highest staining human tissues along with their mouse counterparts are shown at higher resolution.

FIG. 12 shows toxicity assessment of GlyTR1^{L-PHA(2)xCD3} in PBMC humanized NSG-MI/II-mice demonstrating that GlyTR1^{L-PHA(2)xCD3} treatment did not significantly alter weight relative to mock treated mice (Panel b), nor did it affect liver function (AST, ALT, ALP, protein, albumin, total bilirubin), kidney function (BUN, creatinine), electrolytes, glucose, pancreatic function (amylase, precision PSL), thyroid function (total T4, TSH), cholesterol or muscle (CPK) (Panels c-v), blood levels of hemoglobin, RBC, hematocrit, WBC, WBC differential or platelets relative to control, T cell activation markers CD69 or CD25 in either CD4⁺ or CD8⁺ T cells, T cell activation, percentage of PD-1 positive CD4⁺ T and CD8⁺ T cells, percentage of total human CD45⁺ leukocytes, CD4⁺ T cells, CD8⁺ T cells, B cells or T regulatory cells (Treg) or serum hIFNγ or hIL-6 levels. NSG mice deficient for MHC class I and class II (NSG-MI/II⁻) were engrafted with 2×10⁷ human PBMCs intravenously (tail vein) on day 0 and day 15 (Panel a). Starting on day 16, mice were injected subcutaneously twice daily with PBS (n=6) or 10 ug GlyTR1^{L-PHA(2)xCD3} (n=6) for 12 days. Weight of mice during treatment (Panel b) was assessed. One treated mouse had ~15% weight loss at day 18 that recovered with saline injection. One treated mouse developed mild alopecia of the head. (Panels c-Q) Mice were euthanized on day 28, and blood and major organs were harvested. For clinical biochemistry, blood was pooled equally from 2 mice of the same treatment group to ensure sufficient volume for analysis (Panel c-v); each symbol represents two mice. For TSH, 0.03 data points represent <0.03 (i.e., detection limit of the assay) (t). Complete blood count was performed for each mouse, with each symbol representing one mouse (Panels w-D). A portion of each spleen was processed for flow cytometric analysis (Panels E-O), with each symbol representing one mouse (n=5 for some measurements due to technical problem during flow cytometry). Blood plasma from each mouse was used for human IFNγ and IL-6 ELISA, each symbol represents one mouse (Panels P-Q). All p-values by Mann-Whitney test (2-tail).

FIG. 13 shows toxicity assessment of GlyTR1^{LPHA(2)xCD3} in CD34⁺ humanized NSG mice demonstrating that GlyTR1^{L-PHA(2)xCD3} treatment did not induce any overt clinical toxicity nor alter weight, spleen size/cellularity, total human splenic CD4⁺ and CD8⁺ T cells, B cells, Treg cells or T cells positive for CD69, CD25 or PD-1, hemoglobin, RBC, hematocrit, WBC, WBC differential or platelets relative to control, kidney function (BUN, creatinine), liver function (AST, ALT, ALP, protein, albumin, total bilirubin), electrolytes, pancreatic function, (amylase, precision PSL), muscle (CPK) thyroid function (TSH) or cholesterol, Serum hIFNγ or hIL-6 levels. (Panels a-J) CD34⁺ stem cell humanized NSG mice 32 weeks post radiation/engraftment were injected subcutaneously twice daily with PBS (n=3), 2.5 ug (n=3), 5 ug (n=3) or 10 ug (n=3) of GlyTR^{LPHA(2)xCD3} for 10 days. (Panel a) Weight of mice during treatment. P-values by 2-way ANOVA and Bonferroni post test correction for multiple comparisons. *p<0.05. b-J) Mice were euthanized on day 10, and blood and major organs were harvested. A portion of each spleen was processed for flow cytometric analysis (Panels b-k); each symbol represents one mouse. Complete blood count was performed on blood, each symbol representing one mouse (Panels 1-p). For clinical biochemistry, blood was pooled equally from 3 mice of the same treatment group to ensure sufficient volume for analysis (Panel q-H); each symbol represents three mice. Blood plasma from each mouse was used for human IFNγ and IL-6 ELISA, each symbol represents one mouse (Panels I-J). P-values by Kruskal-Wallace test with Dunn's post test correction for multiple comparisons.

FIGS. 14A-D show schematic illustrations of improved GlyTR2 bi-specific proteins for targeting Tn antigens comprising multiple carbohydrate binding domains and their improved binding efficiency to various cells. FIG. 14A shows schematic illustration of various GlyTR2 proteins demonstrating that GlyTR2 proteins are chimeric single polypeptide comprising the variable heavy and light chains of an anti-CD3 monoclonal antibody (CD3 scFv, OKT3 clone) linked to more than one carbohydrate binding domains (CRDs) of CD301 (C-type lectin domain family 10 member A (CLEC10A)). H(6): 6×-Histidine tag. FIGS. 14 B-D show the quantifications of flow cytometric analyses of GlyTR2 bi-specific protein cell surface binding on TCRβ^{-/-} Jurkat T cells with and without inhibitors, Tn antigen, GalNAc, galactose, GalNAc.

FIGS. 15A-D show the improved activity of GlyTR2 bi-specific protein for targeting low-density Tn antigens. FIG. 15A shows chromatographs demonstrating that GlyTR2^{CD301(3)xCD3} was predominantly made up of large multimers and that GlyTR2^{slCD301(4)xCD3} with stiff-linkers was predominantly a monomer. SEC analysis of GlyTR2^{CD301(3)xCD3} and GlyTR2^{slCD301(4)xCD3} was compared to protein standards, GE (Cat #GE28-4038-42), using a GE HiLoad 16/600 Superdex 200 pg column. Molecular weights were calculated from trendlines generated from retention volumes. FIGS. 15B-C show the quantifications of flow cytometric analyses comparing the cell surface binding of GlyTR2^{CD301(3)xCD3} and GlyTR2^{slCD301(4)xCD3} on T cell leukemia TCRβ^{-/-} Jurkat and multiple myeloma MM.1R cells (FIG. 15B) and other indicated cancer types using established cell lines are shown as relative binding to TCRβ^{-/-} Jurkat T cells (FIG. 15C). Acute Monocytic Leukemia (AML): THP-1; ovarian cancer: SKOV3; non-small cell lung cancer NSCLC-1 and -2: H1975 and A549, respectively; colorectal cancer: DLD-1; pancreatic cancer: Hs766T; breast cancer: MDA-MB-231F; prostate cancer: PC3. GlyTR2^{slCD301(4)xCD3} containing 2 repeats of stiff linkers (SL2) were used. FIG. 15D shows the quantitification of a flow cytometric analysis of the surface binding of GlyTR2^{slCD301(4)xCD3} to MM.1R cells in the absence or presence of GalNac or GlcNAc. Data are mean±SEM of triplicate incubations.

FIGS. 16A-I show the quantifications of T cell dependent cancer killing by GlyTR2^{slCD301(4)xCD3} In FIGS. 16A-I, Carboxyfluorescein succinimidyl ester (CFSE)-labelled cancer cells (as indicated) were co-cultured with/without CD8 (FIG. 16A, 16I), T cells (FIG. 16B) or PBMC (FIGS. 16 C-H) for 3 days with E:T at 10:1 (FIGS. 16A, H, I) and 20:1 (FIGS. 16 B-G), followed by flow cytometric analysis for cell death. Cell Death %=100−(live cells treated with GlyTR/live cells not treated with GlyTR)×100, where live cancer cells are CFSETFVD-eFluor780. Fixable Viable Dye -eFluor™-780 is a fixable viability dye that can be used to irreversibly label dead cells prior to cryopreservation, fixation and/or permeabilization procedures.thermofisher.com/order/catalog/product/65-2860-40. Data are mean±SEM of triplicate incubations.

FIGS. 17A-B show the quantifications of GlyTR2^{slCD301(4)xCD3} induced robust T cell activation in the presence but not in the absence of cancer cells. Flow cytometric analysis of CD25 expression on gated T cells was conducted 3-day after stimulation of PBMC (5×10⁵ cells/ml) with and without SKOV3-Luc⁺MI^{-/-} cancer cells (2.5×10⁴ cells/ml) using GlyTR2^{slCD301(4)xCD3}.

FIGS. 18A-E show the in vivo activity of GlyTR2^{slCD301(4)xCD3} in solid cancers. FIGS. 18A-B show that GlyTR2^{slCD301(4)xCD3} induces tumor regression in vivo in NSG mice that were injected i.p. with the indicated cell lines on day 0, then on day 11 (breast cancer) or 6 and 10 (ovarian cancer) treated with i.p. 1×10⁷ CD8⁺ T cells or 4 subsequent injections of 2×10⁶ CD8⁺ T cells (ovarian cancer) every 3-4 days (as indicated in the graphs) as well as with/without i.p. GlyTR2^{slCD301(4)xCD3} injected twice daily. Tumor burden quantiatated by luciferase activity (photons/second (p/s)) and is shown in FIGS. 18C-D. These data illustrate that GlyTR2^{slCD301(4)xCD3} induced marked tumor regression when compared to PBS treatment in both breast cancer and ovarian cancer models. FIG. 18E shows the accumulation of GlyTR2^{slCD301(4)xCD3} in lungs with but not without cancer, demonstrating the specificity of GlyTR2^{slCD301(4)xCD3} for cancer cells in vivo NSG mice with/without lung metastasis (MDA-MB-231-Luc⁺MI^{-/-} C^{-/-}) were injected i.v. with/without fluorophore (VivoTag© 680 XL) labelled GlyTR2^{slCD301(4)xCD3} and extracted lungs were imaged for luminescence (tumor) and fluorescence (GlyTR2).

FIGS. 19A-C show the half-life and the stability of the GlyTR^{CD301(3)xCD3} in human plasma. FIG. 19A shows that the serum half-life of GlyTR2^{CD301(3)xCD3} was about 2 hrs when GlyTR^{CD301(3)xCD3} (100 ug) was injected i.v. into C57BL/6 mice (n=6). Serum was isolated at the indicated times and sampled by sandwich ELISA, with capture by anti-CD301 and detection by anti-HIS. FIGS. 19B-C show the stability of GlyTR^{CD301(3)xCD3} in human plasma at 37° C. for up to 21 hrs. GlyTR^{CD301(3)xCD3} was incubated at 37° C. in human plasma for the indicated times and then detected by sandwich ELISA and quantified (FIG. 19B) or by western blot with anti-HIS (FIG. 19C).

FIGS. 20A-F show GlyTR2^{slCD301xCD3} bio-distribution and safety demonstrating that GlyTR2^{slCD301xCD3} accumulates in the liver but is rapidly cleared by the liver and showed minimal accumulation in kidney, spleen, lung and intestine. FIGS. 20A-B show the localization of fluorescently labeled GlyTR2^{slCD301xCD3} in the respective tissues. GlyTR^{slCD301(4)xCD3} was fluorescently labelled with VivoTag© 680 XL (Perkin Elmer LLC) and injected along with vehicle i.v. into C57BL/6 mice. After 8 hrs, the mice were sacrificed and organs were extracted and imaged. FIG. 20C shows the quantification of the fluorescence of each organ of FIG. 20A was quantified and after background subtraction from vehicle injected and plotted as a percent of total fluorescence of all imaged organs. FIG. 20C shows an image of a formalin-fixed paraffin embedded normal human liver and breast cancer (positive control) co-stained with GlyTR2^{slCD301(4)xCD3}, followed by detection with anti-HIS-HRP antibody and DAB demonstrating GlyTR2^{slCD301(4)xCD} does not significantly binds to human or mouse liver cells. FIG. 20D shows that GlyTR2^{slCD301(4)xCD} does not significantly binds to human or mouse liver cells when compared to Jurkat TCRβ^{-/-} leukemia cells and MM.1R multiple myeloma cells. FIGS. 20 E-F show the quantifications of flow cytometric analyses demonstrating that GlyTR2^{slCD301(4)xCD3} did not induce T cell dependent killing of human hepatocytes, Human renal epithelial cells or normal T cells and B cells at concentrations that trigger cancer cell killing. Flow cytometric analysis of CD8⁺ T cell (FIG. 20E, E:T=20:1, 2-day) or PBMC (FIG. 20F, E:T=10:1, 1-day)-induced killing of Jurkat TCRβ^{-/-} leukemia (20E, 20F), MM.1R multiple myeloma (FIG. 20E), normal human hepatocytes or normal lymphocytes (FIG. 20F) by GlyTR2^{slCD301(4)xCD3} was quantified. Data are mean±SEM of 3 incubations.

FIG. 21 shows the toxicity assessment of GlyTR2^{CD301(4)xCD3} in PBMC humanized NSG-MI/II⁻ mice demonstrating no significant effects on body weight, liver function (AST,ALT,ALP, bilirubin, protein, albumin), kidney function (urea/creatinine), electrolytes (Na⁺, Cl⁻, K⁺, Ca²⁺), pancreatic function (amylase, precision PSL), thyroid function (total T4, TSH), cholesterol, muscle (CPK), WBC, WBC differential or platelets relative to mock injected mice, Serum hIFNγ or hIL-6 levels, minimal reductions in hemoglobin/RBC/hematocrit relative to control, no difference in the number of human CD45⁺ leukocytes or the percentage of CD4⁺ T cells, CD8⁺ T cells, B cells or T regulatory cells (Treg), no difference in the T cell activation markers CD69, CD25 or PD-1 in either CD4⁺ or CD8⁺ T cells, no T cell activation. (Panel a) NSG mice deficient for MHC class I and class II (NSG-MI/II⁻) were engrafted with 2×10⁷ human PBMCs intravenously (tail vein) on day 0 and day 15. Starting on day 16, mice were injected subcutaneously twice daily with PBS (n=6) or 100 ug GlyTR2^{CD301(4)xCD3} (n=6) for 12 days. (Panel b) Weight of mice during treatment. (Panels c-Q) Mice were euthanized on day 28, and blood and major organs were harvested. For clinical biochemistry, blood was pooled equally from 2 mice of the same treatment group to ensure sufficient volume for analysis (Panels c-v); each symbol represents two mice. For TSH, 0.03 data points represent <0.03 (i.e., detection limit of the assay) (Panel t). Complete blood count was performed for each mouse, with each symbol representing one mouse (Panels w-D). A portion of each spleen was processed for flow cytometric analysis (Panels E-O), with each symbol representing one mouse (n=5 for some measurement due to technical problem during flow cytometry). Blood plasma from each mouse was used for human IFNγ and IL-6 ELISA, each symbol represents one mouse (Panels P-Q). All p-values by Mann-Whitney test (2-tail).

FIGS. 22A-22I show schematic representations of GlyTR-CAR designs and GlyTR-CAR T cells expression and cytotoxic activity in vitro and in vivo. FIG. 22A shows the schematic representations of three GlyTR-CARs (GlyTR1^{LPHA(2)}, GlyTR2^{slCD301(4)} and mutGlyTR^{2slCD301(4)}) comprising an antigen binding domain with two LPHA or four CD301 domains, a CD8 transmembrane domain, a 41BB costimulatory domain and a CD3ζ intracellular signaling domain. mutGlyTR2^{slCD301(4)xCD3} CAR has five point mutations critical for sugar and calcium-binding in the antigen binding domain,(Gln267Gly, Asp269Gly, Glu280Gly, Asn292Gly and Asp293Gly in SEQ ID NO: 164 (NCBI RefSeq NP_878910.1)). FIG. 22 B shows a schematic illustrating the generation of GlyTR-CAR T cells. To generate CAR T cells, T cells were transduced using lentivirus, stimulated for 3 days with Dynabeads® Human T-Activator CD3/CD28, then rested from day 4-7. FIGS. 22C-E show that GlyTR1^{LPHA(2)} or GlyTR2^{slCD301(4)} CAR T cells readily killed ovarian and breast cancer cells.

FIG. 22C shows flow cytometry analyses on day 3 and day 7 characterizing the cell size and surface expression of the GlyTR-CARs. FIG. 22D-E show the quantification of GlyTR-CAR-mediated cell death on Day 7 following GlyTR-CAR T cells treatment as indicated. GlyTR-CAR T cells were incubated at increasing ratios with the indicated cancer cells and assessed on 72 hrs later for viable cancer cells by luminescence. Death % was calculated by [1-(cancer+CAR T/cancer)*100. FIGS. 22F-G show Interferon gamma (IFNγ) production in the presence of cancer cells compared to non-transduced (NT) T cells (FIG. 22F) or absence of cancer cells (FIG. 22G). IFNγ in supernatant from cultures in (FIG. 22 D) and (FIG. 22E), respectively, was determined by sandwich ELISA. FIG. 22H shows the in vivo killing of breast cancer cells transplanted into mice by the indicated GlyTR2-CAR T cells. Tumor burden quantiatated by luciferase activity (photons/second (p/s)) is shown in FIGS. 22I. These data illustrate that GlyTR2-CAR T cells induced marked tumor regression when compared to mutGlyTR2-CAR T cells in an breast cancer model.

### DETAILED DESCRIPTION

### I. Overview

Current bi-specific antibodies such as Blincyto and AFM11 (a CD19/CD3 TandAb) induce polyclonal T cell activation in the presence of PBMC in vitro and in vivo. In CAR T immunotherapy, the CAR T cells are activated in vitro prior to infusion into patients. This suggests that peripheral activation of T cells in vivo is important for efficient cancer killing. Consistent with this, animal models for other types of immunotherapy indicate that peripheral T cell activation is required for tumor eradication. However, over-activation can also lead to reduced activity via T cell exhaustion, as reported with standard CAR T cells in animal models. Moreover, exaggerated poly-clonal T cell activation can lead to varying degrees of ‘cytokine release syndrome’ in both CAR T and bi-specific therapies, a life threatening complication resulting from excessive pro-inflammatory cytokine release (e.g., IL-6).

To apply bi-specific fusion proteins and/or CAR T cells to a wide variety of cancer types, a cell surface cancer antigen that can be safely targeted must first be identified. This is a major challenge, particularly for solid cancers. The lack of safe cell-surface protein antigens available for targeting leaves the vast majority of cancer patients without the potential of these emerging immunotherapies. Moreover, even if new safe cell-surface antigens are identified, different bi-specific and/or CAR T cells will likely need to be developed for each different antigen/cancer. This greatly increases development time and costs.

A potential approach to address all of these issues is to target ‘Tumor Associated Carbohydrate Antigens’ (TACAs) that are over-expressed in many diverse cancer types, and even higher in metastatic and invasive disease. Carbohydrates (glycans), as well as glycoproteins and glycolipids, are major cell surface components. Virtually all cell surface proteins are glycosylated, with each protein having multiple glycans. Changes in cellular glycosylation are common in cancer due to aberrant expression of glycosyltransferases, glycosidases, and transporters, as well as differences in the abundancy of carbohydrate building blocks. These glycosylation alterations result in unique antigenic glycans known as Tumor-Associated Carbohydrate Antigens (TACAs). TACAs provide the most abundant and widespread cell surface cancer antigens known, with target density up to about 100-1000 fold greater than typical protein antigens. TACAs are over-expressed in many diverse cancer types, and even higher in metastatic and invasive disease.

Altered glycosylation resulting in TACAs is a near universal feature of cancer. As virtually all cell surface proteins are glycosylated, with each protein having multiple glycans, TACAs provide the most abundant and widespread cell surface cancer antigens known, with target density up to about 100-1000 fold greater than typical protein antigens.

TACAs are not simply markers of cancer, but also often serve as essential drivers of tumor growth and metastasis. For example, aberrant over-expression of β1,6GlcNAc-branched N-glycans in carcinomas drive tumor growth, motility, invasion, and metastasis. See e.g., Fernandes et al., Cancer research 51: 718-723 (1991); Litynska et al., Melanoma research 11: 205-212 (2001); Lau & Dennis, Glycobiology 18: 750-760 (2008); Dennis et al., Science 236: 582-585 (1987); and Demetrious et al., J. Cell Biology 130: 383-392 (1995). Another relevant TACA found on tumor cell is Tn antigen. Although not found on the cell surface of normal human tissue, Tn antigen is expressed in about 90% of human carcinomas and many hematopoietic cancers. Indeed, Tn antigen is one of the most specific human cancer associated structures known and promotes cell motility, invasiveness and metastasis. The Tn antigen is a single N-acetyl-galactosamine (GalNAc) a-O-linked to serine/threonine in proteins like mucins. Tn is a biosynthetic precursor of O-glycans that is normally extended with α1,3 linked galactose. The chaperone protein COSMC, a protein required by T-synthase to add galactose to GalNAc, is frequently altered in cancer. Mislocalization of enzymes within the ER/Golgi may also lead to abnormal Tn antigen expression in human cancer. The Tn antigen can be abnormally extended with Sialic Acid to make the sTn antigen; which is also not typically expressed in normal tissue. Therefore, targeting TACA epitopes could become significant for managing various human cancers.

Although bi-specific proteins and/or CAR T cells that target TACAs have great therapeutic potential, the absence of high affinity and/or high specificity antibodies against carbohydrate targets is a crucial limitation in exploiting glycans as therapeutic targets. Antibodies against carbohydrates are extremely difficult to generate. The generation of monoclonal antibodies with high affinity to complex carbohydrates like TACAs (e.g., Tn antigen or β1,6GlcNAc-branched N-glycans) has proven to be challenging, preventing TACAs from being widely exploited as cancer specific antigens. Furthermore, anti-glycan antibodies typically have affinities 1000-100000 fold lower than antibodies to peptide antigens. Anti-carbohydrate antibodies also typically require additional peptide/lipid epitopes for high affinity binding. In addition, antibodies against carbohydrates have low affinity and specificity. Furthermore, the recognition of the glycan antigen by the antibody depends on glycan density, valency, presentation, and flexibility.

Accordingly, there is a need for TACA-specific bi-specific fusion proteins and/or TACA-specific CARs for treating a disease associated with an aberrant glycosylation of cell surface molecules that are not based on an scFv from an antigen-specific monoclonal antibody.

In the present disclosure, a novel class of immunotherapeutic bi-specific fusion proteins and CARs were developed to effectively target TACA for immunotherapy. Specifically, an antigen-binding domain derived from a lectin rather than a monoclonal antibody or fragment thereof was used to engineer bi-specific fusion proteins and CARs to target TACAs irrespective of the carrier protein. This novel technology is referred to as “Glycan-dependent T cell Recruiter” or GlyTR (pronounced ‘glitter’). One set of GlyTR therapeutics are TACA-bi-specific fusion proteins comprising a TACA binding domain (e.g., carbohydrate recognition domain) from a lectin operably linked, conjugated to or fused to an immune cell recognition domain that specifically binds to a receptor on an immune effector cell. Another set of GlyTR therapeutics are chimeric antigen receptors comprising an antigen binding domain comprising a TACA-binding domain derived from a lectin. In all cases, the TACA-binding domain specifically binds to a TACA expressed on a tumor cell and the TACA-binding domain comprises multiple (e.g., more than one, or at least two) TACA-binding domains derived from a lectin.

### A. Summary of Experimental Results

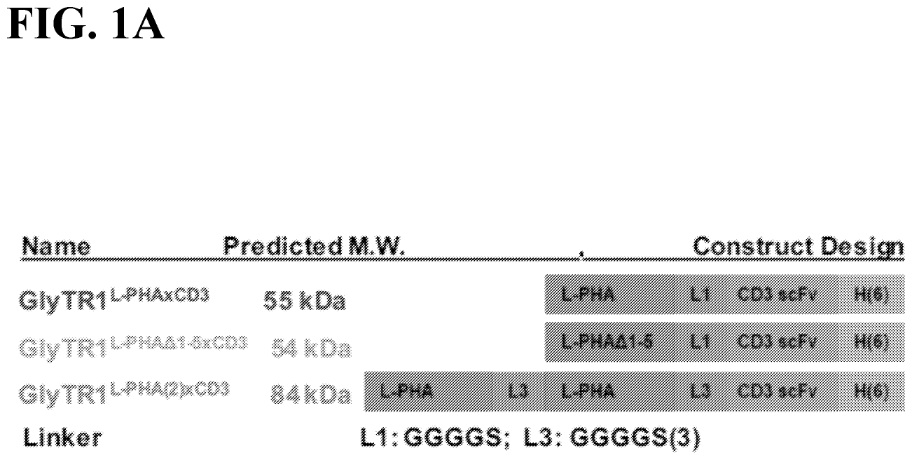

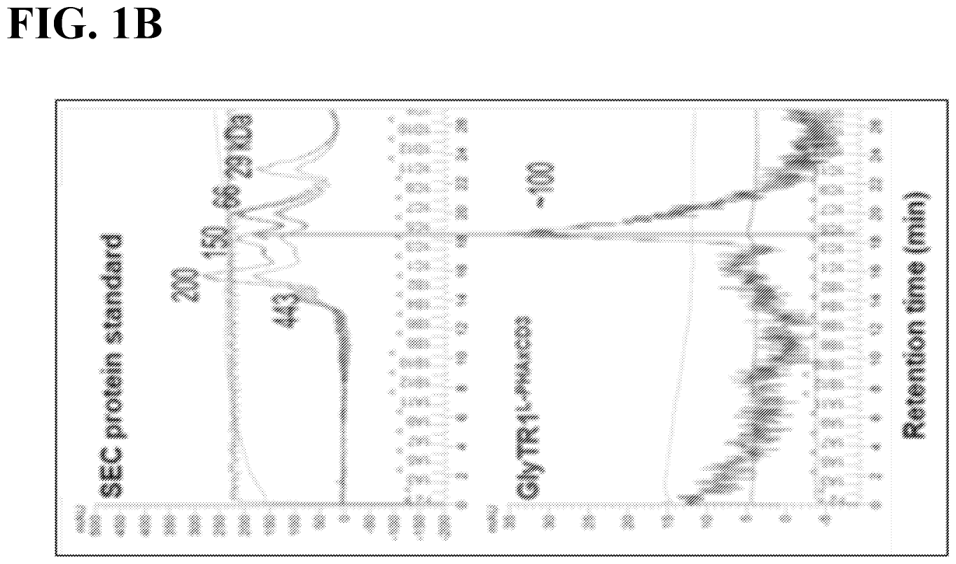

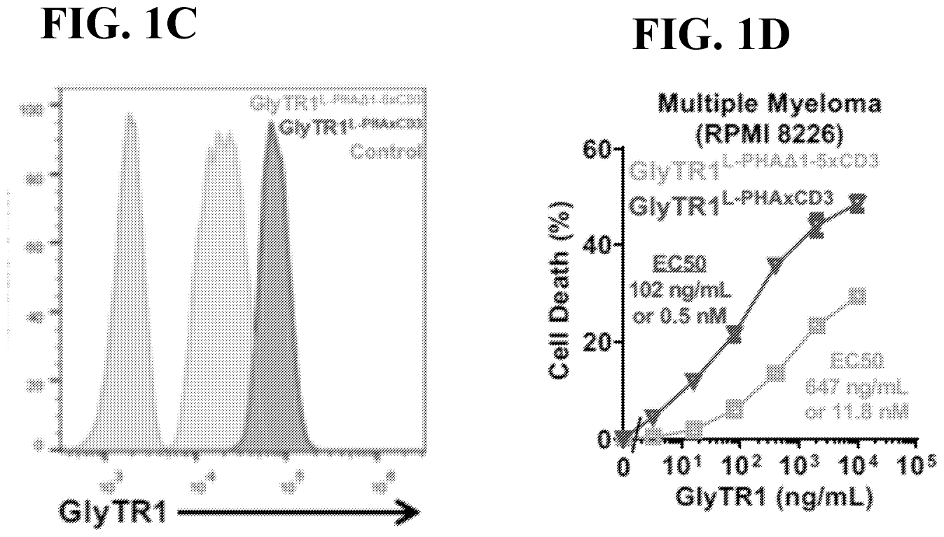

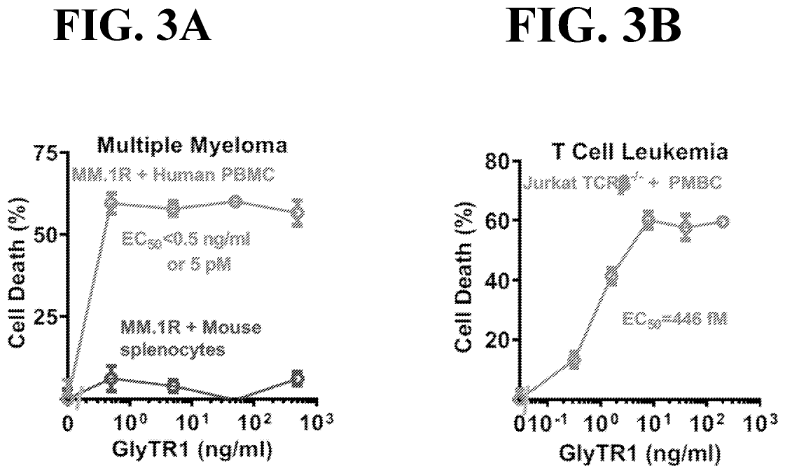

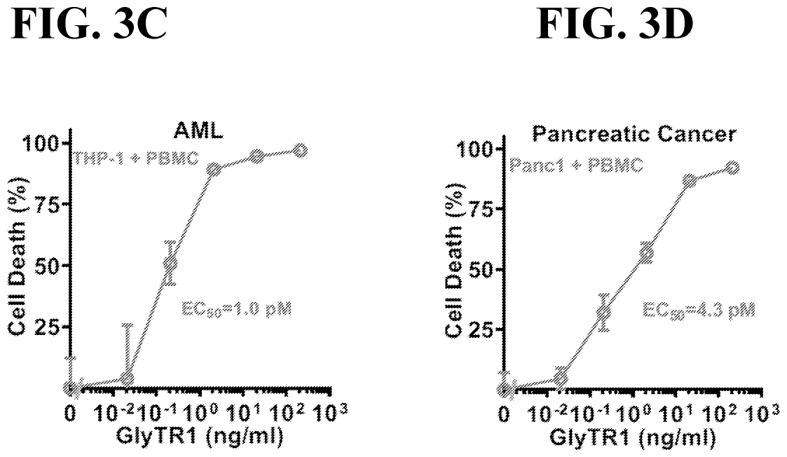

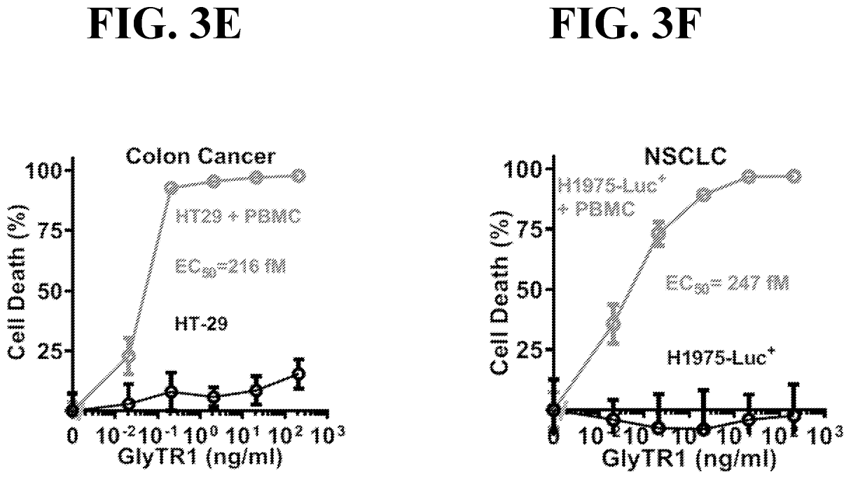

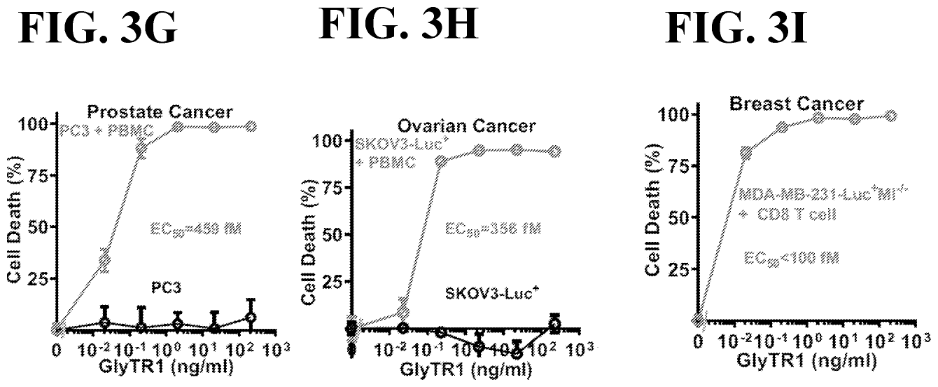

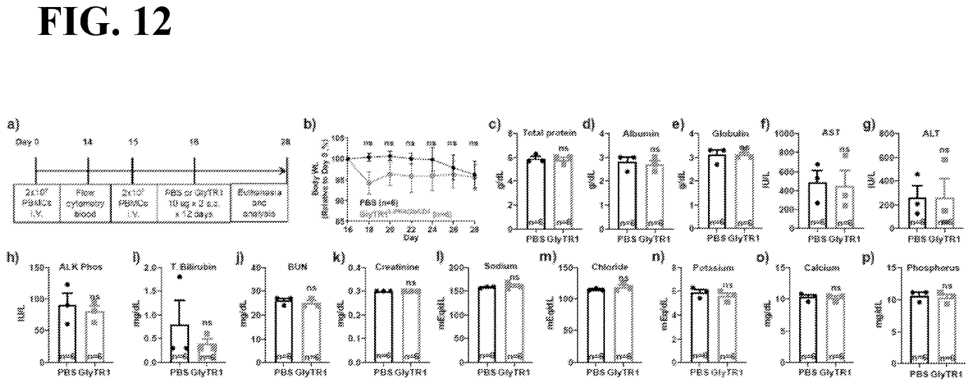

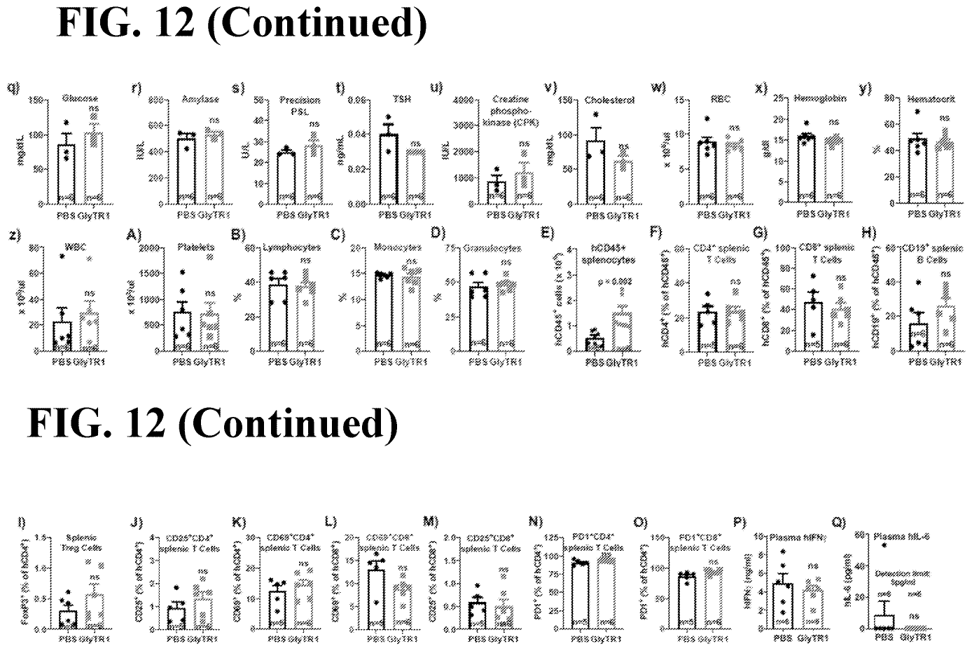

The present disclosure further provides GlyTR therapeutics having enhanced GlyTR binding avidity (FIGS. 1-2, and 14-15), killing activity (FIGS. 3-5, 16, 17-18, and 23) and safety (FIGS. 12-13, and 20-21).

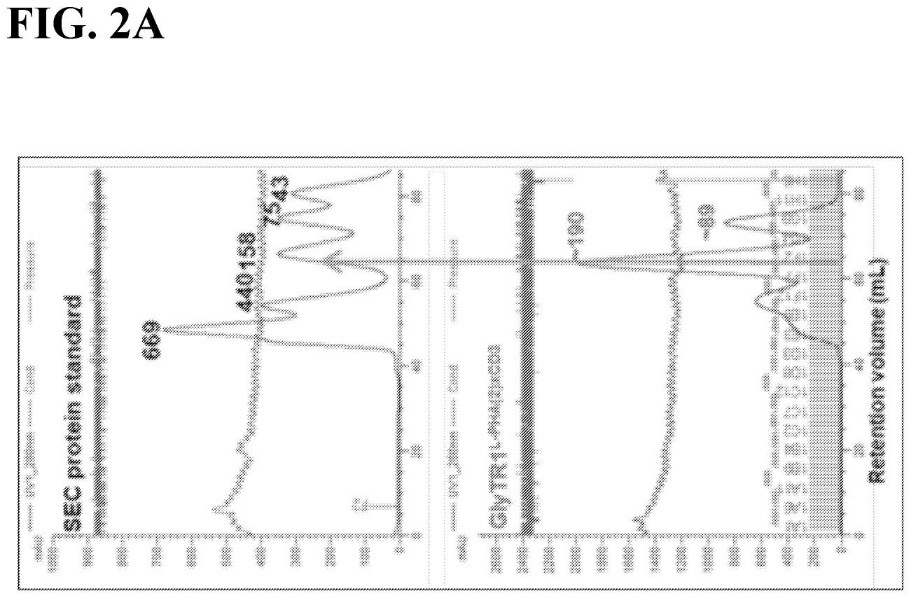

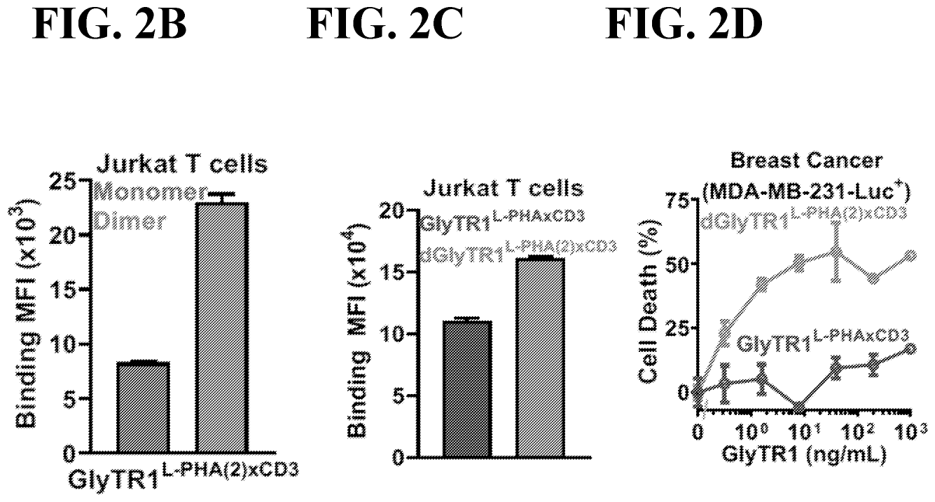

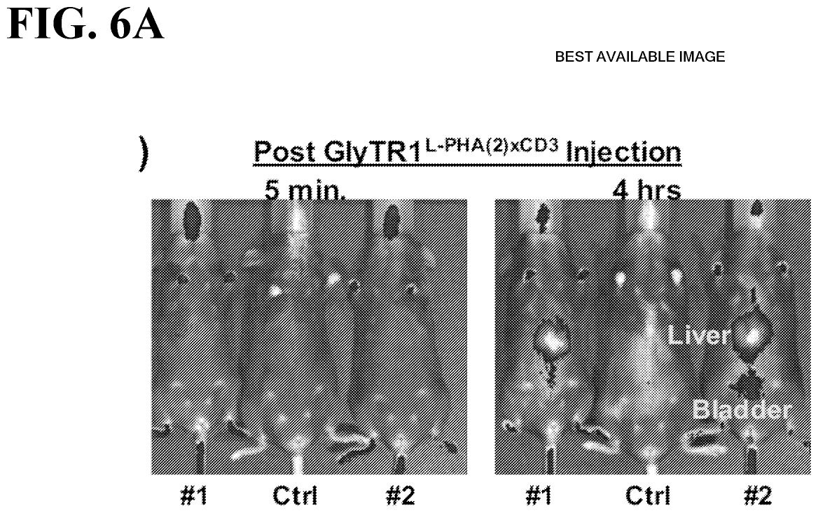

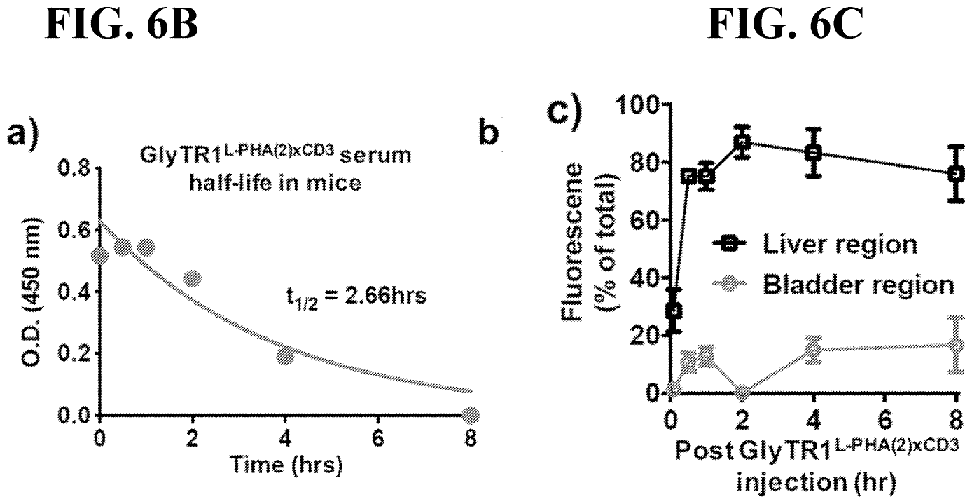

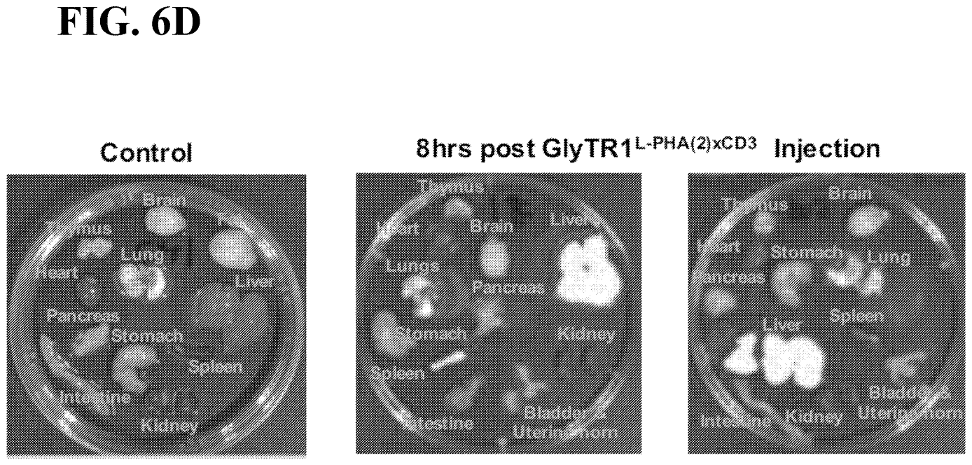

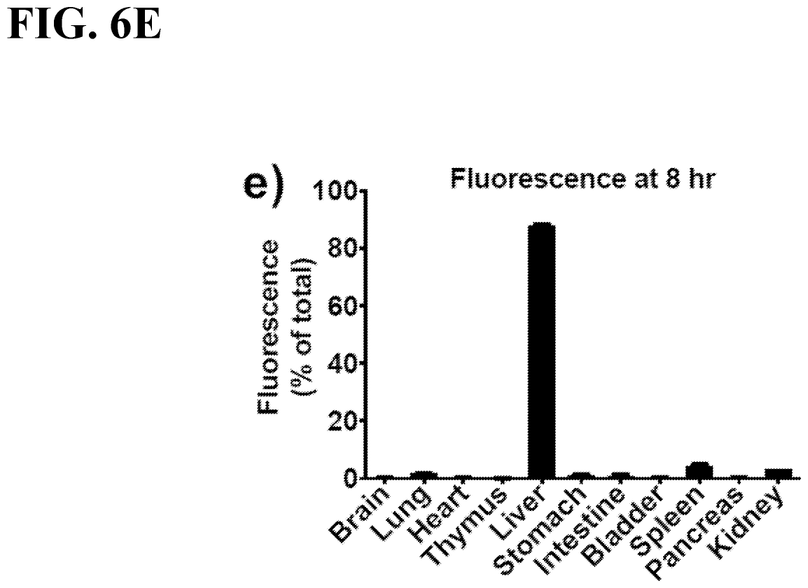

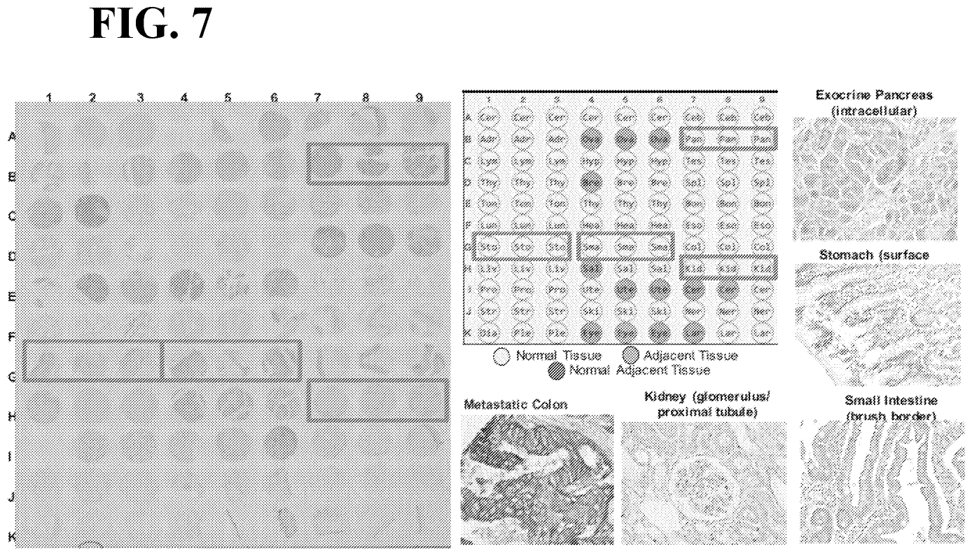

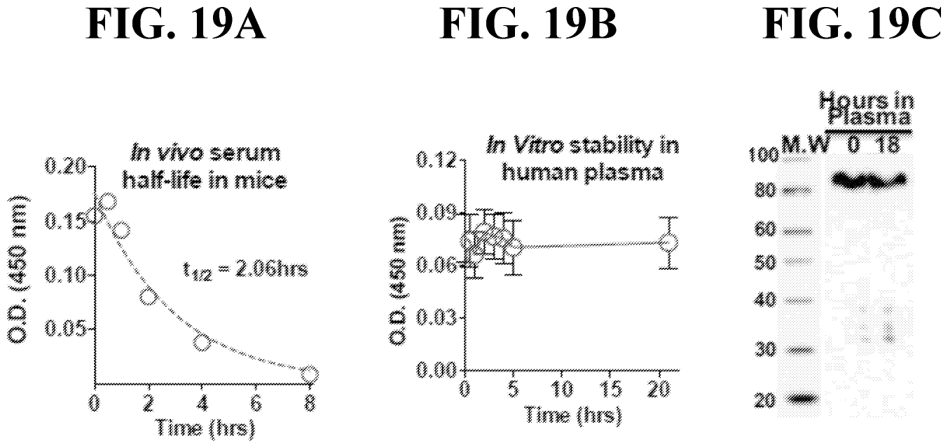

To drive binding avidity to high density TACAs present in cancer cells, GlyTR therapeutics with multiple carbohydrate-binding domains derived from a lectin were generated. Specifically, GlyTR1^{LPHA(2)xCD3} (GlyTR1^{LPHAxLPHAxCD3}. two TACA binding domains) with two L-PHA domains linked in tandem by three flexible linkers (i.e. (GGGGS)₃) were generated (FIG. 1A). SEC revealed that GlyTR1^{LPHA(2)xCD3} was ~50-70% dimer, with the rest being monomer (~30-40%) or larger multimers (~10-20%) (FIG. 2A). Directly comparing the monomeric (two L-PHA domains) and dimeric (four L-PHA domains) fractions of GlyTR1^{LPHA(2)xCD3} revealed significantly higher binding to β1,6GlcNAc-branched N-glycans in the latter (FIG. 2B), further confirming that increasing the number of TACA binding domains within GlyTR1 proteins led to higher potency. Directly comparing the monomeric (two L-PHA domains) and dimeric (four LPHA domains) fractions of GlyTR1^{LPHA(2)xCD3} revealed significantly higher binding to β1,6GlcNAc-branched Nglycans in the latter (FIG. 2B), further confirming that increasing the number of TACA binding domains within GlyTR1 proteins leads to higher potency. Indeed, dimeric GlyTR1^{LPHA(2)xCD3} (four L-PHA domains) bound to target cancer cells significantly better than original dimeric GlyTR1^{LPHAxCD3} (two L-PHA domains), leading to a >3000 fold increase in cancer cell killing activity (FIG. 2C, D). Moreover, dimeric GlyTR1^{LPHA(2)xCD3}potently triggered human T cell dependent killing of many diverse liquid and solid cancer types with an EC₅₀ as low as <100 femtomolar, including multiple myeloma, T cell leukemia, acute myeloid leukemia, (AML), pancreatic cancer, colon cancer, non-small cell lung cancer, prostate cancer, ovarian cancer and breast cancer, (FIG. 3A-I). These improved GlyTR1 binding domains were safe (FIGS. 12-13, and 20-21), stable (FIGS. 6 and 19), and selectively killed cancer cells (FIGS. 3-5, 16, 17-18, and 23). Furthermore, engineered cells comprising the novel and improved TACA-bi-specific fusion proteins and/or TACA-CARs did not exhibit any T cell dependent “on-target/off-cancer” toxicity when compared to control cells (FIGS. 7-11). The combination of high target density and multiple binding sites led to marked specificity for high expressing cancer cells over low expressing normal cells and potent triggering of T cell mediated killing of the former but not the latter.

### B. Exemplary Benefits of the GlyTR Fusion Proteins

One advantage for targeting TACAs for immunotherapy is that virtually all cell surface proteins are glycosylated. The TACA target density is ~100-1000 fold greater than typical protein antigens. As such, increasing the number of TACA binding domains in GlyTR may drive cancer cells specificity by enhancing binding avidity. This is contrast to antibodies, where high affinity is used to achieve specificity. In the present disclosure, high avidity binding was accomplished by the combination of high-density target expression on tumor cells and the presence of multiple carbohydrate-binding domains of the engineered GlyTRs. This combination of high target density and multiple binding sites enhanced the specificity of the improved GlyTRs for high TACA expressing cells (e.g., cancerous cells) over low expressing cells (e.g., normal cells). Accordingly, the specificity of the novel multi-valent GlyTR proteins (TACA bi-specific fusion proteins, or TACA CARs) for TACAs would be determined by a threshold density of target expression specifically detected by GlyTRs with multiple TACA binding domains, rather than the presence or absence of the target antigen. The multi-valent GlyTR proteins of the present disclosure showed improve specificity for high target expressing cancer cells and spare lower-expressing normal tissue. As shown in the examples described herein, the GlyTR-based immunotherapy of the present disclosure induced sufficient T cell activation in vitro and in vivo to maximize cancer killing, but insufficient to induce T cell exhaustion or cytokine release syndrome.

Accordingly, the present disclosure provides bi-specific fusion proteins and chimeric antigen receptors (CAR) that selectively bind a TACA on a target cell; isolated nucleic acid molecules encoding the bi-specific fusion proteins and CARs; expression vectors comprising isolated nucleic acid molecules encoding the bi-specific fusion proteins and CARs, modified cells comprising h isolated nucleic acid molecules encoding the bi-specific fusion proteins and CARs and/or expression vectors; compositions comprising the modified cells; and methods for treating a condition or a disease associated with a tumor-associated carbohydrate antigen (TACA).

In one aspect, the present disclosure provides an isolated nucleic acid molecule encoding a chimeric antigen receptor (CAR) comprising: an antigen binding domain that selectively binds a tumor-associated carbohydrate antigen (TACA), aa transmembrane domain, a costimulatory signaling region, and an intracellular signaling domain, optionally a hinge domain.

Another aspect, the present disclosure provides an isolated nucleic acid molecule encoding a bi-specific fusion protein comprising an antigen binding domain that selectively binds a tumor-associated carbohydrate antigen (TACA), and an immune cell recognition domain that specifically binds a receptor on an immune effector cell. In some embodiments, the antigen binding domain of the CAR or bi-specific fusion protein comprises multiple (more than one, or at least two, three, four, or more TACA-binding domains derived from a lectin).

In another aspect, the present disclosure provides a chimeric antigen receptor or a bi-specific fusion protein that selectively binds a tumor-associated carbohydrate antigen (TACA) comprising an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, or 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146.

The chimeric antigen receptor (CAR) or bi-specific fusion protein of the present disclosure comprises a tumor-associated carbohydrate antigen (TACA)-binding domain derived from a lectin to target TACA expressing cells for killing tumor cell rather than an antibody or antibody fragment. The CAR(TACA-CAR) or bi-specific fusion protein of the present disclosure is an improvement over the art because the CAR or bi-specific fusion protein comprises structural modifications that enhance the specificity the TACA-CAR or bi-specific fusion protein. The first structural modification includes altering the structure of the antigen-binding domain by changing the number, linkage and sequence of TACA-binding domains derived from a lectin. For example, the antigen-binding domain of the TACA-CAR or bi-specific fusion protein comprises one or more TACA-binding domains derived from a lectin. In addition, the linker domains between the more than one TACA-binding domains are modified.

In another aspect, the present disclosure provides a composition comprising the TACA-CAR or bi-specific fusion protein disclosed herein or modified cells comprising the TACA-CAR or bi-specific fusion protein disclosed herein. The compositions of the present disclosure also include additional peptides comprising multiple TACA-binding domains (at least two), nucleic acid molecules encoding a peptide comprising a TACA-binding domain, a cell modified to express a peptide comprising multiple TACA-binding domain, and a substrate comprising the peptide, nucleic acid, cell, or combination thereof.

In another aspect, the present disclosure provides a method of treating a cancer in a subject in need thereof, the method comprising administering to the subject a therapeutically effective amount of a composition comprising a modified cell comprising a chimeric antigen receptor or bi-specific fusion protein that selectively binds a tumor-associated carbohydrate antigen (TACA) of the present disclosure. The disease or condition that may be treated using the he TACA-CAR or bi-specific fusion protein of the present disclosure is for example, a cancer or any condition associated with alteration in protein glycosylation. The cancer may be a hematological malignancy, a solid tumor, a primary or a metastasizing tumor. In another aspect, the present disclosure provides a method of providing an anti-tumor immunity in a mammal, comprising administering to the mammal an effective amount of a population of modified cells of comprising the chimeric antigen receptor or bi-specific fusion protein that selectively binds a tumor-associated carbohydrate antigen (TACA) of the present disclosure.

### II. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present disclosure, the preferred methods and materials are described. It is also to be understood that the terminology used herein is for the purpose of describing particular embodiments only, and is not intended to be limiting.

The practice of the present disclosure will employ, unless otherwise indicated, conventional techniques of tissue culture, immunology, molecular biology, microbiology, cell biology and recombinant DNA, which are within the skill of the art. See e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (TRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; and Herzenberg et al. eds (1996) Weir's Handbook of Experimental Immunology, 5th edition.

As used herein, each of the following terms has the meaning associated with it in this section. The articles “a” and “an” as used herein to refer to one or to more than one (i.e., to at least one) of the grammatical object of the article. By way of example, “an element” means one element or more than one element.

As used herein the term “About” when referring to a measurable value such as an amount, a temporal duration, and the like, is meant to encompass variations of 20%, ±10%, ±5%), ±1%), or ±0.1%) from the specified value, as such variations are appropriate to perform the disclosed methods.

As used herein, the term “Activation”, refers to the state of a T cell that has been sufficiently stimulated to induce detectable cellular proliferation. Activation can also be associated with induced cytokine production, and detectable effector functions. As used herein, the term “activated T cells” refers to, among other things, T cells that are undergoing cell division.

The term “Anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place.

As used herein, the term “Autologous” is meant to refer to any material derived from the same individual to which it is later to be re-introduced into the individual.

As used herein, the term “Antigen” or “Ag” is defined as a molecule that provokes an immune response. This immune response may involve other antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “Allogeneic” refers to a graft derived from a different animal of the same species.

As used herein, the term “Antibody” refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)2, as well as single chain antibodies (scFv) and humanized antibodies. In some embodiments, antibody refers to such assemblies (e.g., intact antibody molecules, immunoadhesins, or variants thereof) which have significant known specific immunoreactive activity to an antigen of interest (e.g., a tumor associated antigen). Antibodies and immunoglobulins comprise light and heavy chains, with or without an interchain covalent linkage between them. Basic immunoglobulin structures in vertebrate systems are relatively well understood.

The term “Antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

As used herein, the term “Antibody heavy chain,” as used herein, refers to the larger of the two types of polypeptide chains present in all antibody molecules in their naturally occurring conformations.

As used herein, an “Antibody light chain,” refers to the smaller of the two types of polypeptide chains present in all antibody molecules in their naturally occurring conformations.

As used herein, the term “Antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bi-specific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

The term “Cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “Cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells (e.g., a lineage marker such as CD19 on B cells). In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present disclosure includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described. For example, TCR-like antibody can be identified from screening a library, such as a human scFv phage displayed library.

In some embodiments, the tumor antigen is selected from the group consisting of a tumor-associated carbohydrate antigen (TACA), alpha fetoprotein (AFP)/HLA-A2, AXL, B7-H3, BCMA, CA-IX, CD2, CD3, CD4, CDS, CD7, CD8, CD19, CD20, CD22, CD30, CD33, CD38, CD44v6, CD70, CD79a, CD79b, CD80, CD86, CDI 17, CD123, CD133, CD147, CDI 71, CD276, CEA, claudin 18.2, c-Met, DLL3, DRS, EGFR, EGFRvlll, EpCAM, EphA2, FAP, folate receptor alpha (FRa)/folate binding protein (FBP), GD-2, Glycolipid F77, glypican-3 (GPC3), HER2, HLA-A2, ICAMI, IL3Ra, IL13Ra2, LAGE-I, Lewis Y, LMPI (EBV), MAGE-A1, MAGE-A3, MAGE-A4, Melan A, mesothelin, MG7 (glycosylated CEA), MMP, MUCI, Nectin4/FAP, NKG2D-Ligands (MIC-A, MIC-B, and the ULBPs I to 6), NY-ESO-1, P1 6, PD-LI, PSCA, PSMA, RORI, ROR2, TIM-3, TM4SF1, TnMuc1, VEGFR2, and any combination thereof.

As used herein, the term “Chimeric antigen receptor” or “CAR,” refers to an artificial T cell receptor that is engineered to be expressed on an immune effector cell or precursor cell thereof and specifically bind an antigen. CARs may be used in adoptive cell therapy with adoptive cell transfer. In some embodiments, adoptive cell transfer (or therapy) comprises removal of T cells from a patient, and modifying the T cells to express the receptors specific to a particular antigen. In some embodiments, the CAR has specificity to a selected target, for example a tumor-associated carbohydrate antigen (TACA). CARs may-also comprise an intracellular activation domain, a transmembrane domain and an extracellular domain comprising an antigen binding region.

As used herein, the term “Co-stimulatory ligand,” includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD2, CD7, B7-1 (CD80), B7-2 (CD86), PD-L1, PD- L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

As used herein, a “Co-stimulatory molecule” refers to the cognate binding partner on a T cell that specifically binds with a co-stimulatory ligand, thereby mediating a co-stimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA, a Toll ligand receptor, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR). In some embodiments, a co-stimulatory molecule includes OX40, CD27, CD2, CD28, ICOS (CD278), and 4-1BB (CD137). Further examples of such costimulatory molecules include CD8, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2Rbeta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD1Ib, ITGAX, CD11c, ITGB1, CD29, ITGB2, CD18, LFA-1, ITGB7, NKG2D, NKG2C, TNFR2, TRANCE RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

As used herein, the term “Co-stimulatory signal” refers to a signal, which in combination with a primary signal, such as TCR/CD3 ligation, leads to T cell proliferation and/or upregulation or downregulation of key molecules. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CD8, CD7, CD287, LIGHT, NKG2C, NKG2D, SLAMF7, NKp80, NKp30, NKp44, NKp46, CD160, B7-H3, and a ligand that specifically binds with CD83, and the like.

As used herein, the term “Derived from” refers to a relationship between a first and a second molecule. It defines a structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, It does not mean that, to provide the intracellular signaling domain, one must start with a CD3zeta sequence and delete unwanted sequence, or impose mutations, to arrive at the intracellular signaling domain.

As used herein a “Disease” refers to a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, “Disease associated with expression of a tumor antigen” includes, but is not limited to, a disease associated with expression of a tumor antigen or condition associated with cells which express a tumor antigen including, but not limited to proliferative diseases such as a cancer or malignancy or a precancerous condition such as a myelodysplasia, a myelodysplastic syndrome or a preleukemia; or a noncancer related indication associated with cells, which express a tumor antigen. In some embodiments, a cancer associated with expression of a tumor antigen is a hematological cancer. In some embodiments, a cancer associated with expression of a tumor antigen is a solid cancer. Further diseases associated with expression of a tumor antigen include, but not limited to, atypical and/or non-classical cancers, malignancies, precancerous conditions or proliferative diseases associated with expression of a tumor antigen. Non-cancer related indications associated with expression of a tumor antigen include, but are not limited to, autoimmune disease, (e.g., lupus), inflammatory disorders (allergy and asthma) and transplantation. In some embodiments, the tumor antigen-expressing cells express, or at any time expressed, mRNA encoding the tumor antigen. In some embodiments, the tumor antigen-expressing cells produce the tumor antigen protein (e.g., wild-type or mutant), and the tumor antigen protein may be present at normal levels or reduced levels. In some embodiment, the tumor antigen-expressing cells produced detectable levels of a tumor antigen protein at one point, and subsequently produced substantially no detectable tumor antigen protein.

As used herein, the term “Downregulation” refers to the decrease or elimination of gene expression of one or more genes.

As used herein “Effective amount” or “Therapeutically effective amount” means an amount of a compound, formulation, material, pharmaceutical agent, or composition, as described herein effective to achieve a desired physiological, therapeutic, or prophylactic outcome in a subject in need thereof. Such results may include but are not limited to an amount that when administered to a mammal, causes a detectable level of immune response compared to the immune response detected in the absence of the composition of the present disclosure. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like. The effective amount may vary among subjects depending on the health and physical condition of the subject to be treated, the taxonomic group of the subjects to be treated, the formulation of the composition, assessment of the subject's medical condition, and other relevant factors.

As used herein, the term “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “Endogenous” refers to any material from or produced inside an organism, cell, tissue or system.

As used herein, the term “Epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. In certain exemplary embodiments, die epitope is about 4-18 amino acids, about 5-16 amino acids, about 6-14 amino acids, about 7-12 amino acids, or about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present disclosure can be an epitope. As used herein, the term “exogenous” refers to any material introduced from or produced outside an organism, cell, tissue or system

As used herein, the term “Expand” as used herein refers to increasing in number, as in an increase in the number of immune cells (e.g., T cells). In some embodiments, the immune cells (e.g., T cells) that are expanded ex vivo increase in number relative to the number originally present in the culture. In another embodiment, the immune cells (e.g., T cells) that are expanded ex vivo increase in number relative to other cell types in the culture.

As used herein, the term “Exogenous” refers to any material introduced from or produced outside an organism, cell, tissue, or system.

As used herein, the term “Expression” as used herein is defined as the transcription and/or translation of a particular nucleotide sequence driven by its promoter.

As used herein, the term “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “Ex vivo,” refers to cells that have been removed from a living organism, (e.g., a human) and propagated outside the organism (e.g., in a culture dish, test tube, or bioreactor).

As used herein, the term “Fc portion” or “Fc monomer” means in connection with this disclosure a polypeptide comprising at least one domain having the function of a CH2 domain and at least one domain having the function of a CH3 domain of an immunoglobulin molecule. As apparent from the term “Fc monomer”, the polypeptide comprising those CH domains is a “polypeptide monomer”. An Fc monomer can be a polypeptide comprising at least a fragment of the constant region of an immunoglobulin excluding the first constant region immunoglobulin domain of the heavy chain (CH1) but maintaining at least a functional part of one CH2 domain and a functional part of one CH3 domain, wherein the CH2 domain is amino terminal to the CH3 domain. In a preferred aspect of this definition, an Fc monomer can be a polypeptide constant region comprising a portion of the Ig-Fc hinge region, a CH2 region and a CH3 region, wherein the hinge region is amino terminal to the CH2 domain.

It is envisaged that the hinge region of the present disclosure promotes dimerization. Such Fc polypeptide molecules can be obtained by papain digestion of an immunoglobulin region (of course resulting in a dimer of two Fc polypeptide), for example and not limitation. In another aspect of this definition, an Fc monomer can be a polypeptide region comprising a portion of a CH2 region and a CH3 region. Such Fc polypeptide molecules can be obtained by pepsin digestion of an immunoglobulin molecule, for example and not limitation. In one embodiment, the polypeptide sequence of an Fc monomer is substantially similar to an Fc polypeptide sequence of: an IgG1 Fc region, an IgG2 Fc region, an IgG3 Fc region, an IgG4 Fc region, an IgM Fc region, an IgA Fc region, an IgD Fc region and an IgE Fc region. (See, e.g., Padlan, Molecular Immunology, 31(3), 169-217 (1993)). Because there is some variation between immunoglobulins, and solely for clarity, Fc monomer refers to the last two heavy chain constant region immunoglobulin domains of IgA, IgD, and IgG, and the last three heavy chain constant region immunoglobulin domains of IgE and IgM.

The Fc monomer can also include the flexible hinge N-terminal to these domains. For IgA and IgM, the Fc monomer may include the J chain. For IgG, the Fc portion comprises immunoglobulin domains CH2 and CH3 and the hinge between the first two domains and CH2. Although the boundaries of the Fc portion may vary an example for a human IgG heavy chain Fc portion comprising a functional hinge, CH2 and CH3 domain can be defined e.g., to comprise residues SEQ ID NOs 153-158.

An IgG hinge region can be identified by analogy using the Kabat. In one embodiment, the hinge domain/region of the present disclosure comprises the amino acid residues corresponding to the IgG1 sequence stretch of D234 to P243 according to the Kabat numbering. In some embodiments, the hinge domain/region of the present disclosure comprises or consists of the IgG1 hinge sequence DKTHTCPPCP (SEQ ID NO: 153 or 154).

In one embodiment, the IgG1 hinge domain/region comprises the amino acid sequence of EPKSCDKTHTCPPCP (SEQ ID NO: 154).

In further embodiments of the present disclosure, the hinge domain/region comprises or consists of the IgG2 subtype hinge sequence ERKCCVECPPCP (SEQ ID NO: 155), the IgG3 subtype hinge sequence ELKTPLDTTHTCPRCP (SEQ ID NO: 156) or ELKTPLGDTTHTCPRCP (SEQ ID NO: 157), and/or the IgG4 subtype hinge sequence ESKYGPPCPSCP (SEQ ID NO: 158).

In some embodiments, the fusion protein further comprises a third domain comprising two polypeptide monomers, where each monomer comprises a hinge, a CH2 domain and a CH3 domain. In one embodiment, the third domain comprises in an amino to carboxyl order: hinge-CH2-CH3-linker-hinge-CH2-CH3. In one embodiment, the CH2 domain comprises an intra-domain cysteine disulfide bridge.

In another embodiment, the two polypeptide monomers are fused to each other via a peptide linker. Yet, in another embodiment, the first and second domain are fused to the third domain via a peptide linker. In some embodiments, the peptide linker of the fusion protein of the present disclosure comprises the amino acid sequence of GGGS (e.g., Gly4Ser (SEQ ID NO: 128)), or polymers thereof (e.g., (Gly4Ser)ₙ, where n is an integer of 5 or greater (e.g., 5, 6, 7, 8 etc. or greater)).

As used herein, the term “Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

As used herein, the term “Identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino adds sequences are 90% identical.

As used herein, the term “Immunoglobulin” or “Ig,” is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing release of mediators from mast cells and basophils upon exposure to allergen.

As used herein, the term “Immune response” is defined as a cellular response to an antigen that occurs when lymphocytes identify antigenic molecules as foreign and induce the formation of antibodies and/or activate lymphocytes to remove the antigen. The term “Immunostimulatory” is used herein to refer to increasing overall immune response. The term “Immunosuppressive” is used herein to refer to reducing overall immune response.

As used herein, the term “Immune response” as used herein is defined as a cellular response to an antigen that occurs when lymphocytes identify antigenic molecules as foreign and induce the formation of antibodies and/or activate lymphocytes to remove the antigen.

As used herein, the term “Immune effector cell,” refers to a cell that is involved in an immune response, e.g., in the promotion of an immune effector response. Examples of immune effector cells include T cells (e.g., alpha/eta T cells and gamma/delta T cells), B cells, natural killer (NK) cells, natural killer T (NKT) cells, mast cells, and myeloic-derived phagocytes.

As used herein, the term “Immune effector function” or “Immune effector response,” refers to a function or response that enhances or promotes an immune attack of a target cell. In some embodiment, an immune effector function or response refers to a property of a T or NK cell that promotes the killing or the inhibition of growth or proliferation, of a target cell. In the case of a T cell, primary stimulation and co-stimulation are examples of immune effector function or response.

As used herein, an “Instructional material” includes a publication, a recording, a diagram, or any other medium of expression which can be used to communicate the usefulness of the compositions and methods of the disclosure. The instructional material of the kit of the disclosure may, for example, be affixed to a container which contains the nucleic acid, peptide, and/or composition of the disclosure or be shipped together with a container which contains the nucleic acid, peptide, and/or composition. Alternatively, the instructional material may be shipped separately from the container with the intention that the instructional material and the compound be used cooperatively by the recipient.

As used herein, the term “Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “Lectin” or “Hemagglutinin” refers to a protein or peptide that binds carbohydrate structures. A skilled artisan will understand that a lectin is a protein or peptide that is highly specific for binding to sugar moieties. Lectins are carbohydrate-binding proteins that are highly specific for carbohydrate found on proteins and/or lipid and so cause agglutination of particular cells or precipitation of glycoconjugates and polysaccharides. Lectins have a role in recognition at the cellular and molecular level and play numerous roles in biological recognition phenomena involving cells, carbohydrates, and proteins. Lectins also mediate attachment and binding of bacteria, viruses, and fungi to their intended targets. In one embodiment, “Lectin” can be defined as a protein or glycoprotein of non-immununoglobulin nature that is capable of specific recognition and of reversible binding to carbohydrate moieties of complex glycoconjugates (proteins and or lipids), without altering the covalent structure of any of the recognized glycosyl ligands. Lectins are glycoproteins with a carbohydrate-binding domain possessing reversible binding ability to specific sugar moieties in glycoproteins or glycolipids as well as the free monosaccharide and glycan structures. Although many living organisms express lectins or lectin-like biomolecules, most of recently identified lectins of scientific significance have been purified from plant sources.

The term “Tumor Associated Carbohydrate Antigen” or “TACA” as used herein refers to a carbohydrate structure found in disorders associated with altered glycosylation, e.g., cancer. Carbohydrate-containing macromolecules (glycans) are ubiquitous in biological systems and are essential for numerous biological functions. The carbohydrates can be attached to proteins (glycoproteins), lipids (glycolipids) and exist as chains of carbohydrates (glycosaminoglycans). Changes in the structure of these carbohydrates-containing macromolecules (glycosylation) have a significant impact on cancer biology and cancer progression. Indeed, altered glycosylation is a common feature of tumor cells and leads to the formation of tumor-associated carbohydrates (TACA). Cancer cells can often be distinguished from normal cells by displaying aberrant levels and types of carbohydrate structures on their surfaces.

Three common changes in carbohydrate-containing macromolecules are associated with cancer: increased expression of truncated or incomplete glycans, increased branching of N-glycans and augmented or changed presence of sialic acid-containing glycans For example, cancer-associated glycans often exhibit an increased amount of sialic acid, and this hypersialylation enhances the activation of sialic acid binding receptors, such as selectins and Siglecs, leading to cancer progression. Another most common cancer-associated changes in glycosylation is the truncation of O-linked carbohydrate chains (truncation of O-glycoproteins), such as mucin, Under normal condition, a N-acetyl-galactosamine (GalNAc) sugar residue is attached to a serine or threonine of a glycoprotein (GalNAcαl-O-Ser/Thr, Tn antigen) and is usually elongated by the T-synthase (core 1 β3-galactosyltransferase) in the Golgi apparatus that attaches a galactose residue to Thomsen-Friedenreich (TF) antigen (Tn antigen) In various cancers, this process is altered and the glycosylation of the Tn antigen or its sialylated form (the sialyl-Tn (STn) antigen) is altered resulting to to truncated T, Tn and STn antigen. In addition, increased branching of N-glycoproteins, which stimulates galactin-3, and alterations of glycolipids, such e.g., as gangliosides (GM3, GM2, CD3 and GD2) have also been observed.

The following TACAs have been observed in various cancers: (i) H/Le^{y}/ILe^{a} in primary non-small cell lung carcinma; (ii) sialyl-Le^{x}(SLe^{x}) and sialyl-Lea (SLea) in various types of cancer; (iii) Tn and sialyl-Tn in colorectal, lung, breast, and many other cancers; (iv) GM2, GD2, and GD3 gangliosides in neuroectodermal turnors (melanoma and neuroblastoma); and (v) globo-H in breast, ovarian, and prostate cancer; (vi) disialylgalactosylgloboside in renal cell carcinoma.

Accordingly, the term “as tumor-associated carbohydrate antigen (TACA)” encompasses all altered carbohydrate structures on proteins and/or lipids that are expressed on tumor tumor cells, promote cancer metastasis, cancer progression, cancer and non-cancer-immunosuppression, and/or promote autoimmune disorders. A skilled artisan will understand that a carbohydrate structures consists of one or more linked sugars or monosaccharides. See e.g., Mantuano et al, J. Immunotherapy Cancer 8(2): 8:e001222 (2020); Hakomori, Si. (2001). Tumor-Associated Carbohydrate Antigens Defining Tumor Malignancy: Basis for Development of Anti-Cancer Vaccines, in The Molecular Immunology of Complex Carbohydrates—2. Advances in Experimental Medicine and Biology, vol 491. Springer, Boston, MA (Wu et al (eds)). A skilled artisan will understand that carbohydrate structures may be free standing and/or attached to proteins or lipids, known as glycoproteins and glycolipids. A skilled artisan will understand that these carbohydrates structures bind to a lectin.

A “Lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “Limited toxicity” as used herein, refers to the peptides, polynucleotides, cells and/or antibodies of the present disclosure manifesting a lack of substantially negative biological effects, anti-tumor effects, or substantially negative physiological symptoms toward a healthy cell, non-tumor cell, non-diseased cell, non-target cell or population of such cells either in vitro or in vivo.

As used herein, the term “Flexible polypeptide linker” or “Linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3. n=4, n=5 and n=6, n=7, n=8, n=9 and n=10 (SEQ ID NO:6592). In one embodiment, the flexible polypeptide linkers include, but are not limited to, (Gly4 Ser)₄ or (Gly4 Ser)₃. In another embodiment, the linkers include multiple repeats of (Gly2Ser), (GlySer) or (Gly3Ser).

As used herein, the term “Modified” means a changed state or structure of a molecule or cell of the present disclosure. Molecules may be modified in many ways, including chemically, structurally, and functionally. Cells may be modified through the introduction of nucleic acids.

The term “Modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Unless otherwise specified, a “Nucleotide sequence encoding an amino acid sequence” includes all nucleotide sequences that are degenerate versions of each other and that encode the same amino acid sequence. Nucleotide sequences that encode proteins and RNA may include introns.

As used here, the term “Operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “Overexpressed tumor antigen” or “overexpression of the tumor antigen” is intended to indicate an abnormal level of expression of the tumor antigen in a cell from a disease area like a solid tumor within a specific tissue or organ of the patient relative to the level of expression in a normal cell from that tissue or organ. Patients having solid tumors, or a hematological malignancy characterized by overexpression of the tumor antigen can be determined by standard assays known in the art.

As used herein, the term “Parenteral” administration of an immunogenic composition includes, e.g., subcutaneous (s.c), intravenous (i.v.), intramuscular (i.m.), or intrasternal injection, or infusion techniques.

As used herein, the terms “Patient,” “Subject,” and “Individual,” and the like are used interchangeably herein, and refer to any animal, or cells thereof whether in vitro or in situ, amenable to the methods described herein. A subject can be a mammal, such as a non-primate (e.g., cows, pigs, horses, cats, dogs, rats, etc.) or a primate (e.g., monkey and human). In certain embodiments, the term “Subject,” as used herein, refers to a vertebrate, such as a mammal. Mammals include, without limitation, humans, non-human primates, wild animals, feral animals, farm animals, sport animals, and pets. Any living organism in which an immune response can be elicited may be a subject or patient. In certain exemplary embodiments, a subject is a human.

As used herein, the term “Polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “Nucleotides.” The monomelic nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “Peptide,” “Polypeptide,” and “Protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “Promoter” as used herein is defined as a DNA sequence recognized by the synthetic machinery of the cell, or introduced synthetic machinery, required to initiate the specific transcription of a polynucleotide sequence.

As used herein, the term “Promoter” or “/Regulatory sequence” means a nucleic acid sequence which is required for expression of a gene product operably linked to the “promoter” or “regulatory sequence.” In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, a “Constitutive promoter” is a nucleotide sequence which, when operably linked with a polynucleotide which encodes or specifies a gene product, causes the gene product to be produced in a cell under most or all physiological conditions of the cell.

As used herein, an “Inducible promoter” is a nucleotide sequence which, when operably linked with a polynucleotide which encodes or specifies a gene product, causes the gene product to be produced in a cell substantially only when an inducer which corresponds to the promoter is present in the cell.

As used herein, a “Tissue-specific promoter” is a nucleotide sequence which, when operably linked with a polynucleotide encodes or specified by a gene, causes the gene product to be produced in a cell substantially only if the cell is a cell of the tissue type corresponding to the promoter.

By the term “Specifically binds,” or “selectively binds,” as used herein with respect to an antibody, antigen-binding domain, a CAR, or a bi-specific fusion protein, is meant an antibody, antigen-binding domain, a CAR, or a bi-specific fusion protein which recognizes a specific antigen (e.g., a TACA), but does not substantially recognize or bind other molecules in a sample. For example, an antibody, antigen-binding domain, a CAR, or a bi-specific fusion protein that specifically binds to an antigen (e.g., a TACA) from one species may also bind to that antigen from one or more species. But, such cross-species reactivity does not itself alter the classification of an antibody as specific. In another example, an antibody, antigen-binding domain, a CAR, or a bi-specific fusion protein that specifically binds to an antigen (e.g., a TACA) may also bind to different allelic forms of the antigen (e.g., TACA). However, such cross reactivity does not itself alter the classification of an antibody, antigen-binding domain, a CAR, or a bi-specific fusion protein as specific. In some instances, the terms “specific binding” or “specifically binding,” can be used in reference to the interaction of an antibody, a protein, a peptide, antigen-binding domain, a CAR, or a bi-specific fusion protein with a second chemical species, to mean that the interaction is dependent upon the presence of a particular structure (e.g., an antigenic determinant or epitope) on the chemical species; for example, an antibody, antigen-binding domain, a CAR, or a bi-specific fusion protein recognizes and binds to a specific protein structure (TACA) rather than to proteins generally. If an antibody, antigen-binding domain, a CAR, or a bi-specific fusion protein is specific for epitope “A”, the presence of a molecule containing epitope A (or free, unlabeled A), in a reaction containing labeled “A” and the antibody, antigen-binding domain, a CAR, or a bi-specific fusion protein, may reduce the amount of labeled A bound to the antibody.

As used herein, the term “Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird, Science 242:423-442 (1988); Huston et al., Proc. Natl. Acad. Sci. USA 85:5879-5883 (1988); Ward et al., Nature 334:54454 (1989); Skerra et al., Science 242:1038-1041 (1988). 10.1851 As used herein, the term “Specificity” refers to the ability to specifically bind (e.g., immunoreact with) a given target antigen (e.g., a human target antigen). A chimeric antigen receptor may be monospecific and contain one or more binding sites which specifically bind a target or a chimeric antigen receptor may be multi-specific and contain two or more binding sites which specifically bind the same or different targets. In certain embodiments, a chimeric antigen receptor is specific for two different (e.g., non-overlapping) portions of the same target. In certain embodiments, a chimeric antigen receptor is specific for more than one target.

As used herein, the term “Specifically binds,” with respect to an antibody, means an antibody or binding fragment thereof (e.g., scFv) which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, a chimeric antigen receptor, or a peptide with a second chemical species, to mean that the interaction is dependent upon the presence of a particular structure (e.g., an antigenic determinant or epitope) on the chemical species; for example, a chimeric antigen receptor recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

As used herein, the term “Stimulation,” means a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, clonal expansion, and differentiation into distinct subsets.

As used herein, the term “Stimulatory molecule” means a molecule on a T cell that specifically binds with a cognate stimulatory ligand present on an antigen presenting cell.

As used herein, the term “Stimulatory ligand” means a ligand that when present on an antigen presenting cell (e.g., an aAPC, a dendritic cell, a B-cell, and the like) can specifically bind with a cognate binding partner (referred to herein as a“stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, a “Substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

As used herein, a “Target site” or “Target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, coupled to three dimeric modules CD3δ/CD3ε, CD3γ/CD3ε, and CD3ζ/CD3ζ. In some cells the TCR consists of gamma and delta (γ/δ) chains (CD3γ/CD3δ). TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

As used herein, the term “Therapeutic” as used herein means a treatment and/or prophylaxis. A therapeutic effect is obtained by suppression, remission, or eradication of a disease state.

The term “Therapeutically effective amount” or “Effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

As used herein, the term “Therapy” refers to any protocol, method and/or agent (e.g., a CAR-T) that can be used in the prevention, management, treatment and/or amelioration of a disease or a symptom related thereto. In some embodiments, the terms “therapies” and “therapy” refer to a biological therapy (e.g., adoptive cell therapy), supportive therapy (e.g., lymphodepleting therapy), and/or other therapies useful in the prevention, management, treatment and/or amelioration of a disease or a symptom related thereto, known to one of skill in the art such as medical personnel.

As used herein, the terms “Treat,” “Treatment” and “Treating” refer to the reduction or amelioration of the progression, severity, frequency and/or duration of a disease or a symptom related thereto, resulting from the administration of one or more therapies (including, but not limited to, a CAR-T therapy directed to the treatment of solid tumors). The term “Treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis. In some embodiments, to “treat” a disease as the term is used herein, means to reduce the frequency or severity of at least one sign or symptom of a disease or disorder experienced by a subject.

The term “Transfected” or “Transformed” or “Transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “Under transcriptional control” or “Operatively linked” as used herein means that the promoter is in the correct location and orientation in relation to a polynucleotide to control the initiation of transcription by RNA polymerase and expression of the polynucleotide.

As used herein, a “Vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “Xenogeneic” refers to a graft derived from an animal of a different species.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### III. TACA Antigen Binding Domain

One aspect of the present disclosure provides a bi-specific fusion protein or a chimeric antigen receptor comprising an antigen binding domain that selectively binds a tumor-associated carbohydrate antigen (TACA). In some embodiments, the antigen binding domain comprises a TACA-binding domain derived from a lectin. In some embodiments, the antigen binding domain comprises more than one (e.g., multiple) TACA binding domains.

Malignant transformation of cells is near universally accompanied with aberrant glycosylation of cell surface proteins or lipids (Kim and Varki, 1997, Glycoconj J, 14: 569-576). Indeed, alteration of cell surface glycosylation has been observed in all types of experimental and human cancers (Hakomori, 2002, PNAS USA, 99: 10231-10233), and these altered sugar structures are called tumor-associated carbohydrate antigens (TACAs) (Table 1). This tumor-specific property makes cell surface TACAs an excellent target antigen for production of monoclonal antibodies targeting many common cancers. However, despite decades of effort, specific antibodies with high-affinity against TACAs are not yet available due to difficulties in generating antibodies to carbohydrate antigens.

Accordingly, the inventors of the present disclosure relied on lectins rather than antibodies to engineer therapeutic bi-specific fusion proteins and CARs that selectively target a TACA on a tumor cell. Lectins and their binding partners are well-known in the art. See e.g., functionalglycomics.org/glycomics/publicdata/primaryscreen.jsp. Lectin binding proteins of the present disclosure provide an opportunity for the development of a novel class of therapeutic drugs for cancer immunotherapy, with significant advantages over existing technology (e.g., GlyTR chimeric proteins). Based on the GlyTR concept and the availability of many different lectins specific for many different TACAs, multiple GlyTRs can be generated by replacing L-PHA with other lectins; or chimeric proteins can be produced composed of lectins and scFv that can recruit other immune effector cells. The functionality of the lectin domain can be improved through mutation. For example, GlyTR L-PHA×CD3 can be further improved by exchanging the carbohydrate binding domain of E-PHA with L-PHA, which increases binding −20-30 fold (Kaneda et al., 2002, J Biol Chem, 277: 16928-16935).

TABLE 1
Exemplary TACAs and their binding partners

| Lectins | TACA |
|---|---|
| L-PHA | β1,6 GlcNAc branching; tri-antennary oligosaccharides; |
| | tetra-antennary oligosaccharides |
| Truncated L-PHA | β1,6 GlcNAc branching; tri- and tetra-antennary |
| | oligosaccharides |
| Mutated L-PHA | β1,6 GlcNAc branching; tri- and tetra-antennary |
| | oligosaccharides |
| Mutated L-PHA | β1,6 GlcNAc branching; tri- and tetra-antennary |
| | oligosaccharides |
| VVA (Vicia villosa | Tn epitopes; Tn Antigen |
| isolectin B | |
| mutated VVA (Vicia | Tn epitopes |
| villosa isolectin B | |
| CD301 (CLEC10A) | Tn epitopes; Sialyl-Tn epitopes; GD2 |
| Siglec 1-17 | β1,6 branching; sialyl-Tn epitopes; Sialylation; di- |
| Extracellular domain | sialyl-Lewis^{x/a}; GD3; GM3; Lewis-y (Le^{y}), Lewis Y, |
| | Globo H |
| Galectin 1-15 | β1,6 branching; sialyl-Tn epitopes; T antigen; Tn epitopes; |
| Extracellular domain | Globo H |
| tomato lectin | β1,6 branching |
| (Lycopersicon esculentum | |
| lectin; LEA | |
| BC2L-CNt (lectin from the | Globo H |
| gram-negative bacteria | |
| Burkholderia | |
| cenocepacia); Siglec, | |
| galectin | |
| CD301 (CLEC10 A, | GD2 |
| macrophage galactose | |
| lectin); | |
| Siglec, galectin | |
| Siglec, galectin, Siglec-7, | GD3 |
| Siglec-9 | |
| Siglec, galectin, Siglec-9 | GM3 |
| Siglec, galectin, Subunit B | Fucosyl GM1 |
| Cholera Toxin | |

The antigen binding domain of the bi-specific fusion protein or the CAR disclosed herein is designed to specifically target glycoprotein and/or glycolipid (i.e., carbohydrate-containing macromolecule) on tumor cell. In some embodiments, the bi-specific fusion protein or the CAR of the present disclosure comprises affinity to a target antigen (e.g., a tumor associated carbohydrate antigen) on a target cell (e.g., a cancer cell). The target antigen may include any type of protein, or epitope thereof, associated with the target cell. For example, the CAR or bi-specific fusion protein may comprise affinity to a target antigen on a target cell that indicates a particular status of the target cell.

In some embodiments, the antigen binding domain comprises multiple (e.g., more than one) TACA binding domains. In some embodiments, the antigen binding domain comprises two, three, four, five, six, seven, eight, nine, ten, or more TACA binding domains. In one embodiment, the antigen binding domain comprises two TACA binding domains. In one embodiment, the antigen binding domain comprises three TACA binding domains. In one embodiment, the antigen binding domain comprises four TACA binding domains.

### A. TACA

The TACA-binding domain may comprise any peptide, protein, lectin, lectin fragment, antibody, antibody fragment, small molecule, nucleic acid, or the like, which can specifically bind to a TACA. In some embodiments, the antigen binding domain selectively targets β1,6GlcNAc-branched N-glycans, Tn epitopes (Tn antigen), sialyl-Tn epitopes (sialyl-Tn antigen), GalNAcα-Serine, GalNAcα-Threonine, GalNAc, or GalNAcβ1.

Exemplary TACAs and their binding partners are listed in Table 1. Exemplary TACAs include, but are not limited to, β1, 6 branching, T antigen, sialyl-T epitopes, Tn epitopes, sialyl-Tn epitopes, α2, 6 sialylation, Sialylation, sialyl-Lewis\hat{ }, di-sialyl-Lewis\hat{ }, sialyl 6-sulfo Lexis^{x}, Lewis-y (Le^{y}), Lewis Y, Globo H, GD2, GD3, GM3, and Fucosyl GMl. In some embodiments, the CAR or bi-specific fusion selectively targets a TACA selected from the group consisting of β1, 6 branching, β1,6GlcNAc-branched N-glycans, T antigen, Tn antigen, sialyl-T epitopes, Tn epitopes, sialyl-Tn epitopes, α2, 6 sialylation, Sialylation, sialyl-Lewis^{x/a}, di-sialyl-Lewis^{x/a}, sialyl 6-sulfo Lexis^{x}, Lewis-y (Le^{y}), Lewis Y, Globo H, GD2, GD3, GM3, and Fucosyl GM1. In some embodiments, the CAR selectively targets β1,6GlcNAc-branched N-glycans, GalNAc, Tn antigen, GalNAcα-ser, GalNAc, or GalNAcβ1.

In one embodiment, the TACA-binding domain binds to an N-glycan In certain embodiments, the TACA-binding domain binds to a tri- and tertra-antennary oligosaccharide In one embodiment, the TACA binding domain binds to β1,6GlcNAc-branched N-glycans. In one embodiment, the TACA binding domain binds to Tn epitopes.

### B. Lectins

In certain embodiments, the TACA-binding domain is a peptide sequence derived from a lectin protein. In certain embodiments the lectin is selected from the group consisting of a mammalian lectin, human lectin, plant lectin, bacterial lectin, viral lectin, fungal lectin, and protozoan lectin. In some embodiments, the antigen binding domain comprises a TACA-binding domain derived from a lectin. In some embodiments, the antigen binding domain comprises at least two TACA binding domains from a lectin.

In some embodiments, the lectin is selected from the group consisting of a galectin, a siglec, a selectin; a C-type lectin; CD301, a polypeptide N-acetylgalactosaminyltransferase (ppGalNAc-T), L-PHA (Phaseolus vulgaris leukoagglutinin); E-PHA (Phaseolus vulgaris erythroagglutinen); tomato lectin (Lycopersicon esculentum lectin; LEA); peanut lectin (Arachis hypogaea Agglutinin; PNA); potato lectin (Solanum tuberosum lectin), pokeweed mitogen (Phytolacca American lectin), wheat germ agglutinin (Triticum Vulgaris lectin); Artocarpus polyphemus lectin (Jacalin letin); Vicia villosa Agglutinin (VVA); Helix pomatia Agglutinin (HPA); Wisteria floribunda Agglutinin (WFA); Sambucus nigra Agglutinin (SNA), BC2L-CNt (lectin from the gram negative bacteria Burkholderia cenocepacia), Maackia amurensis leukoagglutinin (MAL), Psathyrella velutina (PVL), Sclerotium rolfsii lectin (SRL), Eucheuma serra agglutinin (ESA), CLEC17A (Prolectin), Aleuria aurantia lectin, Sambucus sieboldiana lectin (SSA), Glechoma hederacea lectin (Gleheda), Morus nigra agglutinin (Morniga G), Salvia sclarea lectin, Salvia bogotensis lectin, Salvia horminum lectin, Clerodendrum trichotomum lectin, Moluccella laevis lectin, Griffonia simplicifolia (GsLA4), Psophocarpus tetragonolobus (acidic WBAI), Abrus precatorius lectin, Amaranthus caudatus lectin, Amaranthus leucocarpus lectin, Laelia autumnalis lectin, Artocarpus integrifolia lectin, Maclura pomifera lectin, Artocarpus lakoocha lectin, Dolichos biflorus agglutinin, Dolichos biflorus lectin, Glycine max lectin, and Agaricus bisporus lectin.

In some embodiments, the lectin is a galectin that can be selected from the group consisting of galectin-1, galectin-2, galectin-3, galectin-4, galectin-5, galectin-6, galectin-7, galectin-8, galectin-9, galectin-10, galectin-11, galectin-12, galectin-13, galectin-14 and galectin-15. In some embodiments, the lectin is a siglec that can be selected from the group consisting of siglec-1 (sialoadhesion), siglec-2 (CD22), siglec-3 (CD33), siglec-4 (myelin associated glycoprotein), siglec-5, siglec-6, siglec-7, siglec-8, siglec-9, siglec-10, siglec-11, siglec-12, siglec-13, siglec-14, siglec-15, siglec-16, siglec-17, Siglec E, Siglec F, siglec G and siglec H. In some embodiments, the TACA-binding domain is derived from a selecin or a C-type lectin.

In some embodiments, the lectin is a polypeptide N-acetylgalactosaminyltransferase (ppGalNAc-T) that can be selected from the group consisting of ppGalNAc-T1 (GALNT1), ppGalNAc-T2 (GALNT2), ppGalNAc-T3 (GALNT3), ppGalNAc-T4 (GALNT4), ppGalNAc-T5 (GALNT5), ppGalNAc-T6 (GALNT6), ppGalNAc-T7 (GALNT7), ppGalNAc-T8 (GALNT8), ppGalNAc-T9 (GALNT9), ppGalNAc-T10 (GALNT10), ppGalNAc-T12 (GALNT12), ppGalNAc-T13 (GALNT13), ppGalNAc-T14 (GALNT14), ppGalNAc-T15 (GALNT15), ppGalNAc-T16 (GALNT16), ppGalNAc-T17 (GALNT17), ppGalNAc-T18 (GALNT18), ppGalNAc-T Like 5 (GALNTL5), and ppGalNAc-T Like 6 (GALNTL6).

In some embodiments, the antigen binding domain of the CAR or bi-specific fusion protein described herein selectively targets a TACA selected from the group consisting of 01, 6 branching, β1,6GlcNAc-branched N-glycans, T antigen, Tn antigen, sialyl-T epitopes, Thomsen-nouveau (Tn) epitopes (Tn antigen), sialyl-Tn epitopes (sialyl-Tn antigen), α2, 6 sialylation, Sialylation, sialyl-Lewisx/a, di-sialyl-Lewisx/a, sialyl 6-sulfo Lexis^{x}, Lewis-y (Le^{y}), Lewis Y, Globo H, GD2, GD3, GM3, and Fucosyl GM1.

### C. Chimeric Antigen Receptors

One aspect of the present disclosure provides compositions and methods for modified immune cells or precursor cells thereof, e.g., modified T cells, comprising a chimeric antigen receptor (CAR) having affinity for a tumor-associated carbohydrate antigen (TACA). A subject CAR of the present disclosure comprises an antigen binding domain (e.g., a tumor-associated carbohydrate antigen (TACA), a transmembrane domain, a costimulatory signaling domain, and an intracellular signaling domain. A subject CAR of the present disclosure may optionally comprise a hinge domain. In some embodiments, each of the domains of the subject CAR is separated by a linker. In one aspect, the chimeric antigen receptor that selectively binds a tumor-associated carbohydrate antigen (TACA) is encoded by the isolated nucleic acid disclosed herein.

One aspect of the present disclosure provides a chimeric antigen receptor (CAR) that comprises an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99. In some embodiments, the CAR comprises an amino acid sequence having at least 90%, at least 91%, at least 92%, at least 93%, at least 94%, at least 95%, at least 96%, at least 97%, at least 98%, at least 99% sequence identity to an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99.

In another aspect of the present disclosure, the chimeric antigen receptor that selectively binds a tumor-associated carbohydrate antigen (TACA) comprises an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, or 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; a CD8 a hinge domain; a CD8 transmembrane domain; a CD28 costimulatory; and a CD3 zeta intracellular signaling domain.

In another aspect, the chimeric antigen receptor that selectively binds a tumor-associated carbohydrate antigen (TACA) comprises an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, or 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; a CD8 a hinge domain; a CD8 transmembrane domain; a 4-1BB costimulatory domain; and a CD3 zeta intracellular signaling domain.

In another aspect, the chimeric antigen receptor that selectively binds a tumor-associated carbohydrate antigen (TACA) comprises an antigen binding domain selected from the group consisting of SEQ ID NOs: 100, 101, 102, 103, 104, 105, 106, 107, 108, 109, 110, 133, 134, 135, 136, 136, 137, 139, 140, 141, 142, 143, 144, 145, or 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 110, 133, 134, 135, 136, 136, 137, 139, 140, 141, 142, 143, 144, 145, or 146; a CD8 a hinge domain; a CD8 transmembrane domain; a CD28 and a 4-1BB costimulatory domains; and a CD3 zeta intracellular signaling domain.

In some embodiments, the CAR comprises the amino acid sequence of SEQ ID NO: 72, 88, 89, 91, 92, or 93. In some embodiments, the CAR comprises the amino acid sequence of SEQ ID NO: 93, 94, 95, 96, 97, 98, or 99.

In some embodiments, the chimeric antigen receptor (CAR) has affinity for β1, 6 branching, β1,6GlcNAc-branched N-glycans, T antigen, Tn antigen, sialyl-T epitopes, Tn epitopes, sialyl-Tn epitopes, α2, 6 sialylation, Sialylation, sialyl-Lewis^{x/a}, di-sialyl-Lewis^{x/a}, sialyl 6-sulfo Lexis^{x}, Lewis-y (Le^{y}), Lewis Y, Globo H, GD2, GD3, GM3, or Fucosyl GM1. In some embodiments, the chimeric antigen receptor (CAR) has affinity for β1, 6 branching, or β1,6GlcNAc-branched N-glycans. In some embodiments, the chimeric antigen receptor (CAR) has affinity for a Tn antigen or sialyl-Tn epitopes.

### 1. Extracellular Domain

The antigen binding domain of a CAR is an extracellular region of the CAR for binding to a specific target antigen including proteins, carbohydrates, and glycolipids. In some embodiments, the CAR comprises affinity to a target antigen (e.g., a tumor associated antigen) on a target cell (e.g., a cancer cell). The target antigen may include any type of protein, or epitope thereof, associated with the target cell. For example, the CAR may comprise affinity to a target antigen on a target cell that indicates a particular status of the target cell.

In some embodiments, the antigen binding domain comprises the amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152. Alternatively, the antigen binding domain comprises an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152. In some embodiments, the antigen binding domain comprises an amino acid sequence disclosed in Table 2 or 3.

In some embodiments of the antigen binding domain comprises an amino acid sequence having at least 90% homology to SEQ ID NO: 100, 101, 102, 103, 104, 105, 106, 107, 108, 109, 133, 134, 135, 136, 136, 137, 139, 140, 141, 142, 143, 144, 145, 152, or 146.

The antigen binding domain may be operably linked to another domain of the CAR, such as the transmembrane domain, the costimulatory signaling domain or the intracellular signaling domain, each described elsewhere herein, for expression in the cell. The antigen binding domains described herein can be combined with any of the transmembrane domains, any of the costimulatory signaling domains, any of the intracellular signaling domains, or any of the other domains described herein that may be included in a CAR of the present disclosure.

### 2. Linkers

The terms “linker” and “spacer” are used interchangeably herein. The linker is typically rich in glycine for flexibility, as well as serine or threonine for solubility. Multiple linker may be used to connect the more than one TACA binding domains. In some embodiments, the more than one TACA binding domains can be operably linked by a linker, such as a linker may be selected from the group consisting of a peptide linker, a non-peptide linker, a chemical unit, a hindered cross-linker, a non-hindered cross-linker. In one embodiment, the linker is a peptide linker. The peptide linker can be a glycine-serine linker. The peptide linker can be at least about 4, at least about 6, at least about 8, at least about 10, at least about 12, at least about 14, or at least about 15 amino acids in length.

Various linker sequences are known in the art and non-limiting examples of linkers are disclosed in Shen et ai., Anal. Chem. 80(6): 1910-1917 (2008) and WO 2014/087010, the contents of which are hereby incorporated by reference in their entireties. Those of skill in the art would be able to select the appropriate linker sequence for use in the bi-specific fusion protein and/or CAR of the present disclosure.

In some embodiments, the linker comprises the amino acid sequence selected from the group consisting of SEQ ID NO: 128, SEQ ID NO: 129, SEQ ID NO: 127, SEQ ID NO: 130, SEQ ID NO: 131, and SEQ ID NO: 132. In one embodiment, the linker comprises the amino acid sequence of SEQ ID NO: 127, or the amino acid sequence of SEQ ID NO: 131.

### 3. Transmembrane Domain

With respect to the transmembrane domain, the CAR of the present disclosure (e.g., TACA CAR) can be designed to comprise a transmembrane domain that connects the antigen binding domain of the CAR to the intracellular domain. The transmembrane domain of the subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence.

In some embodiments, the chimeric antigen receptor (CAR) comprises a transmembrane domain that may comprise a transmembrane region of a molecule selected from the group consisting of T-cell receptor (TCR)-alpha, TCR-beta, CD3-zeta, CD3-epsilon, CD4, CD5, CD8, CD9, CD16, CD22, CD28, CD33, CD37, CD45, CD64, CD80, CD86, CD134 (Ox40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In one embodiment, the transmembrane domain comprises a CD8 transmembrane domain. Alternatively, the transmembrane domain comprises the amino acid sequence of SEQ ID NO: 148.

In some embodiments, the transmembrane domain may be synthetic, in which case it will comprise predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

### 4. Intracellular Domain

A subject CAR of the present disclosure also includes an intracellular domain. The intracellular domain of the CAR is responsible for activation of at least one of the effector functions of the cell in which the CAR is expressed (e.g., immune cell). The intracellular domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability. In certain embodiments, the intracellular domain comprises a costimulatory signaling domain and an intracellular signaling domain.

Examples of the intracellular signaling domain include, without limitation, the z chain of the T cell receptor complex or any of its homologs, syk family tyrosine kinases (Syk, ZAP 70, etc.), src family tyrosine kinases (Lck, Fyn, Lyn, etc.), and other molecules involved in T cell transduction, such as CD2, CD3 and CD28. In one embodiment, the intracellular signaling domain may be human CD3 zeta chain, FcγRin, FcεRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CDS zeta, CDS gamma, CDS delta, CDS epsilon, CD86, common FcR gamma, FcR beta (Fc Epsilon Rib), CD79a, CD79b, Fc gamma R1 la, DAP10, DAP12, T cell receptor (TCR), CDS, CD27, CD28, 4-1BB (CD137), OX9, 0X40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKpSO (KLRF1), CD127, CD160, CD19, CD4, CDSalpha, CDSbeta, IL2R beta, IL2R gamma, IL7R alpha, ITGA4, VLA1, CD49a, ITGA4, IA4, CD49D, ITGA6, VLA-6, CD49f, ITGAD, CDlId, ITGAE, CD103, ITGAL, CDlla, LFA-1, ITGAM, CD lib, ITGAX, CDllc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, LylOS), SLAM (SLAMF1, CD150, IPO-3), BLAME (SLAMF8), SELPLG (CD 162), LTBR, LAT, GADS, SLP-76, PAG/Cbp, NKp44, NKpSO, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling protdns including CDS, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKpSO (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) GGAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino adds. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, Fc gamma RI, Fc gamma RIIA, Fc gamma RIIC, Fc gamma RIIIA, FcRL5 (see, e.g., Gillis et al., Front (2014) Immunol. 5:254).

A suitable intracellular signaling domain can be an ITAM motif-containing portion that is derived from a polypeptide that contains an GGAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3δ (CD3 delta), CD3ε (CDS epsilon), CD3γ (CDS gamma), CD3ζ (CDS zeta), and CD79A (antigen receptor complex-associated protein alpha chain).

In one embodiment, the intracellular signaling domain is derived from DAP 12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCεR1G (also known as FCRG; Fc epsilon receptor I gamma chain; Fc receptor gamma-chain; fc-epsilon RI-gamma; fcR gamma; fceRl gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3δ; CD3-DELTA; T3D; CDS antigen, delta subunit; CD3d antigen, delta polypeptide (TiT3 complex); OKT3, delta chain; T-cell receptor T3 delta chain; T-cell surface glycoprotein CDS delta chain; etc.). In one embodiment, the intracellular signaling domain is derived from T-cell surface glycoprotein CD3 epsilon chain (also known as CD3ε, T-cell surface antigen T3/Leu-4 epsilon chain, T-cell surface glycoprotein CD3 epsilon chain, AI504783, CDS, CDSepsilon, T3e, etc.).

In one embodiment, the intracellular signaling domain is derived from T-cell surface glycoprotein CD3 gamma chain (also known as CD3γ, T-cell receptor T3 gamma chain, CD3-GAMMA, T3G, gamma polypeptide (TiT3 complex), etc.). In one embodiment, the intracellular signaling domain is derived from T-cell surface glycoprotein CDS zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). In one embodiment, an intracellular signaling domain suitable for use in a subject CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CDS epsilon, CD3, CD22, CD79a, CD79b, or CD66d. In one embodiment, the intracellular signaling domain in the CAR includes a cytoplasmic signaling domain of human CDS zeta.

a. Costimulatory Domain

In certain embodiments, the intracellular domain comprises a costimulatory signaling domain. In some embodiments, the chimeric antigen receptor (CAR) comprises a costimulatory domain that is a costimulatory domain of a molecule selected from the group consisting of CD27, CD28, 4-IBB (CD137), OX40, CD30, CD40, PD-1, ICOS, lymphocyte function-associated antigen-1 (LFA-1), CD2, CD7, CD8, LIGHT, NKG2C, B7-H3, a ligand that specifically binds with CD83, DAP10, DAP12, Lck, Fas, and a combination thereof.

In one embodiment, the costimulatory domain comprises a 4-1BB costimulatory domain, or a CD28 costimulatory domain. In one embodiment, the costimulatory domain comprises a 4-1BB costimulatory domain and a CD28 costimulatory domain. In one embodiment, the costimulatory domain comprises the amino acid sequence of SEQ ID NO: 114 or SEQ ID NO: 113. In one embodiment, the costimulatory domain comprises the amino acid sequence of SEQ ID NO: 114 and SEQ ID NO: 113.

Tolerable variations of the intracellular domain will be known to those of skill in the art, while maintaining specific activity. For example, in some embodiments the intracellular domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 113 or 114.

b. Intracellular Signaling Domain

In certain embodiments, the intracellular domain comprises an intracellular signaling domain. In some embodiments, the isolated nucleic acid molecule encoding the chimeric antigen receptor (CAR) comprises an intracellular domain that may be from the intracellular signalling domain of a molecule selected from the group consisting of T cell receptor (TCR) zeta, FcR-gamma, FcR-beta, CD3-gamma, CD3-delta, CD3-epsilon, CD3-zeta, CD3, CD5, CD22, CD79a, CD79b, and CD66d. In one embodiment, the intracellular signaling domain comprises a CD3zeta signaling domain; or the amino acid sequence of SEQ ID NO: 115.

Tolerable variations of the intracellular domain will be known to those of skill in the art, while maintaining specific activity. For example, in some embodiments the intracellular domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 115.

### 5. Hinge

The hinge region of the CAR is a hydrophilic region which is located between the antigen binding domain and the transmembrane domain. In some embodiments, this domain facilitates proper protein folding for the CAR. The hinge region is an optional component for the CAR. In some embodiments, the chimeric antigen receptor (CAR) may further comprise a hinge domain.

In some embodiment, the hinge domain is a protein selected from the group consisting of a CD8a, an Fc fragment of an antibody, a hinge region of an antibody, a CH2 region of an antibody, a CH3 region of an antibody, and an artificial spacer sequence. In one embodiment, the hinge domain is a CD8a hinge domain. In one embodiment, the hinge domain comprises the amino acid sequence of SEQ ID NO: 147. In some embodiments, the hinge domain comprises an amino acid sequence selected from the group consisting of SEQ ID NO: 119, SEQ ID NO: 124, 127, 128, 129, 130, 131, 132, and 147.

In some embodiments, the CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations. In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 amino acids to about 10 amino acids, from about 10 amino adds to about 15 amino acids, from about 15 amino acids to about 20 amino acids, from about 20 amino acids to about 25 amino acids, from about 25 amino adds to about 30 amino acids, from about 30 amino acids to about 40 amino acids, or from about 40 amino acids to about 50 amino acids.

Suitable hinge regions can be readily selected and can be of any of a number of suitable lengths, such as from 1 amino acid (e.g., Gly) to 20 amino adds, from 2 amino acids to 15 amino acids, from 3 amino acids to 12 amino acids, including 4 amino acids to 10 amino adds, 5 amino acids to 9 amino acids, 6 amino acids to 8 amino acids, or 7 amino adds to 8 amino acids, and can be 1, 2, 3, 4, 5, 6, or 7 amino acids.

### D. Bi-Specific Fusion Proteins

One aspect of the present disclosure provides a bi-specific fusion protein comprising an antigen binding domain that selectively binds a tumor-associated carbohydrate antigen (TACA), and an immune cell recognition domain that specifically binds a receptor on an immune effector cell.

The bi-specific fusion protein comprises two different binding specificities and thus binds to two different antigens. In one embodiment, the bi-specific fusion protein comprises a first antigen recognition domain that binds to a first antigen (e.g., TACA) and a second antigen recognition domain that binds to a second antigen. In one embodiment, the first antigen recognition domain is a TACA-binding domain. Examples of TACAs are described elsewhere herein, all of which may be targeted by the bi-specific fusion protein of the present invention. In certain embodiments, the second antigen recognition domain binds to an immune effector cell. In some embodiments, the antigen binding domain comprises a TACA-binding domain derived from a lectin; and the antigen binding domain comprises more than one TACA binding domains as described herein.

In some embodiments, the bi-specific fusion protein comprises an amino acid sequence selected from SEQ ID NOs: 1-5, 10-34, 39-42, 47-50, 55-58, or 63-66. Alternatively, the bi-specific fusion protein comprises an amino acid sequence having at least 90% sequence identity to an amino acid sequence selected from SEQ ID NOs: 1-5, 10-34, 39-42, 47-50, 55-58, or 63-66. In some embodiments, the amino acid sequence is selected from SEQ ID NO: 3-5, 11-13, 19-21, 28-30, 32-34, 40-42, 48-50, 56-58, or 64-66. In some embodiments, the bi-specific fusion protein comprises an amino acid sequence having at least 90% sequence identity to an amino acid sequence selected from SEQ ID NO: 3-5, 11-13, 19-21, 28-30, 32-34, 40-42, 48-50, 56-58, or 64-66. In some embodiments, the bi-specific fusion protein comprises the amino acid sequence selected from SEQ ID NO: 3-5, 10-13, 18-21, 26-34, 39-42, 47-50, 55-58, or 63-66. In some embodiments, the bi-specific fusion protein comprises the amino acid sequence disclosed in Table 2 or 3. In some embodiments, the bi-specific fusion protein comprises the amino acid sequence of SEQ ID NOs: 31-34, 39-42, 47-50, 55-58, 63, or 64.

In some embodiments, the bi-specific fusion protein exhibits enhanced binding to Thomsen-nouveau (Tn) antigen (Tn antigen) expressing tumor cells when compared to a bi-specific fusion protein comprising a flexible linker in the antigen binding domain. In that embodiment, the antigen binding domain is derived from CD301 (CLEC10A). In that embodiment, the flexible linker is a glycine-serine linker or a linker comprising an amino acid sequence selected from SEQ ID NO: 128, SEQ ID NO: 129, or SEQ ID NO: 127. Alternatively, the flexible linker is a glycine-serine linker or a linker comprising an amino acid sequence having at least 90% sequence identity to an amino acid sequence selected from SEQ ID NO: 128, SEQ ID NO: 129, or SEQ ID NO: 127.

In some embodiments, the bi-specific fusion protein comprises an immune cell recognition domain that selectively binds a receptor on an immune effector cell. In that embodiment, the immune effector cell can be selected from the group consisting of a T cell, a natural killer (NK) cell, a natural killer T (NKT) cell, a macrophage, a monocyte, a dendritic cell, and a neutrophil. In that embodiment, the immune effector cell can be a T cell. In another embodiment, the immune effector cell can be an NK cell. The receptor on the immune effector cell can be selected from the group consisting of T-cell receptor (TCR) alpha, TCR beta, TCR gamma, TCR delta, invariant TCR of NKT cells, CD3, CD2, CD28, CD25, CD16, NKG2D, NKG2A, CD138, KIR3DL, NKp46, MICA, and CEACAM1. Alternatively, the receptor on the immune effector cell is a T cell receptor selected from the group consisting of CD3, CD2, CD28, and CD25. In some embodiments, the immune effector cell is an NK cell, and the NK cell receptor may be selected from the group consisting of NKG2D, NKG2A, CD138, KIR3DL, NKp46, MICA, and CEACAM1.

In some embodiments, the immune cell recognition domain of the bi-specific fusion protein comprises a peptide, a protein, an antibody, a single domain antibody, a nanobody, an antibody fragment, or single-chain variable fragment (scFv) that selectively binds to a receptor on the immune effector cell. The immune cell recognition domain may comprise an scFv that may selectively bind CD3, CD2, CD28, CD25, CD16, NKG2D, NKG2A, CD138, KIR3DL, NKp46, MICA, and CEACAM1. In some embodiments, the immune cell recognition domain specifically binds CD3. Alternatively, the immune cell recognition domain may comprise the amino acid sequence of SEQ ID NOs: 149, 150 or 151. In some embodiments, the immune cell recognition domain may comprise an amino acid sequence having at least 90% sequence identity to the amino acid sequence of SEQ ID NO: 149, 150, or 151.

Alternatively, the immune cell recognition domain comprises an antibody Fc domain, optionally an Fc region of an IgG molecule. In one embodiment, the bi-specific fusion protein is an Fc fusion protein comprising the antigen binding domain that selectively binds a tumor-associated carbohydrate antigen (TACA). In some embodiments, the immune cell recognition domain comprises an antibody Fc domain and a domain that specifically binds CD3. In another embodiment, the immune cell recognition domain comprises the constant region domains CH2 and/or CH3 of an antibody, preferably CH2 and CH3. The constant region domains CH2 and/or CH3 of an antibody may or may not comprise a hinge region.

In some embodiments, the bi-specific fusion protein selectively targets a TACA selected from the group consisting of β1, 6 branching, β1,6GlcNAc-branched N-glycans, T antigen, sialyl-T epitopes, Thomsen-nouveau (Tn) epitopes (Tn antigen), sialyl-Tn epitopes (sialyl-Tn antigen), α2, 6 sialylation, Sialylation, sialyl-Lewis^{x/a}, di-sialyl-Lewis^{x/a}, sialyl 6-sulfo Lexis^{x}, Lewis-y (Le^{y}), Lewis Y, Globo H, GD2, GD3, GM3, and Fucosyl GM1.

In one embodiment, the bi-specific fusion protein selectively targets a Tn antigen or a β1,6GlcNAc-branched N-glycan. In some embodiments, the bi-specific fusion protein that selectively targets a β1,6GlcNAc-branched N-glycan comprises an antigen binding domain having the amino acid sequence selected from SEQ ID NO: 100-102, or 133-141; or an amino acid sequence having at least 90% sequence identity to an amino acid sequence selected from SEQ ID NO: 100-102, or 133-141. In another embodiment, the bi-specific fusion protein that selectively targets a β1,6GlcNAc-branched N-glycan comprises the amino acid sequence selected from SEQ ID NOs: 1-5 and 10-25; or an amino acid sequence having at least 90% sequence identity to an amino acid sequence selected from SEQ ID NOs: 1-25. In some embodiments, the bi-specific fusion protein that selectively targets a β1,6GlcNAc-branched N-glycan comprises the amino acid sequence selected from SEQ ID NOs: 3-5, 11-13, or 19-21; or an amino acid sequence having at least 90% sequence identity to an amino acid sequence selected from SEQ ID NOs: 3-5, 11-13, or 19-21. In some embodiments, the bi-specific fusion protein exhibits enhanced binding to β1,6GlcNAc-branched N-glycans expressing tumor cells when compared to a bi-specific fusion protein comprising a flexible linker in the antigen binding domain. In some embodiments, the flexible linker is a glycine-serine linker or a linker comprising an amino acid sequence selected from SEQ ID NO: 124, SEQ ID NO: 128, SEQ ID NO: 129, or SEQ ID NO: 127; or an amino acid sequence having at least 90% sequence identity to an amino acid sequence selected from SEQ ID NO: 124, SEQ ID NO: 128, SEQ ID NO: 129, or SEQ ID NO: 127.

Another aspect of the present disclosure provides a bi-specific fusion protein that selectively binds a tumor-associated carbohydrate antigen (TACA) comprising an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and (ii) an immune cell recognition domain that specifically binds a receptor on an immune effector cell.

One aspect of the aspect of the present disclosure provides a bi-specific fusion protein that selectively binds a tumor-associated carbohydrate antigen (TACA) comprising an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and an immune cell recognition domain that specifically binds CD3 on an immune effector cell.

Another aspect the present disclosure provides a bi-specific fusion protein that selectively binds a tumor-associated carbohydrate antigen (TACA) comprising a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and a peptide, a protein, an antibody, a single domain antibody, an antibody fragment, or single-chain variable fragment (scFv) that selectively binds to a receptor on the immune effector cell.

In some embodiments, the bi-specific fusion protein that selectively binds a tumor-associated carbohydrate antigen (TACA) comprises a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and an Fc domain of antibody. In one embodiment, the domain is an Fc region of an IgG molecule.

In some embodiments, the bi-specific fusion protein that selectively binds a tumor-associated carbohydrate antigen (TACA) comprises a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and the constant region domains CH2 and/or CH3 of an antibody. In one embodiment, the constant region may be a CH2 and CH3. In one embodiment, the CH2 and/or CH3 domain comprises a hinge region. In one embodiment, the CH2 and/or CH3 domain do not comprise a hinge region. Another aspect of the present disclosure provides a bi-specific fusion protein encoded by the isolated nucleic acid disclosed herein.

### E. Generating the CARs or Bi-Specific Fusion Proteins

The bi-specific fusion proteins, CARs, or peptides of the present disclosure may be made using chemical methods. For example, peptides, bi-specific fusion proteins, or CARs can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

A peptide, a bi-specific fusion protein, or a CAR of the present disclosure may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford 111. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide, a bi-specific fusion protein, or a CAR of the present disclosure conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the peptide of the invention fused to the selected protein or marker protein as described herein. Examples of proteins which may be used to prepare fusion proteins include immunoglobulins, glutathione-S-transferase (GST), hemagglutinin (HA), and truncated myc.

Peptides or bi-specific fusion proteins of the present disclosure may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

In one aspect, the present disclosure provides any form of a peptide, bi-specific fusion proteins, or CARs, having substantial homology to a peptide, a bi-specific fusion protein, or a CAR disclosed herein. Preferably, a peptide, a bi-specific fusion protein, or a CAR, which is “substantially homologous” is about 50% homologous, more preferably about 70% homologous, even more preferably about 80% homologous, more preferably about 90% homologous, even more preferably, about 95% homologous, and even more preferably about 99% homologous to amino acid sequence of a peptide disclosed herein. The peptide, a bi-specific fusion protein, or a CAR may alternatively be made by recombinant means or by cleavage from a longer polypeptide.

The variants of the peptides, bi-specific fusion proteins, or a CAR according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present disclosure, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence and/or the ability to bind to a TACA. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The bi-specific fusion proteins or CARs of the present disclosure can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

In some embodiments, the bi-specific fusion proteins or CARs of the present disclosure may include unnatural amino acids formed by post-translational modification or by introducing unnatural amino acids during translation. A variety of approaches are available for introducing unnatural amino acids during protein translation.

A peptide, a CAR or a bi-specific fusion protein of the present disclosure may be conjugated with other molecules, such as proteins, to prepare fusion proteins. This may be accomplished, for example, by the synthesis of N-terminal or C-terminal fusion proteins provided that the resulting fusion protein retains the functionality of the peptide. A peptide, a CAR, or a bi-specific fusion protein of the disclosure may be phosphorylated using conventional methods such as the method described in Reedijk et al. The EMBO Journal 11(4): 1365 (1992).

Cyclic derivatives of the peptides or bi-sepcific fusion proteins of the disclosure are also contemplated. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the present disclosure, cyclic peptides may comprise a beta-turn in the right position. Beta-turns may be introduced into the peptides of the invention by adding the amino acids Pro-Gly at the right position. It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

One aspect of the present disclosure provides bi-specific fusion proteins, fusion proteins, CARs, or peptides that are fused to, or integrated into, a target protein, and/or a targeting domain capable of directing the bi-specific fusion proteins, fusion proteins, CARs, or peptides to a desired cellular component or cell type or tissue. The bi-specific fusion proteins, fusion proteins, CARs, or peptides may also contain additional amino acid sequences or domains. The bi-specific fusion proteins, fusion proteins, CARs, or peptides are recombinant in the sense that the various components are from different sources, and as such are not found together in nature (i.e., are heterologous).

In one embodiment, the targeting domain can be a membrane spanning domain, a membrane binding domain, or a sequence directing the protein to associate with for example vesicles or with the nucleus. In one embodiment, the targeting domain can target a peptide to a particular cell type or tissue. For example, the targeting domain can be a cell surface ligand or an antibody against cell surface antigens of a target tissue (e.g., bone, regenerating bone, degenerating bone, cartilage). A targeting domain may target the peptide of the invention to a cellular component.

### II. Nucleic Acids and Expression Vectors

### A. Bi-Specific Fusion Proteins

One aspect of the present disclosure provides an isolated nucleic acid molecule encoding a bi-specific fusion protein comprising an antigen binding domain that selectively binds a tumor-associated carbohydrate antigen (TACA), and an immune cell recognition domain that specifically binds a receptor on an immune effector cell, where the antigen binding domain comprises a TACA-binding domain derived from a lectin; and the antigen binding domain comprises more than one (e.g., multiple)TACA binding domains.

In some embodiments, the antigen binding domain comprises two, three, four, five, six, seven, eight, nine, ten, or more TACA binding domains. The more than one (e.g., multiple) TACA binding domains can be operably linked by a linker, such as a linker is selected from the group consisting of a peptide linker, a non-peptide linker, a chemical unit, a hindered cross-linker, a non-hindered cross-linker. In one embodiment, the linker is a peptide linker. The peptide linker can be a glycine-serine linker. The peptide linker can be at least about 4, at least about 6, at least about 8, at least about 10, at least about 12, at least about 14, or at least about 15 amino acids in length.

In some embodiments, the linker comprises the amino acid sequence selected from the group consisting of SEQ ID NO: 128, SEQ ID NO: 129, SEQ ID NO: 127, SEQ ID NO: 130, SEQ ID NO: 131, and SEQ ID NO: 132. In one embodiment, the linker comprises the amino acid sequence of SEQ ID NO: 127, or the amino acid sequence of SEQ ID NO: 131.

In some embodiments, the lectin is selected from the group consisting of a galectin, a siglec, a selectin; a C-type lectin; CD301, a polypeptide N-acetylgalactosaminyltransferase (ppGalNAc-T), L-PHA (Phaseolus vulgaris leukoagglutinin); E-PHA (Phaseolus vulgaris erythroagglutinen); tomato lectin (Lycopersicon esculentum lectin; LEA); peanut lectin (Arachis hypogaea Agglutinin; PNA); potato lectin (Solanum tuberosum lectin), pokeweed mitogen (Phytolacca American lectin), wheat germ agglutinin (Triticum Vulgaris lectin); Artocarpus polyphemus lectin (Jacalin letin); Vicia villosa Agglutinin (VVA); Helix pomatia Agglutinin (HPA); Wisteria floribunda Agglutinin (WFA); Sambucus nigra Agglutinin (SNA), BC2L-CNt (lectin from the gram negative bacteria Burkholderia cenocepacia), Maackia amurensis leukoagglutinin (MAL), Psathyrella velutina (PVL), Sclerotium rolfsii lectin (SRL), Eucheuma serra agglutinin (ESA), CLEC17A (Prolectin), Aleuria aurantia lectin, Sambucus sieboldiana lectin (SSA), Glechoma hederacea lectin (Gleheda), Morus nigra agglutinin (Morniga G), Salvia sclarea lectin, Salvia bogotensis lectin, Salvia horminum lectin, Clerodendrum trichotomum lectin, Moluccella laevis lectin, Griffonia simplicifolia (GsLA4), Psophocarpus tetragonolobus (acidic WBAI), Abrus precatorius lectin, Amaranthus caudatus lectin, Amaranthus leucocarpus lectin, Laelia autumnalis lectin, Artocarpus integrifolia lectin, Maclura pomifera lectin, Artocarpus lakoocha lectin, Dolichos biflorus agglutinin, Dolichos biflorus lectin, Glycine max lectin, and Agaricus bisporus lectin.

In some embodiments, the lectin is a galectin that can be selected from the group consisting of galectin-1, galectin-2, galectin-3, galectin-4, galectin-5, galectin-6, galectin-7, galectin-8, galectin-9, galectin-10, galectin-11, galectin-12, galectin-13, galectin-14 and galectin-15. In some embodiments, the lectin is a siglec that can be selected from the group consisting of siglec-1 (sialoadhesion), siglec-2 (CD22), siglec-3 (CD33), siglec-4 (myelin associated glycoprotein), siglec-5, siglec-6, siglec-7, siglec-8, siglec-9, siglec-10, siglec-11, siglec-12, siglec-13, siglec-14, siglec-15, siglec-16, siglec-17, Siglec E, Siglec F, siglec G and siglec H.

In some embodiments, the lectin is a polypeptide N-acetylgalactosaminyltransferase (ppGalNAc-T) that can be selected from the group consisting of ppGalNAc-T1 (GALNT1), ppGalNAc-T2 (GALNT2), ppGalNAc-T3 (GALNT3), ppGalNAc-T4 (GALNT4), ppGalNAc-T5 (GALNT5), ppGalNAc-T6 (GALNT6), ppGalNAc-T7 (GALNT7), ppGalNAc-T8 (GALNT8), ppGalNAc-T9 (GALNT9), ppGalNAc-T10 (GALNT10), ppGalNAc-T12 (GALNT12), ppGalNAc-T13 (GALNT13), ppGalNAc-T14 (GALNT14), ppGalNAc-T15 (GALNT15), ppGalNAc-T16 (GALNT16), ppGalNAc-T17 (GALNT17), ppGalNAc-T18 (GALNT18), ppGalNAc-T Like 5 (GALNTL5), and ppGalNAc-T Like 6 (GALNTL6).

In some embodiments, the antigen binding domain of the bi-specific fusion protein described herein selectively targets a TACA selected from the group consisting of R1, 6 branching, β1,6GlcNAc-branched N-glycans, T antigen, Tn antigen, sialyl-T epitopes, Thomsen-nouveau (Tn) epitopes (Tn antigen), sialyl-Tn epitopes (sialyl-Tn antigen), α2, 6 sialylation, Sialylation, sialyl-Lewisx/a, di-sialyl-Lewisx/a, sialyl 6-sulfo Lexis^{x}, Lewis-y (Le^{y}), Lewis Y, Globo H, GD2, GD3, GM3, and Fucosyl GM1.

In some embodiments, the antigen binding domain selectively targets β1,6GlcNAc-branched N-glycans, Tn epitopes (Tn antigen), sialyl-Tn epitopes (sialyl-Tn antigen), GalNAcα-Serine, GalNAcα-Threonine, GalNAc, or GalNAcβ1.

In some embodiments, the antigen binding domain comprises the amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152. Alternatively, the antigen binding domain comprises an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152. In some embodiments, the antigen binding domain comprises the amino acid sequence disclosed in Table 2 or 3. In some embodiments of the nucleic acid molecule disclosed herein, the antigen binding domain comprises an amino acid sequence having at least 90% homology to SEQ ID NO: 100, 101, 102, 103, 104, 105, 106, 107, 108, 109, 133, 134, 135, 136, 136, 137, 139, 140, 141, 142, 143, 144, 145, 152, or 146.

In some embodiments, the isolated nucleic acid molecule encodes a bi-specific fusion protein comprising an amino acid sequence selected from SEQ ID NOs: 1-5, 10-34, 39-42, 47-50, 55-58, and 63-66. Alternatively, the isolated nucleic acid molecule encodes a bi-specific fusion protein comprising an amino acid sequence having at least 90% sequence identity to an amino acid sequence selected from SEQ ID NOs: 1-5, 10-34, 39-42, 47-50, 55-58, and 63-66. In some embodiments, the isolated nucleic acid molecule encodes a bi-specific fusion protein comprising the amino acid sequence selected from SEQ ID NO: 3-5, 10-13, 18-21, 26-34, 39-42, 47-50, 55-58, or 63-66. In some embodiments, the isolated nucleic acid molecule encodes a bi-specific fusion protein comprising the amino acid sequence selected from SEQ ID NO: 3-5, 11-13, 19-21, 28-30, 32-34, 40-42, 48-50, 56-58, or 64-66. In some embodiments, the isolated nucleic acid molecule encodes a bi-specific fusion protein comprising an amino acid sequence having at least 90% sequence identity to an amino acid sequence selected from SEQ ID NO: 3-5, 11-13, 19-21, 28-30, 32-34, 40-42, 48-50, 56-58, or 64-66. In some embodiments, the isolated nucleic acid molecule encodes a bi-specific fusion protein disclosed in Table 2 or 3. In some embodiments, the isolated nucleic acid molecule encodes a bi-specific fusion protein comprising the amino acid sequence of SEQ ID NOs: 31-34, 39-42, 47-50, 55-58, 63, or 64.

In some embodiments, the bi-specific fusion protein exhibits enhanced binding to Thomsen-nouveau (Tn) antigen (Tn antigen) expressing tumor cells when compared to a bi-specific fusion protein comprising a flexible linker in the antigen binding domain. In that embodiment, the flexible linker is a glycine-serine linker or a linker comprising an amino acid sequence selected from SEQ ID NO: 128, SEQ ID NO: 129, or SEQ ID NO: 127. Alternatively, the flexible linker is a glycine-serine linker or a linker comprising an amino acid sequence having at least 90% sequence identity to an amino acid sequence selected from SEQ ID NO: 128, SEQ ID NO: 129, or SEQ ID NO: 127.

In some embodiments, the isolated nucleic acid molecule encodes a bi-specific fusion protein comprising an immune cell recognition domain that selectively binds a receptor on an immune effector cell. In that embodiment, the immune effector cell can be selected from the group consisting of a T cell, a natural killer (NK) cell, a natural killer T (NKT) cell, a macrophage, a monocyte, a dendritic cell, and a neutrophil. In that embodiment, the immune effector cell can be a T cell. In another embodiment, the immune effector cell can be an NK cell. The receptor on the immune effector cell can be selected from the group consisting of T-cell receptor (TCR) alpha, TCR beta, TCR gamma, TCR delta, invariant TCR of NKT cells, CD3, CD2, CD28, CD25, CD16, NKG2D, NKG2A, CD138, KIR3DL, NKp46, MICA, and CEACAM1. Alternatively, the receptor on the immune effector cell is a T cell receptor selected from the group consisting of CD3, CD2, CD28, and CD25. In some embodiments, the immune effector cell is an NK cell, and the NK cell receptor may be selected from the group consisting of NKG2D, NKG2A, CD138, KIR3DL, NKp46, MICA, and CEACAM1.

In some embodiments, the immune cell recognition domain of the bi-specific fusion protein comprises a peptide, a protein, an antibody, a single domain antibody, a nanobody, an antibody fragment, or single-chain variable fragment (scFv) that selectively binds to a receptor on the immune effector cell. The immune cell recognition domain may comprise an scFv that may selectively bind CD3, CD2, CD28, CD25, CD16, NKG2D, NKG2A, CD138, KIR3DL, NKp46, MICA, and CEACAM1. In some embodiments, the immune cell recognition domain specifically binds CD3. Alternatively, the immune cell recognition domain may comprise the amino acid sequence of SEQ ID NOs: 149, 150 or 151. In some embodiments, the immune cell recognition domain may comprise amino acid sequence having at least 90% sequence identity to the amino acid sequence of SEQ ID NO: 149, 150, or 151.

Alternatively, the immune cell recognition domain comprises an antibody Fc domain, optionally an Fc region of an IgG molecule. In some embodiments, the immune cell recognition domain is an antibody Fc domain and a domain that specifically binds CD3. In one embodiment, the bi-specific fusion protein is an Fc fusion protein comprising the antigen binding domain that selectively binds a tumor-associated carbohydrate antigen (TACA). In another embodiment, the immune cell recognition domain comprises the constant region domains CH2 and/or CH3 of an antibody, preferably CH2 and CH3. The constant region domains CH2 and/or CH3 of an antibody may or may not comprise a hinge region.

### B. Chimeric Antigen Receptors

One aspect of the present disclosure provides an isolated nucleic acid molecule encoding a chimeric antigen receptor (CAR) comprising an antigen binding domain that selectively binds a tumor-associated carbohydrate antigen (TACA), where the antigen binding domain comprises a TACA-binding domain derived from a lectin; and the antigen binding domain comprises more than one TACA binding domains; a transmembrane domain; a costimulatory signaling region; and an intracellular signaling domain. In one embodiment, a first nucleic acid sequence encoding the antigen binding domain is operably linked to a second nucleic add encoding a transmembrane domain, and further operably linked to a third a nucleic acid sequence encoding a costimulatory signaling domain and/or an intracellular signaling domain.

In some embodiments, the antigen binding domain comprises two, three, four, five, six, seven, eight, nine, ten, or more TACA binding domains. The more than one (e.g., multiple)TACA binding domains can be operably linked by a linker, such as a linker is selected from the group consisting of a peptide linker, a non-peptide linker, a chemical unit, a hindered cross-linker, a non-hindered cross-linker. In one embodiment, the linker is a peptide linker. The peptide linker can be a glycine-serine linker. The peptide linker can be at least about 4, at least about 6, at least about 8, at least about 10, at least about 12, at least about 14, or at least about 15 amino acids in length.

In some embodiments, the linker comprises the amino acid sequence selected from the group consisting of SEQ ID NO: 128, SEQ ID NO: 129, SEQ ID NO: 127, SEQ ID NO: 130, SEQ ID NO: 131, and SEQ ID NO: 132. In one embodiment, the linker comprises the amino acid sequence of SEQ ID NO: 127, or the amino acid sequence of SEQ ID NO: 131.

In some embodiments, the lectin is selected from the group consisting of a galectin, a siglec, a selectin; a C-type lectin; CD301, a polypeptide N-acetylgalactosaminyltransferase (ppGalNAc-T), L-PHA (Phaseolus vulgaris leukoagglutinin); E-PHA (Phaseolus vulgaris erythroagglutinen); tomato lectin (Lycopersicon esculentum lectin; LEA); peanut lectin (Arachis hypogaea Agglutinin; PNA); potato lectin (Solanum tuberosum lectin), pokeweed mitogen (Phytolacca American lectin), wheat germ agglutinin (Triticum Vulgaris lectin); Artocarpus polyphemus lectin (Jacalin letin); Vicia villosa Agglutinin (VVA); Helix pomatia Agglutinin (HPA); Wisteria floribunda Agglutinin (WFA); Sambucus nigra Agglutinin (SNA), BC2L-CNt (lectin from the gram negative bacteria Burkholderia cenocepacia), Maackia amurensis leukoagglutinin (MAL), Psathyrella velutina (PVL), Sclerotium rolfsii lectin (SRL), Eucheuma serra agglutinin (ESA), CLEC17A (Prolectin), Aleuria aurantia lectin, Sambucus sieboldiana lectin (SSA), Glechoma hederacea lectin (Gleheda), Morus nigra agglutinin (Morniga G), Salvia sclarea lectin, Salvia bogotensis lectin, Salvia horminum lectin, Clerodendrum trichotomum lectin, Moluccella laevis lectin, Griffonia simplicifolia (GsLA4), Psophocarpus tetragonolobus (acidic WBAI), Abrus precatorius lectin, Amaranthus caudatus lectin, Amaranthus leucocarpus lectin, Laelia autumnalis lectin, Artocarpus integrifolia lectin, Maclura pomifera lectin, Artocarpus lakoocha lectin, Dolichos biflorus agglutinin, Dolichos biflorus lectin, Glycine max lectin, and Agaricus bisporus lectin.

In some embodiments, the lectin is a galectin that can be selected from the group consisting of galectin-1, galectin-2, galectin-3, galectin-4, galectin-5, galectin-6, galectin-7, galectin-8, galectin-9, galectin-10, galectin-11, galectin-12, galectin-13, galectin-14 and galectin-15. In some embodiments, the lectin is a siglec that can be selected from the group consisting of siglec-1 (sialoadhesion), siglec-2 (CD22), siglec-3 (CD33), siglec-4 (myelin associated glycoprotein), siglec-5, siglec-6, siglec-7, siglec-8, siglec-9, siglec-10, siglec-11, siglec-12, siglec-13, siglec-14, siglec-15, siglec-16, siglec-17, Siglec E, Siglec F, siglec G and siglec H.

In some embodiments, the lectin is a polypeptide N-acetylgalactosaminyltransferase (ppGalNAc-T) that can be selected from the group consisting of ppGalNAc-T1 (GALNT1), ppGalNAc-T2 (GALNT2), ppGalNAc-T3 (GALNT3), ppGalNAc-T4 (GALNT4), ppGalNAc-T5 (GALNT5), ppGalNAc-T6 (GALNT6), ppGalNAc-T7 (GALNT7), ppGalNAc-T8 (GALNT8), ppGalNAc-T9 (GALNT9), ppGalNAc-T10 (GALNT10), ppGalNAc-T12 (GALNT12), ppGalNAc-T13 (GALNT13), ppGalNAc-T14 (GALNT14), ppGalNAc-T15 (GALNT15), ppGalNAc-T16 (GALNT16), ppGalNAc-T17 (GALNT17), ppGalNAc-T18 (GALNT18), ppGalNAc-T Like 5 (GALNTL5), and ppGalNAc-T Like 6 (GALNTL6).

In some embodiments, the antigen binding domain of the CAR described herein selectively targets a TACA selected from the group consisting of β1, 6 branching, β1,6GlcNAc-branched N-glycans, T antigen, Tn antigen, sialyl-T epitopes, Thomsen-nouveau (Tn) epitopes (Tn antigen), sialyl-Tn epitopes (sialyl-Tn antigen), α2, 6 sialylation, Sialylation, sialyl-Lewisx/a, di-sialyl-Lewisx/a, sialyl 6-sulfo Lexis^{x}, Lewis-y (Le^{y}), Lewis Y, Globo H, GD2, GD3, GM3, and Fucosyl GM1.

In some embodiments, the antigen binding domain selectively targets β1,6GlcNAc-branched N-glycans, Tn epitopes (Tn antigen), sialyl-Tn epitopes (sialyl-Tn antigen), GalNAcα-Serine, GalNAcα-Threonine, GalNAc, or GalNAcβ1.

In some embodiments, the antigen binding domain comprises the amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152. Alternatively, the antigen binding domain comprises an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152. In some embodiments, the antigen binding domain comprises the amino acid sequence disclosed in Table 2 or 3.

In some embodiments of the nucleic acid molecule disclosed herein, the antigen binding domain comprises an amino acid sequence having at least 90% homology to SEQ ID NO: 100, 101, 102, 103, 104, 105, 106, 107, 108, 109, 133, 134, 135, 136, 136, 137, 139, 140, 141, 142, 143, 144, 145, 152, or 146.

In some embodiments, the isolated nucleic acid molecule encoding the chimeric antigen receptor (CAR) comprises a transmembrane domain that may comprise a transmembrane region of a molecule selected from the group consisting of T-cell receptor (TCR)-alpha, TCR-beta, CD3-zeta, CD3-epsilon, CD4, CD5, CD8, CD9, CD16, CD22, CD28, CD33, CD37, CD45, CD64, CD80, CD86, CD134 (Ox40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In one embodiment, the transmembrane domain comprises a CD8 transmembrane domain. Alternatively, the transmembrane domain comprises the amino acid sequence of SEQ ID NO: 148.

In some embodiments, the isolated nucleic acid molecule encoding the chimeric antigen receptor (CAR) comprises a costimulatory domain that is a costimulatory domain of a molecule selected from the group consisting of CD27, CD28, 4-IBB (CD137), OX40, CD30, CD40, PD-1, ICOS, lymphocyte function-associated antigen-1 (LFA-1), CD2, CD7, CD8, LIGHT, NKG2C, B7-H3, a ligand that specifically binds with CD83, DAP10, DAP12, Lck, Fas, and a combination thereof. In one embodiment, the costimulatory domain comprises a 4-1BB costimulatory domain, or a CD28 costimulatory domain. In one embodiment, the costimulatory domain comprises a 4-1BB costimulatory domain and a CD28 costimulatory domain. In one embodiment, the costimulatory domain comprises the amino acid sequence of SEQ ID NO: 114 or SEQ ID NO: 113. In one embodiment, the costimulatory domain comprises the amino acid sequence of SEQ ID NO: 114 and SEQ ID NO: 113.

In some embodiments, the isolated nucleic acid molecule encoding the chimeric antigen receptor (CAR) comprises an intracellular domain that may be from the intracellular signalling domain of a molecule selected from the group consisting of T cell receptor (TCR) zeta, FcR-gamma, FcR-beta, CD3-gamma, CD3-delta, CD3-epsilon, CD3-zeta, CDS, CD5, CD22, CD79a, CD79b, and CD66d. In one embodiment, the intracellular signalling domain comprises a CD3zeta signalling domain; or the amino acid sequence of SEQ ID NO: 115.

In some embodiments, the isolated nucleic acid molecule encoding the chimeric antigen receptor (CAR) that further comprises a hinge domain. In some embodiment, the hinge domain is a protein selected from the group consisting of a CD8a, an Fc fragment of an antibody, a hinge region of an antibody, a CH2 region of an antibody, a CH3 region of an antibody, and an artificial spacer sequence. In one embodiment, the hinge domain is a CD8a hinge domain. In one embodiment, the hinge domain comprises the amino acid sequence of SEQ ID NO: 147. In some embodiments, the hinge domain comprises an amino acid sequence selected from the group consisting of SEQ ID NO: 119, SEQ ID NO: 124, 127, 128, 129, 130, 131, 132, and 147.

One aspect of the present disclosure comprises an isolated nucleic acid molecule encoding a chimeric antigen receptor (CAR) that comprises: an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99. In some embodiments, the CAR comprises an amino acid sequence having at least 90%, at least 91%, at least 92%, at least 93%, at least 94%, at least 95%, at least 96%, at least 97%, at least 98%, at least 99% sequence identity to an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99.

### C. Nucleic Acids

The isolated nucleic acid sequence encoding a bi-specific fusion protein or a chimeric antigen receptor of the present disclosure can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding a peptide of the disclosure, or functional fragment thereof. In one embodiment, the composition comprises an isolated RNA molecule encoding the peptide of the disclosure, or a functional fragment thereof.

The nucleic acid molecules of the present disclosure can be modified to improve stability in serum or in growth medium for cell cultures. Modifications can be added to enhance stability, functionality, and/or specificity and to minimize immunostimulatory properties of the nucleic acid molecule of the disclosure. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule. In one embodiment of the present disclosure the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH2, NHR, NR2 or ON, wherein R is Ci-Ce alkyl, alkenyl or alkynyl and halo is F, Cl, Br, or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

For example, the nucleic acid molecule comprises at least one of the following chemical modifications: 2′-H, 2′-0-methyl, or 2′-OH modification of one or more nucleotides.

In certain embodiments, a nucleic acid molecule of the disclosure can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the disclosure can include 2′-0-methyl, 2′-fluorine, 2′-0-methoxyethyl, 2′-0-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-0-methyl, 2′-0-methoxyethyl (2′-0-MOE), 2′-0-aminopropyl (2′-0-AP), 2′-0-dimethylaminoethyl (2′-0-DMAOE), 2′-0-dimethylaminopropyl (2′-0-DMAP), 2′-0-dimethylaminoethyloxyethyl (2′-0-DMAEOE), or 2′-0-N-methylacetamido (2′-0-MA). In one embodiment, the nucleic acid molecule includes at least one 2′-0-methyl-modified nucleotide, and in some embodiments, all of the nucleotides of the nucleic acid molecule include a 2′-0-methyl modification.

In some embodiments, the nucleic acid molecule of the disclosure preferably has one or more of the following properties: Nucleic acid agents discussed herein include otherwise unmodified RNA and DNA as well as RNA and DNA that have been modified, e.g., to improve efficacy, and polymers of nucleoside surrogates.

Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al., Nucleic Acids Res., 1994, 22:2183-2196. Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone. Modifications of the nucleic acid of the disclosure may be present at one or more of, a phosphate group, a sugar group, backbone, N-terminus, C-terminus, or nucleobase.

### D. Expression Vectors

In one aspect, the present disclosure provides an expression construct comprising the isolated nucleic acid encoding a bi-specific fusion protein and/or a CAR disclosed herein. In some embodiments of the present disclosure, the isolated nucleic acid described herein comprises an expression vector; and/or an in vitro transcribed RNA. In another embodiment, the expression construct comprises an isolated nucleic acid encoding a CAR described herein.

The expression of natural or synthetic nucleic acids encoding a peptide of the invention is typically achieved by operably linking a nucleic acid encoding the peptide or portions thereof to a promoter and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present disclosure may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the present disclosure can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

### 1. Viral Based System

In some embodiments, the expression construct is a viral vector selected from the group consisting of a retroviral vector, a lentiviral vector, an adenoviral vector, and an adeno-associated viral vector. In some embodiments, the expression construct is a lentiviral vector. In some embodiments, the expression construct is a self-inactivating lentiviral vector. In some embodiments, the expression construct comprises an isolated nucleic acid encoding a bi-specific fusion protein described herein.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

### 2. Control Elements

In some embodiments, the vector also includes conventional control elements which are operably linked to the transgene in a manner which permits its transcription, translation and/or expression in a cell transfected with the plasmid vector or infected with the virus produced by the invention. As used herein, “Operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (poly A) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

### 3. Promoters

In some embodiments, the expression construct further comprises a promoter. The promoter may be selected from an EF-la promoter, a T cell Receptor alpha (TRAC) promoter, interleukin 2 (IL-2) promoter, or cytomegalovirus (CMV) promoter, a simian virus 40 (SV40) early promoter, a mouse mammary tumor virus (MMTV) promoter, a human immunodeficiency virus (HIV) long terminal repeat (LTR) promoter, a Moloney Murine Leukemia Virus (MoMuLV) promoter, an avian leukemia virus promoter, an Epstein-Barr virus immediate early promoter, or a Rous sarcoma virus promoter.

The immediate early cytomegalovirus (CMV) promoter sequence is an example of a strong constitutive promoter sequence capable of driving high levels of expression of any polynucleotide sequence operatively linked thereto. Another example of a suitable promoter is Elongation Growth Factor-la (EF-la). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney Murine Leukemia Virus (MoMuLV) promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

### 4. Selectable Marker

In order to assess the expression of a peptide, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity.

Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven.

### 5. CAR and Bi-Specific Fusion Protein Combination

In some embodiments, the expression construct comprises an isolated nucleic acid encoding a CAR polypeptide and a bi-specific fusion protein polypeptide. In some embodiments, the isolated nucleic acid molecule encoding a bi-specific fusion protein and an isolated nucleic acid molecule encoding a CAR are operably linked by a nucleic acid molecule encoding a self-cleaving 2A peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picomaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” ‘E2A,” “P2A,” and “T2A,” respectively. Those of skill in the art would be able to select the appropriate self-cleaving peptide for use in the present invention. In one embodiment, the self-cleaving 2A peptide is selected from P2A, T2A, E2A, or F2A.

In some embodiments, the isolated nucleic acid molecule encoding a bi-specific fusion protein and an isolated nucleic acid molecule encoding a CAR are operably linked by a nucleic acid molecule encoding a linker. A linker for use in the present disclosure (e.g., in the context of linking a CAR coding sequence and the bi-specific fusion protein coding sequence) allows for multiple proteins to be encoded by the same nucleic acid sequence (e.g., a multicistronic or bicistronic sequence), which are translated as a polyprotein that is dissociated into separate protein components. For example, a linker for use in a nucleic acid of the present disclosure comprising a CAR coding sequence and a bi-specific fusion protein coding sequence, allows for the CAR and the bi-specific fusion protein to be translated as a polyprotein that is dissociated into separate CAR and bi-specific fusion protein components.

In some embodiments, the linker comprises a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovims, HCV, Friend murine leukemia virus (FrMLV), and Moloney murine leukemia virus (MoMLV). Those of skill in the art would be able to select the appropriate IRES for use in the present invention.

### IV. Modified Cells

One aspect of the present disclosure provides genetically modified (e.g., engineered) cells, which include and stably express a subject CAR or bi-specific fusion protein of the present disclosure. In one aspect, the modified cell comprises an isolated nucleic acid encoding a chimeric antigen receptor (CAR) comprising an antigen-binding domain that selectively binds a tumor-associated carbohydrate antigen (TACA), a transmembrane domain, a costimulatory signaling region, and an intracellular signaling domain. In some embodiments, the modified cell comprises the chimeric antigen receptor that selectively binds a tumor-associated carbohydrate antigen (TACA). In one aspect, the modified cell comprises an isolated nucleic acid encoding a bi-specific fusion protein comprising an antigen binding domain that selectively binds a tumor-associated carbohydrate antigen (TACA), and an immune cell recognition domain that specifically binds a receptor on an immune effector cell.

In some embodiments, the modified cell comprises a bi-specific fusion protein described herein and/or a CAR described herein. In another embodiment, the modified cell comprises an isolated nucleic acid molecule encoding a bi-specific fusion protein described herein, and/or an isolated nucleic acid molecule encoding a CAR described herein. In another embodiment, the modified cell comprises an expression construct described herein.

In some embodiments, the modified cell comprises a bi-specific fusion protein described herein described herein. In another embodiment, the modified cell comprises an isolated nucleic acid molecule encoding a bi-specific fusion protein described herein. In another embodiment, the modified cell comprises an expression constructs described herein. In some embodiments, the modified cell comprises a CAR described herein. In another embodiment, the modified cell comprises an isolated nucleic acid molecule encoding a CAR described herein. In another embodiment, the modified cell comprises an expression constructs described herein.

In some embodiments, the modified cell is a genetically modified immune cell (e.g., T cell) or precursor cell thereof comprising a chimeric antigen receptor (CAR) or a bi-specific fusion protein having affinity for a tumor-associated carbohydrate antigen (TACA). In some embodiments, the genetically modified immune cell (e.g., T cell) or precursor cell thereof of the present invention comprises a CAR or a bi-specific fusion protein having affinity for β1, 6 branching, β1,6GlcNAc-branched N-glycans, T antigen, Tn antigen, sialyl-T epitopes, Tn epitopes, sialyl-Tn epitopes, α2, 6 sialylation, Sialylation, sialyl-Lewis^{x/a}, di-sialyl-Lewis^{x/a}, sialyl 6-sulfo Lexis^{x}, Lewis-y (Le^{y}), Lewis Y, Globo H, GD2, GD3, GM3, or Fucosyl GM1. In some embodiments, the genetically modified immune cell (e.g., T cell) or precursor cell thereof of the present invention comprises a CAR or bi-specific fusion protein having affinity for β1, 6 branching, or β1,6GlcNAc-branched N-glycans. In some embodiments, the genetically modified immune cell (e.g., T cell) or precursor cell thereof of the present invention comprises a CAR or a bi-specific fusion protein having affinity for a Tn antigen or sialyl-Tn epitopes.

One aspect of the present disclosure provides a modified cell comprising the isolated nucleic acid, the fusion protein, or the expression construct described herein. In some embodiment, the modified cell (e.g., a host cell) is selected from the group consisting of a bacterial cell, a fungal cell, an insect cell, or mammalian cell. In some embodiments, the modified cell is a bacterial cell selected from Escherichia coli or Bacillus stearothermophilus. In some embodiments, the modified cell is a fungal cell selected from a yeast cell, Saccharomyces cerevisiae or Pichia pastoris. In some embodiments, the modified cell is an insect cell selected from a lepidopteran insect cell, or Spodoptera frugiperda. In some embodiments, the modified cell is a mammalian cell selected from Chinese hamster ovary (CHO) cell, a baby hamster kidney (BHK) cell, a monkey kidney cells, a HeLa cell, a human hepatocellular carcinoma cell, or Human Embryonic Kidney 293 cell. In one embodiment, the modified cell is a CHO cell or an HEK 293 cell.

### A. Modified Cells

In some embodiments, the modified cell is selected from the group consisting of a T cell, a CD4⁺ T cell, a CD8⁺ T cell, a Natural Killer (NK) cell, a cytotoxic T lymphocyte (CTL), and a regulatory T cell. In some embodiments, the modified cell is a T cell. In certain embodiments, the genetically modified cell is a natural killer (NK) cell. In certain embodiments, the genetically modified cell is a NKT cell. In some embodiments, the modified cell is an autologous cell, a xenogeneic cell, or an allogeneic cell.

In some embodiments, the modified cell described herein comprises a bi-specific fusion protein described herein, and the CAR described herein or a CAR that targets a tumor antigen. In some embodiments, the modified cell described herein comprises an isolated nucleic acid molecule encoding a bi-specific fusion protein described herein, and an isolated nucleic acid molecule encoding a CAR described herein, or a CAR that targets a tumor antigen. In some embodiments, the modified cell described herein comprises expression construct odescribed herein and an expression construct comprising a CAR that targets a tumor antigen.

In some embodiments, the tumor antigen is selected from the group consisting of a tumor-associated carbohydrate antigen (TACA), alpha fetoprotein (AFP)/HLA-A2, AXL, B7-H3, BCMA, CA-IX, CD2, CD3, CD4, CDS, CD7, CD8, CD19, CD20, CD22, CD30, CD33, CD38, CD44v6, CD70, CD79a, CD79b, CD80, CD86, CDI 17, CD123, CD133, CD147, CDI 71, CD276, CEA, claudin 18.2, c-Met, DLL3, DRS, EGFR, EGFRvlll, EpCAM, EphA2, FAP, folate receptor alpha (FRa)/folate binding protein (FBP), GD-2, Glycolipid F77, glypican-3 (GPC3), HER2, HLA-A2, ICAMI, IL3Ra, IL13Ra2, LAGE-I, Lewis Y, LMPI (EBV), MAGE-A1, MAGE-A3, MAGE-A4, Melan A, mesothelin, MG7 (glycosylated CEA), MMP, MUCI, Nectin4/FAP, NKG2D-Ligands (MIC-A, MIC-B, and the ULBPs I to 6), NY-ESO-1, P1 6, PD-LI, PSCA, PSMA, RORI, ROR2, TIM-3, TM4SF1, TnMuc1, VEGFR2, and any combination thereof. In some embodiments, the tumor antigen is a TACA.

In some embodiments, the genetically modified cells are genetically engineered T-lymphocytes (T cells), regulatory T cells (Tregs), naive T cells (TN), memory T cells (for example, central memory T cells (TCM), effector memory cells (TEM)), natural killer cells (NK cells), natural killer T cells (NKT cells) and macrophages capable of giving rise to therapeutically relevant progeny. In some embodiments, the cell is selected from the group consisting of a T cell, a Natural Killer (NK) cell, a cytotoxic T lymphocyte (CTL), and a regulatory T cell. In one embodiment, the cell is a T cell. In one embodiment, the modified cells are autologous cells.

In some embodiments, the modified cell is a T cell, a Natural Killer (NK) cell, a cytotoxic T lymphocyte (CTL), or a regulatory T cell. In that embodiment, the T cell, the Natural Killer (NK) cell, the cytotoxic T lymphocyte (CTL), or a regulatory T cell comprises a chimeric antigen receptor (CAR). In some embodiments, the T cell, the Natural Killer (NK) cell, the cytotoxic T lymphocyte (CTL), or a regulatory T cell comprises a chimeric antigen receptor (CAR) that selectively or specifically binds a tumor antigen. In some embodiments, the tumor antigen is selected from the group consisting of a tumor-associated carbohydrate antigen (TACA), alpha fetoprotein (AFP)/HLA-A2, AXL, B7-H3, BCMA, CA-IX, CD2, CD3, CD4, CDS, CD7, CD8, CD19, CD20, CD22, CD30, CD33, CD38, CD44v6, CD70, CD79a, CD79b, CD80, CD86, CDI 17, CD123, CD133, CD147, CDI 71, CD276, CEA, claudin 18.2, c-Met, DLL3, DRS, EGFR, EGFRvlll, EpCAM, EphA2, FAP, folate receptor alpha (FRa)/folate binding protein (FBP), GD-2, Glycolipid F77, glypican-3 (GPC3), HER2, HLA-A2, ICAMI, IL3Ra, IL13Ra2, LAGE-I, Lewis Y, LMPI (EBV), MAGE-A1, MAGE-A3, MAGE-A4, Melan A, mesothelin, MG7 (glycosylated CEA), MMP, MUCI, Nectin4/FAP, NKG2D-Ligands (MIC-A, MIC-B, and the ULBPs I to 6), NY-ESO-1, P1 6, PD-LI, PSCA, PSMA, RORI, ROR2, TIM-3, TM4SF1, TnMuc1, VEGFR2, and any combination thereof. In one embodiment, the tumor antigen is a tumor-associated carbohydrate antigen (TACA). In some embodiments, the modified cell is a CAR T cell. In some embodiments, the modified cell is a CAR T cell that specifically targets a tumor antigen. In some embodiments, the modified cell is a CAR T cell that specifically targets a tumor-associated carbohydrate antigen (TACA).

In one aspect, the present disclosure provides populations of modified immune cells described herein.

### B. Method of Generating Modified Cells

In one aspect the present disclosure provides a method for generating the modified cell disclosed herein, the method comprising introducing into a cell the isolated nucleic acid encoding a CAR or a bi-specific fusion protein, or the expression construct of the present disclosure.

Modified cells (e.g., comprising a subject CAR or bi-specific fusion protein) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject CAR or bi-specific fusion protein of the present disclosure may be expanded ex vivo.

In some embodiments, the cell is genetically modified by contacting the cell with an isolated nucleic acid encoding the CAR or the bi-specific fusion protein as described herein. In some embodiments, the nucleic acid sequence is delivered into cells using a retroviral or lentiviral vector. For example, retroviral and lentiviral vectors expressing a peptide of the invention can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient.

In other embodiments, the nucleic acid sequence is delivered into cells using in vitro transcribed mRNA. In vitro transcribed mRNA can be delivered into different types of eukaryotic cells as well as into tissues and whole organisms using transfected cells as carriers or cell-free local or systemic delivery of encapsulated, bound or naked mRNA. The method used can be for any purpose where transient expression is required or sufficient.

In certain embodiments, the cell may be of any suitable cell type that can express the desired peptide. In certain embodiments, the modified cell is used in a method where the cell is introduced into a recipient. In certain embodiments, the cell is autologous, allogeneic, syngeneic or xenogeneic with respect to recipient.

The disclosed compositions and methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

In some embodiments, the modified cell is a modified host cell. In some embodiments, the modified cell is selected from the group consisting of a bacterial cell, a fungal, cell, an insect cell, or mammalian cell. In some embodiments, the modified cell is a bacterial cell selected from Escherichia coli or Bacillus stearothermophilus. In some embodiments, the modified cell is a fungal cell selected from a yeast cell, Saccharomyces cerevisiae. In some embodiments, the modified cell (e.g., a host cell) is an insect cell selected from a lepidopteran insect cell, or Spodoptera frugiperda.

In some embodiments, the modified cell is a mammalian cell selected from Chinese hamster ovary (CHO) cell, a baby hamster kidney (BHK) cell, a monkey kidney cells, a HeLa cell, a human hepatocellular carcinoma cell, or Human Embryonic Kidney 293 cell. In one embodiment, the modified cell is a CHO cell or an HEK 293 cell.

### 1. Physical Methods

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

### 2. Biological Methods

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In some embodiments, a nucleic acid encoding a subject CAR or bi-specific fusion protein of the present disclosure is introduced into a cell by an expression vector. Expression vectors comprising a nucleic acid encoding a subject CAR (e.g., TACA CAR) or bi-specific fusion protein are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR in the host cell. In some embodiments, the adenovirus genome is a 36 kb, linear, double stranded DNA, where a foreign DNA sequence (e.g., a nucleic acid encoding the TACA-CAR or bi-specific fusion protein) may be inserted to substitute large pieces of adenoviral DNA in order to make the expression vector of the present invention. See, e.g., Danthinne and Imperiale, Gene Therapy 7(20): 1707-1714(2000).

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a TACA-CAR or bi-specific fusion protein) into the viral genome at certain locations to produce a virus that is replication defective. Though, the retrovirus vectors are able to infect a broad variety of cell types, integration and stable expression of the subject CAR or bi-specific fusion protein, requires the division of host cells.

Lentivirus vectors are derived from lentiviruses, which are complex retroviruses that, in addition to the common retroviral genes gag, pol, and env, contain other genes with regulatory or structural function. See, e.g., U.S. Pat. Nos. 6,013,516 and 5,994,136. Some examples of lentiviruses include the human immunodeficiency viruses (HTV-1, HTV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR or bi-specific fusion protein (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors.

Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof, e.g., a T cell, an NK cell, or an NKT cell.

### 3. Chemical Methods

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds, which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Ghosh et al., Glycobiology 5: 505-10 (1991). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded T cells, transfecting the expanded T cells, and electroporating the expanded T cells. One nucleic acid may be introduced by one method and another nucleic add may be introduced into the T cell by a different method.

### 4. RNA

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of host cells with in vitro-transcribed RNA (TVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA. Some IVT vectors are known in the literature which are utilized in a standardized manner as template for in vitro transcription and which have been genetically modified in such a way that stabilized RNA transcripts are produced.

Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this non-physiological overhang affects the amount of protein produced intracellularly from such a construct.

In one embodiment, the isolated nucleic acid encoding the CAR or bi-specific fusion protein of the disclosure and introduced into a cell of the present disclosure comprises an RNA. In one embodiment, the RNA is mRNA. In one embodiment, the RNA is an in vitro transcribed (IVT) RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

In one embodiment, the DNA to be used for PCR contains an open reading frame. The DNA can be from a naturally occurring DNA sequence from the genome of an organism. In one embodiment, the DNA is a full length gene of interest of a portion of a gene. The gene can include some or all of the 5′ and/or 3′ untranslated regions (UTRs). The gene can include exons and introns. In one embodiment, the DNA to be used for PCR is a human gene. In another embodiment, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

Genes that can be used as sources of DNA for PCR include genes that encode polypeptides that provide a therapeutic or prophylactic effect to an organism or that can be used to diagnose a disease or disorder in an organism. Preferred genes are genes which are useful for a short term treatment, or where there are safety concerns regarding dosage or the expressed gene. For example, for treatment of cancer, autoimmune disorders, parasitic, viral, bacterial, fungal or other infections, the transgene(s) to be expressed may encode a polypeptide that functions as a ligand or receptor for cells of the immune system, or can function to stimulate or inhibit the immune system of an organism. In some embodiments, it is not desirable to have prolonged ongoing stimulation of the immune system, nor necessary to produce changes which last after successful treatment, since this may then elicit a new problem. For treatment of an autoimmune disorder, it may be desirable to inhibit or suppress the immune system during a flare-up, but not long term, which could result in the patient becoming overly sensitive to an infection.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. As used herein, “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art.

“Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources. Chemical structures with the ability to promote stability and/or translation efficiency may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA. The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but do not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is after transcription. On a linear DNA template, phage T7 RNA polymerase can extend the 3′ end of the transcript beyond the last base of the template. Schenborn and Mierendorf, Nuc Acids Res., 13:6223-36 (1985); Nacheva and Berzal-Herranz, Eur. J. Biochem., 270: 1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA. 5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7: 1468-95 (2001); Elango, et al., Biochim. Biophys. Res. Commun, 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

In some embodiments, the RNA is electroporated into the cells, such as in vitro transcribed RNA. The formulations and methodology of electroporation of nucleic acid constructs into mammalian cells as taught in e.g., US 2004/0014645, US 2005/0052630A1, US 2005/0070841 A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and U.S. Pat. No. 7,173,116. Apparatus for therapeutic application of electroporation are available commercially, e.g., the MedPulser™ DNA Electroporation Therapy System (Inovio/Genetronics, San Diego, Calif), and are described in patents such as U.S. Pat. Nos. 6,567,694; 6,516,223, 5,993,434, 6,181,964, 6,241,701, and 6,233,482; electroporation may also be used for transfection of cells in vitro as described e.g., in US20070128708A1. Electroporation may also be utilized to deliver nucleic acids into cells in vitro.

Accordingly, electroporation-mediated administration into cells of nucleic acids including expression constructs utilizing any of the many available devices and electroporation systems known to those of skill in the art presents an exciting new means for delivering an RNA of interest to a target cell.

The disclosed methods can be applied to the modulation of host cell activity in basic research and therapy, in the fields of cancer, stem cells, acute and chronic infections, and autoimmune diseases, including the assessment of the ability of the genetically modified host cell to kill a target cancer cell. The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

### C. Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. In certain exemplary embodiments, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid, or lymphoid cells, including lymphocytes, typically T cells and/or NK cells and/or NKT cells. Other exemplary cells include stem cells, such as multipotent and pluripotent stem cells, including induced pluripotent stem cells (iPSCs). In certain aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8⁺ T cell (e.g., a CD8⁺ naive T cell, central memory T cell, or effector memory T cell), a CD4⁺ T cell, a natural killer T cell (NKT cells), a regulatory T cell (Treg), a stem cell memory T cell, a lymphoid progenitor cell, a hematopoietic stem cell, a natural killer cell (NK cell), a natural killer T cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8⁺ T cell (e.g., a CD8⁺ naive T cell, central memory T cell, or effector memory T cell), a CD4⁺ T cell, a stem cell memory T cell, a lymphoid progenitor cell or a hematopoietic stem cell.

In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4⁺ and/or of CD8⁺ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain aspects, the sample from which the cells are derived or isolated is blood or a blood-derived sample or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in certain aspects, contain lymphocytes, including T cells, monocytes, granulocytes, B cells, other nucleated white blood cells, red blood cells, and/or platelets, and in certain aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some certain, a washing step is accomplished by tangential flow filtration (TFF) according to the manufacturer's instructions. In certain embodiments, the cells are resuspended in a variety of biocompatible buffers after washing. In certain embodiments, components of a blood cell sample are removed, and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or another saline solution with or without buffer. In some embodiments, the undesirable components of the apheresis sample may be removed, and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in certain aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use.

In certain aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells. In certain exemplary embodiments, multiple rounds of separation steps are carried out, where the positively or negatively selected fraction from one step is subjected to another separation step, such as a subsequent positive or negative selection.

In certain exemplary embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of tire T cell populations is enriched for or depleted of cells that are positive for (markeri-) or express high levels (markerhigh) of one or more particular markers, such as surface markers, or that are negative for (marker-) or express relatively low levels (markerlow) of one or more markers. For example, in certain aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8⁺ cells or the T cells, e.g., CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD 127). In certain exemplary embodiments, CD8⁺ T cells are enriched for cells positive for CD45RO (or negative for CD45RA) and for CD62L. For example, CD3⁺, CD28⁺ T cells can be positively selected using CD3/CD28 conjugated magnetic beads (e.g., DYNABEADS© M-450 CD3/CD28 T Cell Expander).

In some embodiments, T cells are separated from a PBMC sample by negative selection of markers expressed on non-T cells, such as B cells, monocytes, or other white blood cells, such as CD14. In certain aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations. In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in certain aspects is particularly robust in such sub-populations.

In some embodiments, combining TCM-enriched CD8⁺ T cells and CD4⁺ T cells further enhances efficacy. In some embodiments, memory T cells are present in both CD62L+ and CD62L-subsets of CD8⁺ peripheral blood lymphocytes. PBMC can be enriched for or depleted of CD62L-CD8⁺ and/or CD62L*CD8⁺ fractions, such as using anti-CD8 and anti-CD62L antibodies. In some embodiments, a CD4⁺ T cell population and/or a CD8⁺ T population is enriched for central memory (TCM) cells. In some embodiments, the enrichment for central memory T (TCM) cells is based on positive or high surface expression of CD45RO, CD62L, CCR7, CD28, CD8, and/or CD127. In certain aspects, the enrichment is based on negative selection for cells expressing or highly expressing CD45RA and/or granzyme B. In certain aspects, isolation of a CD8⁺ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in certain aspects are carried out simultaneously and in other aspects are carried out sequentially, in either order. In some certain, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

CD4⁺ T helper cells are sorted into naive, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA+, CD62L+, CD4⁺ T cells. In some embodiments, central memory CD4⁺ cells are CD62L+ and CD45RO⁺. In some embodiments, effector CD4⁺ cells are CD62L- and CD45RO. In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex.

In certain aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques. The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention.

In certain embodiments, it may be desirable to perform the selection procedure and use the “unselected” cells in the activation and expansion process. “Unselected” cells can also be subjected to further rounds of selection. Enrichment of a T cell population by negative selection can be accomplished using a combination of antibodies directed to surface markers unique to the negatively selected cells. An exemplary method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

In certain embodiments, it may be desirable to enrich for or positively select for regulatory T cells which typically express CD4⁺, CD25⁺, CD62L^{M}, GITR⁺, and FoxP3⁺.

Alternatively, in certain embodiments, T regulatory cells are depleted by anti-C25 conjugated beads or other similar method of selection. For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature. T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen. In certain embodiments, cryopreserved cells are thawed and washed as described herein and allowed to rest for one hour at room temperature prior to activation using the methods of the present invention.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, Cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present disclosure, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### D. Expansion of Immune Cells

Whether prior to or after modification of cells to express a subject CAR or bi-specific fusion protein, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005.

For example, the immune cells of the present disclosure may be expanded by contact with a surface having attached thereto an agent that stimulates a CD3/TCR complex associated signal and a ligand that stimulates a co-stimulatory molecule on the surface of the immune cells. In particular, immune cell populations may be stimulated by contact with an anti-CD3 antibody, or an antigen-binding fragment thereof, or an anti-CD2 antibody immobilized on a surface, or by contact with a protein kinase C activator (e.g., biyostatin) in conjunction with a calcium ionophore. For co-stimulation of an accessory molecule on the surface of the immune cells, a ligand that binds the accessory molecule is used. For example, immune cells can be contacted with an anti-CD3 antibody and an anti-CD28 antibody, under conditions appropriate for stimulating proliferation of the immune cells. Examples of an anti-CD28 antibody include 9.3, B-T3, XR-CD28 (Diaclone, Bes ancon, France) and these can be used in the invention, as can other methods and reagents known in the art. See, e.g., ten Berge et al., Transplant Proc. 30(8): 3975-3977 (1998); Haanen et al., J. Exp. Med. 190(9): 1319-1328 (1999); and Garland et al., J Immunol. Methods 227(1-2): 53-63 (1999).

Expanding the immune cells by the methods disclosed herein can be multiplied by about 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700 fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater, and any and all whole or partial integers therebetween. In one embodiment, the immune cells expand in the range of about 20-fold to about 50-fold.

Following culturing, the immune cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. In certain exemplary embodiments, the level of confluence is 70% or greater before passing the cells to another culture apparatus. In particularly exemplary embodiments, the level of confluence is 90% or greater. A period of time can be any time suitable for the culture of cells in vitro. The immune cell medium may be replaced during the culture of the immune cells at any time. In certain exemplary embodiments, the immune cell medium is replaced about every 2 to 3 days. The immune cells are then harvested from the culture apparatus whereupon the immune cells can be used immediately or cryopreserved to be stored for use at a later time. In one embodiment, the invention includes cryopreserving the expanded immune cells. The cryopreserved immune cells are thawed prior to introducing nucleic adds into the immune cell.

In another embodiment, the method comprises isolating immune cells and expanding the immune cells. In another embodiment, the invention further comprises cryopreserving the immune cells prior to expansion. In yet another embodiment, the cryopreserved immune cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of immune cells comprises the addition to the cellular growth factors, such as those described in U.S. Pat. No. 5,199,942, or other factors, such as flt3-L, IL-1, IL-3 and c-kit ligand. In one embodiment, expanding the immune cells comprises culturing the immune cells with a factor selected from the group consisting of flt3-L, IL-1, IL-3 and c-kit ligand. The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (PI or passage 1). After the second subculture, the cells become a tertiary culture (P2 or passage 2), and so on. It will be understood by those of skill in the art that there may be many population doublings during the period of passaging. Therefore the number of population doublings of a culture is greater than the passage number. The expansion of cells (i.e., the number of population doublings) during the period between passaging depends on many factors, including but is not limited to the seeding density, substrate, medium, and time between passaging.

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle: cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type-In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 104 to 109 T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8⁺ T cells that normally have weaker CD28 expression.

In one embodiment, the cells may be cultured for several hours (about 3 hours) to about 14 days or any hourly integer value in between. Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02).

The medium used to culture the immune cells may include an agent that can costimulate the immune cells. For example, an agent that can stimulate CDS is an antibody to CDS, and an agent that can stimulate CD28 is an antibody to CD28. This is because, as demonstrated by the data disclosed herein, a cell isolated by the methods disclosed herein can be expanded approximately 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10,000-fold, 100,000-fold, 1,000,000-fold, 10,000,000-fold, or greater. In one embodiment, the immune cells expand in the range of about 2-fold to about 50-fold, or more by culturing the electroporated population. In one embodiment, human T regulatory cells are expanded via anti-CD8 antibody coated KT64.86 artificial antigen presenting cells (aAPCs). Methods for expanding and activating immune cells can be found in U.S. Pat. Nos. 7,754,482, 8,722,400, and 9,555,105, the contents of which are incorporated herein in their entirety.

In one embodiment, the method of expanding the immune cells can further comprise isolating the expanded immune cells for further applications. In another embodiment, the method of expanding can further comprise a subsequent electroporation of the expanded immune cells followed by culturing. The subsequent electroporation may include introducing a nucleic acid encoding an agent, such as a transducing the expanded immune cells, transfecting the expanded immune cells, or electroporating the expanded immune cells with a nucleic acid, into the expanded population of immune cells, wherein the agent further stimulates the immune cell. The agent may stimulate the immune cells, such as by stimulating further expansion, effector function, or another immune cell function.

T cells that have been exposed to varied stimulation times may exhibit different characteristics. For example, typical blood or apheresed peripheral blood mononuclear cell products have a helper T cell population (TH, CD4⁺) that is greater than the cytotoxic or suppressor T cell population (Tc, CD8⁺). Ex vivo expansion of T cells by stimulating CD3 and CD28 receptors produces a population of T cells that prior to about days 8-9 consists predominately of TH cells, while after about days 8-9, the population of T cells comprises an increasingly greater population of Tc cells. Accordingly, depending on the purpose of treatment, infusing a subject with a T cell population comprising predominately of TH cells may be advantageous. Similarly, if an antigen-specific subset of Tc cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### E. Scaffolds

In another aspect, the present disclosure provides a scaffold or substrate composition comprising a peptide comprising a TACA-binding domain, a nucleic acid molecule encoding a peptide comprising a TACA-binding domain, a cell modified to express a peptide comprising a TACA-binding domain, or a combination thereof. For example, in one embodiment, a peptide comprising a TACA-binding domain, a nucleic acid molecule encoding a peptide comprising a TACA-binding domain, a cell modified to express a peptide comprising a TACA-binding domain, or a combination thereof is present within a scaffold. In another embodiment, a peptide comprising a TACA-binding domain, a nucleic acid molecule encoding a peptide comprising a TACA-binding domain, a cell modified to express a peptide comprising a TACA-binding domain, or a combination thereof is applied to the surface of a scaffold. The scaffold of the invention may be of any type known in the art. Non-limiting examples of such a scaffold includes a, hydrogel, electrospun scaffold, foam, mesh, sheet, patch, and sponge.

### V. Compositions

### A. TACA Compositions

In one aspect, the present disclosure provides a composition comprising the isolated nucleic acid encoding the bi-specific antibody or CAR disclosed herein; the bi-specific antibody or CAR disclosed herein; the expression construct disclosed herein; or the modified cell disclosed herein. In one aspect, the present disclosure provides a composition comprising the modified cells described herein or a population of modified cells described herein.

In one aspect of the present disclosure, the composition comprises a modified cell or a population of modified cells comprising a bi-specific fusion protein comprising an antigen binding domain that selectively binds a tumor-associated carbohydrate antigen (TACA), and an immune cell recognition domain that specifically binds a receptor on an immune effector cell, where the antigen binding domain comprises more than one (multiple) TACA-binding domain derived from a lectin.

In another aspect of the present disclosure, the composition comprises a modified cell or a population of modified cells comprising a chimeric antigen receptor (CAR) comprising: an antigen binding domain that selectively binds a tumor-associated carbohydrate antigen (TACA), where the antigen binding domain comprises more than one TACA-binding domain derived from a lectin; a transmembrane domain; a costimulatory signaling region; and an intracellular signaling domain.

In yet another aspect of the present disclosure, the composition comprises a modified cell or a population of modified cells comprising a chimeric antigen receptor (CAR) comprising an antigen binding domain that selectively binds a tumor-associated carbohydrate antigen (TACA) and a bi-specific fusion protein comprising an antigen binding domain that selectively binds a tumor-associated carbohydrate antigen (TACA). In some embodiments, the antigen binding domain comprises two, three, four, five, six, seven, eight, nine, ten, or more TACA binding domains.

In some embodiments, the lectin is selected from the group consisting of a galectin, a siglec, a selectin; a C-type lectin; CD301, a polypeptide N-acetylgalactosaminyltransferase (ppGalNAc-T), L-PHA (Phaseolus vulgaris leukoagglutinin); E-PHA (Phaseolus vulgaris erythroagglutinen); tomato lectin (Lycopersicon esculentum lectin; LEA); peanut lectin (Arachis hypogaea Agglutinin; PNA); potato lectin (Solanum tuberosum lectin), pokeweed mitogen (Phytolacca American lectin), wheat germ agglutinin (Triticum Vulgaris lectin); Artocarpus polyphemus lectin (Jacalin letin); Vicia villosa Agglutinin (VVA); Helix pomatia Agglutinin (HPA); Wisteria floribunda Agglutinin (WFA); Sambucus nigra Agglutinin (SNA), BC2L-CNt (lectin from the gram negative bacteria Burkholderia cenocepacia), Maackia amurensis leukoagglutinin (MAL), Psathyrella velutina (PVL), Sclerotium rolfsii lectin (SRL), Eucheuma serra agglutinin (ESA), CLEC17A (Prolectin), Aleuria aurantia lectin, Sambucus sieboldiana lectin (SSA), Glechoma hederacea lectin (Gleheda), Morus nigra agglutinin (Morniga G), Salvia sclarea lectin, Salvia bogotensis lectin, Salvia horminum lectin, Clerodendrum trichotomum lectin, Moluccella laevis lectin, Griffonia simplicifolia (GsLA4), Psophocarpus tetragonolobus (acidic WBAI), Abrus precatorius lectin, Amaranthus caudatus lectin, Amaranthus leucocarpus lectin, Laelia autumnalis lectin, Artocarpus integrifolia lectin, Maclura pomifera lectin, Artocarpus lakoocha lectin, Dolichos biflorus agglutinin, Dolichos biflorus lectin, Glycine max lectin, and Agaricus bisporus lectin.

In some embodiments, the antigen binding domain of the CAR or bi-specific fusion protein comprises the amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152.

In some embodiments, the antigen binding domain of the CAR or bi-specific fusion protein comprises an amino acid sequence having at least 90% homology to SEQ ID NO: 100, 101, 102, 103, 104, 105, 106, 107, 108, 109, 133, 134, 135, 136, 136, 137, 139, 140, 141, 142, 143, 144, 145, 152, or 146.

In one aspect, the bi-specific fusion protein that selectively binds a tumor-associated carbohydrate antigen (TACA) comprising (i) an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and (ii) an immune cell recognition domain that specifically binds a receptor on an immune effector cell.

In one aspect, the bi-specific fusion protein that selectively binds a tumor-associated carbohydrate antigen (TACA) comprises an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and an immune cell recognition domain that specifically binds CD3 on an immune effector cell.

In one aspect, the bi-specific fusion protein that selectively binds a tumor-associated carbohydrate antigen (TACA) comprises a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and a peptide, a protein, an antibody, a single domain antibody, an antibody fragment, or single-chain variable fragment (scFv) that selectively binds to a receptor on the immune effector cell.

In some embodiments, the bi-specific fusion protein that selectively binds a tumor-associated carbohydrate antigen (TACA) comprises a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and an Fc domain of antibody, optionally an Fc region of an IgG molecule.

In some embodiments, the bi-specific fusion protein that selectively binds a tumor-associated carbohydrate antigen (TACA) comprises a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and the constant region domains CH2 and/or CH3 of an antibody, preferably CH2 and CH3, optionally with or without a hinge region.

In some embodiments, the bi-specific fusion protein or the CAR selectively targets a TACA selected from the group consisting of R1, 6 branching, β1,6GlcNAc-branched N-glycans, T antigen, sialyl-T epitopes, Thomsen-nouveau (Tn) epitopes (Tn antigen), sialyl-Tn epitopes (sialyl-Tn antigen), α2, 6 sialylation, Sialylation, sialyl-Lewis^{x/a}, di-sialyl-Lewis^{x/a}, sialyl 6-sulfo Lexis^{x}, Lewis-y (Le^{y}), Lewis Y, Globo H, GD2, GD3, GM3, and Fucosyl GM1.

In one aspect, the chimeric antigen receptor that selectively binds a tumor-associated carbohydrate antigen (TACA) comprises an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, or 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; a CD8 a hinge domain; a CD8 transmembrane domain; a CD28 costimulatory and/or a 4-1BB costimulatory domain; and a CD3 zeta intracellular signaling domain.

In some embodiments, the CAR comprises an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99; or an amino acid sequence having at least 90%, at least 91%, at least 92%, at least 93%, at least 94%, at least 95%, at least 96%, at least 97%, at least 98%, at least 99% sequence identity to an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99. In some embodiments, the CAR comprises the amino acid sequence of SEQ ID NO: 72, 88, 89, 91, 92, or 93. In some embodiments, the CAR comprises the amino acid sequence of SEQ ID NO: 93, 94, 95, 96, 97, 98, or 99.

In some embodiments, the compositions of the present disclosure may comprise a modified unstimulated immune cell (e.g., a T cell, a Natural Killer (NK) cell, a cytotoxic T lymphocyte (CTL), and a regulatory T cell) or a modified stimulated immune cell as described herein.

### B. Pharmaceutical Compositions

In some embodiments, the composition is a pharmaceutical composition. In some embodiments, the composition may include a pharmaceutical composition and further comprises one or more pharmaceutically or physiologically acceptably carriers, diluents, adjuvants, or excipients. Such compositions may comprise buffers such as neutral buffered saline, phosphate buffered saline and the like; carbohydrates such as glucose, mannose, sucrose, or dextrans, mannitol; proteins; polypeptides or amino acids such as glycine, antioxidants; chelating agents such as EDTA or glutathione; adjuvants (e.g., aluminum hydroxide); and preservatives. Compositions of the present disclosure are preferably formulated for parenteral administration (e.g., intravenous administration).

As used herein, a “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain aspects, the choice of carrier is determined in part by the particular cell and/or by the method of administration.

### C. Formulations

Accordingly, there are a variety of suitable formulations. Formulations may be employed in admixtures with conventional excipients, i.e., pharmaceutically acceptable organic or inorganic carrier substances suitable for administration to the wound or treatment site. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined where desired with other active agents, e.g., other analgesic agents. Administration of the compositions of this disclosure may be carried out, for example, by parenteral, by intravenous, intratumoral, subcutaneous, intramuscular, or intraperitoneal injection, or by infusion or by any other acceptable systemic method.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the disclosure are known in the art and described, for example in Genaro, ed. (1985, Remington's Pharmaceutical Sciences, Mack Publishing Co., Easton, PA), which is incorporated herein by reference.

In some embodiments, the compositions of the present disclosure are provided as sterile liquid preparations, e.g., isotonic aqueous solutions, suspensions, emulsions, dispersions, or viscous compositions, which may in certain aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in certain aspects be consulted to prepare suitable preparations.

In some embodiments, the composition of the disclosure may comprise a preservative from about 0.005% to 2.0% by total weight of the composition. The preservative is used to prevent spoilage in the case of exposure to contaminants in the environment. Examples of preservatives useful in accordance with the disclosure included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

In some embodiments, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g., disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the peptide or other composition of the disclosure in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing, or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

In some embodiments, a therapeutically effective amount of the pharmaceutical composition comprising the modified immune cells of the present disclosure may be administered to a subject in need thereof.

The pharmaceutical compositions or formulations include those for oral, intravenous, intraperitoneal, subcutaneous, pulmonary, transdermal, intramuscular, intranasal, buccal, sublingual, or suppository administration. In some embodiments, the modified immune cell populations are administered parenterally. The term “parenteral,” as used herein, includes intravenous, intramuscular, subcutaneous, rectal, vaginal, and intraperitoneal administration. In some embodiments, the immune cells of the present disclosure are administered to the subject using peripheral systemic delivery by intravenous, intraperitoneal, or subcutaneous injection.

The cells of the present disclosure may be administered to a subject by aerosol inhalation, injection, ingestion, transfusion, implantation, or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, die cells of the disclosure are injected directiy into a site of inflammation in the subject, a local disease site in the subject, a lymph node, an organ, a tumor, and the like. It should be understood that the method and compositions that would be useful in the present disclosure are not limited to the particular formulations set forth in the examples. The following examples are put forth so as to provide those of ordinary skill in the art with a complete disclosure and description of how to make and use the cells, expansion and culture methods, and therapeutic methods of the disclosure, and are not intended to limit the scope of what the inventors regard as their invention.

The formulations to be used for in vivo administration are generally sterile. Sterility may be readily accomplished, e.g., by filtration through sterile filtration membranes. It can generally be stated that a pharmaceutical composition comprising the modified immune cells described herein may be administered at a dosage of 10⁴ to 10⁹ cells/kg body weight, in some instances 10⁴ to 10⁶ cells/kg body weight, including all integer values within those ranges. Immune cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

### VI. Method of Treatment

One aspect of the present disclosure provides a method of treating cancer in a subject having a cancer, the method comprising administering to the subject a pharmaceutically effective amount of a composition disclosed herein. In some embodiments, the composition comprises a modified cell or a population of modified cells comprising the bi-specific fusion protein, or the CAR disclosed herein. In some embodiments, the composition comprises a modified cell or a population of modified cells comprising an isolated nucleic acid encoding the CAR or the bi-specific fusion protein disclosed herein.

One aspect of the present disclosure provides a method of treating cancer in a subject having a cancer, the method comprising introducing a nucleic acid encoding a bi-specific fusion protein or a CAR of the present disclosure, introducing a CAR or a bi-specific fusion protein of the present disclosure, or introducing an expression vector comprising the nucleic acid encoding the CAR or the bi-specific fusion protein of the present disclosure into a cell (e.g., an immune cell) to produce a modified cell; and administering the modified cell to the subject. In some embodiments, the cell is obtained from the subject (i.e., cell is autologous), engineered ex vivo, and administered to the same subject. In some embodiments, the cell is obtained from a different subject, engineered ex vivo, and administered to a second suitable subject (i.e., the cell is allogeneic).

In one aspect, a method is provided including retrieving immune cells from a subject, genetically modifying the immune cells by introducing a nucleic acid encoding the CAR or bi-specific fusion protein of the present disclosure into the immune cells and administering the modified immune cells to the subject. In some embodiments, the immune cells are selected from T cells, naive T cells, memory T cells, effector T cells, natural killer cells (NK cells), or macrophages. In one embodiment, the immune cells are T cells.

In one embodiment, the immune cells are obtained from a subject. Immune cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present invention, any number of immune cell lines available in the art, may be used. In some embodiments of the present disclosure, immune cells can be obtained from blood collected from a subject using any number of techniques known to the skilled artisan, such as Ficoll™ separation. For example, in one embodiment, immune cells are isolated by incubation with anti-CD3/anti-CD28 (i.e., 3×28)-conjugated beads, such as DYNABEADS® M-450 CD3/CD28 T, for a time period sufficient for positive selection of the desired T cells.

In one embodiment, the time period is about 30 minutes. In one embodiment, the time period ranges from 30 minutes to 36 hours or longer and all integer values there between. In one embodiment, the time period is at least 1, 2, 3, 4, 5, or 6 hours. In one embodiment, the time period is 10 to 24 hours. In one embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled person would recognize that multiple rounds of selection can also be used in the context of this invention.

In some embodiments, it may be desirable to perform the selection procedure and use the “unselected” cells in the activation and expansion process. “Unselected” cells can also be subjected to further rounds of selection.

The obtained cells are then modified as described herein. A nucleic acid encoding the CAR or bi-specific fusion protein of the present disclosure, typically located in an expression vector, is introduced into the immune cells such that the immune cells will express, preferably stably, the CAR or the bi-specific fusion protein.

Depending upon the nature of the immune cells and the diseases to be treated, the modified immune cells (e.g., modified T cells or NK cells) may be introduced into the subject, e.g., a mammal, in a wide variety of ways. The genetically engineered immune cells may be introduced at the site of the tumor. In one embodiment, the genetically engineered immune cells navigate to the cancer or are modified to navigate to the cancer. The number of modified immune cells that are employed will depend upon a number of factors such as the circumstances, the purpose for the introduction, the lifetime of the cells, the protocol to be used. For example, the number of modified immune cells that are employed may depend upon the number of administrations, the ability of the cells to multiply, and the stability of the recombinant construct. The modified immune cells may be applied as a dispersion injected at or near the site of interest. In one embodiment, the cells may be in a physiologically-acceptable medium It should be appreciated that the treatment method is subject to many variables, such as the cellular response to the TACA-CAR or bi-specific fusion protein, the efficiency of expression of the TACA-CAR or bi-specific fusion protein by the immune cells and, as appropriate, the level of secretion, the activity of the expressed CAR or bi-specific fusion protein, the particular need of the subject, which may vary with time and circumstances, the rate of loss of the cellular activity as a result of loss of modified immune cells or the expression activity of individual cells, and the like. Therefore, it is expected that for each individual patient, even if there were universal cells, which could be administered to the population at large, each patient would be monitored for the proper dosage for the individual, and such practices of monitoring a patient are routine in the art.

In one embodiment, the modified T cells of the invention can undergo robust in vivo T cell expansion and can persist for an extended amount of time. In another embodiment, the modified T cells of the invention evolve into specific memory T cells that can be reactivated to inhibit any additional tumor formation or growth. For example, modified T cells of the invention can undergo robust in vivo T cell expansion and persist at high levels for an extended amount of time in blood and bone marrow and form specific memory T cells.

One aspect of the present disclosure provides a method of providing an anti-tumor immunity in a mammal, the method comprising administering to the mammal an effective amount of a population of modified cells described herein.

### A. Bi-Specific Fusion Proteins

In one aspect, the present disclosure provides a method of treating cancer in a subject in need thereof, the method comprising administering to the subject an immunotherapeutic composition comprising the isolated nucleic acid encoding a bi-specific fusion protein of the present disclosure; the bi-specific fusion protein of the present disclosure; the modified cell comprising the isolated nucleic acid encoding the bi-specific fusion protein of the present disclosure; or a composition comprising the isolated nucleic acid encoding a bi-specific fusion protein or the modified cell or population of modified cells comprising the bi-specific fusion protein of the present disclosure.

In some embodiments, the method comprises administering to the subject an immunotherapeutic composition comprising an isolated nucleic acid molecule encoding a bi-specific fusion protein comprising an antigen-binding domain that selectively binds a tumor-associated carbohydrate antigen (TACA) and an immune cell recognition domain that specifically binds a receptor on an immune effector cell.

In some embodiments, the antigen binding domain comprises a TACA-binding domain derived from a lectin. In one embodiment, the antigen binding domain comprises more than one (e.g., multiple) TACA binding domains. In one embodiment, the antigen binding domain comprises two, three, four, five, six, seven, eight, nine, or ten TACA binding domains. In one embodiment, the TACA binding domains are operably linked by a linker. In one embodiment, the linker is selected from the group consisting of a peptide linker, a non-peptide linker, a chemical unit, a hindered cross-linker, a non-hindered cross-linker; optional a peptide linker. In one embodiment, the peptide linker is at least 4, at least 6, at least 8, at least 10, at least 12, at least 15, or at least 15 amino acids in length. In one embodiment, the peptide linker is a glycine-serine linker. In one embodiment, the linker comprises the amino acid sequence selected from the group consisting of SEQ ID NO: 128, SEQ ID NO: 129, SEQ ID NO: 127, SEQ ID NO: 130, SEQ ID NO: 131, and SEQ ID NO: 132. In one embodiment, the linker comprises the amino acid sequence of SEQ ID NO: 127. In one embodiment, the linker comprises the amino acid sequence of SEQ ID NO: 131.

In some embodiments, the antigen binding domain that selectively binds a tumor-associated carbohydrate antigen (TACA) of the present disclosure comprises at least two TACA binding domains from a lectin selected from a galectin, a siglec, a selectin; a C-type lectin; CD301, a polypeptide N-acetylgalactosaminyltransferase (ppGacNA-T), L-PHA (Phaseolus vulgaris leukoagglutinin); E-PHA (Phaseolus vulgaris erythroagglutinen); tomato lectin (Lycopersicon esculentum lectin; LEA); peanut lectin (Arachis hypogaea Agglutinin; PNA); potato lectin (Solanum tuberosum lectin), pokeweed mitogen (Phytolacca American lectin), wheat germ agglutinin (Triticum Vulgaris lectin); Artocarpus polyphemus lectin (Jacalin letin); Vicia villosa Agglutinin (VVA); Helix pomatia Agglutinin (HPA); Wisteria floribunda Agglutinin (WFA); Sambucus nigra Agglutinin (SNA), BC2L-CNt (lectin from the gram negative bacteria Burkholderia cenocepacia), Maackia amurensis leukoagglutinin (MAL), Psathyrella velutina (PVL), Sclerotium rolfsii lectin (SRL), Eucheuma serra agglutinin (ESA), CLEC17A (Prolectin), Aleuria aurantia lectin, Sambucus sieboldiana lectin (SSA), Glechoma hederacea lectin (Gleheda), Morus nigra agglutinin (Morniga G), Salvia sclarea lectin, Salvia bogotensis lectin, Salvia horminum lectin, Clerodendrum trichotomum lectin, Moluccella laevis lectin, Griffonia simplicifolia (GsLA4), Psophocarpus tetragonolobus (acidic WBAI), Abrus precatorius lectin, Amaranthus caudatus lectin, Amaranthus leucocarpus lectin, Laelia autumnalis lectin, Artocarpus integrifolia lectin, Maclura pomifera lectin, Artocarpus lakoocha lectin, Dolichos biflorus agglutinin, Dolichos biflorus lectin, Glycine max lectin, and Agaricus bisporus lectin.

In some embodiments of the method of treating cancer disclosed herein, the antigen binding domain of the bi-specific fusion protein comprises the amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146. In one embodiment, the antigen-binding domain comprises an amino acid sequence having at least 90% homology to SEQ ID NO: 100, 101, 102, 103, 104, 105, 106, 107, 108, 109, 133, 134, 135, 136, 136, 137, 139, 140, 141, 142, 143, 144, 145, 152, or 146.

In some embodiments of the method of treating cancer disclosed herein, the bi-specific fusion protein comprises an amino acid sequence selected from SEQ ID NOs: 1-5, 10-34, 39-42, 47-50, 55-58, and 63-66. Alternatively, the bi-specific fusion protein comprises an amino acid sequence having at least 90% sequence identity to an amino acid sequence selected from SEQ ID NOs: 1-5, 10-34, 39-42, 47-50, 55-58, and 63-66. In some embodiments, the bi-specific fusion protein comprises the amino acid sequence selected from SEQ ID NO: 3-5, 10-13, 18-21, 26-34, 39-42, 47-50, 55-58, or 63-66. In some embodiments, the bi-specific fusion protein comprises the amino acid sequence selected from SEQ ID NO: 3-5, 11-13, 19-21, 28-30, 32-34, 40-42, 48-50, 56-58, or 64-66. In some embodiments, the bi-specific fusion protein comprises an amino acid sequence having at least 90% sequence identity to an amino acid sequence selected from SEQ ID NO: 3-5, 11-13, 19-21, 28-30, 32-34, 40-42, 48-50, 56-58, or 64-66. In some embodiments, the bi-specific fusion protein disclosed in Table 2 or 3. In some embodiments, the bi-specific fusion protein comprising the amino acid sequence of SEQ ID NOs: 31-34, 39-42, 47-50, 55-58, 63, or 64.

In some embodiments, the bi-specific fusion protein exhibits enhanced binding to Thomsen-nouveau (Tn) antigen (Tn antigen) expressing tumor cells when compared to a bi-specific fusion protein comprising a flexible linker in the antigen binding domain. In that embodiment, the flexible linker is a glycine-serine linker or a linker comprising an amino acid sequence selected from SEQ ID NO: 128, SEQ ID NO: 129, or SEQ ID NO: 127. Alternatively, the flexible linker is a glycine-serine linker or a linker comprising an amino acid sequence having at least 90% sequence identity to an amino acid sequence selected from SEQ ID NO: 128, SEQ ID NO: 129, or SEQ ID NO: 127.

In some embodiments, the bi-specific fusion protein selectively targets a Tn antigen or a β1,6GlcNAc-branched N-glycan. In some embodiments, the bi-specific fusion protein that selectively targets a Tn antigen comprises an antigen binding domain having the amino acid sequence selected from SEQ ID NO: 103-109, 142-146, or 152; or an amino acid sequence having at least 90% sequence identity to an amino acid sequence selected from SEQ ID NO: 103-109, 142-146, or 152.

In some embodiments, the bi-specific fusion protein that selectively targets a Tn antigen comprises the amino acid sequence selected from SEQ ID NOs: 26-34, 39-42, 47-50, 55-58, or 63-66; or an amino acid sequence having at least 90% sequence identity to an amino acid sequence selected from SEQ ID NOs: 26-34, 39-42, 47-50, 55-58, or 63-66. In some embodiments, the bi-specific fusion protein that selectively targets a Tn antigen comprises the amino acid sequence selected from SEQ ID NOs: 28-30, 32-34, 40-42, 48-50, 56-58, or 64-66; or an amino acid sequence having at least 90% sequence identity to an amino acid sequence selected from SEQ ID NOs: 28-30, 32-34, 40-42, 48-50, 56-58, or 64-66.

In some embodiments, the bi-specific fusion protein that selectively targets a β1,6GlcNAc-branched N-glycan comprises an antigen binding domain having the amino acid sequence selected from SEQ ID NO: 100-102, or 133-141; or an amino acid sequence having at least 90% sequence identity to an amino acid sequence selected from SEQ ID NO: 100-102, or 133-141.

In some embodiments, the bi-specific fusion protein that selectively targets a β1,6GlcNAc-branched N-glycan comprises the amino acid sequence selected from SEQ ID NOs: 1-5 and 10-25; or an amino acid sequence having at least 90% sequence identity to an amino acid sequence selected from SEQ ID NOs: 1-25. In some embodiments, the bi-specific fusion protein that selectively targets a β1,6GlcNAc-branched N-glycan comprises the amino acid sequence selected from SEQ ID NOs: 3-5, 11-13, or 19-21; or an amino acid sequence having at least 90% sequence identity to an amino acid sequence selected from SEQ ID NOs: 3-5, 11-13, or 19-21. In some embodiments, the bi-specific fusion protein exhibits enhanced binding to β1,6GlcNAc-branched N-glycans expressing tumor cells when compared to a bi-specific fusion protein comprising a flexible linker in the antigen binding domain. In some embodiments, the flexible linker is a glycine-serine linker or a linker comprising an amino acid sequence selected from SEQ ID NO: 124, SEQ ID NO: 128, SEQ ID NO: 129, or SEQ ID NO: 127; or an amino acid sequence having at least 90% sequence identity to an amino acid sequence selected from SEQ ID NO: 124, SEQ ID NO: 128, SEQ ID NO: 129, or SEQ ID NO: 127.

In some embodiments, the bi-specific fusion protein comprises an immune cell recognition domain that selectively binds a receptor on an immune effector cell. In that embodiment, the immune effector cell can be selected from the group consisting of a T cell, a natural killer (NK) cell, a natural killer T (NKT) cell, a macrophage, a monocyte, a dendritic cell, and a neutrophil. In that embodiment, the immune effector cell can be a T cell. In another embodiment, the immune effector cell can be an NK cell. The receptor on the immune effector cell can be selected from the group consisting of T-cell receptor (TCR) alpha, TCR beta, TCR gamma, TCR delta, invariant TCR of NKT cells, CD3, CD2, CD28, CD25, CD16, NKG2D, NKG2A, CD138, KIR3DL, NKp46, MICA, and CEACAM1. Alternatively, the receptor on the immune effector cell is a T cell receptor selected from the group consisting of CD3, CD2, CD28, and CD25. In some embodiments, the immune effector cell is an NK cell, and the NK cell receptor may be selected from the group consisting of NKG2D, NKG2A, CD138, KIR3DL, NKp46, MICA, and CEACAM1.

In some embodiments, the immune cell recognition domain of the bi-specific fusion protein comprises a peptide, a protein, an antibody, a single domain antibody, a nanobody, an antibody fragment, or single-chain variable fragment (scFv) that selectively binds to a receptor on the immune effector cell. The immune cell recognition domain may comprise an scFv that may selectively bind CD3, CD2, CD28, CD25, CD16, NKG2D, NKG2A, CD138, KIR3DL, NKp46, MICA, and CEACAM1. In some embodiments, the immune cell recognition domain specifically binds CD3. Alternatively, the immune cell recognition domain may comprise the amino acid sequence of SEQ ID NOs: 149, 150 or 151. In some embodiments, the immune cell recognition domain may comprise amino acid sequence having at least 90% sequence identity to the amino acid sequence of SEQ ID NO: 149, 150, or 151.

Alternatively, the immune cell recognition domain comprises an antibody Fc domain, optionally an Fc region of an IgG molecule. In one embodiment, the bi-specific fusion protein is an Fc fusion protein comprising the antigen binding domain that selectively binds a tumor-associated carbohydrate antigen (TACA). In some embodiments, the immune cell recognition domain is an antibody Fc domain and a domain that specifically binds CD3. In another embodiment, the immune cell recognition domain comprises the constant region domains CH2 and/or CH3 of an antibody, preferably CH2 and CH3. The constant region domains CH2 and/or CH3 of an antibody may or may not comprise a hinge region.

In one aspect, the present disclosure provides a method of treating cancer in a subject in need thereof, the method comprising administering to the subject an immunotherapeutic composition comprising

In one aspect, the present disclosure provides a method of treating a cancer in a subject in need thereof, the method comprising administering to the subject a therapeutically effective composition comprising a modified cell or a population of modified cells comprising a bi-specific fusion protein that selectively binds a tumor-associated carbohydrate antigen (TACA), where the bi-specific fusion protein comprises an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, or 146; oran amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and an immune cell recognition domain that specifically binds a receptor on an immune effector cell.

In one aspect, the present disclosure provides a method of treating a cancer in a subject in need thereof, the method comprising administering to the subject a therapeutically effective composition comprising a modified cell or a population of modified cells comprising a bi-specific fusion protein that selectively binds a tumor-associated carbohydrate antigen (TACA) comprising an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and an immune cell recognition domain that specifically binds CD3 on an immune effector cell.

In one aspect, the present disclosure provides a method of treating a cancer in a subject in need thereof, the method comprising administering to the subject a therapeutically effective composition comprising a modified cell or a population of modified cells comprising a bi-specific fusion protein that selectively binds a tumor-associated carbohydrate antigen (TACA) comprising a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and a peptide, a protein, an antibody, a single domain antibody, an antibody fragment, or single-chain variable fragment (scFv) that selectively binds to a receptor on the immune effector cell.

In one aspect, the present disclosure provides a method of treating a cancer in a subject in need thereof, the method comprising administering to the subject a therapeutically effective composition comprising a modified cell or a population of modified cells comprising a bi-specific fusion protein that selectively binds a tumor-associated carbohydrate antigen (TACA) comprising a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and an Fc domain of antibody, optionally an Fc region of an IgG molecule.

In one aspect, the present disclosure provides a method of treating a cancer in a subject in need thereof, the method comprising administering to the subject a therapeutically effective composition comprising a modified cell or a population of modified cells comprising a bi-specific fusion protein that selectively binds a tumor-associated carbohydrate antigen (TACA) comprising a TACA binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 152, and 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 152, or 146; and the constant region domains CH2 and/or CH3 of an antibody, preferably CH2 and CH3, optionally with or without a hinge region.

In some embodiments of the method of treating cancer disclosed herein, the isolated nucleic acid encoding the bi-specific fusion protein comprises an expression vector; and/or an in vitro transcribed RNA. In some embodiments, the CAR selectively targets a TACA selected from the group consisting of β1, 6 branching, β1,6GlcNAc-branched N-glycans, T antigen, Tn antigen, sialyl-T epitopes, Tn epitopes, sialyl-Tn epitopes, α2, 6 sialylation, Sialylation, sialyl-Lewis^{x/a}, di-sialyl-Lewis^{x/a}, sialyl 6-sulfo Lexis^{x}, Lewis-y (Le^{y}), Lewis Y, Globo H, GD2, GD3, GM3, and Fucosyl GM1. In one embodiment, the CAR selectively targets β1,6GlcNAc-branched N-glycans, GalNAc, Tn antigen, GalNAcα-ser, GalNAc, or GalNAco 1.

### B. CAR

In one aspect, the present disclosure provides a method of treating cancer in a subject in need thereof, the method comprising administering to the subject an immunotherapeutic composition comprising: the isolated nucleic acid encoding a TACA-CAR of the present disclosure; the TACA chimeric antigen receptor (CAR) of the present disclosure; the modified cell comprising the isolated nucleic acid encoding the TACA-CAR or the TACA CAR of the present disclosure; or a composition comprising the isolated nucleic acid encoding a TACA-CAR or the modified cell comprising the TACA-CAR of the present disclosure.

In some embodiments, the method comprises administering to the subject an immunotherapeutic composition comprising an isolated nucleic acid molecule encoding a chimeric antigen receptor (CAR) comprising: an antigen-binding domain that selectively binds a tumor-associated carbohydrate antigen (TACA), a hinge domain, a transmembrane domain, a costimulatory signaling region, and an intracellular signaling domain.

In some embodiments, the antigen binding domain comprises a TACA-binding domain derived from a lectin. In one embodiment, the antigen binding domain comprises more than one TACA binding domains. In one embodiment, the antigen binding domain comprises two, three, four, five, six, seven, eight, nine, or ten TACA binding domains. In one embodiment, the TACA binding domains are operably linked by a linker. In one embodiment, the linker is selected from the group consisting of a peptide linker, a non-peptide linker, a chemical unit, a hindered cross-linker, a non-hindered cross-linker; optional a peptide linker. In one embodiment, the peptide linker is at least 4, at least 6, at least 8, at least 10, at least 12, at least 15, or at least 15 amino acids in length. In one embodiment, the peptide linker is a glycine-serine linker. In one embodiment, the linker comprises the amino acid sequence selected from the group consisting of SEQ ID NO: 128, SEQ ID NO: 129, SEQ ID NO: 127, SEQ ID NO: 130, SEQ ID NO: 131, and SEQ ID NO: 132. In one embodiment, the linker comprises the amino acid sequence of SEQ ID NO: 127. In one embodiment, the linker comprises the amino acid sequence of SEQ ID NO: 131.

In some embodiments, the antigen binding domain that selectively binds a tumor-associated carbohydrate antigen (TACA) of the present disclosure comprises at least two TACA binding domains from a lectin selected from a galectin, a siglec, a selectin; a C-type lectin; CD301, L-PHA (Phaseolus vulgaris leukoagglutinin); E-PHA (Phaseolus vulgaris erythroagglutinen); tomato lectin (Lycopersicon esculentum lectin; LEA); peanut lectin (Arachis hypogaea Agglutinin; PNA); potato lectin (Solanum tuberosum lectin), pokeweed mitogen (Phytolacca American lectin), wheat germ agglutinin (Triticum Vulgaris lectin); Artocarpus polyphemus lectin (Jacalin letin); Vicia villosa Agglutinin (VVA); Helix pomatia Agglutinin (HPA); Wisteria floribunda Agglutinin (WFA); Sambucus nigra Agglutinin (SNA), BC2L-CNt (lectin from the gram negative bacteria Burkholderia cenocepacia), Maackia amurensis leukoagglutinin (MAL), Psathyrella velutina (PVL), Sclerotium rolfsii lectin (SRL), Eucheuma serra agglutinin (ESA), CLEC17A (Prolectin), Aleuria aurantia lectin, Sambucus sieboldiana lectin (SSA), Glechoma hederacea lectin (Gleheda), Morus nigra agglutinin (Morniga G), Salvia sclarea lectin, Salvia bogotensis lectin, Salvia horminum lectin, Clerodendrum trichotomum lectin, Moluccella laevis lectin, Griffonia simplicifolia (GsLA4), Psophocarpus tetragonolobus (acidic WBAI), Abrus precatorius lectin, Amaranthus caudatus lectin, Amaranthus leucocarpus lectin, Laelia autumnalis lectin, Artocarpus integrifolia lectin, Maclura pomifera lectin, Artocarpus lakoocha lectin, Dolichos biflorus agglutinin, Dolichos biflorus lectin, Glycine max lectin, and Agaricus bisporus lectin.

In some embodiments of the method of treating cancer disclosed herein, the antigen binding domain of the TACA CAR comprises the amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, or 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 152, 133, 134, 135, 136, 136, 137, 139, 140, 141, 142, 143, 144, 145, or 146. In one embodiment, the antigen-binding domain comprises an amino acid sequence having at least 90% homology to SEQ ID NO: 100, 101, 102, 103, 104, 105, 106, 107, 108, 109, 152, 133, 134, 135, 136, 136, 137, 139, 140, 141, 142, 143, 144, 145, or 146.

In some embodiments of the method of treating cancer disclosed herein, the transmembrane domain comprises a transmembrane region of a molecule selected from the group consisting of T-cell receptor (TCR)-alpha, TCR-beta, CD3-zeta, CD3-epsilon, CD4, CD5, CD8, CD9, CD16, CD22, CD28, CD33, CD37, CD45, CD64, CD80, CD86, CD134 (Ox40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, and TLR9. In one embodiment, the transmembrane domain comprises a CD8 transmembrane domain. In one embodiment, the transmembrane domain comprises the amino acid sequence of SEQ ID NO: 148.

In some embodiments of the method of treating cancer disclosed herein, the costimulatory domain is a costimulatory domain of a molecule selected from the group consisting of CD27, CD28, 4-IBB (CD137), OX40, CD30, CD40, PD-1, ICOS, lymphocyte function-associated antigen-1 (LFA-1), CD2, CD7, CD8, LIGHT, NKG2C, B7-H3, a ligand that specifically binds with CD83, DAP10, DAP12, Lck, Fas, and a combination thereof. In one embodiment, the costimulatory domain comprises a 4-1BB costimulatory domain or the amino acid sequence of SEQ ID NO: 114; or a CD28 costimulatory domain or the amino acid sequence of SEQ ID NO: 113; or a 4-1BB costimulatory domain and a CD28 costimulatory domain.

In some embodiments of the method of treating cancer disclosed herein, the intracellular domain comprises the intracellular signaling domain of a molecule selected from the group consisting of T cell receptor (TCR) zeta, FcR-gamma, FcR-beta, CD3-gamma, CD3-delta, CD3-epsilon, CD3-zeta, CD8, CD5, CD22, CD79a, CD79b, and CD66d. In one embodiment, the intracellular signaling domain comprises a CD3zeta signalling domain; or the amino acid sequence of SEQ ID NO: 115.

In some embodiments, the TACA CAR further comprises a hinge domain. In one embodiment, the hinge domain is a protein selected from the group consisting of a CD8a, an Fc fragment of an antibody, a hinge region of an antibody, a CH2 region of an antibody, a CH3 region of an antibody, and an artificial spacer sequence. In one embodiment, the hinge domain is a CD8a hinge domain or wherein the hinge domain comprises the amino acid sequence of SEQ ID NO: 147. In one embodiment, the hinge domain comprises an amino acid sequence selected from the group consisting of SEQ ID NO: 119, 124, 127, 128, 129, 130, 131, 132, and 147.

In one aspect, the present disclosure provides a method of treating cancer in a subject in need thereof, the method comprising administering to the subject an immunotherapeutic composition comprising an isolated nucleic acid molecule encoding a chimeric antigen receptor (CAR) comprising an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99; or an amino acid sequence having at least 90%, at least 91%, at least 92%, at least 93%, at least 94%, at least 95%, at least 96%, at least 97%, at least 98%, at least 99% sequence identity to an amino acid sequence set forth in SEQ ID NOs: 71, 72, 73, 74, 75, 76, 77, 78, 79, 80, 81, 82, 83, 84, 85, 86, 87, 88, 89, 90, 91, 92, 93, 94, 95, 96, 97, 98, or 99.

In some embodiments of the method of treating cancer disclosed herein, the isolated nucleic acid encoding the CAR comprises an expression vector; and/or an in vitro transcribed RNA. In some embodiments, the CAR selectively targets a TACA selected from the group consisting of β1, 6 branching, β1,6GlcNAc-branched N-glycans, T antigen, Tn antigen, sialyl-T epitopes, Tn epitopes, sialyl-Tn epitopes, α2, 6 sialylation, Sialylation, sialyl-Lewis^{x/a}, di-sialyl-Lewis^{x/a}, sialyl 6-sulfo Lexis^{x}, Lewis-y (Le^{y}), Lewis Y, Globo H, GD2, GD3, GM3, and Fucosyl GM1. In one embodiment, the CAR selectively targets β1,6GlcNAc-branched N-glycans, GalNAc, Tn antigen, GalNAcα-ser, GalNAc, or GalNAco 1.

In one aspect, the present disclosure provides a method of treating a cancer in a subject in need thereof, the method comprising administering to the subject a therapeutically effective composition comprising a modified cell comprising a chimeric antigen receptor that selectively binds a tumor-associated carbohydrate antigen (TACA), wherein the CAR comprises an antigen binding domain selected from the group consisting of SEQ ID NOs: 100, 101, 102, 103, 104, 105, 106, 107, 108, 109, 152, 133, 134, 135, 136, 136, 137, 139, 140, 141, 142, 143, 144, 145, or 146; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 152, 133, 134, 135, 136, 136, 137, 139, 140, 141, 142, 143, 144, 145, or 146; a CD8 a hinge domain; a CD8 transmembrane domain; a CD28 costimulatory and/or a 4-1BB costimulatory domain; and a CD3 zeta intracellular signaling domain.

### C. Cancers

In one aspect, the present disclosure provides a method of treating cancer. The method may be used to treat any cancer, including a hematological malignancy, a solid tumor, a primary or a metastasizing tumor.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumor s/cancers and pediatric tumors/cancers are also included.

In some embodiments, the cancer is selected from the group consisting of a hematological malignancy, a solid tumor, a primary or a metastasizing tumor, a leukemia, a carcinoma, a blastoma, a sarcoma, a leukemia, lymphoid malignancies, a melanoma and a lymphoma, benign and malignant tumors, and malignancies e.g., sarcomas, carcinomas, and melanomas. Adult tumor s/cancers and pediatric tumors/cancers are also included. In some embodiment, the cancer may be tumors that are not vascularized, or not yet substantially vascularized, as well as vascularized tumors. The cancer may comprise non-solid tumors (such as hematological tumors, for example, leukemias and lymphomas) or may comprise solid tumors. In some embodiments, the cancer is selected from the group consisting of a hematological malignancy, a solid tumor, a primary or a metastasizing tumor, a leukemia, a carcinoma, a blastoma, a sarcoma, a leukemia, lymphoid malignancies, a melanoma and a lymphoma.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

An exemplary type of cancer to be treated with the modified immune cells (e.g., modified T cells comprising a TACA CAR) or pharmaceutical compositions of the present disclosure include non-small cell lung cancer. Lung cancer is a leading cause of cancer-related mortality around the world and remains a significant unmet need despite advances in therapy. Non-small cell lung cancer (NSCLC) accounts for 85% of all lung cancer cases in the US, with a significant proportion of the remaining 15% being small cell lung cancers (SCLC). Zappa et al. Transl Lung Cancer Res, 5(3):288-300 (2016); Alvarado-Luna et al. Transl Lung Cancer Res, 5(1):26-38) (2016). Surgical resection remains the single most consistent and successful option for localized NSCLC; however, close to 70% of patients with lung cancer present with locally advanced or metastatic disease at the time of diagnosis. Molina et al., Mayo Clin Proc, 83(5):584-594 (2008). Overall, the prognosis for lung cancer patients is poor, with 5-year relative survival less than 18%. The median OS time for patients with stage IV NSCLC is 4 months, while 1- and 5-year survival is less than 16% and 2%, respectively. Cetin et al. Clin Epidemiol, 3:139-148 (2011).

An exemplary type of cancer to be treated with the modified immune cells (e.g., modified T cells comprising a TACA CAR) or pharmaceutical compositions of the invention include pancreatic adenocarcinoma. Pancreatic ductal adenocarcinoma is a highly lethal malignancy. It is the fourth leading cause of cancer-related death in the United States with approximately 45,000 new cases per year. Surgical resection is the only potentially curative treatment, however with tire majority of patients presenting with advanced disease only 15-20% of patients are candidates for surgical intervention. Fogel et al., Am J Gastroenterology, 112(4):537-555 (2017). Overall, prognosis is poor even with surgical intervention: the five-year survival with surgery is approximately 25% for node-negative and 10% for node-positive disease. With the majority of patients presenting with unresectable disease, chemotherapy is the mainstay of treatment.

An exemplary type of cancer to be treated with the modified immune cells (e.g., modified T cells comprising a TACA CAR) or pharmaceutical compositions of the invention include epithelial ovarian cancer. Epithelial ovarian cancers generally include fallopian tube malignancies as well as primary peritoneal cancers. More than 70% of women with epithelial ovarian cancer present with advanced disease at the time of first diagnosis. Although patients with advanced disease can achieve complete remission after surgical cytoreduction and platinum- and taxane-based chemotherapy, up to 80% eventually experience recurrence. Herzog et al., Gynecol Oncol Res Pract, 4:13 (2017).

In some embodiments, it may be advantageous to administer a TACA-binding lectin and a lectin-binding composition (e.g., T cell engineered to express an anti-lectin CAR). First, this method can have the ability to time limit the T cell response as the half-life of the lectin is much shorter than the engineered T cell. The engineered T cells may remain for years, but without the lectin, the T cells would be inactive, thereby allowing for easier targeting of solid cancers by limiting persistence of the response.

In some embodiments, a population of modified immune cells are administered to the subject. In one embodiment, the population of modified immune cells comprises immune cells selected from the group consisting of natural killer (NK) cells, NKT cells, and T cells. In some exemplary embodiments, the population of modified immune cells comprises modified T cells. In some embodiments, the modified immune cells are autologous or heterologous immune cells.

The present disclosure provides a type of cellular therapy where T cells are genetically modified to express a peptide of the invention, and the cell is infused to a recipient in need thereof. In certain embodiments, the infused cell is able to kill tumor cells in the recipient. Unlike antibody therapies, the modified cells are able to replicate in vivo resulting in long-term persistence that can lead to sustained tumor control. In one embodiment, the modified cells disclosed herein can undergo robust in vivo T cell expansion and can persist for an extended amount of time. In another embodiment, the modified T cells of the invention evolve into specific memory T cells that can be reactivated to inhibit any additional tumor formation or growth. For example, modified T cells of the invention can undergo robust in vivo T cell expansion and persist at high levels for an extended amount of time in blood and bone marrow and form specific memory T cells.

### D. Administration

The administration of the modified immune cells of the present disclosure may be administered by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrap eri cardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal. In some embodiments, the administration of the modified immune cells of the present disclosure may be carried out in any convenient manner known to those of skill in the art.

In some embodiments, the administering may be performed via intratumoral delivery, via intravenous delivery, or via intraperitoneal delivery. The amount of modified immune cells (e.g., modified T cells) to be administered to a subject in need is, generally, a therapeutically effective amount. Administration of the cells of the present disclosure may be combined with other methods useful to treat the desired disease or condition as determined by those of skill in the art.

The modified immune cells of the present disclosure to be administered may be autologous, with respect to the subject undergoing therapy or heterologous. The administration of the immune cells of the present disclosure may be carried out in any convenient manner known to those of skill in the art. The immune cells of the present disclosure may be administered to a subject by aerosol inhalation, injection, ingestion, transfusion, implantation, or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the immune cells of the present disclosure are injected directly into a site of inflammation in the subject, a local disease site in the subject, a lymph node, an organ, a tumor, and the like.

The compositions of the present invention may be administered either alone, or as a pharmaceutical composition in combination with diluents and/or with other components such as IL-2 or other cytokines or cell populations. Briefly, pharmaceutical compositions of the present invention may comprise a composition as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount,” “an anti-tumor effective amount,” “an tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (iv.) injection, or intraperitoneally. In one embodiment, the compositions of the present invention are administered to a patient by intradermal or subcutaneous injection. In another embodiment, the compositions of the present invention are administered by i.v. injection. In certain embodiments, the compositions of be injected directly into a tumor or lymph node.

In certain embodiments, the compositions are administered to a patient in conjunction with (e.g., before, simultaneously or following) any number of relevant treatment modalities, including but not limited to treatment with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321 (1991); Bierer et al., Curr. Opin. Immun. 5:763-773 (1993). In a further embodiment, the compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In certain embodiments, the composition of the invention is administered during surgical resection or debulking of a tumor or diseased tissue. For example, in subjects undergoing surgical treatment of diseased tissue or tumor, the composition may be administered to the site in order to further treat the tumor. In one embodiment, the method comprises administering to the subject a scaffold comprising a peptide comprising a TACA-binding domain, a nucleic acid molecule encoding a peptide comprising a TACA-binding domain, a cell modified to express a peptide comprising a TACA-binding domain, or a combination thereof.

Subjects to which administration of the compositions and pharmaceutical compositions of the invention is contemplated include, but are not limited to, humans and other primates, mammals including commercially relevant mammals such as non-human primates, cattle, pigs, horses, sheep, cats, and dogs.

### E. Dosage

In some embodiments, the modified immune cells are administered at a desired dosage, which in certain aspects include a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In certain aspects, the desired dose is a desired number of cells or a desired number of cells per unit of body weight of the subject to whom the cells are administered, e.g., cells/kg. In certain aspects, the desired dose is at or above a minimum number of cells or minimum number of cells per unit of body weight. In certain aspects, among the total cells, administered at the desired dose, the individual populations or sub-types are present at or near a desired output ratio (sudi as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4⁺ cells and/or a desired dose of CD8⁺ cells. In certain aspects, the desired dose is a desired number of cells of the sub-type or population, or a desired number of such cells per unit of body weight of the subject to whom the cells are administered, e.g., cells/kg.

In certain aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the modified immune cells, or individual populations of sub-types of immune cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual subpopulations of cells is within a range of between at or about 1×10⁵ cells/kg to about 1×10f11 cells/kg, 10⁴, and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁴ and 10⁶ T cells/kg body weight, for example, at or about 1×10⁴ T cells/kg, 1.5×10⁴ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁴ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10s cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁴ total cells to about 5×10⁴ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells. In an exemplary embodiment, a suitable dosage is from about 1×10⁷ total cells to about 3×10⁷ total cells.

In some embodiments, the dose of total cells and/or dose of individual subpopulations of cells is within a range of between at or about 1×10⁴ cells/m2 to about 1×10¹¹ cells/m². In an exemplary embodiment, the dose of total cells and/or dose of individual sub-populations of cells is within a range of between at or about 1×10⁷/m² to at or about 3×10⁷/m². In an exemplary embodiment, the dose of total cells and/or dose of individual sub-populations of cells is within a range of between at or about 1×10⁸/m² to at or about 3×10⁴/m². In some embodiments, the dose of total cells and/or dose of individual sub-populations of cells is the maximum tolerated dose by a given patient.

In some embodiments, the cells are administered at or within a certain range of error of between at or about 10⁴ and at or about 10⁹ CD4⁺ and/or CD8⁺ cells/kilograms (kg) body weight, such as between 10⁴ and 10⁶ CD4⁺ and/or CD8⁺ cells/kg body weight, for example, at or about 1×10⁴ CD4⁺ and/or CD8⁺ cells/kg, 1.5×10⁴ CD4⁺ and/or CD8⁺ cells/kg, 2×10⁴ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8⁺ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the modified immune cells are administered at or within a tolerated range of a desired output ratio of multiple cell populations or sub-types, such as CD4⁺ and CD8⁺ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9, 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion. In some embodiments, a dose of modified cells is administered to a subject in need thereof, in a fractionated dose or split dose. In such embodiments, the first dose is administered, and a subsequent dose is administered 1 or more days, 2 or more days, 3 or more days, 4 or more days, 5 or more days, 6 or more days, 7 or more days, 8 or more days, 9 or more days, 10 or more days, 11 or more days, 12 or more days, 13 or more days, 2 or more weeks, 3 or more weeks, 4 or more weeks, 5 or more weeks, or any period in between, after the first dose. For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the modified immune cells are administered as part of a combination treatment, such as simultaneously with or sequentially with, in any order, another therapeutic intervention, such as an antibody or engineered cell or receptor or agent, such as a cytotoxic or therapeutic agent. The modified immune cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al., J Immunological Methods, 285(1): 25-40 (2004). In certain embodiments, the biological activity of the cells is measured by assaying expression and/or secretion of one or more cytokines, such as CD 107a, IFNγ, IL-2, and TNF. In certain aspects, the biological activity is measured by assessing clinical outcome, such as reduction in tumor burden or load. In certain embodiments, the subject can be administered, in addition to the CAR, a secondary treatment.

In some embodiments, the subject can be administered conditioning therapy prior to CAR T cell therapy. Accordingly, the present disclosure provides a method of treatment comprising administering a conditioning therapy prior to administering CAR T therapy (e.g., modified T cells comprising a TACA CAR of the present disclosure). Administration of a conditioning therapy prior to TACA CAR T cell therapy may increase the efficacy of the TACA CAR T cell therapy. Methods of conditioning patients for T cell therapy are described in U.S. Pat. No. 9,855,298.

The dosage of the above treatments to be administered to a patient will vary with the precise nature of the condition being treated and the recipient of the treatment. The scaling of dosages for human administration can be performed according to art-accepted practices. Strategies for T cell dosing and scheduling have been discussed (Ertl et al, 2011, Cancer Res, 71:3175-81; Junghans, 2010, Journal of Translational Medicine, 8:55).

Sequences of individual domains of the CAR and the Bite of the present disclosure are found in Table 3

TABLE 3
Sequences of individual domains of
the CAR and Bi-specific fusion proteins

| SEQ ID | | |
|---|---|---|
| NO: | Description | Sequence |
| 100 | L-PHA | SNDIYFNFQRFNETNLILQRDASVSSSGQLRLTNLNGNG |
| | | EPRVGSLGRAFYSAPIQIWDNTTGTVASFATSFTFNIQVP |
| | | NNAGPADGLAFALVPVGSQPKDKGGFLGLFDGSNSNF |
| | | HTVAVEFDTLYNKDWDPTERHIGIDVNSIRSIKTTRWDF |
| | | VNGENAEVLITYDSSTNLLVASLVYPSQKTSFIVSDTVD |
| | | LKSVLPEWVSVGFSATTGINKGNVETNDVLSWSFASKL |
| | | SDGTTSEGLNLANLVLNKIL |
| 101 | L-PHAΔ1-3 | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHTV |
| | | AVEFDTLYNKDWDPTERHIGIDVNSIRSIKTTRWDFVNG |
| | | ENAEVLITYDSSTNLLVASLVYPSQKTSFIVSDTVDLKS |
| | | VLPEWVSVGFSATTGINKGNVETNDVLSWSFASKLSDG |
| | | TTSEGLNLANLVLNKIL |
| 102 | L-PHAΔ1-5 | FNFQRFNETNLILQRDASVSSSGQLRLTNLNGNGEPRVG |
| | | SLGRAFYSAPIQIWDNTTGTVASFATSFTFNIQVPNNAG |
| | | PADGLAFALVPVGSQPKDKGGFLGLFDGSNSNFHTVAV |
| | | EFDTLYNKDWDPTERHIGIDVNSIRSIKTTRWDFVNGEN |
| | | AEVLITYDSSTNLLVASLVYPSQKTSFIVSDTVDLKSVLP |
| | | EWVSVGFSATTGINKGNVETNDVLSWSFASKLSDGTTS |
| | | EGLNLANLVLNKIL |
| 103 | Extended | VHSEMLLRVQQLVQDLKKLTCQVATLNNNASTEGTCC |
| | CD301CRD | PVNWVEHQDSCYWFSHSGMSWAEAEKYCQLKNAHLV |
| | | VINSREEQNFVQKYLGSAYTWMGLSDPEGAWKWVDG |
| | | TDYATGFQNWKPGQPDDWQGHGLGGGEDCAHFHPDG |
| | | RWNDDVCQRPYHWVCEAGLGQTSQESH |
| 104 | Mutated | VHSEMLLRVQQLVQDLKKLTCQVATLNNNASTEGTCC |
| | extended | PVNWVEHQDSCYWFSHSGMSWAEAEKYCQLKNAHLV |
| | CD301CRD | VINSREEQNFVQKYLGSAYTWMGLSDPEGAWKWVDG |
| | | TDYATGFQNWKPGGPGDWQGHGLGGGGDCAHFHPDG |
| | | RWGGDVCQRPYHWVCEAGLGQTSQESH |
| 105 | CD301CRD | LTCQVATLNNNASTEGTCCPVNWVEHQDSCYWFSHSG |
| | (same as | MSWAEAEKYCQLKNAHLVVINSREEQNFVQKYLGSAY |
| | CD301CRD163- | TWMGLSDPEGAWKWVDGTDYATGFQNWKPGQPDDW |
| | 316) | QGHGLGGGEDCAHFHPDGRWNDDVCQRPYHWVCEA |
| | | GLGQTSQESH |
| 106 | mutCD301CRD | LTCQVATLNNNASTEGTCCPVNWVEHQDSCYWFSHSG |
| | | MSWAEAEKYCQLKNAHLVVINSREEQNFVQKYLGSAY |
| | | TWMGLSDPEGAWKWVDGTDYATGFQNWKPGGPGDW |
| | | QGHGLGGGGDCAHFHPDGRWGGDVCQRPYHWVCEA |
| | | GLGQTSQESH |
| 107 | CD301CRD163- | LTCQVATLNNNASTEGTCCPVNWVEHQDSCYWFSHSG |
| | 306 | MSWAEAEKYCQLKNAHLVVINSREEQNFVQKYLGSAY |
| | | TWMGLSDPEGAWKWVDGTDYATGFQNWKPGQPDDW |
| | | QGHGLGGGEDCAHFHPDGRWNDDVCQRPYHWVCEA |
| 108 | CD301CRD181- | CPVNWVEHQDSCYWFSHSGMSWAEAEKYCQLKNAHL |
| | 316 | VVINSREEQNFVQKYLGSAYTWMGLSDPEGAWKWVD |
| | | GTDYATGFQNWKPGQPDDWQGHGLGGGEDCAHFHPD |
| | | GRWNDDVCQRPYHWVCEAGLGQTSQESH |
| 109 | CD301CRD181- | CPVNWVEHQDSCYWFSHSGMSWAEAEKYCQLKNAHL |
| | 306 | VVINSREEQNFVQKYLGSAYTWMGLSDPEGAWKWVD |
| | | GTDYATGFQNWKPGQPDDWQGHGLGGGEDCAHFHPD |
| | | GRWNDDVCQRPYHWVCEA |
| 110 | Human Serum | DAHKSEVAHRFKDLGEENFKALVLIAFAQYLQQCPFED |
| | Albumin (HSA) | HVKLVNEVTEFAKTCVADESAENCDKSLHTLFGDKLCT |
| | domain | VATLRETYGEMADCCAKQEPERNECFLQHKDDNPNLP |
| | | RLVRPEVDVMCTAFHDNEETFLKKYLYEIARRHPYFYA |
| | | PELLFFAKRYKAAFTECCQAADKAACLLPKLDELRDEG |
| | | KASSAKQRLKCASLQKFGERAFKAWAVARLSQRFPKA |
| | | EFAEVSKLVTDLTKVHTECCHGDLLECADDRADLAKYI |
| | | CENQDSISSKLKECCEKPLLEKSHCIAEVENDEMPADLP |
| | | SLAADFVESKDVCKNYAEAKDVFLGMFLYEYARRHPD |
| | | YSVVLLLRLAKTYETTLEKCCAAADPHECYAKVFDEFK |
| | | PLVEEPQNLIKQNCELFEQLGEYKFQNALLVRYTKKVP |
| | | QVSTPTLVEVSRNLGKVGSKCCKHPEAKRMPCAEDYLS |
| | | VVLNQLCVLHEKTPVSDRVTKCCTESLVNRRPCFSALE |
| | | VDETYVPKEFNAETFTFHADICTLSEKERQIKKQTALVE |
| | | LVKHKPKATKEQLKAVMDDFAAFVEKCCKADDKETCF |
| | | AEEGKKLVAASQAALGL |
| 111 | CD8 hinge and | TTTPAPRPPTPAPTIASQPLSLRPEACRPAAGGAVHTRGL |
| | transmembrane | DFACDIYIWAPLAGTCGVLLLSLVITLYC |
| | region | |
| 112 | CD8 hinge and | FVPVFLPAKPTTTPAPRPPTPAPTIASQPLSLRPEACRPA |
| | transmembrane | AGGAVHTRGLDFACDIYIWAPLAGTCGVLLLSLVITLY |
| | region | C |
| 113 | CD28 region | IEVMYPPPYLDNEKSNGTIIHVKGKHLCPSPLFPGPSKPF |
| | | WVLVVVGGVLACYSLLVTVAFIIFWVRSKRSRLLHSDY |
| | | MNMTPRRPGPTRKHYQPYAPPRDFAAYRS |
| 114 | 4-1BB signaling | KRGRKKLLYIFKQPFMRPVQTTQEEDGCSCRFPEEEEG |
| | region | GCEL |
| 115 | CD3 zeta | RVKFSRSADAPAYQQGQNQLYNELNLGRREEYDVLDK |
| | signaling region | RRGRDPEMGGKPRRKNPQEGLYNELQKDKMAEAYSEI |
| | | GMKGERRRGKGHDGLYQGLSTATKDTYDALHMQALP |
| | | PR |
| 116 | Rigid linker | (AEAAAKA)ₙ |
| 117 | Rigid linker | A(EAAAK)ₙA |
| 117 | Rigid linker | (EAAAK)ₙ |
| 119 | Rigid linker | A(EAAAK)₄ALEA(EAAAK)₄A |
| 120 | Rigid linker | PAPAP |
| 121 | Rigid linker | (AP)ₙ |
| 122 | Flexible linker | (GGGS)ₙ |
| 123 | Flexible linker | (GGGGS)ₙ |
| 124 | Flexible linker | GGGSGGGGSGGGS |
| 125 | Flexible linker | (G)ₙ |
| 126 | Flexible linker | (GS)ₙ |
| 127 | Flexible linker | GGGGSGGGGSGGGGS |
| 128 | Flexible linker | GGGGS |
| 129 | Flexible linker | GGGGSGGGGS |
| 130 | Rigid linker | AEAAAKA |
| 131 | Rigid linker | AEAAAKAAEAAAKA |
| 132 | Rigid linker | AEAAAKAAEAAAKAAEAAAKA |
| 133 | L-PHA (2) | SNDIYFNFQRFNETNLILQRDASVSSSGQLRLTNLNGNG |
| | | EPRVGSLGRAFYSAPIQIWDNTTGTVASFATSFTFNIQVP |
| | | NNAGPADGLAFALVPVGSQPKDKGGFLGLFDGSNSNF |
| | | HTVAVEFDTLYNKDWDPTERHIGIDVNSIRSIKTTRWDF |
| | | VNGENAEVLITYDSSTNLLVASLVYPSQKTSFIVSDTVD |
| | | LKSVLPEWVSVGFSATTGINKGNVETNDVLSWSFASKL |
| | | SDGTTSEGLNLANLVLNKILGGGGSGGGGSGGGGSSND |
| | | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHTV |
| | | AVEFDTLYNKDWDPTERHIGIDVNSIRSIKTTRWDFVNG |
| | | ENAEVLITYDSSTNLLVASLVYPSQKTSFIVSDTVDLKS |
| | | VLPEWVSVGFSATTGINKGNVETNDVLSWSFASKLSDG |
| | | TTSEGLNLANLVLNKIL |
| 134 | L-PHA (3) | SNDIYFNFQRFNETNLILQRDASVSSSGQLRLTNLNGNG |
| | | EPRVGSLGRAFYSAPIQIWDNTTGTVASFATSFTFNIQVP |
| | | NNAGPADGLAFALVPVGSQPKDKGGFLGLFDGSNSNF |
| | | HTVAVEFDTLYNKDWDPTERHIGIDVNSIRSIKTTRWDF |
| | | VNGENAEVLITYDSSTNLLVASLVYPSQKTSFIVSDTVD |
| | | LKSVLPEWVSVGFSATTGINKGNVETNDVLSWSFASKL |
| | | SDGTTSEGLNLANLVLNKILGGGGSGGGGSGGGGSSND |
| | | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHTV |
| | | AVEFDTLYNKDWDPTERHIGIDVNSIRSIKTTRWDFVNG |
| | | ENAEVLITYDSSTNLLVASLVYPSQKTSFIVSDTVDLKS |
| | | VLPEWVSVGFSATTGINKGNVETNDVLSWSFASKLSDG |
| | | TTSEGLNLANLVLNKILGGGGSGGGGSGGGGSSNDIYF |
| | | NFQRFNETNLILQRDASVSSSGQLRLTNLNGNGEPRVGS |
| | | LGRAFYSAPIQIWDNTTGTVASFATSFTFNIQVPNNAGP |
| | | ADGLAFALVPVGSQPKDKGGFLGLFDGSNSNFHTVAVE |
| | | FDTLYNKDWDPTERHIGIDVNSIRSIKTTRWDFVNGENA |
| | | EVLITYDSSTNLLVASLVYPSQKTSFIVSDTVDLKSVLPE |
| | | WVSVGFSATTGINKGNVETNDVLSWSFASKLSDGTTSE |
| | | GLNLANLVLNKIL |
| 135 | L-PHA (4) | SNDIYFNFQRFNETNLILQRDASVSSSGQLRLTNLNGNG |
| | | EPRVGSLGRAFYSAPIQIWDNTTGTVASFATSFTFNIQVP |
| | | NNAGPADGLAFALVPVGSQPKDKGGFLGLFDGSNSNF |
| | | HTVAVEFDTLYNKDWDPTERHIGIDVNSIRSIKTTRWDF |
| | | VNGENAEVLITYDSSTNLLVASLVYPSQKTSFIVSDTVD |
| | | LKSVLPEWVSVGFSATTGINKGNVETNDVLSWSFASKL |
| | | SDGTTSEGLNLANLVLNKILGGGGSGGGGSGGGGSSND |
| | | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHTV |
| | | AVEFDTLYNKDWDPTERHIGIDVNSIRSIKTTRWDFVNG |
| | | ENAEVLITYDSSTNLLVASLVYPSQKTSFIVSDTVDLKS |
| | | VLPEWVSVGFSATTGINKGNVETNDVLSWSFASKLSDG |
| | | TTSEGLNLANLVLNKILGGGGSGGGGSGGGGSSNDIYF |
| | | NFQRFNETNLILQRDASVSSSGQLRLTNLNGNGEPRVGS |
| | | LGRAFYSAPIQIWDNTTGTVASFATSFTFNIQVPNNAGP |
| | | ADGLAFALVPVGSQPKDKGGFLGLFDGSNSNFHTVAVE |
| | | FDTLYNKDWDPTERHIGIDVNSIRSIKTTRWDFVNGENA |
| | | EVLITYDSSTNLLVASLVYPSQKTSFIVSDTVDLKSVLPE |
| | | WVSVGFSATTGINKGNVETNDVLSWSFASKLSDGTTSE |
| | | GLNLANLVLNKILGGGGSGGGGSGGGGSSNDIYFNFQR |
| | | FNETNLILQRDASVSSSGQLRLTNLNGNGEPRVGSLGRA |
| | | FYSAPIQIWDNTTGTVASFATSFTFNIQVPNNAGPADGL |
| | | AFALVPVGSQPKDKGGFLGLFDGSNSNFHTVAVEFDTL |
| | | YNKDWDPTERHIGIDVNSIRSIKTTRWDFVNGENAEVLI |
| | | TYDSSTNLLVASLVYPSQKTSFIVSDTVDLKSVLPEWVS |
| | | VGFSATTGINKGNVETNDVLSWSFASKLSDGTTSEGLN |
| | | LANLVLNKIL |
| 136 | L-PHAΔ1-3 (2) | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHTV |
| | | AVEFDTLYNKDWDPTERHIGIDVNSIRSIKTTRWDFVNG |
| | | ENAEVLITYDSSTNLLVASLVYPSQKTSFIVSDTVDLKS |
| | | VLPEWVSVGFSATTGINKGNVETNDVLSWSFASKLSDG |
| | | TTSEGLNLANLVLNKILGGGGSGGGGSGGGGSIYFNFQ |
| | | RFNETNLILQRDASVSSSGQLRLTNLNGNGEPRVGSLGR |
| | | AFYSAPIQIWDNTTGTVASFATSFTFNIQVPNNAGPADG |
| | | LAFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKIL |
| 137 | L-PHAΔ1-3 (3) | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHTV |
| | | AVEFDTLYNKDWDPTERHIGIDVNSIRSIKTTRWDFVNG |
| | | ENAEVLITYDSSTNLLVASLVYPSQKTSFIVSDTVDLKS |
| | | VLPEWVSVGFSATTGINKGNVETNDVLSWSFASKLSDG |
| | | TTSEGLNLANLVLNKILGGGGSGGGGSGGGGSIYFNFQ |
| | | RFNETNLILQRDASVSSSGQLRLTNLNGNGEPRVGSLGR |
| | | AFYSAPIQIWDNTTGTVASFATSFTFNIQVPNNAGPADG |
| | | LAFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKILGGGGSGGGGSGGGGSIYFNFQRFNET |
| | | NLILQRDASVSSSGQLRLTNLNGNGEPRVGSLGRAFYS |
| | | APIQIWDNTTGTVASFATSFTFNIQVPNNAGPADGLAFA |
| | | LVPVGSQPKDKGGFLGLFDGSNSNFHTVAVEFDTLYNK |
| | | DWDPTERHIGIDVNSIRSIKTTRWDFVNGENAEVLITYD |
| | | SSTNLLVASLVYPSQKTSFIVSDTVDLKSVLPEWVSVGF |
| | | SATTGINKGNVETNDVLSWSFASKLSDGTTSEGLNLAN |
| | | LVLNKIL |
| 138 | L-PHAΔ1-3 (4) | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHTV |
| | | AVEFDTLYNKDWDPTERHIGIDVNSIRSIKTTRWDFVNG |
| | | ENAEVLITYDSSTNLLVASLVYPSQKTSFIVSDTVDLKS |
| | | VLPEWVSVGFSATTGINKGNVETNDVLSWSFASKLSDG |
| | | TTSEGLNLANLVLNKILGGGGSGGGGSGGGGSIYFNFQ |
| | | RFNETNLILQRDASVSSSGQLRLTNLNGNGEPRVGSLGR |
| | | AFYSAPIQIWDNTTGTVASFATSFTFNIQVPNNAGPADG |
| | | LAFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKILGGGGSGGGGSGGGGSIYFNFQRFNET |
| | | NLILQRDASVSSSGQLRLTNLNGNGEPRVGSLGRAFYS |
| | | APIQIWDNTTGTVASFATSFTFNIQVPNNAGPADGLAFA |
| | | LVPVGSQPKDKGGFLGLFDGSNSNFHTVAVEFDTLYNK |
| | | DWDPTERHIGIDVNSIRSIKTTRWDFVNGENAEVLITYD |
| | | SSTNLLVASLVYPSQKTSFIVSDTVDLKSVLPEWVSVGF |
| | | SATTGINKGNVETNDVLSWSFASKLSDGTTSEGLNLAN |
| | | LVLNKILGGGGSGGGGSGGGGSIYFNFQRFNETNLILQR |
| | | DASVSSSGQLRLTNLNGNGEPRVGSLGRAFYSAPIQIWD |
| | | NTTGTVASFATSFTFNIQVPNNAGPADGLAFALVPVGS |
| | | QPKDKGGFLGLFDGSNSNFHTVAVEFDTLYNKDWDPT |
| | | ERHIGIDVNSIRSIKTTRWDFVNGENAEVLITYDSSTNLL |
| | | VASLVYPSQKTSFIVSDTVDLKSVLPEWVSVGFSATTGI |
| | | NKGNVETNDVLSWSFASKLSDGTTSEGLNLANLVLNKI |
| | | L |
| 139 | L-PHAΔ1-5 (2) | FNFQRFNETNLILQRDASVSSSGQLRLTNLNGNGEPRVG |
| | | SLGRAFYSAPIQIWDNTTGTVASFATSFTFNIQVPNNAG |
| | | PADGLAFALVPVGSQPKDKGGFLGLFDGSNSNFHTVAV |
| | | EFDTLYNKDWDPTERHIGIDVNSIRSIKTTRWDFVNGEN |
| | | AEVLITYDSSTNLLVASLVYPSQKTSFIVSDTVDLKSVLP |
| | | EWVSVGFSATTGINKGNVETNDVLSWSFASKLSDGTTS |
| | | EGLNLANLVLNKILGGGGSGGGGSGGGGSFNFQRFNET |
| | | NLILQRDASVSSSGQLRLTNLNGNGEPRVGSLGRAFYS |
| | | APIQIWDNTTGTVASFATSFTFNIQVPNNAGPADGLAFA |
| | | LVPVGSQPKDKGGFLGLFDGSNSNFHTVAVEFDTLYNK |
| | | DWDPTERHIGIDVNSIRSIKTTRWDFVNGENAEVLITYD |
| | | SSTNLLVASLVYPSQKTSFIVSDTVDLKSVLPEWVSVGF |
| | | SATTGINKGNVETNDVLSWSFASKLSDGTTSEGLNLAN |
| | | LVLNKIL |
| 140 | L-PHAΔ1-5 (3) | FNFQRFNETNLILQRDASVSSSGQLRLTNLNGNGEPRVG |
| | | SLGRAFYSAPIQIWDNTTGTVASFATSFTFNIQVPNNAG |
| | | PADGLAFALVPVGSQPKDKGGFLGLFDGSNSNFHTVAV |
| | | EFDTLYNKDWDPTERHIGIDVNSIRSIKTTRWDFVNGEN |
| | | AEVLITYDSSTNLLVASLVYPSQKTSFIVSDTVDLKSVLP |
| | | EWVSVGFSATTGINKGNVETNDVLSWSFASKLSDGTTS |
| | | EGLNLANLVLNKILGGGGSGGGGSGGGGSFNFQRFNET |
| | | NLILQRDASVSSSGQLRLTNLNGNGEPRVGSLGRAFYS |
| | | APIQIWDNTTGTVASFATSFTFNIQVPNNAGPADGLAFA |
| | | LVPVGSQPKDKGGFLGLFDGSNSNFHTVAVEFDTLYNK |
| | | DWDPTERHIGIDVNSIRSIKTTRWDFVNGENAEVLITYD |
| | | SSTNLLVASLVYPSQKTSFIVSDTVDLKSVLPEWVSVGF |
| | | SATTGINKGNVETNDVLSWSFASKLSDGTTSEGLNLAN |
| | | LVLNKILGGGGSGGGGSGGGGSFNFQRFNETNLILQRD |
| | | ASVSSSGQLRLTNLNGNGEPRVGSLGRAFYSAPIQIWDN |
| | | TTGTVASFATSFTFNIQVPNNAGPADGLAFALVPVGSQP |
| | | KDKGGFLGLFDGSNSNFHTVAVEFDTLYNKDWDPTER |
| | | HIGIDVNSIRSIKTTRWDFVNGENAEVLITYDSSTNLLVA |
| | | SLVYPSQKTSFIVSDTVDLKSVLPEWVSVGFSATTGINK |
| | | GNVETNDVLSWSFASKLSDGTTSEGLNLANLVLNKIL |
| 141 | L-PHAΔ1-5 (3) | FNFQRFNETNLILQRDASVSSSGQLRLTNLNGNGEPRVG |
| | | SLGRAFYSAPIQIWDNTTGTVASFATSFTFNIQVPNNAG |
| | | PADGLAFALVPVGSQPKDKGGFLGLFDGSNSNFHTVAV |
| | | EFDTLYNKDWDPTERHIGIDVNSIRSIKTTRWDFVNGEN |
| | | AEVLITYDSSTNLLVASLVYPSQKTSFIVSDTVDLKSVLP |
| | | EWVSVGFSATTGINKGNVETNDVLSWSFASKLSDGTTS |
| | | EGLNLANLVLNKILGGGGSGGGGSGGGGSFNFQRFNET |
| | | NLILQRDASVSSSGQLRLTNLNGNGEPRVGSLGRAFYS |
| | | APIQIWDNTTGTVASFATSFTFNIQVPNNAGPADGLAFA |
| | | LVPVGSQPKDKGGFLGLFDGSNSNFHTVAVEFDTLYNK |
| | | DWDPTERHIGIDVNSIRSIKTTRWDFVNGENAEVLITYD |
| | | SSTNLLVASLVYPSQKTSFIVSDTVDLKSVLPEWVSVGF |
| | | SATTGINKGNVETNDVLSWSFASKLSDGTTSEGLNLAN |
| | | LVLNKILGGGGSGGGGSGGGGSFNFQRFNETNLILQRD |
| | | ASVSSSGQLRLTNLNGNGEPRVGSLGRAFYSAPIQIWDN |
| | | TTGTVASFATSFTFNIQVPNNAGPADGLAFALVPVGSQP |
| | | KDKGGFLGLFDGSNSNFHTVAVEFDTLYNKDWDPTER |
| | | HIGIDVNSIRSIKTTRWDFVNGENAEVLITYDSSTNLLVA |
| | | SLVYPSQKTSFIVSDTVDLKSVLPEWVSVGFSATTGINK |
| | | GNVETNDVLSWSFASKLSDGTTSEGLNLANLVLNKILG |
| | | GGGSGGGGSGGGGSFNFQRFNETNLILQRDASVSSSGQ |
| | | LRLTNLNGNGEPRVGSLGRAFYSAPIQIWDNTTGTVASF |
| | | ATSFTFNIQVPNNAGPADGLAFALVPVGSQPKDKGGFL |
| | | GLFDGSNSNFHTVAVEFDTLYNKDWDPTERHIGIDVNSI |
| | | RSIKTTRWDFVNGENAEVLITYDSSTNLLVASLVYPSQK |
| | | TSFIVSDTVDLKSVLPEWVSVGFSATTGINKGNVETNDV |
| | | LSWSFASKLSDGTTSEGLNLANLVLNKIL |
| 142 | Extended | VHSEMLLRVQQLVQDLKKLTCQVATLNNNASTEGTCC |
| | CD301CRD- | PVNWVEHQDSCYWFSHSGMSWAEAEKYCQLKNAHLV |
| | Linker- | VINSREEQNFVQKYLGSAYTWMGLSDPEGAWKWVDG |
| | CD301CRD- | TDYATGFQNWKPGQPDDWQGHGLGGGEDCAHFHPDG |
| | | RWNDDVCQRPYHWVCEAGLGQTSQESHGGGGSGGGG |
| | | SGGGGSLTCQVATLNNNASTEGTCCPVNWVEHQDSCY |
| | | WFSHSGMSWAEAEKYCQLKNAHLVVINSREEQNFVQK |
| | | YLGSAYTWMGLSDPEGAWKWVDGTDYATGFQNWKP |
| | | GQPDDWQGHGLGGGEDCAHFHPDGRWNDDVCQRPY |
| | | HWVCEAGLGQTSQESH |
| 143 | Extended | VHSEMLLRVQQLVQDLKKLTCQVATLNNNASTEGTCC |
| | CD301CRD- | PVNWVEHQDSCYWFSHSGMSWAEAEKYCQLKNAHLV |
| | Linker- | VINSREEQNFVQKYLGSAYTWMGLSDPEGAWKWVDG |
| | CD301CRD(2) | TDYATGFQNWKPGQPDDWQGHGLGGGEDCAHFHPDG |
| | | RWNDDVCQRPYHWVCEAGLGQTSQESHGGGGSGGGG |
| | | SGGGGSLTCQVATLNNNASTEGTCCPVNWVEHQDSCY |
| | | WFSHSGMSWAEAEKYCQLKNAHLVVINSREEQNFVQK |
| | | YLGSAYTWMGLSDPEGAWKWVDGTDYATGFQNWKP |
| | | GQPDDWQGHGLGGGEDCAHFHPDGRWNDDVCQRPY |
| | | HWVCEAGLGQTSQESHGGGGSGGGGSGGGGSLTCQV |
| | | ATLNNNASTEGTCCPVNWVEHQDSCYWFSHSGMSWA |
| | | EAEKYCQLKNAHLVVINSREEQNFVQKYLGSAYTWMG |
| | | LSDPEGAWKWVDGTDYATGFQNWKPGQPDDWQGHG |
| | | LGGGEDCAHFHPDGRWNDDVCQRPYHWVCEAGLGQT |
| | | SQESH |
| 144 | CD301CRD(2) | LTCQVATLNNNASTEGTCCPVNWVEHQDSCYWFSHSG |
| | | MSWAEAEKYCQLKNAHLVVINSREEQNFVQKYLGSAY |
| | | TWMGLSDPEGAWKWVDGTDYATGFQNWKPGQPDDW |
| | | QGHGLGGGEDCAHFHPDGRWNDDVCQRPYHWVCEA |
| | | GLGQTSQESHGGGGSGGGGSGGGGSLTCQVATLNNNA |
| | | STEGTCCPVNWVEHQDSCYWFSHSGMSWAEAEKYCQ |
| | | LKNAHLVVINSREEQNFVQKYLGSAYTWMGLSDPEGA |
| | | WKWVDGTDYATGFQNWKPGQPDDWQGHGLGGGEDC |
| | | AHFHPDGRWNDDVCQRPYHWVCEAGLGQTSQESH |
| 145 | Extended | VHSEMLLRVQQLVQDLKKLTCQVATLNNNASTEGTCC |
| | CD301CRD- | PVNWVEHQDSCYWFSHSGMSWAEAEKYCQLKNAHLV |
| | Linker- | VINSREEQNFVQKYLGSAYTWMGLSDPEGAWKWVDG |
| | CD301CRD(3) | TDYATGFQNWKPGQPDDWQGHGLGGGEDCAHFHPDG |
| | | RWNDDVCQRPYHWVCEAGLGQTSQESHGGGGSGGGG |
| | | SGGGGSLTCQVATLNNNASTEGTCCPVNWVEHQDSCY |
| | | WFSHSGMSWAEAEKYCQLKNAHLVVINSREEQNFVQK |
| | | YLGSAYTWMGLSDPEGAWKWVDGTDYATGFQNWKP |
| | | GQPDDWQGHGLGGGEDCAHFHPDGRWNDDVCQRPY |
| | | HWVCEAGLGQTSQESHGGGGSGGGGSGGGGSLTCQV |
| | | ATLNNNASTEGTCCPVNWVEHQDSCYWFSHSGMSWA |
| | | EAEKYCQLKNAHLVVINSREEQNFVQKYLGSAYTWMG |
| | | LSDPEGAWKWVDGTDYATGFQNWKPGQPDDWQGHG |
| | | LGGGEDCAHFHPDGRWNDDVCQRPYHWVCEAGLGQT |
| | | SQESHGGGGSGGGGSGGGGSLTCQVATLNNNASTEGT |
| | | CCPVNWVEHQDSCYWFSHSGMSWAEAEKYCQLKNAH |
| | | LVVINSREEQNFVQKYLGSAYTWMGLSDPEGAWKWV |
| | | DGTDYATGFQNWKPGQPDDWQGHGLGGGEDCAHFHP |
| | | DGRWNDDVCQRPYHWVCEAGLGQTSQESH |
| 146 | CD301CRD(3) | LTCQVATLNNNASTEGTCCPVNWVEHQDSCYWFSHSG |
| | | MSWAEAEKYCQLKNAHLVVINSREEQNFVQKYLGSAY |
| | | TWMGLSDPEGAWKWVDGTDYATGFQNWKPGQPDDW |
| | | QGHGLGGGEDCAHFHPDGRWNDDVCQRPYHWVCEA |
| | | GLGQTSQESHGGGGSGGGGSGGGGSLTCQVATLNNNA |
| | | STEGTCCPVNWVEHQDSCYWFSHSGMSWAEAEKYCQ |
| | | LKNAHLVVINSREEQNFVQKYLGSAYTWMGLSDPEGA |
| | | WKWVDGTDYATGFQNWKPGQPDDWQGHGLGGGEDC |
| | | AHFHPDGRWNDDVCQRPYHWVCEAGLGQTSQESHGG |
| | | GGSGGGGSGGGGSLTCQVATLNNNASTEGTCCPVNWV |
| | | EHQDSCYWFSHSGMSWAEAEKYCQLKNAHLVVINSRE |
| | | EQNFVQKYLGSAYTWMGLSDPEGAWKWVDGTDYAT |
| | | GFQNWKPGQPDDWQGHGLGGGEDCAHFHPDGRWND |
| | | DVCQRPYHWVCEAGLGQTSQESH |
| 147 | CD8 hinge | TTTPAPRPPTPAPTIASQPLSLRPEACRPAAGGAVHTRGL |
| | | DFACD |
| 148 | CD8 | IYIWAPLAGTCGVLLLSLVITLYC |
| | transmembrane | |
| 149 | CD3 scFv | DIKLQQSGAELARPGASVKMSCKTSGYTFTRYTMHWV |
| | | KQRPGQGLEWIGYINPSRGYTNYNQKFKDKATLTTDKS |
| | | SSTAYMQLSSLTSEDSAVYYCARYYDDHYCLDYWGQG |
| | | TTLTVSSVEGGSGGSGGSGGSGGVDDIQLTQSPAIMSAS |
| | | PGEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 150 | CD3 Heavy | DIKLQQSGAELARPGASVKMSCKTSGYTFTRYTMHWV |
| | Chain variable | KQRPGQGLEWIGYINPSRGYTNYNQKFKDKATLTTDKS |
| | domain | SSTAYMQLSSLTSEDSAVYYCARYYDDHYCLDYWGQG |
| | | TTLTVSS |
| 151 | CD3 light chain | DIQLTQSPAIMSASPGEKVTMTCRASSSVSYMNWYQQK |
| | variable domain | SGTSPKRWIYDTSKVASGVPYRFSGSGSGTSYSLTISSM |
| | | EAEDAATYYCQQWSSNPLTFGAGTKLELK |
| 152 | CD301CRD | LTCQVATLNNNASTEGTCCPVNWVEHQDSCYWFSHSG |
| | | MSWAEAEKYCQLKNAHLVVINSREEQNFVQKYLGSAY |
| | | TWMGLSDPEGAWKWVDGTDYATGFQNWKPGQPDDW |
| | | QGHGLGGGEDCAHFHPDGRWNDDVCQRPYHWVCEA |
| | | GLGQTSQESH |
| 153 | IgG1 | DKTHTCPPCP [Comments: please confirm] |
| | hinge/region | |
| 154 | IgG1 | EPKSCDKTHTCPPCP |
| | hinge/region | |
| 155 | IgG2 | ERKCCVECPPCP |
| | hinge/region | |
| 156 | IgG3 | ELKTPLDTTHTCPRCP |
| | hinge/region | |
| 157 | IgG3 | ELKTPLGDTTHTCPRCP |
| | hinge/region | |
| 158 | IgG4 | ESKYGPPCPSCP |
| | hinge/region | |
| 159 | Fc region | |
| 160 | Serum albumin | Asp-Xaa-Cys-Leu-Pro-Xaa-Trp-Gly-Cys-Leu-Trp |
| | binding domain | D-Xaa-CLP-Xaa-WGCLW |
| 161 | Serum albumin | QGLIGDICLPRWGCLWGDSVK |
| | binding domain | |
| 162 | Serum albumin | RLIEDICLPRWGCLWEDD |
| | binding domain | |
| 163 | Serum albumin | EDICLPRWGCLWED |
| | binding domain | |
| 164 | NP_878910.1 | MTRTYENFQYLENKVKVQGFKNGPLPLQSLLQRLCSGP |
| | C-type lectin | CHLLLSLGLGLLLLVIICVVGFQNSKFQRDLVTLRTDFS |
| | domain family | NFTSNTVAEIQALTSQGSSLEETIASLKAEVEGFKQERQ |
| | 10 member A | AGVSELQEHTTQKAHLGHCPHCPSVCVPVHSEMLLRV |
| | isoform 1 | QQLVQDLKKLTCQVATLNNNASTEGTCCPVNWVEHQ |
| | | DSCYWFSHSGMSWAEAEKYCQLKNAHLVVINSREEQN |
| | | FVQKYLGSAYTWMGLSDPEGAWKWVDGTDYATGFQ |
| | | NWKPGQPDDWQGHGLGGGEDCAHFHPDGRWNDDVC |
| | | QRPYHWVCEAGLGQTSQESH |

Sequences of the CARs and Bi-specific fusion proteins of the present disclosure are found in Table 2

TABLE 2
Sequences of the CARs and Bi-specific fusion proteins

| | | |
|---|---|---|
| SEQ ID | | |
| NO: | Description | Sequence |

GlyTR1

| | | |
|---|---|---|
| 1 | GlyTR1^{L-PHAxCD3}: | SNDIYFNFQRFNETNLILQRDASVSSSGQLRLTNLNGN |
| | L-PHA- | GEPRVGSLGRAFYSAPIQIWDNTTGTVASFATSFTFNIQ |
| | Linker-CD3 | VPNNAGPADGLAFALVPVGSQPKDKGGFLGLFDGSNS |
| | scFv | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSDIKLQQSG |
| | | AELARPGASVKMSCKTSGYTFTRYTMHWVKQRPGQG |
| | | LEWIGYINPSRGYTNYNQKFKDKATLTTDKSSSTAYM |
| | | QLSSLTSEDSAVYYCARYYDDHYCLDYWGQGTTLTVS |
| | | SVEGGSGGSGGSGGSGGVDDIQLTQSPAIMSASPGEKV |
| | | TMTCRASSSVSYMNWYQQKSGTSPKRWIYDTSKVAS |
| | | GVPYRFSGSGSGTSYSLTISSMEAEDAATYYCQQWSSN |
| | | PLTFGAGTKLELK |
| 2 | GlyTR1^{L-PHAxCD3}: | SNDIYFNFQRFNETNLILQRDASVSSSGQLRLTNLNGN |
| | L-PHA- | GEPRVGSLGRAFYSAPIQIWDNTTGTVASFATSFTFNIQ |
| | Linker-CD3 | VPNNAGPADGLAFALVPVGSQPKDKGGFLGLFDGSNS |
| | scFv | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSDIKLQQSGAELARPGASVKMSCKTSGYTFTRYTM |
| | | HWVKQRPGQGLEWIGYINPSRGYTNYNQKFKDKATLT |
| | | TDKSSSTAYMQLSSLTSEDSAVYYCARYYDDHYCLDY |
| | | WGQGTTLTVSSVEGGSGGSGGSGGSGGVDDIQLTQSP |
| | | AIMSASPGEKVTMTCRASSSVSYMNWYQQKSGTSPKR |
| | | WIYDTSKVASGVPYRFSGSGSGTSYSLTISSMEAEDAA |
| | | TYYCQQWSSNPLTFGAGTKLELK |
| 3 | GlyTR1^{L-PHA(2)xCD3}: | SNDIYFNFQRFNETNLILQRDASVSSSGQLRLTNLNGN |
| | L-PHA(2)- | GEPRVGSLGRAFYSAPIQIWDNTTGTVASFATSFTFNIQ |
| | Linker-CD3 | VPNNAGPADGLAFALVPVGSQPKDKGGFLGLFDGSNS |
| | scFv | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSSNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILGGGGSGGGGS |
| | | GGGGSDIKLQQSGAELARPGASVKMSCKTSGYTFTRY |
| | | TMHWVKQRPGQGLEWIGYINPSRGYTNYNQKFKDKA |
| | | TLTTDKSSSTAYMQLSSLTSEDSAVYYCARYYDDHYC |
| | | LDYWGQGTTLTVSSVEGGSGGSGGSGGSGGVDDIQLT |
| | | QSPAIMSASPGEKVTMTCRASSSVSYMNWYQQKSGTS |
| | | PKRWIYDTSKVASGVPYRFSGSGSGTSYSLTISSMEAE |
| | | DAATYYCQQWSSNPLTFGAGTKLELK |
| 4 | GlyTR1^{L-PHA(3)xCD3}: | SNDIYFNFQRFNETNLILQRDASVSSSGQLRLTNLNGN |
| | L-PHA(3)- | GEPRVGSLGRAFYSAPIQIWDNTTGTVASFATSFTFNIQ |
| | Linker-CD3 | VPNNAGPADGLAFALVPVGSQPKDKGGFLGLFDGSNS |
| | scFv | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSSNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSDIKLQQSGAELARPGASVKMSCKTSGYTFTRY |
| | | TMHWVKQRPGQGLEWIGYINPSRGYTNYNQKFKDKA |
| | | TLTTDKSSSTAYMQLSSLTSEDSAVYYCARYYDDHYC |
| | | LDYWGQGTTLTVSSVEGGSGGSGGSGGSGGVDDIQLT |
| | | QSPAIMSASPGEKVTMTCRASSSVSYMNWYQQKSGTS |
| | | PKRWIYDTSKVASGVPYRFSGSGSGTSYSLTISSMEAE |
| | | DAATYYCQQWSSNPLTFGAGTKLELK |
| 5 | GlyTR1^{L-PHA(4)xCD3}: | SNDIYFNFQRFNETNLILQRDASVSSSGQLRLTNLNGN |
| | L-PHA(4)- | GEPRVGSLGRAFYSAPIQIWDNTTGTVASFATSFTFNIQ |
| | Linker-CD3 | VPNNAGPADGLAFALVPVGSQPKDKGGFLGLFDGSNS |
| | scFv | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSSNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSDIKLQQSGAELARPGASVKMSCKTSGYTFTRY |
| | | TMHWVKQRPGQGLEWIGYINPSRGYTNYNQKFKDKA |
| | | TLTTDKSSSTAYMQLSSLTSEDSAVYYCARYYDDHYC |
| | | LDYWGQGTTLTVSSVEGGSGGSGGSGGSGGVDDIQLT |
| | | QSPAIMSASPGEKVTMTCRASSSVSYMNWYQQKSGTS |
| | | PKRWIYDTSKVASGVPYRFSGSGSGTSYSLTISSMEAE |
| | | DAATYYCQQWSSNPLTFGAGTKLELK |
| 6 | HSA-GlyTR1^{L-PHAxCD3}: | DAHKSEVAHRFKDLGEENFKALVLIAFAQYLQQCPFE |
| | HSA- | DHVKLVNEVTEFAKTCVADESAENCDKSLHTLFGDKL |
| | Linker-L-PHA- | CTVATLRETYGEMADCCAKQEPERNECFLQHKDDNPN |
| | Linker-CD3 | LPRLVRPEVDVMCTAFHDNEETFLKKYLYEIARRHPYF |
| | scFv | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKASNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSDIKLQQSGAELARPGASVKMSCKTSGYTFTRY |
| | | TMHWVKQRPGQGLEWIGYINPSRGYTNYNQKFKDKA |
| | | TLTTDKSSSTAYMQLSSLTSEDSAVYYCARYYDDHYC |
| | | LDYWGQGTTLTVSSVEGGSGGSGGSGGSGGVDDIQLT |
| | | QSPAIMSASPGEKVTMTCRASSSVSYMNWYQQKSGTS |
| | | PKRWIYDTSKVASGVPYRFSGSGSGTSYSLTISSMEAE |
| | | DAATYYCQQWSSNPLTFGAGTKLELK |
| 7 | HSA-GlyTR1^{L-PHA(2)xCD3}: | DAHKSEVAHRFKDLGEENFKALVLIAFAQYLQQCPFE |
| | HSA- | DHVKLVNEVTEFAKTCVADESAENCDKSLHTLFGDKL |
| | Linker-L- | CTVATLRETYGEMADCCAKQEPERNECFLQHKDDNPN |
| | PHA(2)-Linker- | LPRLVRPEVDVMCTAFHDNEETFLKKYLYEIARRHPYF |
| | CD3 scFv | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKASNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSDIKLQQSGAELARPGASVKMSCKTSGYTFTRY |
| | | TMHWVKQRPGQGLEWIGYINPSRGYTNYNQKFKDKA |
| | | TLTTDKSSSTAYMQLSSLTSEDSAVYYCARYYDDHYC |
| | | LDYWGQGTTLTVSSVEGGSGGSGGSGGSGGVDDIQLT |
| | | QSPAIMSASPGEKVTMTCRASSSVSYMNWYQQKSGTS |
| | | PKRWIYDTSKVASGVPYRFSGSGSGTSYSLTISSMEAE |
| | | DAATYYCQQWSSNPLTFGAGTKLELK |
| 8 | HSA-GlyTR1^{L-PHA(3)xCD3}: | DAHKSEVAHRFKDLGEENFKALVLIAFAQYLQQCPFE |
| | HSA- | DHVKLVNEVTEFAKTCVADESAENCDKSLHTLFGDKL |
| | Linker-L- | CTVATLRETYGEMADCCAKQEPERNECFLQHKDDNPN |
| | PHA(3)-Linker- | LPRLVRPEVDVMCTAFHDNEETFLKKYLYEIARRHPYF |
| | CD3 scFv | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKASNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSDIKLQQSGAELARPGASVKMSCKTSGYTFTRY |
| | | TMHWVKQRPGQGLEWIGYINPSRGYTNYNQKFKDKA |
| | | TLTTDKSSSTAYMQLSSLTSEDSAVYYCARYYDDHYC |
| | | LDYWGQGTTLTVSSVEGGSGGSGGSGGSGGVDDIQLT |
| | | QSPAIMSASPGEKVTMTCRASSSVSYMNWYQQKSGTS |
| | | PKRWIYDTSKVASGVPYRFSGSGSGTSYSLTISSMEAE |
| | | DAATYYCQQWSSNPLTFGAGTKLELK |
| 9 | HSA-GlyTR1^{L-PHA(4)xCD3}: | DAHKSEVAHRFKDLGEENFKALVLIAFAQYLQQCPFE |
| | HSA- | DHVKLVNEVTEFAKTCVADESAENCDKSLHTLFGDKL |
| | Linker-L- | CTVATLRETYGEMADCCAKQEPERNECFLQHKDDNPN |
| | PHA(4)-Linker- | LPRLVRPEVDVMCTAFHDNEETFLKKYLYEIARRHPYF |
| | CD3 scFv | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKASNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSDIKLQQSGAELARPGASVKMSCKTSGYTFTRY |
| | | TMHWVKQRPGQGLEWIGYINPSRGYTNYNQKFKDKA |
| | | TLTTDKSSSTAYMQLSSLTSEDSAVYYCARYYDDHYC |
| | | LDYWGQGTTLTVSSVEGGSGGSGGSGGSGGVDDIQLT |
| | | QSPAIMSASPGEKVTMTCRASSSVSYMNWYQQKSGTS |
| | | PKRWIYDTSKVASGVPYRFSGSGSGTSYSLTISSMEAE |
| | | DAATYYCQQWSSNPLTFGAGTKLELK |
| 10 | GlyTR1^{L-PHAΔ1-3xCD3} | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHT |
| | | VAVEFDTLYNKDWDPTERHIGIDVNSIRSIKTTRWDFV |
| | | NGENAEVLITYDSSTNLLVASLVYPSQKTSFIVSDTVDL |
| | | KSVLPEWVSVGFSATTGINKGNVETNDVLSWSFASKLS |
| | | DGTTSEGLNLANLVLNKILGGGGSGGGGSGGGGSDIK |
| | | LQQSGAELARPGASVKMSCKTSGYTFTRYTMHWVKQ |
| | | RPGQGLEWIGYINPSRGYTNYNQKFKDKATLTTDKSSS |
| | | TAYMQLSSLTSEDSAVYYCARYYDDHYCLDYWGQGT |
| | | TLTVSSVEGGSGGSGGSGGSGGVDDIQLTQSPAIMSAS |
| | | PGEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 11 | GlyTR1^{L-PHAΔ1-3(2)xCD3} | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHT |
| | | VAVEFDTLYNKDWDPTERHIGIDVNSIRSIKTTRWDFV |
| | | NGENAEVLITYDSSTNLLVASLVYPSQKTSFIVSDTVDL |
| | | KSVLPEWVSVGFSATTGINKGNVETNDVLSWSFASKLS |
| | | DGTTSEGLNLANLVLNKILGGGGSGGGGSGGGGSIYFN |
| | | FQRFNETNLILQRDASVSSSGQLRLTNLNGNGEPRVGS |
| | | LGRAFYSAPIQIWDNTTGTVASFATSFTFNIQVPNNAGP |
| | | ADGLAFALVPVGSQPKDKGGFLGLFDGSNSNFHTVAV |
| | | EFDTLYNKDWDPTERHIGIDVNSIRSIKTTRWDFVNGE |
| | | NAEVLITYDSSTNLLVASLVYPSQKTSFIVSDTVDLKSV |
| | | LPEWVSVGFSATTGINKGNVETNDVLSWSFASKLSDG |
| | | TTSEGLNLANLVLNKILGGGGSGGGGSGGGGSDIKLQ |
| | | QSGAELARPGASVKMSCKTSGYTFTRYTMHWVKQRP |
| | | GQGLEWIGYINPSRGYTNYNQKFKDKATLTTDKSSSTA |
| | | YMQLSSLTSEDSAVYYCARYYDDHYCLDYWGQGTTL |
| | | TVSSVEGGSGGSGGSGGSGGVDDIQLTQSPAIMSASPG |
| | | EKVTMTCRASSSVSYMNWYQQKSGTSPKRWIYDTSK |
| | | VASGVPYRFSGSGSGTSYSLTISSMEAEDAATYYCQQ |
| | | WSSNPLTFGAGTKLELK |
| 12 | GlyTR1^{L-PHAΔ1-3(3)xCD3} | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHT |
| | | VAVEFDTLYNKDWDPTERHIGIDVNSIRSIKTTRWDFV |
| | | NGENAEVLITYDSSTNLLVASLVYPSQKTSFIVSDTVDL |
| | | KSVLPEWVSVGFSATTGINKGNVETNDVLSWSFASKLS |
| | | DGTTSEGLNLANLVLNKILGGGGSGGGGSGGGGSIYFN |
| | | FQRFNETNLILQRDASVSSSGQLRLTNLNGNGEPRVGS |
| | | LGRAFYSAPIQIWDNTTGTVASFATSFTFNIQVPNNAGP |
| | | ADGLAFALVPVGSQPKDKGGFLGLFDGSNSNFHTVAV |
| | | EFDTLYNKDWDPTERHIGIDVNSIRSIKTTRWDFVNGE |
| | | NAEVLITYDSSTNLLVASLVYPSQKTSFIVSDTVDLKSV |
| | | LPEWVSVGFSATTGINKGNVETNDVLSWSFASKLSDG |
| | | TTSEGLNLANLVLNKILGGGGSGGGGSGGGGSIYFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSDIKLQQS |
| | | GAELARPGASVKMSCKTSGYTFTRYTMHWVKQRPGQ |
| | | GLEWIGYINPSRGYTNYNQKFKDKATLTTDKSSSTAY |
| | | MQLSSLTSEDSAVYYCARYYDDHYCLDYWGQGTTLT |
| | | VSSVEGGSGGSGGSGGSGGVDDIQLTQSPAIMSASPGE |
| | | KVTMTCRASSSVSYMNWYQQKSGTSPKRWIYDTSKV |
| | | ASGVPYRFSGSGSGTSYSLTISSMEAEDAATYYCQQWS |
| | | SNPLTFGAGTKLELK |
| 13 | GlyTR1^{L-PHAΔ1-3(4)xCD3} | IYFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHT |
| | | VAVEFDTLYNKDWDPTERHIGIDVNSIRSIKTTRWDFV |
| | | NGENAEVLITYDSSTNLLVASLVYPSQKTSFIVSDTVDL |
| | | KSVLPEWVSVGFSATTGINKGNVETNDVLSWSFASKLS |
| | | DGTTSEGLNLANLVLNKILGGGGSGGGGSGGGGSIYFN |
| | | FQRFNETNLILQRDASVSSSGQLRLTNLNGNGEPRVGS |
| | | LGRAFYSAPIQIWDNTTGTVASFATSFTFNIQVPNNAGP |
| | | ADGLAFALVPVGSQPKDKGGFLGLFDGSNSNFHTVAV |
| | | EFDTLYNKDWDPTERHIGIDVNSIRSIKTTRWDFVNGE |
| | | NAEVLITYDSSTNLLVASLVYPSQKTSFIVSDTVDLKSV |
| | | LPEWVSVGFSATTGINKGNVETNDVLSWSFASKLSDG |
| | | TTSEGLNLANLVLNKILGGGGSGGGGSGGGGSIYFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSIYFNFQRF |
| | | NETNLILQRDASVSSSGQLRLTNLNGNGEPRVGSLGRA |
| | | FYSAPIQIWDNTTGTVASFATSFTFNIQVPNNAGPADGL |
| | | AFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKILGGGGSGGGGSGGGGSDIKLQQSGAE |
| | | LARPGASVKMSCKTSGYTFTRYTMHWVKQRPGQGLE |
| | | WIGYINPSRGYTNYNQKFKDKATLTTDKSSSTAYMQL |
| | | SSLTSEDSAVYYCARYYDDHYCLDYWGQGTTLTVSSV |
| | | EGGSGGSGGSGGSGGVDDIQLTQSPAIMSASPGEKVTM |
| | | TCRASSSVSYMNWYQQKSGTSPKRWIYDTSKVASGVP |
| | | YRFSGSGSGTSYSLTISSMEAEDAATYYCQQWSSNPLT |
| | | FGAGTKLELK |
| 14 | HSA-GlyTR1^{L-PHAΔ1-3xCD3} | DAHKSEVAHRFKDLGEENFKALVLIAFAQYLQQCPFE |
| | | DHVKLVNEVTEFAKTCVADESAENCDKSLHTLFGDKL |
| | | CTVATLRETYGEMADCCAKQEPERNECFLQHKDDNPN |
| | | LPRLVRPEVDVMCTAFHDNEETFLKKYLYEIARRHPYF |
| | | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAIYFNFQRFNETNLILQRDASVSSSGQLRLTNLNG |
| | | NGEPRVGSLGRAFYSAPIQIWDNTTGTVASFATSFTFNI |
| | | QVPNNAGPADGLAFALVPVGSQPKDKGGFLGLFDGSN |
| | | SNFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSDIKLQQSGAELARPGASVKMSCKTSGYTFTRYTM |
| | | HWVKQRPGQGLEWIGYINPSRGYTNYNQKFKDKATLT |
| | | TDKSSSTAYMQLSSLTSEDSAVYYCARYYDDHYCLDY |
| | | WGQGTTLTVSSVEGGSGGSGGSGGSGGVDDIQLTQSP |
| | | AIMSASPGEKVTMTCRASSSVSYMNWYQQKSGTSPKR |
| | | WIYDTSKVASGVPYRFSGSGSGTSYSLTISSMEAEDAA |
| | | TYYCQQWSSNPLTFGAGTKLELK |
| 15 | HSA- | DAHKSEVAHRFKDLGEENFKALVLIAFAQYLQQCPFE |
| | GlyTR1^{L-PHAΔ1-3(2)xCD3} | DHVKLVNEVTEFAKTCVADESAENCDKSLHTLFGDKL |
| | | CTVATLRETYGEMADCCAKQEPERNECFLQHKDDNPN |
| | | LPRLVRPEVDVMCTAFHDNEETFLKKYLYEIARRHPYF |
| | | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAIYFNFQRFNETNLILQRDASVSSSGQLRLTNLNG |
| | | NGEPRVGSLGRAFYSAPIQIWDNTTGTVASFATSFTFNI |
| | | QVPNNAGPADGLAFALVPVGSQPKDKGGFLGLFDGSN |
| | | SNFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSIYFNFQRFNETNLILQRDASVSSSGQLRLTNLNGN |
| | | GEPRVGSLGRAFYSAPIQIWDNTTGTVASFATSFTFNIQ |
| | | VPNNAGPADGLAFALVPVGSQPKDKGGFLGLFDGSNS |
| | | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSDIKLQQSGAELARPGASVKMSCKTSGYTFTRYTM |
| | | HWVKQRPGQGLEWIGYINPSRGYTNYNQKFKDKATLT |
| | | TDKSSSTAYMQLSSLTSEDSAVYYCARYYDDHYCLDY |
| | | WGQGTTLTVSSVEGGSGGSGGSGGSGGVDDIQLTQSP |
| | | AIMSASPGEKVTMTCRASSSVSYMNWYQQKSGTSPKR |
| | | WIYDTSKVASGVPYRFSGSGSGTSYSLTISSMEAEDAA |
| | | TYYCQQWSSNPLTFGAGTKLELK |
| 16 | HSA- | DAHKSEVAHRFKDLGEENFKALVLIAFAQYLQQCPFE |
| | GlyTR1^{L-PHAΔ1-3(3)xCD3} | DHVKLVNEVTEFAKTCVADESAENCDKSLHTLFGDKL |
| | | CTVATLRETYGEMADCCAKQEPERNECFLQHKDDNPN |
| | | LPRLVRPEVDVMCTAFHDNEETFLKKYLYEIARRHPYF |
| | | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAIYFNFQRFNETNLILQRDASVSSSGQLRLTNLNG |
| | | NGEPRVGSLGRAFYSAPIQIWDNTTGTVASFATSFTFNI |
| | | QVPNNAGPADGLAFALVPVGSQPKDKGGFLGLFDGSN |
| | | SNFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSIYFNFQRFNETNLILQRDASVSSSGQLRLTNLNGN |
| | | GEPRVGSLGRAFYSAPIQIWDNTTGTVASFATSFTFNIQ |
| | | VPNNAGPADGLAFALVPVGSQPKDKGGFLGLFDGSNS |
| | | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSIYFNFQRFNETNLILQRDASVSSSGQLRLTNLNGN |
| | | GEPRVGSLGRAFYSAPIQIWDNTTGTVASFATSFTFNIQ |
| | | VPNNAGPADGLAFALVPVGSQPKDKGGFLGLFDGSNS |
| | | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSDIKLQQSGAELARPGASVKMSCKTSGYTFTRYTM |
| | | HWVKQRPGQGLEWIGYINPSRGYTNYNQKFKDKATLT |
| | | TDKSSSTAYMQLSSLTSEDSAVYYCARYYDDHYCLDY |
| | | WGQGTTLTVSSVEGGSGGSGGSGGSGGVDDIQLTQSP |
| | | AIMSASPGEKVTMTCRASSSVSYMNWYQQKSGTSPKR |
| | | WIYDTSKVASGVPYRFSGSGSGTSYSLTISSMEAEDAA |
| | | TYYCQQWSSNPLTFGAGTKLELK |
| 17 | HSA- | DAHKSEVAHRFKDLGEENFKALVLIAFAQYLQQCPFE |
| | GlyTR1^{L-PHAΔ1-3(4)xCD3} | DHVKLVNEVTEFAKTCVADESAENCDKSLHTLFGDKL |
| | | CTVATLRETYGEMADCCAKQEPERNECFLQHKDDNPN |
| | | LPRLVRPEVDVMCTAFHDNEETFLKKYLYEIARRHPYF |
| | | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAIYFNFQRFNETNLILQRDASVSSSGQLRLTNLNG |
| | | NGEPRVGSLGRAFYSAPIQIWDNTTGTVASFATSFTFNI |
| | | QVPNNAGPADGLAFALVPVGSQPKDKGGFLGLFDGSN |
| | | SNFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSIYFNFQRFNETNLILQRDASVSSSGQLRLTNLNGN |
| | | GEPRVGSLGRAFYSAPIQIWDNTTGTVASFATSFTFNIQ |
| | | VPNNAGPADGLAFALVPVGSQPKDKGGFLGLFDGSNS |
| | | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSIYFNFQRFNETNLILQRDASVSSSGQLRLTNLNGN |
| | | GEPRVGSLGRAFYSAPIQIWDNTTGTVASFATSFTFNIQ |
| | | VPNNAGPADGLAFALVPVGSQPKDKGGFLGLFDGSNS |
| | | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSIYFNFQRFNETNLILQRDASVSSSGQLRLTNLNGN |
| | | GEPRVGSLGRAFYSAPIQIWDNTTGTVASFATSFTFNIQ |
| | | VPNNAGPADGLAFALVPVGSQPKDKGGFLGLFDGSNS |
| | | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSDIKLQQSGAELARPGASVKMSCKTSGYTFTRYTM |
| | | HWVKQRPGQGLEWIGYINPSRGYTNYNQKFKDKATLT |
| | | TDKSSSTAYMQLSSLTSEDSAVYYCARYYDDHYCLDY |
| | | WGQGTTLTVSSVEGGSGGSGGSGGSGGVDDIQLTQSP |
| | | AIMSASPGEKVTMTCRASSSVSYMNWYQQKSGTSPKR |
| | | WIYDTSKVASGVPYRFSGSGSGTSYSLTISSMEAEDAA |
| | | TYYCQQWSSNPLTFGAGTKLELK |
| 18 | GlyTR1^{L-PHAΔ1-5xCD3} | FNFQRFNETNLILQRDASVSSSGQLRLTNLNGNGEPRV |
| | | GSLGRAFYSAPIQIWDNTTGTVASFATSFTFNIQVPNNA |
| | | GPADGLAFALVPVGSQPKDKGGFLGLFDGSNSNFHTV |
| | | AVEFDTLYNKDWDPTERHIGIDVNSIRSIKTTRWDFVN |
| | | GENAEVLITYDSSTNLLVASLVYPSQKTSFIVSDTVDLK |
| | | SVLPEWVSVGFSATTGINKGNVETNDVLSWSFASKLSD |
| | | GTTSEGLNLANLVLNKILGGGGSGGGGSGGGGSDIKL |
| | | QQSGAELARPGASVKMSCKTSGYTFTRYTMHWVKQR |
| | | PGQGLEWIGYINPSRGYTNYNQKFKDKATLTTDKSSST |
| | | AYMQLSSLTSEDSAVYYCARYYDDHYCLDYWGQGTT |
| | | LTVSSVEGGSGGSGGSGGSGGVDDIQLTQSPAIMSASP |
| | | GEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 19 | GlyTR1^{L-PHAΔ1-5(2)xCD3} | FNFQRFNETNLILQRDASVSSSGQLRLTNLNGNGEPRV |
| | | GSLGRAFYSAPIQIWDNTTGTVASFATSFTFNIQVPNNA |
| | | GPADGLAFALVPVGSQPKDKGGFLGLFDGSNSNFHTV |
| | | AVEFDTLYNKDWDPTERHIGIDVNSIRSIKTTRWDFVN |
| | | GENAEVLITYDSSTNLLVASLVYPSQKTSFIVSDTVDLK |
| | | SVLPEWVSVGFSATTGINKGNVETNDVLSWSFASKLSD |
| | | GTTSEGLNLANLVLNKILGGGGSGGGGSGGGGSFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSDIKLQQS |
| | | GAELARPGASVKMSCKTSGYTFTRYTMHWVKQRPGQ |
| | | GLEWIGYINPSRGYTNYNQKFKDKATLTTDKSSSTAY |
| | | MQLSSLTSEDSAVYYCARYYDDHYCLDYWGQGTTLT |
| | | VSSVEGGSGGSGGSGGSGGVDDIQLTQSPAIMSASPGE |
| | | KVTMTCRASSSVSYMNWYQQKSGTSPKRWIYDTSKV |
| | | ASGVPYRFSGSGSGTSYSLTISSMEAEDAATYYCQQWS |
| | | SNPLTFGAGTKLELK |
| 20 | GlyTR1^{L-PHAΔ1-5(3)xCD3} | FNFQRFNETNLILQRDASVSSSGQLRLTNLNGNGEPRV |
| | | GSLGRAFYSAPIQIWDNTTGTVASFATSFTFNIQVPNNA |
| | | GPADGLAFALVPVGSQPKDKGGFLGLFDGSNSNFHTV |
| | | AVEFDTLYNKDWDPTERHIGIDVNSIRSIKTTRWDFVN |
| | | GENAEVLITYDSSTNLLVASLVYPSQKTSFIVSDTVDLK |
| | | SVLPEWVSVGFSATTGINKGNVETNDVLSWSFASKLSD |
| | | GTTSEGLNLANLVLNKILGGGGSGGGGSGGGGSFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSFNFQRFN |
| | | ETNLILQRDASVSSSGQLRLTNLNGNGEPRVGSLGRAF |
| | | YSAPIQIWDNTTGTVASFATSFTFNIQVPNNAGPADGL |
| | | AFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKILGGGGSGGGGSGGGGSDIKLQQSGAE |
| | | LARPGASVKMSCKTSGYTFTRYTMHWVKQRPGQGLE |
| | | WIGYINPSRGYTNYNQKFKDKATLTTDKSSSTAYMQL |
| | | SSLTSEDSAVYYCARYYDDHYCLDYWGQGTTLTVSSV |
| | | EGGSGGSGGSGGSGGVDDIQLTQSPAIMSASPGEKVTM |
| | | TCRASSSVSYMNWYQQKSGTSPKRWIYDTSKVASGVP |
| | | YRFSGSGSGTSYSLTISSMEAEDAATYYCQQWSSNPLT |
| | | FGAGTKLELK |
| 21 | GlyTR1^{L-PHAΔ1-5(4)xCD3} | FNFQRFNETNLILQRDASVSSSGQLRLTNLNGNGEPRV |
| | | GSLGRAFYSAPIQIWDNTTGTVASFATSFTFNIQVPNNA |
| | | GPADGLAFALVPVGSQPKDKGGFLGLFDGSNSNFHTV |
| | | AVEFDTLYNKDWDPTERHIGIDVNSIRSIKTTRWDFVN |
| | | GENAEVLITYDSSTNLLVASLVYPSQKTSFIVSDTVDLK |
| | | SVLPEWVSVGFSATTGINKGNVETNDVLSWSFASKLSD |
| | | GTTSEGLNLANLVLNKILGGGGSGGGGSGGGGSFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSFNFQRFN |
| | | ETNLILQRDASVSSSGQLRLTNLNGNGEPRVGSLGRAF |
| | | YSAPIQIWDNTTGTVASFATSFTFNIQVPNNAGPADGL |
| | | AFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKILGGGGSGGGGSGGGGSFNFQRFNETN |
| | | LILQRDASVSSSGQLRLTNLNGNGEPRVGSLGRAFYSA |
| | | PIQIWDNTTGTVASFATSFTFNIQVPNNAGPADGLAFAL |
| | | VPVGSQPKDKGGFLGLFDGSNSNFHTVAVEFDTLYNK |
| | | DWDPTERHIGIDVNSIRSIKTTRWDFVNGENAEVLITYD |
| | | SSTNLLVASLVYPSQKTSFIVSDTVDLKSVLPEWVSVG |
| | | FSATTGINKGNVETNDVLSWSFASKLSDGTTSEGLNLA |
| | | NLVLNKILGGGGSGGGGSGGGGSDIKLQQSGAELARP |
| | | GASVKMSCKTSGYTFTRYTMHWVKQRPGQGLEWIGY |
| | | INPSRGYTNYNQKFKDKATLTTDKSSSTAYMQLSSLTS |
| | | EDSAVYYCARYYDDHYCLDYWGQGTTLTVSSVEGGS |
| | | GGSGGSGGSGGVDDIQLTQSPAIMSASPGEKVTMTCR |
| | | ASSSVSYMNWYQQKSGTSPKRWIYDTSKVASGVPYRF |
| | | SGSGSGTSYSLTISSMEAEDAATYYCQQWSSNPLTFGA |
| | | GTKLELK |
| 22 | HSA-GlyTR1^{L-PHAΔ1-5xCD3} | DAHKSEVAHRFKDLGEENFKALVLIAFAQYLQQCPFE |
| | | DHVKLVNEVTEFAKTCVADESAENCDKSLHTLFGDKL |
| | | CTVATLRETYGEMADCCAKQEPERNECFLQHKDDNPN |
| | | LPRLVRPEVDVMCTAFHDNEETFLKKYLYEIARRHPYF |
| | | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAFNFQRFNETNLILQRDASVSSSGQLRLTNLNGNG |
| | | EPRVGSLGRAFYSAPIQIWDNTTGTVASFATSFTFNIQV |
| | | PNNAGPADGLAFALVPVGSQPKDKGGFLGLFDGSNSN |
| | | FHTVAVEFDTLYNKDWDPTERHIGIDVNSIRSIKTTRW |
| | | DFVNGENAEVLITYDSSTNLLVASLVYPSQKTSFIVSDT |
| | | VDLKSVLPEWVSVGFSATTGINKGNVETNDVLSWSFA |
| | | SKLSDGTTSEGLNLANLVLNKILGGGGSGGGGSGGGG |
| | | SDIKLQQSGAELARPGASVKMSCKTSGYTFTRYTMHW |
| | | VKQRPGQGLEWIGYINPSRGYTNYNQKFKDKATLTTD |
| | | KSSSTAYMQLSSLTSEDSAVYYCARYYDDHYCLDYW |
| | | GQGTTLTVSSVEGGSGGSGGSGGSGGVDDIQLTQSPAI |
| | | MSASPGEKVTMTCRASSSVSYMNWYQQKSGTSPKRW |
| | | IYDTSKVASGVPYRFSGSGSGTSYSLTISSMEAEDAATY |
| | | YCQQWSSNPLTFGAGTKLELK |
| 23 | HSA- | DAHKSEVAHRFKDLGEENFKALVLIAFAQYLQQCPFE |
| | GlyTR1^{L-PHAΔ1-5(2)xCD3} | DHVKLVNEVTEFAKTCVADESAENCDKSLHTLFGDKL |
| | | CTVATLRETYGEMADCCAKQEPERNECFLQHKDDNPN |
| | | LPRLVRPEVDVMCTAFHDNEETFLKKYLYEIARRHPYF |
| | | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAFNFQRFNETNLILQRDASVSSSGQLRLTNLNGNG |
| | | EPRVGSLGRAFYSAPIQIWDNTTGTVASFATSFTFNIQV |
| | | PNNAGPADGLAFALVPVGSQPKDKGGFLGLFDGSNSN |
| | | FHTVAVEFDTLYNKDWDPTERHIGIDVNSIRSIKTTRW |
| | | DFVNGENAEVLITYDSSTNLLVASLVYPSQKTSFIVSDT |
| | | VDLKSVLPEWVSVGFSATTGINKGNVETNDVLSWSFA |
| | | SKLSDGTTSEGLNLANLVLNKILGGGGSGGGGSGGGG |
| | | SFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHT |
| | | VAVEFDTLYNKDWDPTERHIGIDVNSIRSIKTTRWDFV |
| | | NGENAEVLITYDSSTNLLVASLVYPSQKTSFIVSDTVDL |
| | | KSVLPEWVSVGFSATTGINKGNVETNDVLSWSFASKLS |
| | | DGTTSEGLNLANLVLNKILGGGGSGGGGSGGGGSDIK |
| | | LQQSGAELARPGASVKMSCKTSGYTFTRYTMHWVKQ |
| | | RPGQGLEWIGYINPSRGYTNYNQKFKDKATLTTDKSSS |
| | | TAYMQLSSLTSEDSAVYYCARYYDDHYCLDYWGQGT |
| | | TLTVSSVEGGSGGSGGSGGSGGVDDIQLTQSPAIMSAS |
| | | PGEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 24 | HSA- | DAHKSEVAHRFKDLGEENFKALVLIAFAQYLQQCPFE |
| | GlyTR1^{L-PHAΔ1-5(3)xCD3} | DHVKLVNEVTEFAKTCVADESAENCDKSLHTLFGDKL |
| | | CTVATLRETYGEMADCCAKQEPERNECFLQHKDDNPN |
| | | LPRLVRPEVDVMCTAFHDNEETFLKKYLYEIARRHPYF |
| | | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAFNFQRFNETNLILQRDASVSSSGQLRLTNLNGNG |
| | | EPRVGSLGRAFYSAPIQIWDNTTGTVASFATSFTFNIQV |
| | | PNNAGPADGLAFALVPVGSQPKDKGGFLGLFDGSNSN |
| | | FHTVAVEFDTLYNKDWDPTERHIGIDVNSIRSIKTTRW |
| | | DFVNGENAEVLITYDSSTNLLVASLVYPSQKTSFIVSDT |
| | | VDLKSVLPEWVSVGFSATTGINKGNVETNDVLSWSFA |
| | | SKLSDGTTSEGLNLANLVLNKILGGGGSGGGGSGGGG |
| | | SFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHT |
| | | VAVEFDTLYNKDWDPTERHIGIDVNSIRSIKTTRWDFV |
| | | NGENAEVLITYDSSTNLLVASLVYPSQKTSFIVSDTVDL |
| | | KSVLPEWVSVGFSATTGINKGNVETNDVLSWSFASKLS |
| | | DGTTSEGLNLANLVLNKILGGGGSGGGGSGGGGSFNF |
| | | QRFNETNLILQRDASVSSSGQLRLTNLNGNGEPRVGSL |
| | | GRAFYSAPIQIWDNTTGTVASFATSFTFNIQVPNNAGPA |
| | | DGLAFALVPVGSQPKDKGGFLGLFDGSNSNFHTVAVE |
| | | FDTLYNKDWDPTERHIGIDVNSIRSIKTTRWDFVNGEN |
| | | AEVLITYDSSTNLLVASLVYPSQKTSFIVSDTVDLKSVL |
| | | PEWVSVGFSATTGINKGNVETNDVLSWSFASKLSDGT |
| | | TSEGLNLANLVLNKILGGGGSGGGGSGGGGSDIKLQQS |
| | | GAELARPGASVKMSCKTSGYTFTRYTMHWVKQRPGQ |
| | | GLEWIGYINPSRGYTNYNQKFKDKATLTTDKSSSTAY |
| | | MQLSSLTSEDSAVYYCARYYDDHYCLDYWGQGTTLT |
| | | VSSVEGGSGGSGGSGGSGGVDDIQLTQSPAIMSASPGE |
| | | KVTMTCRASSSVSYMNWYQQKSGTSPKRWIYDTSKV |
| | | ASGVPYRFSGSGSGTSYSLTISSMEAEDAATYYCQQWS |
| | | SNPLTFGAGTKLELK |
| 25 | HSA- | DAHKSEVAHRFKDLGEENFKALVLIAFAQYLQQCPFE |
| | GlyTR1^{L-PHAΔ1-5(4)xCD3} | DHVKLVNEVTEFAKTCVADESAENCDKSLHTLFGDKL |
| | | CTVATLRETYGEMADCCAKQEPERNECFLQHKDDNPN |
| | | LPRLVRPEVDVMCTAFHDNEETFLKKYLYEIARRHPYF |
| | | YAPELLFFAKRYKAAFTECCQAADKAACLLPKLDELR |
| | | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAFNFQRFNETNLILQRDASVSSSGQLRLTNLNGNG |
| | | EPRVGSLGRAFYSAPIQIWDNTTGTVASFATSFTFNIQV |
| | | PNNAGPADGLAFALVPVGSQPKDKGGFLGLFDGSNSN |
| | | FHTVAVEFDTLYNKDWDPTERHIGIDVNSIRSIKTTRW |
| | | DFVNGENAEVLITYDSSTNLLVASLVYPSQKTSFIVSDT |
| | | VDLKSVLPEWVSVGFSATTGINKGNVETNDVLSWSFA |
| | | SKLSDGTTSEGLNLANLVLNKILGGGGSGGGGSGGGG |
| | | SFNFQRFNETNLILQRDASVSSSGQLRLTNLNGNGEPR |
| | | VGSLGRAFYSAPIQIWDNTTGTVASFATSFTFNIQVPNN |
| | | AGPADGLAFALVPVGSQPKDKGGFLGLFDGSNSNFHT |
| | | VAVEFDTLYNKDWDPTERHIGIDVNSIRSIKTTRWDFV |
| | | NGENAEVLITYDSSTNLLVASLVYPSQKTSFIVSDTVDL |
| | | KSVLPEWVSVGFSATTGINKGNVETNDVLSWSFASKLS |
| | | DGTTSEGLNLANLVLNKILGGGGSGGGGSGGGGSFNF |
| | | QRFNETNLILQRDASVSSSGQLRLTNLNGNGEPRVGSL |
| | | GRAFYSAPIQIWDNTTGTVASFATSFTFNIQVPNNAGPA |
| | | DGLAFALVPVGSQPKDKGGFLGLFDGSNSNFHTVAVE |
| | | FDTLYNKDWDPTERHIGIDVNSIRSIKTTRWDFVNGEN |
| | | AEVLITYDSSTNLLVASLVYPSQKTSFIVSDTVDLKSVL |
| | | PEWVSVGFSATTGINKGNVETNDVLSWSFASKLSDGT |
| | | TSEGLNLANLVLNKILGGGGGGGGSGGGGSFNFQRF |
| | | NETNLILQRDASVSSSGQLRLTNLNGNGEPRVGSLGRA |
| | | FYSAPIQIWDNTTGTVASFATSFTFNIQVPNNAGPADGL |
| | | AFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKILGGGGSGGGGSGGGGSDIKLQQSGAE |
| | | LARPGASVKMSCKTSGYTFTRYTMHWVKQRPGQGLE |
| | | WIGYINPSRGYTNYNQKFKDKATLTTDKSSSTAYMQL |
| | | SSLTSEDSAVYYCARYYDDHYCLDYWGQGTTLTVSSV |
| | | EGGSGGSGGSGGSGGVDDIQLTQSPAIMSASPGEKVTM |
| | | TCRASSSVSYMNWYQQKSGTSPKRWIYDTSKVASGVP |
| | | YRFSGSGSGTSYSLTISSMEAEDAATYYCQQWSSNPLT |
| | | FGAGTKLELK |

GlyTR2

| | | |
|---|---|---|
| 26 | GlyTR2^{CD301xCD3}: | VHSEMLLRVQQLVQDLKKLTCQVATLNNNASTEGTC |
| | Extended | CPVNWVEHQDSCYWFSHSGMSWAEAEKYCQLKNAH |
| | CD301CRD- | LVVINSREEQNFVQKYLGSAYTWMGLSDPEGAWKWV |
| | Linker-CD3 | DGTDYATGFQNWKPGQPDDWQGHGLGGGEDCAHFH |
| | scFv | PDGRWNDDVCQRPYHWVCEAGLGQTSQESHGGGGSG |
| | | GGGSGGGGSDIKLQQSGAELARPGASVKMSCKTSGYT |
| | | FTRYTMHWVKQRPGQGLEWIGYINPSRGYTNYNQKF |
| | | KDKATLTTDKSSSTAYMQLSSLTSEDSAVYYCARYYD |
| | | DHYCLDYWGQGTTLTVSSVEGGSGGSGGSGGSGGVD |
| | | DIQLTQSPAIMSASPGEKVTMTCRASSSVSYMNWYQQ |
| | | KSGTSPKRWIYDTSKVASGVPYRFSGSGSGTSYSLTISS |
| | | MEAEDAATYYCQQWSSNPLTFGAGTKLELK |
| 27 | mutGlyTR2^{CD301xCD3}: | VHSEMLLRVQQLVQDLKKLTCQVATLNNNASTEGTC |
| | Mutated | CPVNWVEHQDSCYWFSHSGMSWAEAEKYCQLKNAH |
| | extended | LVVINSREEQNFVQKYLGSAYTWMGLSDPEGAWKWV |
| | CD301CRD- | DGTDYATGFQNWKPGGPGDWQGHGLGGGGDCAHFH |
| | Linker-CD3 | PDGRWGGDVCQRPYHWVCEAGLGQTSQESHGGGGSG |
| | scFv | GGGSGGGGSDIKLQQSGAELARPGASVKMSCKTSGYT |
| | | FTRYTMHWVKQRPGQGLEWIGYINPSRGYTNYNQKF |
| | | KDKATLTTDKSSSTAYMQLSSLTSEDSAVYYCARYYD |
| | | DHYCLDYWGQGTTLTVSSVEGGSGGSGGSGGSGGVD |
| | | DIQLTQSPAIMSASPGEKVTMTCRASSSVSYMNWYQQ |
| | | KSGTSPKRWIYDTSKVASGVPYRFSGSGSGTSYSLTISS |
| | | MEAEDAATYYCQQWSSNPLTFGAGTKLELK |
| 28 | GlyTR2^{CD301(2)xCD3}: | VHSEMLLRVQQLVQDLKKLTCQVATLNNNASTEGTC |
| | Extended | CPVNWVEHQDSCYWFSHSGMSWAEAEKYCQLKNAH |
| | CD301CRD- | LVVINSREEQNFVQKYLGSAYTWMGLSDPEGAWKWV |
| | Linker- | DGTDYATGFQNWKPGQPDDWQGHGLGGGEDCAHFH |
| | CD301CRD- | PDGRWNDDVCQRPYHWVCEAGLGQTSQESHGGGGSG |
| | Linker-CD3 | GGGSGGGGSLTCQVATLNNNASTEGTCCPVNWVEHQ |
| | scFv | DSCYWFSHSGMSWAEAEKYCQLKNAHLVVINSREEQ |
| | | NFVQKYLGSAYTWMGLSDPEGAWKWVDGTDYATGF |
| | | QNWKPGQPDDWQGHGLGGGEDCAHFHPDGRWNDDV |
| | | CQRPYHWVCEAGLGQTSQESHGGGGSGGGGSGGGGS |
| | | DIKLQQSGAELARPGASVKMSCKTSGYTFTRYTMHWV |
| | | KQRPGQGLEWIGYINPSRGYTNYNQKFKDKATLTTDK |
| | | SSSTAYMQLSSLTSEDSAVYYCARYYDDHYCLDYWG |
| | | QGTTLTVSSVEGGSGGSGGSGGSGGVDDIQLTQSPAIM |
| | | SASPGEKVTMTCRASSSVSYMNWYQQKSGTSPKRWIY |
| | | DTSKVASGVPYRFSGSGSGTSYSLTISSMEAEDAATYY |
| | | CQQWSSNPLTFGAGTKLELK |
| 29 | GlyTR2^{CD301(3)xCD3}: | VHSEMLLRVQQLVQDLKKLTCQVATLNNNASTEGTC |
| | Extended | CPVNWVEHQDSCYWFSHSGMSWAEAEKYCQLKNAH |
| | CD301CRD- | LVVINSREEQNFVQKYLGSAYTWMGLSDPEGAWKWV |
| | Linker- | DGTDYATGFQNWKPGQPDDWQGHGLGGGEDCAHFH |
| | CD301CRD(2)- | PDGRWNDDVCQRPYHWVCEAGLGQTSQESHGGGGSG |
| | Linker-CD3 | GGGSGGGGSLTCQVATLNNNASTEGTCCPVNWVEHQ |
| | scFv | DSCYWFSHSGMSWAEAEKYCQLKNAHLVVINSREEQ |
| | | NFVQKYLGSAYTWMGLSDPEGAWKWVDGTDYATGF |
| | | QNWKPGQPDDWQGHGLGGGEDCAHFHPDGRWNDDV |
| | | CQRPYHWVCEAGLGQTSQESHGGGGSGGGGSGGGGS |
| | | LTCQVATLNNNASTEGTCCPVNWVEHQDSCYWFSHS |
| | | GMSWAEAEKYCQLKNAHLVVINSREEQNFVQKYLGS |
| | | AYTWMGLSDPEGAWKWVDGTDYATGFQNWKPGQPD |
| | | DWQGHGLGGGEDCAHFHPDGRWNDDVCQRPYHWVC |
| | | EAGLGQTSQESHGGGGSGGGGSGGGGSDIKLQQSGAE |
| | | LARPGASVKMSCKTSGYTFTRYTMHWVKQRPGQGLE |
| | | WIGYINPSRGYTNYNQKFKDKATLTTDKSSSTAYMQL |
| | | SSLTSEDSAVYYCARYYDDHYCLDYWGQGTTLTVSSV |
| | | EGGSGGSGGSGGSGGVDDIQLTQSPAIMSASPGEKVTM |
| | | TCRASSSVSYMNWYQQKSGTSPKRWIYDTSKVASGVP |
| | | YRFSGSGSGTSYSLTISSMEAEDAATYYCQQWSSNPLT |
| | | FGAGTKLELK |
| 30 | GlyTR2^{CD301(4)xCD3}: | VHSEMLLRVQQLVQDLKKLTCQVATLNNNASTEGTC |
| | Extended | CPVNWVEHQDSCYWFSHSGMSWAEAEKYCQLKNAH |
| | CD301CRD- | LVVINSREEQNFVQKYLGSAYTWMGLSDPEGAWKWV |
| | Linker- | DGTDYATGFQNWKPGQPDDWQGHGLGGGEDCAHFH |
| | CD301CRD(3)- | PDGRWNDDVCQRPYHWVCEAGLGQTSQESHGGGGSG |
| | Linker-CD3 | GGGSGGGGSLTCQVATLNNNASTEGTCCPVNWVEHQ |
| | scFv | DSCYWFSHSGMSWAEAEKYCQLKNAHLVVINSREEQ |
| | | NFVQKYLGSAYTWMGLSDPEGAWKWVDGTDYATGF |
| | | QNWKPGQPDDWQGHGLGGGEDCAHFHPDGRWNDDV |
| | | CQRPYHWVCEAGLGQTSQESHGGGGSGGGGSGGGGS |
| | | LTCQVATLNNNASTEGTCCPVNWVEHQDSCYWFSHS |
| | | GMSWAEAEKYCQLKNAHLVVINSREEQNFVQKYLGS |
| | | AYTWMGLSDPEGAWKWVDGTDYATGFQNWKPGQPD |
| | | DWQGHGLGGGEDCAHFHPDGRWNDDVCQRPYHWVC |
| | | EAGLGQTSQESHGGGGSGGGGSGGGGSLTCQVATLNN |
| | | NASTEGTCCPVNWVEHQDSCYWFSHSGMSWAEAEKY |
| | | CQLKNAHLVVINSREEQNFVQKYLGSAYTWMGLSDPE |
| | | GAWKWVDGTDYATGFQNWKPGQPDDWQGHGLGGG |
| | | EDCAHFHPDGRWNDDVCQRPYHWVCEAGLGQTSQES |
| | | HGGGGSGGGGSGGGGSDIKLQQSGAELARPGASVKMS |
| | | CKTSGYTFTRYTMHWVKQRPGQGLEWIGYINPSRGYT |
| | | NYNQKFKDKATLTTDKSSSTAYMQLSSLTSEDSAVYY |
| | | CARYYDDHYCLDYWGQGTTLTVSSVEGGSGGSGGSG |
| | | GSGGVDDIQLTQSPAIMSASPGEKVTMTCRASSSVSYM |
| | | NWYQQKSGTSPKRWIYDTSKVASGVPYRFSGSGSGTS |
| | | YSLTISSMEAEDAATYYCQQWSSNPLTFGAGTKLELK |
| 31 | GlyTR2^{slCD301xCD3}: | VHSEMLLRVQQLVQDLKKLTCQVATLNNNASTEGTC |
| | Extended | CPVNWVEHQDSCYWFSHSGMSWAEAEKYCQLKNAH |
| | CD301CRD- | LVVINSREEQNFVQKYLGSAYTWMGLSDPEGAWKWV |
| | Linker-CD3 | DGTDYATGFQNWKPGQPDDWQGHGLGGGEDCAHFH |
| | scFv | PDGRWNDDVCQRPYHWVCEAGLGQTSQESHAEAAA |
| | | KAAEAAAKADIKLQQSGAELARPGASVKMSCKTSGYT |
| | | FTRYTMHWVKQRPGQGLEWIGYINPSRGYTNYNQKF |
| | | KDKATLTTDKSSSTAYMQLSSLTSEDSAVYYCARYYD |
| | | DHYCLDYWGQGTTLTVSSVEGGSGGSGGSGGSGGVD |
| | | DIQLTQSPAIMSASPGEKVTMTCRASSSVSYMNWYQQ |
| | | KSGTSPKRWIYDTSKVASGVPYRFSGSGSGTSYSLTISS |
| | | MEAEDAATYYCQQWSSNPLTFGAGTKLELK |
| 32 | GlyTR2^{slCD301(2)xCD3}: | VHSEMLLRVQQLVQDLKKLTCQVATLNNNASTEGTC |
| | Extended | CPVNWVEHQDSCYWFSHSGMSWAEAEKYCQLKNAH |
| | CD301CRD- | LVVINSREEQNFVQKYLGSAYTWMGLSDPEGAWKWV |
| | Linker- | DGTDYATGFQNWKPGQPDDWQGHGLGGGEDCAHFH |
| | CD301CRD- | PDGRWNDDVCQRPYHWVCEAGLGQTSQESHAEAAA |
| | Linker-CD3 | KAAEAAAKALTCQVATLNNNASTEGTCCPVNWVEHQ |
| | scFv | DSCYWFSHSGMSWAEAEKYCQLKNAHLVVINSREEQ |
| | | NFVQKYLGSAYTWMGLSDPEGAWKWVDGTDYATGF |
| | | QNWKPGQPDDWQGHGLGGGEDCAHFHPDGRWNDDV |
| | | CQRPYHWVCEAGLGQTSQESHAEAAAKAAEAAAKAD |
| | | IKLQQSGAELARPGASVKMSCKTSGYTFTRYTMHWVK |
| | | QRPGQGLEWIGYINPSRGYTNYNQKFKDKATLTTDKS |
| | | SSTAYMQLSSLTSEDSAVYYCARYYDDHYCLDYWGQ |
| | | GTTLTVSSVEGGSGGSGGSGGSGGVDDIQLTQSPAIMS |
| | | ASPGEKVTMTCRASSSVSYMNWYQQKSGTSPKRWIY |
| | | DTSKVASGVPYRFSGSGSGTSYSLTISSMEAEDAATYY |
| | | CQQWSSNPLTFGAGTKLELK |
| 33 | GlyTR2^{slCD301(3)xCD3}: | VHSEMLLRVQQLVQDLKKLTCQVATLNNNASTEGTC |
| | Extended | CPVNWVEHQDSCYWFSHSGMSWAEAEKYCQLKNAH |
| | CD301CRD | LVVINSREEQNFVQKYLGSAYTWMGLSDPEGAWKWV |
| | Linker- | DGTDYATGFQNWKPGQPDDWQGHGLGGGEDCAHFH |
| | CD301CRD(2)- | PDGRWNDDVCQRPYHWVCEAGLGQTSQESHAEAAA |
| | Linker-CD3 | KAAEAAAKALTCQVATLNNNASTEGTCCPVNWVEHQ |
| | scFv | DSCYWFSHSGMSWAEAEKYCQLKNAHLVVINSREEQ |
| | | NFVQKYLGSAYTWMGLSDPEGAWKWVDGTDYATGF |
| | | QNWKPGQPDDWQGHGLGGGEDCAHFHPDGRWNDDV |
| | | CQRPYHWVCEAGLGQTSQESHAEAAAKAAEAAAKAL |
| | | TCQVATLNNNASTEGTCCPVNWVEHQDSCYWFSHSG |
| | | MSWAEAEKYCQLKNAHLVVINSREEQNFVQKYLGSA |
| | | YTWMGLSDPEGAWKWVDGTDYATGFQNWKPGQPDD |
| | | WQGHGLGGGEDCAHFHPDGRWNDDVCQRPYHWVCE |
| | | AGLGQTSQESHAEAAAKAAEAAAKADIKLQQSGAELA |
| | | RPGASVKMSCKTSGYTFTRYTMHWVKQRPGQGLEWI |
| | | GYINPSRGYTNYNQKFKDKATLTTDKSSSTAYMQLSSL |
| | | TSEDSAVYYCARYYDDHYCLDYWGQGTTLTVSSVEG |
| | | GSGGSGGSGGSGGVDDIQLTQSPAIMSASPGEKVTMTC |
| | | RASSSVSYMNWYQQKSGTSPKRWIYDTSKVASGVPYR |
| | | FSGSGSGTSYSLTISSMEAEDAATYYCQQWSSNPLTFG |
| | | AGTKLELK |
| 34 | GlyTR2^{slCD301(4)xCD3}: | VHSEMLLRVQQLVQDLKKLTCQVATLNNNASTEGTC |
| | Extended | CPVNWVEHQDSCYWFSHSGMSWAEAEKYCQLKNAH |
| | CD301CRD- | LVVINSREEQNFVQKYLGSAYTWMGLSDPEGAWKWV |
| | Linker- | DGTDYATGFQNWKPGQPDDWQGHGLGGGEDCAHFH |
| | CD301CRD(3)- | PDGRWNDDVCQRPYHWVCEAGLGQTSQESHAEAAA |
| | Linker-CD3 | KAAEAAAKALTCQVATLNNNASTEGTCCPVNWVEHQ |
| | scFv | DSCYWFSHSGMSWAEAEKYCQLKNAHLVVINSREEQ |
| | | NFVQKYLGSAYTWMGLSDPEGAWKWVDGTDYATGF |
| | | QNWKPGQPDDWQGHGLGGGEDCAHFHPDGRWNDDV |
| | | CQRPYHWVCEAGLGQTSQESHAEAAAKAAEAAAKAL |
| | | TCQVATLNNNASTEGTCCPVNWVEHQDSCYWFSHSG |
| | | MSWAEAEKYCQLKNAHLVVINSREEQNFVQKYLGSA |
| | | YTWMGLSDPEGAWKWVDGTDYATGFQNWKPGQPDD |
| | | WQGHGLGGGEDCAHFHPDGRWNDDVCQRPYHWVCE |
| | | AGLGQTSQESHAEAAAKAAEAAAKALTCQVATLNNN |
| | | ASTEGTCCPVNWVEHQDSCYWFSHSGMSWAEAEKYC |
| | | QLKNAHLVVINSREEQNFVQKYLGSAYTWMGLSDPEG |
| | | AWKWVDGTDYATGFQNWKPGQPDDWQGHGLGGGE |
| | | DCAHFHPDGRWNDDVCQRPYHWVCEAGLGQTSQESH |
| | | AEAAAKAAEAAAKADIKLQQSGAELARPGASVKMSC |
| | | KTSGYTFTRYTMHWVKQRPGQGLEWIGYINPSRGYTN |
| | | YNQKFKDKATLTTDKSSSTAYMQLSSLTSEDSAVYYC |
| | | ARYYDDHYCLDYWGQGTTLTVSSVEGGSGGSGGSGG |
| | | SGGVDDIQLTQSPAIMSASPGEKVTMTCRASSSVSYMN |
| | | WYQQKSGTSPKRWIYDTSKVASGVPYRFSGSGSGTSY |
| | | SLTISSMEAEDAATYYCQQWSSNPLTFGAGTKLELK |
| 35 | HSA- | DAHKSEVAHRFKDLGEENFKALVLIAFAQYLQQCPFE |
| | GlyTR2^{slCD301xCD3}: | DHVKLVNEVTEFAKTCVADESAENCDKSLHTLFGDKL |
| | HSA-Linker- | CTVATLRETYGEMADCCAKQEPERNECFLQHKDDNPN |
| | Extended | LPRLVRPEVDVMCTAFHDNEETFLKKYLYEIARRHPYF |
| | CD301CRD- | YAPELLFFAKRYKAAFTECCQAADKAACLLPKLDELR |
| | Linker-CD3 scFv | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAVHSEMLLRVQQLVQDLKKLTCQVATLNNNAST |
| | | EGTCCPVNWVEHQDSCYWFSHSGMSWAEAEKYCQLK |
| | | NAHLVVINSREEQNFVQKYLGSAYTWMGLSDPEGAW |
| | | KWVDGTDYATGFQNWKPGQPDDWQGHGLGGGEDCA |
| | | HFHPDGRWNDDVCQRPYHWVCEAGLGQTSQESHAEA |
| | | AAKAAEAAAKADIKLQQSGAELARPGASVKMSCKTS |
| | | GYTFTRYTMHWVKQRPGQGLEWIGYINPSRGYTNYN |
| | | QKFKDKATLTTDKSSSTAYMQLSSLTSEDSAVYYCAR |
| | | YYDDHYCLDYWGQGTTLTVSSVEGGSGGSGGSGGSG |
| | | GVDDIQLTQSPAIMSASPGEKVTMTCRASSSVSYMNW |
| | | YQQKSGTSPKRWIYDTSKVASGVPYRFSGSGSGTSYSL |
| | | TISSMEAEDAATYYCQQWSSNPLTFGAGTKLELK |
| 36 | HSA- | DAHKSEVAHRFKDLGEENFKALVLIAFAQYLQQCPFE |
| | GlyTR2^{slCD301(2)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker-Extended | LPRLVRPEVDVMCTAFHDNEETFLKKYLYEIARRHPYF |
| | CD301CRD- | YAPELLFFAKRYKAAFTECCQAADKAACLLPKLDELR |
| | Linker- | DEGKASSAKQRLKCASLQKFGERAFKAWAVARLSQRF |
| | CD301CRD- | PKAEFAEVSKLVTDLTKVHTECCHGDLLECADDRADL |
| | Linker-CD3 | AKYICENQDSISSKLKECCEKPLLEKSHCIAEVENDEMP |
| | scFv | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAVHSEMLLRVQQLVQDLKKLTCQVATLNNNAST |
| | | EGTCCPVNWVEHQDSCYWFSHSGMSWAEAEKYCQLK |
| | | NAHLVVINSREEQNFVQKYLGSAYTWMGLSDPEGAW |
| | | KWVDGTDYATGFQNWKPGQPDDWQGHGLGGGEDCA |
| | | HFHPDGRWNDDVCQRPYHWVCEAGLGQTSQESHAEA |
| | | AAKAAEAAAKALTCQVATLNNNASTEGTCCPVNWVE |
| | | HQDSCYWFSHSGMSWAEAEKYCQLKNAHLVVINSRE |
| | | EQNFVQKYLGSAYTWMGLSDPEGAWKWVDGTDYAT |
| | | GFQNWKPGQPDDWQGHGLGGGEDCAHFHPDGRWND |
| | | DVCQRPYHWVCEAGLGQTSQESHAEAAAKAAEAAAK |
| | | ADIKLQQSGAELARPGASVKMSCKTSGYTFTRYTMHW |
| | | VKQRPGQGLEWIGYINPSRGYTNYNQKFKDKATLTTD |
| | | KSSSTAYMQLSSLTSEDSAVYYCARYYDDHYCLDYW |
| | | GQGTTLTVSSVEGGSGGSGGSGGSGGVDDIQLTQSPAI |
| | | MSASPGEKVTMTCRASSSVSYMNWYQQKSGTSPKRW |
| | | IYDTSKVASGVPYRFSGSGSGTSYSLTISSMEAEDAATY |
| | | YCQQWSSNPLTFGAGTKLELK |
| 37 | HSA- | DAHKSEVAHRFKDLGEENFKALVLIAFAQYLQQCPFE |
| | GlyTR2^{slCD301(3)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker-Extended | LPRLVRPEVDVMCTAFHDNEETFLKKYLYEIARRHPYF |
| | CD301CRD- | YAPELLFFAKRYKAAFTECCQAADKAACLLPKLDELR |
| | Linker- | DEGKASSAKQRLKCASLQKFGERAFKAWAVARLSQRF |
| | CD301CRD(2)- | PKAEFAEVSKLVTDLTKVHTECCHGDLLECADDRADL |
| | Linker-CD3 | AKYICENQDSISSKLKECCEKPLLEKSHCIAEVENDEMP |
| | scFv | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAVHSEMLLRVQQLVQDLKKLTCQVATLNNNAST |
| | | EGTCCPVNWVEHQDSCYWFSHSGMSWAEAEKYCQLK |
| | | NAHLVVINSREEQNFVQKYLGSAYTWMGLSDPEGAW |
| | | KWVDGTDYATGFQNWKPGQPDDWQGHGLGGGEDCA |
| | | HFHPDGRWNDDVCQRPYHWVCEAGLGQTSQESHAEA |
| | | AAKAAEAAAKALTCQVATLNNNASTEGTCCPVNWVE |
| | | HQDSCYWFSHSGMSWAEAEKYCQLKNAHLVVINSRE |
| | | EQNFVQKYLGSAYTWMGLSDPEGAWKWVDGTDYAT |
| | | GFQNWKPGQPDDWQGHGLGGGEDCAHFHPDGRWND |
| | | DVCQRPYHWVCEAGLGQTSQESHAEAAAKAAEAAAK |
| | | ALTCQVATLNNNASTEGTCCPVNWVEHQDSCYWFSH |
| | | SGMSWAEAEKYCQLKNAHLVVINSREEQNFVQKYLG |
| | | SAYTWMGLSDPEGAWKWVDGTDYATGFQNWKPGQP |
| | | DDWQGHGLGGGEDCAHFHPDGRWNDDVCQRPYHW |
| | | VCEAGLGQTSQESHAEAAAKAAEAAAKADIKLQQSG |
| | | AELARPGASVKMSCKTSGYTFTRYTMHWVKQRPGQG |
| | | LEWIGYINPSRGYTNYNQKFKDKATLTTDKSSSTAYM |
| | | QLSSLTSEDSAVYYCARYYDDHYCLDYWGQGTTLTVS |
| | | SVEGGSGGSGGSGGSGGVDDIQLTQSPAIMSASPGEKV |
| | | TMTCRASSSVSYMNWYQQKSGTSPKRWIYDTSKVAS |
| | | GVPYRFSGSGSGTSYSLTISSMEAEDAATYYCQQWSSN |
| | | PLTFGAGTKLELK |
| 38 | HSA- | DAHKSEVAHRFKDLGEENFKALVLIAFAQYLQQCPFE |
| | GlyTR2^{slCD301(4)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker-Extended | LPRLVRPEVDVMCTAFHDNEETFLKKYLYEIARRHPYF |
| | CD301CRD- | YAPELLFFAKRYKAAFTECCQAADKAACLLPKLDELR |
| | Linker- | DEGKASSAKQRLKCASLQKFGERAFKAWAVARLSQRF |
| | CD301CRD(3)- | PKAEFAEVSKLVTDLTKVHTECCHGDLLECADDRADL |
| | Linker-CD3 | AKYICENQDSISSKLKECCEKPLLEKSHCIAEVENDEMP |
| | scFv | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKAVHSEMLLRVQQLVQDLKKLTCQVATLNNNAST |
| | | EGTCCPVNWVEHQDSCYWFSHSGMSWAEAEKYCQLK |
| | | NAHLVVINSREEQNFVQKYLGSAYTWMGLSDPEGAW |
| | | KWVDGTDYATGFQNWKPGQPDDWQGHGLGGGEDCA |
| | | HFHPDGRWNDDVCQRPYHWVCEAGLGQTSQESHAEA |
| | | AAKAAEAAAKALTCQVATLNNNASTEGTCCPVNWVE |
| | | HQDSCYWFSHSGMSWAEAEKYCQLKNAHLVVINSRE |
| | | EQNFVQKYLGSAYTWMGLSDPEGAWKWVDGTDYAT |
| | | GFQNWKPGQPDDWQGHGLGGGEDCAHFHPDGRWND |
| | | DVCQRPYHWVCEAGLGQTSQESHAEAAAKAAEAAAK |
| | | ALTCQVATLNNNASTEGTCCPVNWVEHQDSCYWFSH |
| | | SGMSWAEAEKYCQLKNAHLVVINSREEQNFVQKYLG |
| | | SAYTWMGLSDPEGAWKWVDGTDYATGFQNWKPGQP |
| | | DDWQGHGLGGGEDCAHFHPDGRWNDDVCQRPYHW |
| | | VCEAGLGQTSQESHAEAAAKAAEAAAKALTCQVATL |
| | | NNNASTEGTCCPVNWVEHQDSCYWFSHSGMSWAEAE |
| | | KYCQLKNAHLVVINSREEQNFVQKYLGSAYTWMGLS |
| | | DPEGAWKWVDGTDYATGFQNWKPGQPDDWQGHGL |
| | | GGGEDCAHFHPDGRWNDDVCQRPYHWVCEAGLGQT |
| | | SQESHAEAAAKAAEAAAKADIKLQQSGAELARPGASV |
| | | KMSCKTSGYTFTRYTMHWVKQRPGQGLEWIGYINPSR |
| | | GYTNYNQKFKDKATLTTDKSSSTAYMQLSSLTSEDSA |
| | | VYYCARYYDDHYCLDYWGQGTTLTVSSVEGGSGGSG |
| | | GSGGSGGVDDIQLTQSPAIMSASPGEKVTMTCRASSSV |
| | | SYMNWYQQKSGTSPKRWIYDTSKVASGVPYRFSGSGS |
| | | GTSYSLTISSMEAEDAATYYCQQWSSNPLTFGAGTKLE |
| | | LK |
| 39 | GlyTR2^{slCD301xCD3}: | LTCQVATLNNNASTEGTCCPVNWVEHQDSCYWFSHS |
| | CD301(163- | GMSWAEAEKYCQLKNAHLVVINSREEQNFVQKYLGS |
| | 316)-Linker- | AYTWMGLSDPEGAWKWVDGTDYATGFQNWKPGQPD |
| | CD3 scFv | DWQGHGLGGGEDCAHFHPDGRWNDDVCQRPYHWVC |
| | | EAGLGQTSQESHAEAAAKAAEAAAKADIKLQQSGAEL |
| | | ARPGASVKMSCKTSGYTFTRYTMHWVKQRPGQGLEW |
| | | IGYINPSRGYTNYNQKFKDKATLTTDKSSSTAYMQLSS |
| | | LTSEDSAVYYCARYYDDHYCLDYWGQGTTLTVSSVE |
| | | GGSGGSGGSGGSGGVDDIQLTQSPAIMSASPGEKVTMT |
| | | CRASSSVSYMNWYQQKSGTSPKRWIYDTSKVASGVPY |
| | | RFSGSGSGTSYSLTISSMEAEDAATYYCQQWSSNPLTF |
| | | GAGTKLELK |
| 40 | GlyTR2^{slCD301(2)xCD3}: | LTCQVATLNNNASTEGTCCPVNWVEHQDSCYWFSHS |
| | CD301(163- | GMSWAEAEKYCQLKNAHLVVINSREEQNFVQKYLGS |
| | 316)x2-Linker- | AYTWMGLSDPEGAWKWVDGTDYATGFQNWKPGQPD |
| | CD3 scFv | DWQGHGLGGGEDCAHFHPDGRWNDDVCQRPYHWVC |
| | | EAGLGQTSQESHAEAAAKAAEAAAKALTCQVATLNN |
| | | NASTEGTCCPVNWVEHQDSCYWFSHSGMSWAEAEKY |
| | | CQLKNAHLVVINSREEQNFVQKYLGSAYTWMGLSDPE |
| | | GAWKWVDGTDYATGFQNWKPGQPDDWQGHGLGGG |
| | | EDCAHFHPDGRWNDDVCQRPYHWVCEAGLGQTSQES |
| | | HAEAAAKAAEAAAKADIKLQQSGAELARPGASVKMS |
| | | CKTSGYTFTRYTMHWVKQRPGQGLEWIGYINPSRGYT |
| | | NYNQKFKDKATLTTDKSSSTAYMQLSSLTSEDSAVYY |
| | | CARYYDDHYCLDYWGQGTTLTVSSVEGGSGGSGGSG |
| | | GSGGVDDIQLTQSPAIMSASPGEKVTMTCRASSSVSYM |
| | | NWYQQKSGTSPKRWIYDTSKVASGVPYRFSGSGSGTS |
| | | YSLTISSMEAEDAATYYCQQWSSNPLTFGAGTKLELK |
| 41 | GlyTR2^{slCD301(3)xCD3}: | LTCQVATLNNNASTEGTCCPVNWVEHQDSCYWFSHS |
| | CD301(163- | GMSWAEAEKYCQLKNAHLVVINSREEQNFVQKYLGS |
| | 316)x3-Linker- | AYTWMGLSDPEGAWKWVDGTDYATGFQNWKPGQPD |
| | CD3 scFv | DWQGHGLGGGEDCAHFHPDGRWNDDVCQRPYHWVC |
| | | EAGLGQTSQESHAEAAAKAAEAAAKALTCQVATLNN |
| | | NASTEGTCCPVNWVEHQDSCYWFSHSGMSWAEAEKY |
| | | CQLKNAHLVVINSREEQNFVQKYLGSAYTWMGLSDPE |
| | | GAWKWVDGTDYATGFQNWKPGQPDDWQGHGLGGG |
| | | EDCAHFHPDGRWNDDVCQRPYHWVCEAGLGQTSQES |
| | | HAEAAAKAAEAAAKALTCQVATLNNNASTEGTCCPV |
| | | NWVEHQDSCYWFSHSGMSWAEAEKYCQLKNAHLVVI |
| | | NSREEQNFVQKYLGSAYTWMGLSDPEGAWKWVDGT |
| | | DYATGFQNWKPGQPDDWQGHGLGGGEDCAHFHPDG |
| | | RWNDDVCQRPYHWVCEAGLGQTSQESHAEAAAKAA |
| | | EAAAKADIKLQQSGAELARPGASVKMSCKTSGYTFTR |
| | | YTMHWVKQRPGQGLEWIGYINPSRGYTNYNQKFKDK |
| | | ATLTTDKSSSTAYMQLSSLTSEDSAVYYCARYYDDHY |
| | | CLDYWGQGTTLTVSSVEGGSGGSGGSGGSGGVDDIQL |
| | | TQSPAIMSASPGEKVTMTCRASSSVSYMNWYQQKSGT |
| | | SPKRWIYDTSKVASGVPYRFSGSGSGTSYSLTISSMEAE |
| | | DAATYYCQQWSSNPLTFGAGTKLELK |
| 42 | GlyTR2^{slCD301(4)xCD3}: | LTCQVATLNNNASTEGTCCPVNWVEHQDSCYWFSHS |
| | CD301(163- | GMSWAEAEKYCQLKNAHLVVINSREEQNFVQKYLGS |
| | 316)x4-Linker- | AYTWMGLSDPEGAWKWVDGTDYATGFQNWKPGQPD |
| | CD3 scFv | DWQGHGLGGGEDCAHFHPDGRWNDDVCQRPYHWVC |
| | | EAGLGQTSQESHAEAAAKAAEAAAKALTCQVATLNN |
| | | NASTEGTCCPVNWVEHQDSCYWFSHSGMSWAEAEKY |
| | | CQLKNAHLVVINSREEQNFVQKYLGSAYTWMGLSDPE |
| | | GAWKWVDGTDYATGFQNWKPGQPDDWQGHGLGGG |
| | | EDCAHFHPDGRWNDDVCQRPYHWVCEAGLGQTSQES |
| | | HAEAAAKAAEAAAKALTCQVATLNNNASTEGTCCPV |
| | | NWVEHQDSCYWFSHSGMSWAEAEKYCQLKNAHLVVI |
| | | NSREEQNFVQKYLGSAYTWMGLSDPEGAWKWVDGT |
| | | DYATGFQNWKPGQPDDWQGHGLGGGEDCAHFHPDG |
| | | RWNDDVCQRPYHWVCEAGLGQTSQESHAEAAAKAA |
| | | EAAAKALTCQVATLNNNASTEGTCCPVNWVEHQDSC |
| | | YWFSHSGMSWAEAEKYCQLKNAHLVVINSREEQNFV |
| | | QKYLGSAYTWMGLSDPEGAWKWVDGTDYATGFQN |
| | | WKPGQPDDWQGHGLGGGEDCAHFHPDGRWNDDVCQ |
| | | RPYHWVCEAGLGQTSQESHAEAAAKAAEAAAKADIK |
| | | LQQSGAELARPGASVKMSCKTSGYTFTRYTMHWVKQ |
| | | RPGQGLEWIGYINPSRGYTNYNQKFKDKATLTTDKSSS |
| | | TAYMQLSSLTSEDSAVYYCARYYDDHYCLDYWGQGT |
| | | TLTVSSVEGGSGGSGGSGGSGGVDDIQLTQSPAIMSAS |
| | | PGEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 43 | HSA- | DAHKSEVAHRFKDLGEENFKALVLIAFAQYLQQCPFE |
| | GlyTR2^{slCD301xCD3}: | DHVKLVNEVTEFAKTCVADESAENCDKSLHTLFGDKL |
| | HSA-Linker- | CTVATLRETYGEMADCCAKQEPERNECFLQHKDDNPN |
| | CD301(163- | LPRLVRPEVDVMCTAFHDNEETFLKKYLYEIARRHPYF |
| | 316)-Linker- | YAPELLFFAKRYKAAFTECCQAADKAACLLPKLDELR |
| | CD3 scFv | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKALTCQVATLNNNASTEGTCCPVNWVEHQDSCYW |
| | | FSHSGMSWAEAEKYCQLKNAHLVVINSREEQNFVQKY |
| | | LGSAYTWMGLSDPEGAWKWVDGTDYATGFQNWKPG |
| | | QPDDWQGHGLGGGEDCAHFHPDGRWNDDVCQRPYH |
| | | WVCEAGLGQTSQESHAEAAAKAAEAAAKADIKLQQS |
| | | GAELARPGASVKMSCKTSGYTFTRYTMHWVKQRPGQ |
| | | GLEWIGYINPSRGYTNYNQKFKDKATLTTDKSSSTAY |
| | | MQLSSLTSEDSAVYYCARYYDDHYCLDYWGQGTTLT |
| | | VSSVEGGSGGSGGSGGSGGVDDIQLTQSPAIMSASPGE |
| | | KVTMTCRASSSVSYMNWYQQKSGTSPKRWIYDTSKV |
| | | ASGVPYRFSGSGSGTSYSLTISSMEAEDAATYYCQQWS |
| | | SNPLTFGAGTKLELK |
| 44 | HSA- | DAHKSEVAHRFKDLGEENFKALVLIAFAQYLQQCPFE |
| | GlyTR2^{slCD301(2)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(163- | YAPELLFFAKRYKAAFTECCQAADKAACLLPKLDELR |
| | 316)x2-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKALTCQVATLNNNASTEGTCCPVNWVEHQDSCYW |
| | | FSHSGMSWAEAEKYCQLKNAHLVVINSREEQNFVQKY |
| | | LGSAYTWMGLSDPEGAWKWVDGTDYATGFQNWKPG |
| | | QPDDWQGHGLGGGEDCAHFHPDGRWNDDVCQRPYH |
| | | WVCEAGLGQTSQESHAEAAAKAAEAAAKALTCQVAT |
| | | LNNNASTEGTCCPVNWVEHQDSCYWFSHSGMSWAEA |
| | | EKYCQLKNAHLVVINSREEQNFVQKYLGSAYTWMGL |
| | | SDPEGAWKWVDGTDYATGFQNWKPGQPDDWQGHGL |
| | | GGGEDCAHFHPDGRWNDDVCQRPYHWVCEAGLGQT |
| | | SQESHAEAAAKAAEAAAKADIKLQQSGAELARPGASV |
| | | KMSCKTSGYTFTRYTMHWVKQRPGQGLEWIGYINPSR |
| | | GYTNYNQKFKDKATLTTDKSSSTAYMQLSSLTSEDSA |
| | | VYYCARYYDDHYCLDYWGQGTTLTVSSVEGGSGGSG |
| | | GSGGSGGVDDIQLTQSPAIMSASPGEKVTMTCRASSSV |
| | | SYMNWYQQKSGTSPKRWIYDTSKVASGVPYRFSGSGS |
| | | GTSYSLTISSMEAEDAATYYCQQWSSNPLTFGAGTKLE |
| | | LK |
| 45 | HSA- | DAHKSEVAHRFKDLGEENFKALVLIAFAQYLQQCPFE |
| | GlyTR2^{slCD301(3)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(163- | YAPELLFFAKRYKAAFTECCQAADKAACLLPKLDELR |
| | 316)x3-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKALTCQVATLNNNASTEGTCCPVNWVEHQDSCYW |
| | | FSHSGMSWAEAEKYCQLKNAHLVVINSREEQNFVQKY |
| | | LGSAYTWMGLSDPEGAWKWVDGTDYATGFQNWKPG |
| | | QPDDWQGHGLGGGEDCAHFHPDGRWNDDVCQRPYH |
| | | WVCEAGLGQTSQESHAEAAAKAAEAAAKALTCQVAT |
| | | LNNNASTEGTCCPVNWVEHQDSCYWFSHSGMSWAEA |
| | | EKYCQLKNAHLVVINSREEQNFVQKYLGSAYTWMGL |
| | | SDPEGAWKWVDGTDYATGFQNWKPGQPDDWQGHGL |
| | | GGGEDCAHFHPDGRWNDDVCQRPYHWVCEAGLGQT |
| | | SQESHAEAAAKAAEAAAKALTCQVATLNNNASTEGT |
| | | CCPVNWVEHQDSCYWFSHSGMSWAEAEKYCQLKNA |
| | | HLVVINSREEQNFVQKYLGSAYTWMGLSDPEGAWKW |
| | | VDGTDYATGFQNWKPGQPDDWQGHGLGGGEDCAHF |
| | | HPDGRWNDDVCQRPYHWVCEAGLGQTSQESHAEAA |
| | | AKAAEAAAKADIKLQQSGAELARPGASVKMSCKTSG |
| | | YTFTRYTMHWVKQRPGQGLEWIGYINPSRGYTNYNQ |
| | | KFKDKATLTTDKSSSTAYMQLSSLTSEDSAVYYCARY |
| | | YDDHYCLDYWGQGTTLTVSSVEGGSGGSGGSGGSGG |
| | | VDDIQLTQSPAIMSASPGEKVTMTCRASSSVSYMNWY |
| | | QQKSGTSPKRWIYDTSKVASGVPYRFSGSGSGTSYSLTI |
| | | SSMEAEDAATYYCQQWSSNPLTFGAGTKLELK |
| 46 | HSA- | DAHKSEVAHRFKDLGEENFKALVLIAFAQYLQQCPFE |
| | GlyTR2^{slCD301(4)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(163- | YAPELLFFAKRYKAAFTECCQAADKAACLLPKLDELR |
| | 316)x4-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKALTCQVATLNNNASTEGTCCPVNWVEHQDSCYW |
| | | FSHSGMSWAEAEKYCQLKNAHLVVINSREEQNFVQKY |
| | | LGSAYTWMGLSDPEGAWKWVDGTDYATGFQNWKPG |
| | | QPDDWQGHGLGGGEDCAHFHPDGRWNDDVCQRPYH |
| | | WVCEAGLGQTSQESHAEAAAKAAEAAAKALTCQVAT |
| | | LNNNASTEGTCCPVNWVEHQDSCYWFSHSGMSWAEA |
| | | EKYCQLKNAHLVVINSREEQNFVQKYLGSAYTWMGL |
| | | SDPEGAWKWVDGTDYATGFQNWKPGQPDDWQGHGL |
| | | GGGEDCAHFHPDGRWNDDVCQRPYHWVCEAGLGQT |
| | | SQESHAEAAAKAAEAAAKALTCQVATLNNNASTEGT |
| | | CCPVNWVEHQDSCYWFSHSGMSWAEAEKYCQLKNA |
| | | HLVVINSREEQNFVQKYLGSAYTWMGLSDPEGAWKW |
| | | VDGTDYATGFQNWKPGQPDDWQGHGLGGGEDCAHF |
| | | HPDGRWNDDVCQRPYHWVCEAGLGQTSQESHAEAA |
| | | AKAAEAAAKALTCQVATLNNNASTEGTCCPVNWVEH |
| | | QDSCYWFSHSGMSWAEAEKYCQLKNAHLVVINSREE |
| | | QNFVQKYLGSAYTWMGLSDPEGAWKWVDGTDYATG |
| | | FQNWKPGQPDDWQGHGLGGGEDCAHFHPDGRWNDD |
| | | VCQRPYHWVCEAGLGQTSQESHAEAAAKAAEAAAKA |
| | | DIKLQQSGAELARPGASVKMSCKTSGYTFTRYTMHWV |
| | | KQRPGQGLEWIGYINPSRGYTNYNQKFKDKATLTTDK |
| | | SSSTAYMQLSSLTSEDSAVYYCARYYDDHYCLDYWG |
| | | QGTTLTVSSVEGGSGGSGGSGGSGGVDDIQLTQSPAIM |
| | | SASPGEKVTMTCRASSSVSYMNWYQQKSGTSPKRWIY |
| | | DTSKVASGVPYRFSGSGSGTSYSLTISSMEAEDAATYY |
| | | CQQWSSNPLTFGAGTKLELK |
| 47 | GlyTR2^{slCD301xCD3}: | LTCQVATLNNNASTEGTCCPVNWVEHQDSCYWFSHS |
| | CD301(163- | GMSWAEAEKYCQLKNAHLVVINSREEQNFVQKYLGS |
| | 306)-Linker- | AYTWMGLSDPEGAWKWVDGTDYATGFQNWKPGQPD |
| | CD3 scFv | DWQGHGLGGGEDCAHFHPDGRWNDDVCQRPYHWVC |
| | | EAAEAAAKAAEAAAKADIKLQQSGAELARPGASVKM |
| | | SCKTSGYTFTRYTMHWVKQRPGQGLEWIGYINPSRGY |
| | | TNYNQKFKDKATLTTDKSSSTAYMQLSSLTSEDSAVY |
| | | YCARYYDDHYCLDYWGQGTTLTVSSVEGGSGGSGGS |
| | | GGSGGVDDIQLTQSPAIMSASPGEKVTMTCRASSSVSY |
| | | MNWYQQKSGTSPKRWIYDTSKVASGVPYRFSGSGSGT |
| | | SYSLTISSMEAEDAATYYCQQWSSNPLTFGAGTKLELK |
| 48 | GlyTR2^{slCD301(2)xCD3}: | LTCQVATLNNNASTEGTCCPVNWVEHQDSCYWFSHS |
| | CD301(163- | GMSWAEAEKYCQLKNAHLVVINSREEQNFVQKYLGS |
| | 306)x2-Linker- | AYTWMGLSDPEGAWKWVDGTDYATGFQNWKPGQPD |
| | CD3 scFv | DWQGHGLGGGEDCAHFHPDGRWNDDVCQRPYHWVC |
| | | EAAEAAAKAAEAAAKALTCQVATLNNNASTEGTCCP |
| | | VNWVEHQDSCYWFSHSGMSWAEAEKYCQLKNAHLV |
| | | VINSREEQNFVQKYLGSAYTWMGLSDPEGAWKWVDG |
| | | TDYATGFQNWKPGQPDDWQGHGLGGGEDCAHFHPD |
| | | GRWNDDVCQRPYHWVCEAAEAAAKAAEAAAKADIK |
| | | LQQSGAELARPGASVKMSCKTSGYTFTRYTMHWVKQ |
| | | RPGQGLEWIGYINPSRGYTNYNQKFKDKATLTTDKSSS |
| | | TAYMQLSSLTSEDSAVYYCARYYDDHYCLDYWGQGT |
| | | TLTVSSVEGGSGGSGGSGGSGGVDDIQLTQSPAIMSAS |
| | | PGEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 49 | GlyTR2^{slCD301(3)xCD3}: | LTCQVATLNNNASTEGTCCPVNWVEHQDSCYWFSHS |
| | CD301(163- | GMSWAEAEKYCQLKNAHLVVINSREEQNFVQKYLGS |
| | 306)x3-Linker- | AYTWMGLSDPEGAWKWVDGTDYATGFQNWKPGQPD |
| | CD3 scFv | DWQGHGLGGGEDCAHFHPDGRWNDDVCQRPYHWVC |
| | | EAAEAAAKAAEAAAKALTCQVATLNNNASTEGTCCP |
| | | VNWVEHQDSCYWFSHSGMSWAEAEKYCQLKNAHLV |
| | | VINSREEQNFVQKYLGSAYTWMGLSDPEGAWKWVDG |
| | | TDYATGFQNWKPGQPDDWQGHGLGGGEDCAHFHPD |
| | | GRWNDDVCQRPYHWVCEAAEAAAKAAEAAAKALTC |
| | | QVATLNNNASTEGTCCPVNWVEHQDSCYWFSHSGMS |
| | | WAEAEKYCQLKNAHLVVINSREEQNFVQKYLGSAYT |
| | | WMGLSDPEGAWKWVDGTDYATGFQNWKPGQPDDW |
| | | QGHGLGGGEDCAHFHPDGRWNDDVCQRPYHWVCEA |
| | | AEAAAKAAEAAAKADIKLQQSGAELARPGASVKMSC |
| | | KTSGYTFTRYTMHWVKQRPGQGLEWIGYINPSRGYTN |
| | | YNQKFKDKATLTTDKSSSTAYMQLSSLTSEDSAVYYC |
| | | ARYYDDHYCLDYWGQGTTLTVSSVEGGSGGSGGSGG |
| | | SGGVDDIQLTQSPAIMSASPGEKVTMTCRASSSVSYMN |
| | | WYQQKSGTSPKRWIYDTSKVASGVPYRFSGSGSGTSY |
| | | SLTISSMEAEDAATYYCQQWSSNPLTFGAGTKLELK |
| 50 | GlyTR2^{slCD301(4)xCD3}: | LTCQVATLNNNASTEGTCCPVNWVEHQDSCYWFSHS |
| | CD301(163- | GMSWAEAEKYCQLKNAHLVVINSREEQNFVQKYLGS |
| | 306)x4-Linker- | AYTWMGLSDPEGAWKWVDGTDYATGFQNWKPGQPD |
| | CD3 scFv | DWQGHGLGGGEDCAHFHPDGRWNDDVCQRPYHWVC |
| | | EAAEAAAKAAEAAAKALTCQVATLNNNASTEGTCCP |
| | | VNWVEHQDSCYWFSHSGMSWAEAEKYCQLKNAHLV |
| | | VINSREEQNFVQKYLGSAYTWMGLSDPEGAWKWVDG |
| | | TDYATGFQNWKPGQPDDWQGHGLGGGEDCAHFHPD |
| | | GRWNDDVCQRPYHWVCEAAEAAAKAAEAAAKALTC |
| | | QVATLNNNASTEGTCCPVNWVEHQDSCYWFSHSGMS |
| | | WAEAEKYCQLKNAHLVVINSREEQNFVQKYLGSAYT |
| | | WMGLSDPEGAWKWVDGTDYATGFQNWKPGQPDDW |
| | | QGHGLGGGEDCAHFHPDGRWNDDVCQRPYHWVCEA |
| | | AEAAAKAAEAAAKALTCQVATLNNNASTEGTCCPVN |
| | | WVEHQDSCYWFSHSGMSWAEAEKYCQLKNAHLVVIN |
| | | SREEQNFVQKYLGSAYTWMGLSDPEGAWKWVDGTD |
| | | YATGFQNWKPGQPDDWQGHGLGGGEDCAHFHPDGR |
| | | WNDDVCQRPYHWVCEAAEAAAKAAEAAAKADIKLQ |
| | | QSGAELARPGASVKMSCKTSGYTFTRYTMHWVKQRP |
| | | GQGLEWIGYINPSRGYTNYNQKFKDKATLTTDKSSSTA |
| | | YMQLSSLTSEDSAVYYCARYYDDHYCLDYWGQGTTL |
| | | TVSSVEGGSGGSGGSGGSGGVDDIQLTQSPAIMSASPG |
| | | EKVTMTCRASSSVSYMNWYQQKSGTSPKRWIYDTSK |
| | | VASGVPYRFSGSGSGTSYSLTISSMEAEDAATYYCQQ |
| | | WSSNPLTFGAGTKLELK |
| 51 | HSA- | DAHKSEVAHRFKDLGEENFKALVLIAFAQYLQQCPFE |
| | GlyTR2^{slCD301xCD3}: | DHVKLVNEVTEFAKTCVADESAENCDKSLHTLFGDKL |
| | HSA-Linker- | CTVATLRETYGEMADCCAKQEPERNECFLQHKDDNPN |
| | CD301(163- | LPRLVRPEVDVMCTAFHDNEETFLKKYLYEIARRHPYF |
| | 306)-Linker- | YAPELLFFAKRYKAAFTECCQAADKAACLLPKLDELR |
| | CD3 scFv | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKALTCQVATLNNNASTEGTCCPVNWVEHQDSCYW |
| | | FSHSGMSWAEAEKYCQLKNAHLVVINSREEQNFVQKY |
| | | LGSAYTWMGLSDPEGAWKWVDGTDYATGFQNWKPG |
| | | QPDDWQGHGLGGGEDCAHFHPDGRWNDDVCQRPYH |
| | | WVCEAAEAAAKAAEAAAKADIKLQQSGAELARPGAS |
| | | VKMSCKTSGYTFTRYTMHWVKQRPGQGLEWIGYINPS |
| | | RGYTNYNQKFKDKATLTTDKSSSTAYMQLSSLTSEDS |
| | | AVYYCARYYDDHYCLDYWGQGTTLTVSSVEGGSGGS |
| | | GGSGGSGGVDDIQLTQSPAIMSASPGEKVTMTCRASSS |
| | | VSYMNWYQQKSGTSPKRWIYDTSKVASGVPYRFSGSG |
| | | SGTSYSLTISSMEAEDAATYYCQQWSSNPLTFGAGTKL |
| | | ELK |
| 52 | HSA- | DAHKSEVAHRFKDLGEENFKALVLIAFAQYLQQCPFE |
| | GlyTR2^{slCD301(2)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(163- | YAPELLFFAKRYKAAFTECCQAADKAACLLPKLDELR |
| | 306)x2-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKALTCQVATLNNNASTEGTCCPVNWVEHQDSCYW |
| | | FSHSGMSWAEAEKYCQLKNAHLVVINSREEQNFVQKY |
| | | LGSAYTWMGLSDPEGAWKWVDGTDYATGFQNWKPG |
| | | QPDDWQGHGLGGGEDCAHFHPDGRWNDDVCQRPYH |
| | | WVCEAAEAAAKAAEAAAKALTCQVATLNNNASTEGT |
| | | CCPVNWVEHQDSCYWFSHSGMSWAEAEKYCQLKNA |
| | | HLVVINSREEQNFVQKYLGSAYTWMGLSDPEGAWKW |
| | | VDGTDYATGFQNWKPGQPDDWQGHGLGGGEDCAHF |
| | | HPDGRWNDDVCQRPYHWVCEAAEAAAKAAEAAAKA |
| | | DIKLQQSGAELARPGASVKMSCKTSGYTFTRYTMHWV |
| | | KQRPGQGLEWIGYINPSRGYTNYNQKFKDKATLTTDK |
| | | SSSTAYMQLSSLTSEDSAVYYCARYYDDHYCLDYWG |
| | | QGTTLTVSSVEGGSGGSGGSGGSGGVDDIQLTQSPAIM |
| | | SASPGEKVTMTCRASSSVSYMNWYQQKSGTSPKRWIY |
| | | DTSKVASGVPYRFSGSGSGTSYSLTISSMEAEDAATYY |
| | | CQQWSSNPLTFGAGTKLELK |
| 53 | HSA- | DAHKSEVAHRFKDLGEENFKALVLIAFAQYLQQCPFE |
| | GlyTR2^{slCD301(3)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(163- | YAPELLFFAKRYKAAFTECCQAADKAACLLPKLDELR |
| | 306)x3-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKALTCQVATLNNNASTEGTCCPVNWVEHQDSCYW |
| | | FSHSGMSWAEAEKYCQLKNAHLVVINSREEQNFVQKY |
| | | LGSAYTWMGLSDPEGAWKWVDGTDYATGFQNWKPG |
| | | QPDDWQGHGLGGGEDCAHFHPDGRWNDDVCQRPYH |
| | | WVCEAAEAAAKAAEAAAKALTCQVATLNNNASTEGT |
| | | CCPVNWVEHQDSCYWFSHSGMSWAEAEKYCQLKNA |
| | | HLVVINSREEQNFVQKYLGSAYTWMGLSDPEGAWKW |
| | | VDGTDYATGFQNWKPGQPDDWQGHGLGGGEDCAHF |
| | | HPDGRWNDDVCQRPYHWVCEAAEAAAKAAEAAAKA |
| | | LTCQVATLNNNASTEGTCCPVNWVEHQDSCYWFSHS |
| | | GMSWAEAEKYCQLKNAHLVVINSREEQNFVQKYLGS |
| | | AYTWMGLSDPEGAWKWVDGTDYATGFQNWKPGQPD |
| | | DWQGHGLGGGEDCAHFHPDGRWNDDVCQRPYHWVC |
| | | EAAEAAAKAAEAAAKADIKLQQSGAELARPGASVKM |
| | | SCKTSGYTFTRYTMHWVKQRPGQGLEWIGYINPSRGY |
| | | TNYNQKFKDKATLTTDKSSSTAYMQLSSLTSEDSAVY |
| | | YCARYYDDHYCLDYWGQGTTLTVSSVEGGSGGSGGS |
| | | GGSGGVDDIQLTQSPAIMSASPGEKVTMTCRASSSVSY |
| | | MNWYQQKSGTSPKRWIYDTSKVASGVPYRFSGSGSGT |
| | | SYSLTISSMEAEDAATYYCQQWSSNPLTFGAGTKLELK |
| 54 | HSA- | DAHKSEVAHRFKDLGEENFKALVLIAFAQYLQQCPFE |
| | GlyTR2^{slCD301(4)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(163- | YAPELLFFAKRYKAAFTECCQAADKAACLLPKLDELR |
| | 306)x4-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKALTCQVATLNNNASTEGTCCPVNWVEHQDSCYW |
| | | FSHSGMSWAEAEKYCQLKNAHLVVINSREEQNFVQKY |
| | | LGSAYTWMGLSDPEGAWKWVDGTDYATGFQNWKPG |
| | | QPDDWQGHGLGGGEDCAHFHPDGRWNDDVCQRPYH |
| | | WVCEAAEAAAKAAEAAAKALTCQVATLNNNASTEGT |
| | | CCPVNWVEHQDSCYWFSHSGMSWAEAEKYCQLKNA |
| | | HLVVINSREEQNFVQKYLGSAYTWMGLSDPEGAWKW |
| | | VDGTDYATGFQNWKPGQPDDWQGHGLGGGEDCAHF |
| | | HPDGRWNDDVCQRPYHWVCEAAEAAAKAAEAAAKA |
| | | LTCQVATLNNNASTEGTCCPVNWVEHQDSCYWFSHS |
| | | GMSWAEAEKYCQLKNAHLVVINSREEQNFVQKYLGS |
| | | AYTWMGLSDPEGAWKWVDGTDYATGFQNWKPGQPD |
| | | DWQGHGLGGGEDCAHFHPDGRWNDDVCQRPYHWVC |
| | | EAAEAAAKAAEAAAKALTCQVATLNNNASTEGTCCP |
| | | VNWVEHQDSCYWFSHSGMSWAEAEKYCQLKNAHLV |
| | | VINSREEQNFVQKYLGSAYTWMGLSDPEGAWKWVDG |
| | | TDYATGFQNWKPGQPDDWQGHGLGGGEDCAHFHPD |
| | | GRWNDDVCQRPYHWVCEAAEAAAKAAEAAAKADIK |
| | | LQQSGAELARPGASVKMSCKTSGYTFTRYTMHWVKQ |
| | | RPGQGLEWIGYINPSRGYTNYNQKFKDKATLTTDKSSS |
| | | TAYMQLSSLTSEDSAVYYCARYYDDHYCLDYWGQGT |
| | | TLTVSSVEGGSGGSGGSGGSGGVDDIQLTQSPAIMSAS |
| | | PGEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 55 | GlyTR2^{slCD301xCD3}: | CPVNWVEHQDSCYWFSHSGMSWAEAEKYCQLKNAH |
| | CD301(181- | LVVINSREEQNFVQKYLGSAYTWMGLSDPEGAWKWV |
| | 316)-Linker- | DGTDYATGFQNWKPGQPDDWQGHGLGGGEDCAHFH |
| | CD3 scFv | PDGRWNDDVCQRPYHWVCEAGLGQTSQESHAEAAA |
| | | KAAEAAAKADIKLQQSGAELARPGASVKMSCKTSGYT |
| | | FTRYTMHWVKQRPGQGLEWIGYINPSRGYTNYNQKF |
| | | KDKATLTTDKSSSTAYMQLSSLTSEDSAVYYCARYYD |
| | | DHYCLDYWGQGTTLTVSSVEGGSGGSGGSGGSGGVD |
| | | DIQLTQSPAIMSASPGEKVTMTCRASSSVSYMNWYQQ |
| | | KSGTSPKRWIYDTSKVASGVPYRFSGSGSGTSYSLTISS |
| | | MEAEDAATYYCQQWSSNPLTFGAGTKLELK |
| 56 | GlyTR2^{slCD301(2)xCD3}: | CPVNWVEHQDSCYWFSHSGMSWAEAEKYCQLKNAH |
| | CD301(181- | LVVINSREEQNFVQKYLGSAYTWMGLSDPEGAWKWV |
| | 316)x2-Linker- | DGTDYATGFQNWKPGQPDDWQGHGLGGGEDCAHFH |
| | CD3 scFv | PDGRWNDDVCQRPYHWVCEAGLGQTSQESHAEAAA |
| | | KAAEAAAKACPVNWVEHQDSCYWFSHSGMSWAEAE |
| | | KYCQLKNAHLVVINSREEQNFVQKYLGSAYTWMGLS |
| | | DPEGAWKWVDGTDYATGFQNWKPGQPDDWQGHGL |
| | | GGGEDCAHFHPDGRWNDDVCQRPYHWVCEAGLGQT |
| | | SQESHAEAAAKAAEAAAKADIKLQQSGAELARPGASV |
| | | KMSCKTSGYTFTRYTMHWVKQRPGQGLEWIGYINPSR |
| | | GYTNYNQKFKDKATLTTDKSSSTAYMQLSSLTSEDSA |
| | | VYYCARYYDDHYCLDYWGQGTTLTVSSVEGGSGGSG |
| | | GSGGSGGVDDIQLTQSPAIMSASPGEKVTMTCRASSSV |
| | | SYMNWYQQKSGTSPKRWIYDTSKVASGVPYRFSGSGS |
| | | GTSYSLTISSMEAEDAATYYCQQWSSNPLTFGAGTKLE |
| | | LK |
| 57 | GlyTR2^{slCD301(3)xCD3}: | CPVNWVEHQDSCYWFSHSGMSWAEAEKYCQLKNAH |
| | CD301(181- | LVVINSREEQNFVQKYLGSAYTWMGLSDPEGAWKWV |
| | 316)x3-Linker- | DGTDYATGFQNWKPGQPDDWQGHGLGGGEDCAHFH |
| | CD3 scFv | PDGRWNDDVCQRPYHWVCEAGLGQTSQESHAEAAA |
| | | KAAEAAAKACPVNWVEHQDSCYWFSHSGMSWAEAE |
| | | KYCQLKNAHLVVINSREEQNFVQKYLGSAYTWMGLS |
| | | DPEGAWKWVDGTDYATGFQNWKPGQPDDWQGHGL |
| | | GGGEDCAHFHPDGRWNDDVCQRPYHWVCEAGLGQT |
| | | SQESHAEAAAKAAEAAAKACPVNWVEHQDSCYWFSH |
| | | SGMSWAEAEKYCQLKNAHLVVINSREEQNFVQKYLG |
| | | SAYTWMGLSDPEGAWKWVDGTDYATGFQNWKPGQP |
| | | DDWQGHGLGGGEDCAHFHPDGRWNDDVCQRPYHW |
| | | VCEAGLGQTSQESHAEAAAKAAEAAAKADIKLQQSG |
| | | AELARPGASVKMSCKTSGYTFTRYTMHWVKQRPGQG |
| | | LEWIGYINPSRGYTNYNQKFKDKATLTTDKSSSTAYM |
| | | QLSSLTSEDSAVYYCARYYDDHYCLDYWGQGTTLTVS |
| | | SVEGGSGGSGGSGGSGGVDDIQLTQSPAIMSASPGEKV |
| | | TMTCRASSSVSYMNWYQQKSGTSPKRWIYDTSKVAS |
| | | GVPYRFSGSGSGTSYSLTISSMEAEDAATYYCQQWSSN |
| | | PLTFGAGTKLELK |
| 58 | GlyTR2^{slCD301(4)xCD3}: | CPVNWVEHQDSCYWFSHSGMSWAEAEKYCQLKNAH |
| | CD301(181- | LVVINSREEQNFVQKYLGSAYTWMGLSDPEGAWKWV |
| | 316)x4-Linker- | DGTDYATGFQNWKPGQPDDWQGHGLGGGEDCAHFH |
| | CD3 scFv | PDGRWNDDVCQRPYHWVCEAGLGQTSQESHAEAAA |
| | | KAAEAAAKACPVNWVEHQDSCYWFSHSGMSWAEAE |
| | | KYCQLKNAHLVVINSREEQNFVQKYLGSAYTWMGLS |
| | | DPEGAWKWVDGTDYATGFQNWKPGQPDDWQGHGL |
| | | GGGEDCAHFHPDGRWNDDVCQRPYHWVCEAGLGQT |
| | | SQESHAEAAAKAAEAAAKACPVNWVEHQDSCYWFSH |
| | | SGMSWAEAEKYCQLKNAHLVVINSREEQNFVQKYLG |
| | | SAYTWMGLSDPEGAWKWVDGTDYATGFQNWKPGQP |
| | | DDWQGHGLGGGEDCAHFHPDGRWNDDVCQRPYHW |
| | | VCEAGLGQTSQESHAEAAAKAAEAAAKACPVNWVEH |
| | | QDSCYWFSHSGMSWAEAEKYCQLKNAHLVVINSREE |
| | | QNFVQKYLGSAYTWMGLSDPEGAWKWVDGTDYATG |
| | | FQNWKPGQPDDWQGHGLGGGEDCAHFHPDGRWNDD |
| | | VCQRPYHWVCEAGLGQTSQESHAEAAAKAAEAAAKA |
| | | DIKLQQSGAELARPGASVKMSCKTSGYTFTRYTMHWV |
| | | KQRPGQGLEWIGYINPSRGYTNYNQKFKDKATLTTDK |
| | | SSSTAYMQLSSLTSEDSAVYYCARYYDDHYCLDYWG |
| | | QGTTLTVSSVEGGSGGSGGSGGSGGVDDIQLTQSPAIM |
| | | SASPGEKVTMTCRASSSVSYMNWYQQKSGTSPKRWIY |
| | | DTSKVASGVPYRFSGSGSGTSYSLTISSMEAEDAATYY |
| | | CQQWSSNPLTFGAGTKLELK |
| 59 | HSA- | DAHKSEVAHRFKDLGEENFKALVLIAFAQYLQQCPFE |
| | GlyTR2^{slCD301xCD3}: | DHVKLVNEVTEFAKTCVADESAENCDKSLHTLFGDKL |
| | HSA-Linker- | CTVATLRETYGEMADCCAKQEPERNECFLQHKDDNPN |
| | CD301(181- | LPRLVRPEVDVMCTAFHDNEETFLKKYLYEIARRHPYF |
| | 316)-Linker- | YAPELLFFAKRYKAAFTECCQAADKAACLLPKLDELR |
| | CD3 scFv | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAGLGQTSQESHA |
| | | EAAAKAAEAAAKADIKLQQSGAELARPGASVKMSCK |
| | | TSGYTFTRYTMHWVKQRPGQGLEWIGYINPSRGYTNY |
| | | NQKFKDKATLTTDKSSSTAYMQLSSLTSEDSAVYYCA |
| | | RYYDDHYCLDYWGQGTTLTVSSVEGGSGGSGGSGGS |
| | | GGVDDIQLTQSPAIMSASPGEKVTMTCRASSSVSYMN |
| | | WYQQKSGTSPKRWIYDTSKVASGVPYRFSGSGSGTSY |
| | | SLTISSMEAEDAATYYCQQWSSNPLTFGAGTKLELK |
| 60 | HSA- | DAHKSEVAHRFKDLGEENFKALVLIAFAQYLQQCPFE |
| | GlyTR2^{slCD301(2)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(181- | YAPELLFFAKRYKAAFTECCQAADKAACLLPKLDELR |
| | 316)x2-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAGLGQTSQESHA |
| | | EAAAKAAEAAAKACPVNWVEHQDSCYWFSHSGMSW |
| | | AEAEKYCQLKNAHLVVINSREEQNFVQKYLGSAYTW |
| | | MGLSDPEGAWKWVDGTDYATGFQNWKPGQPDDWQ |
| | | GHGLGGGEDCAHFHPDGRWNDDVCQRPYHWVCEAG |
| | | LGQTSQESHAEAAAKAAEAAAKADIKLQQSGAELARP |
| | | GASVKMSCKTSGYTFTRYTMHWVKQRPGQGLEWIGY |
| | | INPSRGYTNYNQKFKDKATLTTDKSSSTAYMQLSSLTS |
| | | EDSAVYYCARYYDDHYCLDYWGQGTTLTVSSVEGGS |
| | | GGSGGSGGSGGVDDIQLTQSPAIMSASPGEKVTMTCR |
| | | ASSSVSYMNWYQQKSGTSPKRWIYDTSKVASGVPYRF |
| | | SGSGSGTSYSLTISSMEAEDAATYYCQQWSSNPLTFGA |
| | | GTKLELK |
| 61 | HSA- | DAHKSEVAHRFKDLGEENFKALVLIAFAQYLQQCPFE |
| | GlyTR2^{slCD301(3)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(181- | YAPELLFFAKRYKAAFTECCQAADKAACLLPKLDELR |
| | 316)x3-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAGLGQTSQESHA |
| | | EAAAKAAEAAAKACPVNWVEHQDSCYWFSHSGMSW |
| | | AEAEKYCQLKNAHLVVINSREEQNFVQKYLGSAYTW |
| | | MGLSDPEGAWKWVDGTDYATGFQNWKPGQPDDWQ |
| | | GHGLGGGEDCAHFHPDGRWNDDVCQRPYHWVCEAG |
| | | LGQTSQESHAEAAAKAAEAAAKACPVNWVEHQDSCY |
| | | WFSHSGMSWAEAEKYCQLKNAHLVVINSREEQNFVQ |
| | | KYLGSAYTWMGLSDPEGAWKWVDGTDYATGFQNW |
| | | KPGQPDDWQGHGLGGGEDCAHFHPDGRWNDDVCQR |
| | | PYHWVCEAGLGQTSQESHAEAAAKAAEAAAKADIKL |
| | | QQSGAELARPGASVKMSCKTSGYTFTRYTMHWVKQR |
| | | PGQGLEWIGYINPSRGYTNYNQKFKDKATLTTDKSSST |
| | | AYMQLSSLTSEDSAVYYCARYYDDHYCLDYWGQGTT |
| | | LTVSSVEGGSGGSGGSGGSGGVDDIQLTQSPAIMSASP |
| | | GEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 62 | HSA- | DAHKSEVAHRFKDLGEENFKALVLIAFAQYLQQCPFE |
| | GlyTR2^{slCD301(4)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(181- | YAPELLFFAKRYKAAFTECCQAADKAACLLPKLDELR |
| | 316)x4-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAGLGQTSQESHA |
| | | EAAAKAAEAAAKACPVNWVEHQDSCYWFSHSGMSW |
| | | AEAEKYCQLKNAHLVVINSREEQNFVQKYLGSAYTW |
| | | MGLSDPEGAWKWVDGTDYATGFQNWKPGQPDDWQ |
| | | GHGLGGGEDCAHFHPDGRWNDDVCQRPYHWVCEAG |
| | | LGQTSQESHAEAAAKAAEAAAKACPVNWVEHQDSCY |
| | | WFSHSGMSWAEAEKYCQLKNAHLVVINSREEQNFVQ |
| | | KYLGSAYTWMGLSDPEGAWKWVDGTDYATGFQNW |
| | | KPGQPDDWQGHGLGGGEDCAHFHPDGRWNDDVCQR |
| | | PYHWVCEAGLGQTSQESHAEAAAKAAEAAAKACPVN |
| | | WVEHQDSCYWFSHSGMSWAEAEKYCQLKNAHLVVIN |
| | | SREEQNFVQKYLGSAYTWMGLSDPEGAWKWVDGTD |
| | | YATGFQNWKPGQPDDWQGHGLGGGEDCAHFHPDGR |
| | | WNDDVCQRPYHWVCEAGLGQTSQESHAEAAAKAAE |
| | | AAAKADIKLQQSGAELARPGASVKMSCKTSGYTFTRY |
| | | TMHWVKQRPGQGLEWIGYINPSRGYTNYNQKFKDKA |
| | | TLTTDKSSSTAYMQLSSLTSEDSAVYYCARYYDDHYC |
| | | LDYWGQGTTLTVSSVEGGSGGSGGSGGSGGVDDIQLT |
| | | QSPAIMSASPGEKVTMTCRASSSVSYMNWYQQKSGTS |
| | | PKRWIYDTSKVASGVPYRFSGSGSGTSYSLTISSMEAE |
| | | DAATYYCQQWSSNPLTFGAGTKLELK |
| 63 | GlyTR2^{slCD301xCD3}: | CPVNWVEHQDSCYWFSHSGMSWAEAEKYCQLKNAH |
| | CD301(181- | LVVINSREEQNFVQKYLGSAYTWMGLSDPEGAWKWV |
| | 306)-Linker- | DGTDYATGFQNWKPGQPDDWQGHGLGGGEDCAHFH |
| | CD3 scFv | PDGRWNDDVCQRPYHWVCEAAEAAAKAAEAAAKAD |
| | | IKLQQSGAELARPGASVKMSCKTSGYTFTRYTMHWVK |
| | | QRPGQGLEWIGYINPSRGYTNYNQKFKDKATLTTDKS |
| | | SSTAYMQLSSLTSEDSAVYYCARYYDDHYCLDYWGQ |
| | | GTTLTVSSVEGGSGGSGGSGGSGGVDDIQLTQSPAIMS |
| | | ASPGEKVTMTCRASSSVSYMNWYQQKSGTSPKRWIY |
| | | DTSKVASGVPYRFSGSGSGTSYSLTISSMEAEDAATYY |
| | | CQQWSSNPLTFGAGTKLELK |
| 64 | GlyTR2^{slCD301(2)xCD3}: | CPVNWVEHQDSCYWFSHSGMSWAEAEKYCQLKNAH |
| | CD301(181- | LVVINSREEQNFVQKYLGSAYTWMGLSDPEGAWKWV |
| | 306)x2-Linker- | DGTDYATGFQNWKPGQPDDWQGHGLGGGEDCAHFH |
| | CD3 scFv | PDGRWNDDVCQRPYHWVCEAAEAAAKAAEAAAKAC |
| | | PVNWVEHQDSCYWFSHSGMSWAEAEKYCQLKNAHL |
| | | VVINSREEQNFVQKYLGSAYTWMGLSDPEGAWKWVD |
| | | GTDYATGFQNWKPGQPDDWQGHGLGGGEDCAHFHP |
| | | DGRWNDDVCQRPYHWVCEAAEAAAKAAEAAAKADI |
| | | KLQQSGAELARPGASVKMSCKTSGYTFTRYTMHWVK |
| | | QRPGQGLEWIGYINPSRGYTNYNQKFKDKATLTTDKS |
| | | SSTAYMQLSSLTSEDSAVYYCARYYDDHYCLDYWGQ |
| | | GTTLTVSSVEGGSGGSGGSGGSGGVDDIQLTQSPAIMS |
| | | ASPGEKVTMTCRASSSVSYMNWYQQKSGTSPKRWIY |
| | | DTSKVASGVPYRFSGSGSGTSYSLTISSMEAEDAATYY |
| | | CQQWSSNPLTFGAGTKLELK |
| 65 | GlyTR2^{slCD301(3)xCD3}: | CPVNWVEHQDSCYWFSHSGMSWAEAEKYCQLKNAH |
| | CD301(181- | LVVINSREEQNFVQKYLGSAYTWMGLSDPEGAWKWV |
| | 306)x3-Linker- | DGTDYATGFQNWKPGQPDDWQGHGLGGGEDCAHFH |
| | CD3 scFv | PDGRWNDDVCQRPYHWVCEAAEAAAKAAEAAAKAC |
| | | PVNWVEHQDSCYWFSHSGMSWAEAEKYCQLKNAHL |
| | | VVINSREEQNFVQKYLGSAYTWMGLSDPEGAWKWVD |
| | | GTDYATGFQNWKPGQPDDWQGHGLGGGEDCAHFHP |
| | | DGRWNDDVCQRPYHWVCEAAEAAAKAAEAAAKACP |
| | | VNWVEHQDSCYWFSHSGMSWAEAEKYCQLKNAHLV |
| | | VINSREEQNFVQKYLGSAYTWMGLSDPEGAWKWVDG |
| | | TDYATGFQNWKPGQPDDWQGHGLGGGEDCAHFHPD |
| | | GRWNDDVCQRPYHWVCEAAEAAAKAAEAAAKADIK |
| | | LQQSGAELARPGASVKMSCKTSGYTFTRYTMHWVKQ |
| | | RPGQGLEWIGYINPSRGYTNYNQKFKDKATLTTDKSSS |
| | | TAYMQLSSLTSEDSAVYYCARYYDDHYCLDYWGQGT |
| | | TLTVSSVEGGSGGSGGSGGSGGVDDIQLTQSPAIMSAS |
| | | PGEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 66 | GlyTR2^{slCD301(4)xCD3}: | CPVNWVEHQDSCYWFSHSGMSWAEAEKYCQLKNAH |
| | CD301(181- | LVVINSREEQNFVQKYLGSAYTWMGLSDPEGAWKWV |
| | 306)x4-Linker- | DGTDYATGFQNWKPGQPDDWQGHGLGGGEDCAHFH |
| | CD3 scFv | PDGRWNDDVCQRPYHWVCEAAEAAAKAAEAAAKAC |
| | | PVNWVEHQDSCYWFSHSGMSWAEAEKYCQLKNAHL |
| | | VVINSREEQNFVQKYLGSAYTWMGLSDPEGAWKWVD |
| | | GTDYATGFQNWKPGQPDDWQGHGLGGGEDCAHFHP |
| | | DGRWNDDVCQRPYHWVCEAAEAAAKAAEAAAKACP |
| | | VNWVEHQDSCYWFSHSGMSWAEAEKYCQLKNAHLV |
| | | VINSREEQNFVQKYLGSAYTWMGLSDPEGAWKWVDG |
| | | TDYATGFQNWKPGQPDDWQGHGLGGGEDCAHFHPD |
| | | GRWNDDVCQRPYHWVCEAAEAAAKAAEAAAKACPV |
| | | NWVEHQDSCYWFSHSGMSWAEAEKYCQLKNAHLVVI |
| | | NSREEQNFVQKYLGSAYTWMGLSDPEGAWKWVDGT |
| | | DYATGFQNWKPGQPDDWQGHGLGGGEDCAHFHPDG |
| | | RWNDDVCQRPYHWVCEAAEAAAKAAEAAAKADIKL |
| | | QQSGAELARPGASVKMSCKTSGYTFTRYTMHWVKQR |
| | | PGQGLEWIGYINPSRGYTNYNQKFKDKATLTTDKSSST |
| | | AYMQLSSLTSEDSAVYYCARYYDDHYCLDYWGQGTT |
| | | LTVSSVEGGSGGSGGSGGSGGVDDIQLTQSPAIMSASP |
| | | GEKVTMTCRASSSVSYMNWYQQKSGTSPKRWIYDTS |
| | | KVASGVPYRFSGSGSGTSYSLTISSMEAEDAATYYCQQ |
| | | WSSNPLTFGAGTKLELK |
| 67 | HSA- | DAHKSEVAHRFKDLGEENFKALVLIAFAQYLQQCPFE |
| | GlyTR2^{slCD301xCD3}: | DHVKLVNEVTEFAKTCVADESAENCDKSLHTLFGDKL |
| | HSA-Linker- | CTVATLRETYGEMADCCAKQEPERNECFLQHKDDNPN |
| | CD301(181- | LPRLVRPEVDVMCTAFHDNEETFLKKYLYEIARRHPYF |
| | 306)-Linker- | YAPELLFFAKRYKAAFTECCQAADKAACLLPKLDELR |
| | CD3 scFv | DEGKASSAKQRLKCASLQKFGERAFKAWAVARLSQRF |
| | | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKADIKLQQSGAELARPGASVKMSCKTSGYTFTRYT |
| | | MHWVKQRPGQGLEWIGYINPSRGYTNYNQKFKDKAT |
| | | LTTDKSSSTAYMQLSSLTSEDSAVYYCARYYDDHYCL |
| | | DYWGQGTTLTVSSVEGGSGGSGGSGGSGGVDDIQLTQ |
| | | SPAIMSASPGEKVTMTCRASSSVSYMNWYQQKSGTSP |
| | | KRWIYDTSKVASGVPYRFSGSGSGTSYSLTISSMEAED |
| | | AATYYCQQWSSNPLTFGAGTKLELK |
| 68 | HSA- | DAHKSEVAHRFKDLGEENFKALVLIAFAQYLQQCPFE |
| | GlyTR2^{slCD301(2)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(181- | YAPELLFFAKRYKAAFTECCQAADKAACLLPKLDELR |
| | 306)x2-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKADIKLQQSGAELARPGASVKMSCKTSGYTFTRYT |
| | | MHWVKQRPGQGLEWIGYINPSRGYTNYNQKFKDKAT |
| | | LTTDKSSSTAYMQLSSLTSEDSAVYYCARYYDDHYCL |
| | | DYWGQGTTLTVSSVEGGSGGSGGSGGSGGVDDIQLTQ |
| | | SPAIMSASPGEKVTMTCRASSSVSYMNWYQQKSGTSP |
| | | KRWIYDTSKVASGVPYRFSGSGSGTSYSLTISSMEAED |
| | | AATYYCQQWSSNPLTFGAGTKLELK |
| 69 | HSA- | DAHKSEVAHRFKDLGEENFKALVLIAFAQYLQQCPFE |
| | GlyTR2^{slCD301(3)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(181- | YAPELLFFAKRYKAAFTECCQAADKAACLLPKLDELR |
| | 306)x3-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKADIKLQQSGAELARPGASVKMSCKTSGYTFTRYT |
| | | MHWVKQRPGQGLEWIGYINPSRGYTNYNQKFKDKAT |
| | | LTTDKSSSTAYMQLSSLTSEDSAVYYCARYYDDHYCL |
| | | DYWGQGTTLTVSSVEGGSGGSGGSGGSGGVDDIQLTQ |
| | | SPAIMSASPGEKVTMTCRASSSVSYMNWYQQKSGTSP |
| | | KRWIYDTSKVASGVPYRFSGSGSGTSYSLTISSMEAED |
| | | AATYYCQQWSSNPLTFGAGTKLELK |
| 70 | HSA- | DAHKSEVAHRFKDLGEENFKALVLIAFAQYLQQCPFE |
| | GlyTR2^{slCD301(4)xCD3}: | DHVKLVNEVTEFAKTCVADESAENCDKSLHTLFGDKL |
| | HSA- | CTVATLRETYGEMADCCAKQEPERNECFLQHKDDNPN |
| | Linker- | LPRLVRPEVDVMCTAFHDNEETFLKKYLYEIARRHPYF |
| | CD301(181- | YAPELLFFAKRYKAAFTECCQAADKAACLLPKLDELR |
| | 306)x4-Linker- | DEGKASSAKQRLKCASLQKFGERAFKAWAVARLSQRF |
| | CD3 scFv | PKAEFAEVSKLVTDLTKVHTECCHGDLLECADDRADL |
| | | AKYICENQDSISSKLKECCEKPLLEKSHCIAEVENDEMP |
| | | ADLPSLAADFVESKDVCKNYAEAKDVFLGMFLYEYA |
| | | RRHPDYSVVLLLRLAKTYETTLEKCCAAADPHECYAK |
| | | VFDEFKPLVEEPQNLIKQNCELFEQLGEYKFQNALLVR |
| | | YTKKVPQVSTPTLVEVSRNLGKVGSKCCKHPEAKRMP |
| | | CAEDYLSVVLNQLCVLHEKTPVSDRVTKCCTESLVNR |
| | | RPCFSALEVDETYVPKEFNAETFTFHADICTLSEKERQI |
| | | KKQTALVELVKHKPKATKEQLKAVMDDFAAFVEKCC |
| | | KADDKETCFAEEGKKLVAASQAALGLAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKACPVNWVEHQDSCYWFSHSGMSWAEAEKYCQL |
| | | KNAHLVVINSREEQNFVQKYLGSAYTWMGLSDPEGA |
| | | WKWVDGTDYATGFQNWKPGQPDDWQGHGLGGGED |
| | | CAHFHPDGRWNDDVCQRPYHWVCEAAEAAAKAAEA |
| | | AAKADIKLQQSGAELARPGASVKMSCKTSGYTFTRYT |
| | | MHWVKQRPGQGLEWIGYINPSRGYTNYNQKFKDKAT |
| | | LTTDKSSSTAYMQLSSLTSEDSAVYYCARYYDDHYCL |
| | | DYWGQGTTLTVSSVEGGSGGSGGSGGSGGVDDIQLTQ |
| | | SPAIMSASPGEKVTMTCRASSSVSYMNWYQQKSGTSP |
| | | KRWIYDTSKVASGVPYRFSGSGSGTSYSLTISSMEAED |
| | | AATYYCQQWSSNPLTFGAGTKLELK |

CAR

| | | |
|---|---|---|
| 71 | GlyTR1^{L-PHA} | SNDIYFNFQRFNETNLILQRDASVSSSGQLRLTNLNGN |
| | CAR: (L-PHA- | GEPRVGSLGRAFYSAPIQIWDNTTGTVASFATSFTFNIQ |
| | Linker-CD8aH + | VPNNAGPADGLAFALVPVGSQPKDKGGFLGLFDGSNS |
| | TM-4-1BB- | NFHTVAVEFDTLYNKDWDPTERHIGIDVNSIRSIKTTR |
| | CD3z) | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSTTTPAPRPPTPAPTIASQPLSLRPEACRPAAGGAVH |
| | | TRGLDFACDIYIWAPLAGTCGVLLLSLVITLYCKRGRK |
| | | KLLYIFKQPFMRPVQTTQEEDGCSCRFPEEEEGGCELR |
| | | VKFSRSADAPAYQQGQNQLYNELNLGRREEYDVLDK |
| | | RRGRDPEMGGKPRRKNPQEGLYNELQKDKMAEAYSEI |
| | | GMKGERRRGKGHDGLYQGLSTATKDTYDALHMQALP |
| | | PR |
| 72 | GlyTR1^{L-PHA(2)} | SNDIYFNFQRFNETNLILQRDASVSSSGQLRLTNLNGN |
| | CAR (L-PHA(2)- | GEPRVGSLGRAFYSAPIQIWDNTTGTVASFATSFTFNIQ |
| | Linker-CD8aH + | VPNNAGPADGLAFALVPVGSQPKDKGGFLGLFDGSNS |
| | TM-4-1BB | NFHTVAVEFDTLYNKDWDPTERHIGIDVNSIRSIKTTR |
| | CD3z) | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSSNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILGGGGSGGGGS |
| | | GGGGSTTTPAPRPPTPAPTIASQPLSLRPEACRPAAGGA |
| | | VHTRGLDFACDIYIWAPLAGTCGVLLLSLVITLYCKRG |
| | | RKKLLYIFKQPFMRPVQTTQEEDGCSCRFPEEEEGGCE |
| | | LRVKFSRSADAPAYQQGQNQLYNELNLGRREEYDVLD |
| | | KRRGRDPEMGGKPRRKNPQEGLYNELQKDKMAEAYS |
| | | EIGMKGERRRGKGHDGLYQGLSTATKDTYDALHMQA |
| | | LPPR |
| 73 | GlyTR1^{L-PHA(3)} | SNDIYFNFQRFNETNLILQRDASVSSSGQLRLTNLNGN |
| | CAR (L-PHA(3)- | GEPRVGSLGRAFYSAPIQIWDNTTGTVASFATSFTFNIQ |
| | Linker-CD8aH + | VPNNAGPADGLAFALVPVGSQPKDKGGFLGLFDGSNS |
| | TM-4-1BB- | NFHTVAVEFDTLYNKDWDPTERHIGIDVNSIRSIKTTR |
| | CD3z) | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSSNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSTTTPAPRPPTPAPTIASQPLSLRPEACRPAAGGA |
| | | VHTRGLDFACDIYIWAPLAGTCGVLLLSLVITLYCKRG |
| | | RKKLLYIFKQPFMRPVQTTQEEDGCSCRFPEEEEGGCE |
| | | LRVKFSRSADAPAYQQGQNQLYNELNLGRREEYDVLD |
| | | KRRGRDPEMGGKPRRKNPQEGLYNELQKDKMAEAYS |
| | | EIGMKGERRRGKGHDGLYQGLSTATKDTYDALHMQA |
| | | LPPR |
| 74 | GlyTR^{1L-PHA(4)} | SNDIYFNFQRFNETNLILQRDASVSSSGQLRLTNLNGN |
| | CAR (L-PHA(4)- | GEPRVGSLGRAFYSAPIQIWDNTTGTVASFATSFTFNIQ |
| | Linker-CD8aH + | VPNNAGPADGLAFALVPVGSQPKDKGGFLGLFDGSNS |
| | TM-4-1BB- | NFHTVAVEFDTLYNKDWDPTERHIGIDVNSIRSIKTTR |
| | CD3z) | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSSNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSTTTPAPRPPTPAPTIASQPLSLRPEACRPAAGGA |
| | | VHTRGLDFACDIYIWAPLAGTCGVLLLSLVITLYCKRG |
| | | RKKLLYIFKQPFMRPVQTTQEEDGCSCRFPEEEEGGCE |
| | | LRVKFSRSADAPAYQQGQNQLYNELNLGRREEYDVLD |
| | | KRRGRDPEMGGKPRRKNPQEGLYNELQKDKMAEAYS |
| | | EIGMKGERRRGKGHDGLYQGLSTATKDTYDALHMQA |
| | | LPPR |
| 75 | GlyTR^{1L-PHA} | GGGGSGGGGSGGGGSSNDIYFNFQRFNETNLILQRDAS |
| | CAR (L-PHA- | VSSSGQLRLTNLNGNGEPRVGSLGRAFYSAPIQIWDNT |
| | Linker-CD28-4- | TGTVASFATSFTFNIQVPNNAGPADGLAFALVPVGSQP |
| | 1BB-CD3z) | KDKGGFLGLFDGSNSNFHTVAVEFDTLYNKDWDPTER |
| | | HIGIDVNSIRSIKTTRWDFVNGENAEVLITYDSSTNLLV |
| | | ASLVYPSQKTSFIVSDTVDLKSVLPEWVSVGFSATTGIN |
| | | KGNVETNDVLSWSFASKLSDGTTSEGLNLANLVLNKIL |
| | | GGGGSGGGGSGGGGSIEVMYPPPYLDNEKSNGTIIHVK |
| | | GKHLCPSPLFPGPSKPFWVLVVVGGVLACYSLLVTVAF |
| | | IIFWVRSKRSRLLHSDYMNMTPRRPGPTRKHYQPYAPP |
| | | RDFAAYRSKRGRKKLLYIFKQPFMRPVQTTQEEDGCS |
| | | CRFPEEEEGGCELRVKFSRSADAPAYQQGQNQLYNEL |
| | | NLGRREEYDVLDKRRGRDPEMGGKPRRKNPQEGLYN |
| | | ELQKDKMAEAYSEIGMKGERRRGKGHDGLYQGLSTA |
| | | TKDTYDALHMQALPPR |
| 76 | GlyTR^{1L-PHA(2)} | SNDIYFNFQRFNETNLILQRDASVSSSGQLRLTNLNGN |
| | CAR (L-PHA(2)- | GEPRVGSLGRAFYSAPIQIWDNTTGTVASFATSFTFNIQ |
| | Linker-CD28-4- | VPNNAGPADGLAFALVPVGSQPKDKGGFLGLFDGSNS |
| | 1BB-CD3z) | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSSNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILGGGGSGGGGS |
| | | GGGGSIEVMYPPPYLDNEKSNGTIIHVKGKHLCPSPLFP |
| | | GPSKPFWVLVVVGGVLACYSLLVTVAFIIFWVRSKRSR |
| | | LLHSDYMNMTPRRPGPTRKHYQPYAPPRDFAAYRSKR |
| | | GRKKLLYIFKQPFMRPVQTTQEEDGCSCRFPEEEEGGC |
| | | ELRVKFSRSADAPAYQQGQNQLYNELNLGRREEYDVL |
| | | DKRRGRDPEMGGKPRRKNPQEGLYNELQKDKMAEAY |
| | | SEIGMKGERRRGKGHDGLYQGLSTATKDTYDALHMQ |
| | | ALPPR |
| 77 | GlyTR^{1L-PHA(3)} | SNDIYFNFQRFNETNLILQRDASVSSSGQLRLTNLNGN |
| | CAR (L-PHA(3)- | GEPRVGSLGRAFYSAPIQIWDNTTGTVASFATSFTFNIQ |
| | Linker-CD28-4- | VPNNAGPADGLAFALVPVGSQPKDKGGFLGLFDGSNS |
| | 1BB-CD3z) | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSSNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSIEVMYPPPYLDNEKSNGTIIHVKGKHLCPSPLFP |
| | | GPSKPFWVLVVVGGVLACYSLLVTVAFIIFWVRSKRSR |
| | | LLHSDYMNMTPRRPGPTRKHYQPYAPPRDFAAYRSKR |
| | | GRKKLLYIFKQPFMRPVQTTQEEDGCSCRFPEEEEGGC |
| | | ELRVKFSRSADAPAYQQGQNQLYNELNLGRREEYDVL |
| | | DKRRGRDPEMGGKPRRKNPQEGLYNELQKDKMAEAY |
| | | SEIGMKGERRRGKGHDGLYQGLSTATKDTYDALHMQ |
| | | ALPPR |
| 78 | GlyTR^{1L-PHA(4)} | SNDIYFNFQRFNETNLILQRDASVSSSGQLRLTNLNGN |
| | CAR (L-PHA(4)- | GEPRVGSLGRAFYSAPIQIWDNTTGTVASFATSFTFNIQ |
| | Linker-CD28-4- | VPNNAGPADGLAFALVPVGSQPKDKGGFLGLFDGSNS |
| | 1BB-CD3z) | NFHTVAVEFDTLYNKDWDPTERHIGIDVNSIRSIKTTR |
| | | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILGGGGSGGGGSGG |
| | | GGSSNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSSNDIYFNFQRFNETNLILQRDASVSSSGQLRLTN |
| | | LNGNGEPRVGSLGRAFYSAPIQIWDNTTGTVASFATSF |
| | | TFNIQVPNNAGPADGLAFALVPVGSQPKDKGGFLGLF |
| | | DGSNSNFHTVAVEFDTLYNKDWDPTERHIGIDVNSIRSI |
| | | KTTRWDFVNGENAEVLITYDSSTNLLVASLVYPSQKTS |
| | | FIVSDTVDLKSVLPEWVSVGFSATTGINKGNVETNDVL |
| | | SWSFASKLSDGTTSEGLNLANLVLNKILGGGGSGGGGS |
| | | GGGGSIEVMYPPPYLDNEKSNGTIIHVKGKHLCPSPLFP |
| | | GPSKPFWVLVVVGGVLACYSLLVTVAFIIFWVRSKRSR |
| | | LLHSDYMNMTPRRPGPTRKHYQPYAPPRDFAAYRSKR |
| | | GRKKLLYIFKQPFMRPVQTTQEEDGCSCRFPEEEEGGC |
| | | ELRVKFSRSADAPAYQQGQNQLYNELNLGRREEYDVL |
| | | DKRRGRDPEMGGKPRRKNPQEGLYNELQKDKMAEAY |
| | | SEIGMKGERRRGKGHDGLYQGLSTATKDTYDALHMQ |
| | | ALPPR |
| 79 | GlyTR^{1L-PHAΔ1-5} | FNFQRFNETNLILQRDASVSSSGQLRLTNLNGNGEPRV |
| | CAR (L-PHAΔ1- | GSLGRAFYSAPIQIWDNTTGTVASFATSFTFNIQVPNNA |
| | 5-Linker-CD8aH + | GPADGLAFALVPVGSQPKDKGGFLGLFDGSNSNFHTV |
| | TM-4-1BB- | AVEFDTLYNKDWDPTERHIGIDVNSIRSIKTTRWDFVN |
| | CD3z) | GENAEVLITYDSSTNLLVASLVYPSQKTSFIVSDTVDLK |
| | | SVLPEWVSVGFSATTGINKGNVETNDVLSWSFASKLSD |
| | | GTTSEGLNLANLVLNKILGGGGSGGGGSGGGGSTTTP |
| | | APRPPTPAPTIASQPLSLRPEACRPAAGGAVHTRGLDFA |
| | | CDIYIWAPLAGTCGVLLLSLVITLYCKRGRKKLLYIFKQ |
| | | PFMRPVQTTQEEDGCSCRFPEEEEGGCELRVKFSRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPR |
| 80 | GlyTR^{1L-PHAΔ1-5(2)} | FNFQRFNETNLILQRDASVSSSGQLRLTNLNGNGEPRV |
| | CAR (L- | GSLGRAFYSAPIQIWDNTTGTVASFATSFTFNIQVPNNA |
| | PHAΔ11-5(2)- | GPADGLAFALVPVGSQPKDKGGFLGLFDGSNSNFHTV |
| | Linker-CD8aH + | AVEFDTLYNKDWDPTERHIGIDVNSIRSIKTTRWDFVN |
| | TM-4-1BB- | GENAEVLITYDSSTNLLVASLVYPSQKTSFIVSDTVDLK |
| | CD3z) | SVLPEWVSVGFSATTGINKGNVETNDVLSWSFASKLSD |
| | | GTTSEGLNLANLVLNKILGGGGSGGGGSGGGGSFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSTTTPAPRP |
| | | PTPAPTIASQPLSLRPEACRPAAGGAVHTRGLDFACDIY |
| | | IWAPLAGTCGVLLLSLVITLYCKRGRKKLLYIFKQPFM |
| | | RPVQTTQEEDGCSCRFPEEEEGGCELRVKFSRSADAPA |
| | | YQQGQNQLYNELNLGRREEYDVLDKRRGRDPEMGGK |
| | | PRRKNPQEGLYNELQKDKMAEAYSEIGMKGERRRGK |
| | | GHDGLYQGLSTATKDTYDALHMQALPPR |
| 81 | GlyTR^{1L-PHAΔ1-5(3)} | FNFQRFNETNLILQRDASVSSSGQLRLTNLNGNGEPRV |
| | CAR (L- | GSLGRAFYSAPIQIWDNTTGTVASFATSFTFNIQVPNNA |
| | PHAΔ11-5(3) | GPADGLAFALVPVGSQPKDKGGFLGLFDGSNSNFHTV |
| | Linker-CD8aH + | AVEFDTLYNKDWDPTERHIGIDVNSIRSIKTTRWDFVN |
| | TM-4-1BB- | GENAEVLITYDSSTNLLVASLVYPSQKTSFIVSDTVDLK |
| | CD3z) | SVLPEWVSVGFSATTGINKGNVETNDVLSWSFASKLSD |
| | | GTTSEGLNLANLVLNKILGGGGSGGGGSGGGGSFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSFNFQRFN |
| | | ETNLILQRDASVSSSGQLRLTNLNGNGEPRVGSLGRAF |
| | | YSAPIQIWDNTTGTVASFATSFTFNIQVPNNAGPADGL |
| | | AFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKILGGGGSGGGGSGGGGSTTTPAPRPPTP |
| | | APTIASQPLSLRPEACRPAAGGAVHTRGLDFACDIYIW |
| | | APLAGTCGVLLLSLVITLYCKRGRKKLLYIFKQPFMRP |
| | | VQTTQEEDGCSCRFPEEEEGGCELRVKFSRSADAPAYQ |
| | | QGQNQLYNELNLGRREEYDVLDKRRGRDPEMGGKPR |
| | | RKNPQEGLYNELQKDKMAEAYSEIGMKGERRRGKGH |
| | | DGLYQGLSTATKDTYDALHMQALPPR |
| 82 | GlyTR^{1L-PHAΔ1-5(4)} | FNFQRFNETNLILQRDASVSSSGQLRLTNLNGNGEPRV |
| | CAR (L- | GSLGRAFYSAPIQIWDNTTGTVASFATSFTFNIQVPNNA |
| | PHAΔ11-5(4)- | GPADGLAFALVPVGSQPKDKGGFLGLFDGSNSNFHTV |
| | Linker-CD8aH + | AVEFDTLYNKDWDPTERHIGIDVNSIRSIKTTRWDFVN |
| | TM-4-1BB | GENAEVLITYDSSTNLLVASLVYPSQKTSFIVSDTVDLK |
| | CD3z) | SVLPEWVSVGFSATTGINKGNVETNDVLSWSFASKLSD |
| | | GTTSEGLNLANLVLNKILGGGGSGGGGSGGGGSFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSFNFQRFN |
| | | ETNLILQRDASVSSSGQLRLTNLNGNGEPRVGSLGRAF |
| | | YSAPIQIWDNTTGTVASFATSFTFNIQVPNNAGPADGL |
| | | AFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKILGGGGSGGGGSGGGGSFNFQRFNETN |
| | | LILQRDASVSSSGQLRLTNLNGNGEPRVGSLGRAFYSA |
| | | PIQIWDNTTGTVASFATSFTFNIQVPNNAGPADGLAFAL |
| | | VPVGSQPKDKGGFLGLFDGSNSNFHTVAVEFDTLYNK |
| | | DWDPTERHIGIDVNSIRSIKTTRWDFVNGENAEVLITYD |
| | | SSTNLLVASLVYPSQKTSFIVSDTVDLKSVLPEWVSVG |
| | | FSATTGINKGNVETNDVLSWSFASKLSDGTTSEGLNLA |
| | | NLVLNKILGGGGSGGGGSGGGGSTTTPAPRPPTPAPTIA |
| | | SQPLSLRPEACRPAAGGAVHTRGLDFACDIYIWAPLAG |
| | | TCGVLLLSLVITLYCKRGRKKLLYIFKQPFMRPVQTTQ |
| | | EEDGCSCRFPEEEEGGCELRVKFSRSADAPAYQQGQN |
| | | QLYNELNLGRREEYDVLDKRRGRDPEMGGKPRRKNP |
| | | QEGLYNELQKDKMAEAYSEIGMKGERRRGKGHDGLY |
| | | QGLSTATKDTYDALHMQALPPR |
| 83 | GlyTR^{1L-PHAΔ1-3} | IYFNFQRFNETNLILQRDASVSSSGQLRLTNLNGNGEPR |
| | CAR (L-PHAΔ1- | VGSLGRAFYSAPIQIWDNTTGTVASFATSFTFNIQVPNN |
| | 3-Linker-CD8aH + | AGPADGLAFALVPVGSQPKDKGGFLGLFDGSNSNFHT |
| | TM-4-1BB- | VAVEFDTLYNKDWDPTERHIGIDVNSIRSIKTTRWDFV |
| | CD3z) | NGENAEVLITYDSSTNLLVASLVYPSQKTSFIVSDTVDL |
| | | KSVLPEWVSVGFSATTGINKGNVETNDVLSWSFASKLS |
| | | DGTTSEGLNLANLVLNKILGGGGSGGGGSGGGGSTTT |
| | | PAPRPPTPAPTIASQPLSLRPEACRPAAGGAVHTRGLDF |
| | | ACDIYIWAPLAGTCGVLLLSLVITLYCKRGRKKLLYIFK |
| | | QPFMRPVQTTQEEDGCCRFPEEEEGGCELRVKFSRSA |
| | | DAPAYQQGQNQLYNELNLGRREEYDVLDKRRGRDPE |
| | | MGGKPRRKNPQEGLYNELQKDKMAEAYSEIGMKGER |
| | | RRGKGHDGLYQGLSTATKDTYDALHMQALPPR |
| 84 | GlyTR^{1L-PHAΔ1-3(2)} | IYFNFQRFNETNLILQRDASVSSSGQLRLTNLNGNGEPR |
| | CAR (L- | VGSLGRAFYSAPIQIWDNTTGTVASFATSFTFNIQVPNN |
| | PHAΔ11-3(2)- | AGPADGLAFALVPVGSQPKDKGGFLGLFDGSNSNFHT |
| | Linker-CD8aH + | VAVEFDTLYNKDWDPTERHIGIDVNSIRSIKTTRWDFV |
| | TM-4-1BB- | NGENAEVLITYDSSTNLLVASLVYPSQKTSFIVSDTVDL |
| | CD3z) | KSVLPEWVSVGFSATTGINKGNVETNDVLSWSFASKLS |
| | | DGTTSEGLNLANLVLNKILGGGGSGGGGSGGGGSIYFN |
| | | FQRFNETNLILQRDASVSSSGQLRLTNLNGNGEPRVGS |
| | | LGRAFYSAPIQIWDNTTGTVASFATSFTFNIQVPNNAGP |
| | | ADGLAFALVPVGSQPKDKGGFLGLFDGSNSNFHTVAV |
| | | EFDTLYNKDWDPTERHIGIDVNSIRSIKTTRWDFVNGE |
| | | NAEVLITYDSSTNLLVASLVYPSQKTSFIVSDTVDLKSV |
| | | LPEWVSVGFSATTGINKGNVETNDVLSWSFASKLSDG |
| | | TTSEGLNLANLVLNKILGGGGSGGGGSGGGGSTTTPAP |
| | | RPPTPAPTIASQPLSLRPEACRPAAGGAVHTRGLDFAC |
| | | DIYIWAPLAGTCGVLLLSLVITLYCKRGRKKLLYIFKQP |
| | | FMRPVQTTQEEDGCSCRFPEEEEGGCELRVKFSRSADA |
| | | PAYQQGQNQLYNELNLGRREEYDVLDKRRGRDPEMG |
| | | GKPRRKNPQEGLYNELQKDKMAEAYSEIGMKGERRR |
| | | GKGHDGLYQGLSTATKDTYDALHMQALPPR |
| 85 | GlyTR^{1L-PHAΔ1-3(3)} | IYFNFQRFNETNLILQRDASVSSSGQLRLTNLNGNGEPR |
| | CAR (L- | VGSLGRAFYSAPIQIWDNTTGTVASFATSFTFNIQVPNN |
| | PHAΔ11-3(3)- | AGPADGLAFALVPVGSQPKDKGGFLGLFDGSNSNFHT |
| | Linker-CD8aH + | VAVEFDTLYNKDWDPTERHIGIDVNSIRSIKTTRWDFV |
| | TM-4-1BB- | NGENAEVLITYDSSTNLLVASLVYPSQKTSFIVSDTVDL |
| | CD3z) | KSVLPEWVSVGFSATTGINKGNVETNDVLSWSFASKLS |
| | | DGTTSEGLNLANLVLNKILGGGGSGGGGSGGGGSIYFN |
| | | FQRFNETNLILQRDASVSSSGQLRLTNLNGNGEPRVGS |
| | | LGRAFYSAPIQIWDNTTGTVASFATSFTFNIQVPNNAGP |
| | | ADGLAFALVPVGSQPKDKGGFLGLFDGSNSNFHTVAV |
| | | EFDTLYNKDWDPTERHIGIDVNSIRSIKTTRWDFVNGE |
| | | NAEVLITYDSSTNLLVASLVYPSQKTSFIVSDTVDLKSV |
| | | LPEWVSVGFSATTGINKGNVETNDVLSWSFASKLSDG |
| | | TTSEGLNLANLVLNKILGGGGSGGGGSGGGGSIYFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSTTTPAPRP |
| | | PTPAPTIASQPLSLRPEACRPAAGGAVHTRGLDFACDIY |
| | | IWAPLAGTCGVLLLSLVITLYCKRGRKKLLYIFKQPFM |
| | | RPVQTTQEEDGCSCRFPEEEEGGCELRVKFSRSADAPA |
| | | YQQGQNQLYNELNLGRREEYDVLDKRRGRDPEMGGK |
| | | PRRKNPQEGLYNELQKDKMAEAYSEIGMKGERRRGK |
| | | GHDGLYQGLSTATKDTYDALHMQALPPR |
| 86 | GlyTR ^{1L-PHAΔ1-3(4)} | IYFNFQRFNETNLILQRDASVSSSGQLRLTNLNGNGEPR |
| | CAR (L- | VGSLGRAFYSAPIQIWDNTTGTVASFATSFTFNIQVPNN |
| | PHAΔ11-3(4)- | AGPADGLAFALVPVGSQPKDKGGFLGLFDGSNSNFHT |
| | Linker-CD8aH + | VAVEFDTLYNKDWDPTERHIGIDVNSIRSIKTTRWDFV |
| | TM-4-1BB- | NGENAEVLITYDSSTNLLVASLVYPSQKTSFIVSDTVDL |
| | CD3z) | KSVLPEWVSVGFSATTGINKGNVETNDVLSWSFASKLS |
| | | DGTTSEGLNLANLVLNKILGGGGSGGGGSGGGGSIYFN |
| | | FQRFNETNLILQRDASVSSSGQLRLTNLNGNGEPRVGS |
| | | LGRAFYSAPIQIWDNTTGTVASFATSFTFNIQVPNNAGP |
| | | ADGLAFALVPVGSQPKDKGGFLGLFDGSNSNFHTVAV |
| | | EFDTLYNKDWDPTERHIGIDVNSIRSIKTTRWDFVNGE |
| | | NAEVLITYDSSTNLLVASLVYPSQKTSFIVSDTVDLKSV |
| | | LPEWVSVGFSATTGINKGNVETNDVLSWSFASKLSDG |
| | | TTSEGLNLANLVLNKILGGGGSGGGGSGGGGSIYFNFQ |
| | | RFNETNLILQRDASVSSSGQLRLTNLNGNGEPRVGSLG |
| | | RAFYSAPIQIWDNTTGTVASFATSFTFNIQVPNNAGPAD |
| | | GLAFALVPVGSQPKDKGGFLGLFDGSNSNFHTVAVEF |
| | | DTLYNKDWDPTERHIGIDVNSIRSIKTTRWDFVNGENA |
| | | EVLITYDSSTNLLVASLVYPSQKTSFIVSDTVDLKSVLP |
| | | EWVSVGFSATTGINKGNVETNDVLSWSFASKLSDGTT |
| | | SEGLNLANLVLNKILGGGGSGGGGSGGGGSIYFNFQRF |
| | | NETNLILQRDASVSSSGQLRLTNLNGNGEPRVGSLGRA |
| | | FYSAPIQIWDNTTGTVASFATSFTFNIQVPNNAGPADGL |
| | | AFALVPVGSQPKDKGGFLGLFDGSNSNFHTVAVEFDT |
| | | LYNKDWDPTERHIGIDVNSIRSIKTTRWDFVNGENAEV |
| | | LITYDSSTNLLVASLVYPSQKTSFIVSDTVDLKSVLPEW |
| | | VSVGFSATTGINKGNVETNDVLSWSFASKLSDGTTSEG |
| | | LNLANLVLNKILGGGGSGGGGSGGGGSTTTPAPRPPTP |
| | | APTIASQPLSLRPEACRPAAGGAVHTRGLDFACDIYIW |
| | | APLAGTCGVLLLSLVITLYCKRGRKKLLYIFKQPFMRP |
| | | VQTTQEEDGCSCRFPEEEEGGCELRVKFSRSADAPAYQ |
| | | QGQNQLYNELNLGRREEYDVLDKRRGRDPEMGGKPR |
| | | RKNPQEGLYNELQKDKMAEAYSEIGMKGERRRGKGH |
| | | DGLYQGLSTATKDTYDALHMQALPPR |
| 87 | GlyTR^{1L-PHA(2)} | SNDIYFNFQRFNETNLILQRDASVSSSGQLRLTNLNGN |
| | CAR (L-PHA(2)- | GEPRVGSLGRAFYSAPIQIWDNTTGTVASFATSFTFNIQ |
| | Stiff Linker- | VPNNAGPADGLAFALVPVGSQPKDKGGFLGLFDGSNS |
| | CD28-4-1BB- | NFHTVAVEFDTLYNKDWDPTERHIGIDVNSIRSIKTTR |
| | CD3z) | WDFVNGENAEVLITYDSSTNLLVASLVYPSQKTSFIVS |
| | | DTVDLKSVLPEWVSVGFSATTGINKGNVETNDVLSWS |
| | | FASKLSDGTTSEGLNLANLVLNKILAEAAAKAAEAAA |
| | | KASNDIYFNFQRFNETNLILQRDASVSSSGQLRLTNLN |
| | | GNGEPRVGSLGRAFYSAPIQIWDNTTGTVASFATSFTF |
| | | NIQVPNNAGPADGLAFALVPVGSQPKDKGGFLGLFDG |
| | | SNSNFHTVAVEFDTLYNKDWDPTERHIGIDVNSIRSIKT |
| | | TRWDFVNGENAEVLITYDSSTNLLVASLVYPSQKTSFI |
| | | VSDTVDLKSVLPEWVSVGFSATTGINKGNVETNDVLS |
| | | WSFASKLSDGTTSEGLNLANLVLNKILAEAAAKAAEA |
| | | AAKAIEVMYPPPYLDNEKSNGTIIHVKGKHLCPSPLFPG |
| | | PSKPFWVLVVVGGVLACYSLLVTVAFIIFWVRSKRSRL |
| | | LHSDYMNMTPRRPGPTRKHYQPYAPPRDFAAYRSKRG |
| | | RKKLLYIFKQPFMRPVQTTQEEDGCSCRFPEEEEGGCE |
| | | LRVKFSRSADAPAYQQGQNQLYNELNLGRREEYDVLD |
| | | KRRGRDPEMGGKPRRKNPQEGLYNELQKDKMAEAYS |
| | | EIGMKGERRRGKGHDGLYQGLSTATKDTYDALHMQA |
| | | LPPR |
| 88 | GlyTR2^{SlCD301(4)} | VHSEMLLRVQQLVQDLKKLTCQVATLNNNASTEGTC |
| | CAR (Extended | CPVNWVEHQDSCYWFSHSGMSWAEAEKYCQLKNAH |
| | CD301CRD- | LVVINSREEQNFVQKYLGSAYTWMGLSDPEGAWKWV |
| | linker- | DGTDYATGFQNWKPGQPDDWQGHGLGGGEDCAHFH |
| | CD301CRD(3)- | PDGRWNDDVCQRPYHWVCEAGLGQTSQESHAEAAA |
| | linker-CD8aH + | KAAEAAAKALTCQVATLNNNASTEGTCCPVNWVEHQ |
| | TM-4-1BB- | DSCYWFSHSGMSWAEAEKYCQLKNAHLVVINSREEQ |
| | CD3z) | NFVQKYLGSAYTWMGLSDPEGAWKWVDGTDYATGF |
| | | QNWKPGQPDDWQGHGLGGGEDCAHFHPDGRWNDDV |
| | | CQRPYHWVCEAGLGQTSQESHAEAAAKAAEAAAKAL |
| | | TCQVATLNNNASTEGTCCPVNWVEHQDSCYWFSHSG |
| | | MSWAEAEKYCQLKNAHLVVINSREEQNFVQKYLGSA |
| | | YTWMGLSDPEGAWKWVDGTDYATGFQNWKPGQPDD |
| | | WQGHGLGGGEDCAHFHPDGRWNDDVCQRPYHWVCE |
| | | AGLGQTSQESHAEAAAKAAEAAAKALTCQVATLNNN |
| | | ASTEGTCCPVNWVEHQDSCYWFSHSGMSWAEAEKYC |
| | | QLKNAHLVVINSREEQNFVQKYLGSAYTWMGLSDPEG |
| | | AWKWVDGTDYATGFQNWKPGQPDDWQGHGLGGGE |
| | | DCAHFHPDGRWNDDVCQRPYHWVCEAGLGQTSQESH |
| | | AEAAAKAAEAAAKATTTPAPRPPTPAPTIASQPLSLRPE |
| | | ACRPAAGGAVHTRGLDFACDIYIWAPLAGTCGVLLLS |
| | | LVITLYCKRGRKKLLYIFKQPFMRPVQTTQEEDGCSCR |
| | | FPEEEEGGCELRVKFSRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPR |
| 89 | GlyTR2^{slCD301(4)} | VHSEMLLRVQQLVQDLKKLTCQVATLNNNASTEGTC |
| | CAR mutant | CPVNWVEHQDSCYWFSHSGMSWAEAEKYCQLKNAH |
| | (Mutated | LVVINSREEQNFVQKYLGSAYTWMGLSDPEGAWKWV |
| | extended | DGTDYATGFQNWKPGGPGDWQGHGLGGGGDCAHFH |
| | CD301CRD- | PDGRWGGDVCQRPYHWVCEAGLGQTSQESHAEAAA |
| | linker- | KAAEAAAKALTCQVATLNNNASTEGTCCPVNWVEHQ |
| | mutCD301CRD(3)- | DSCYWFSHSGMSWAEAEKYCQLKNAHLVVINSREEQ |
| | linker-CD8aH + | NFVQKYLGSAYTWMGLSDPEGAWKWVDGTDYATGF |
| | TM-4-1BB- | QNWKPGGPGDWQGHGLGGGGDCAHFHPDGRWGGD |
| | CD3z) | VCQRPYHWVCEAGLGQTSQESHAEAAAKAAEAAAKA |
| | | LTCQVATLNNNASTEGTCCPVNWVEHQDSCYWFSHS |
| | | GMSWAEAEKYCQLKNAHLVVINSREEQNFVQKYLGS |
| | | AYTWMGLSDPEGAWKWVDGTDYATGFQNWKPGGPG |
| | | DWQGHGLGGGGDCAHFHPDGRWGGDVCQRPYHWV |
| | | CEAGLGQTSQESHAEAAAKAAEAAAKALTCQVATLN |
| | | NNASTEGTCCPVNWVEHQDSCYWFSHSGMSWAEAEK |
| | | YCQLKNAHLVVINSREEQNFVQKYLGSAYTWMGLSD |
| | | PEGAWKWVDGTDYATGFQNWKPGGPGDWQGHGLG |
| | | GGGDCAHFHPDGRWGGDVCQRPYHWVCEAGLGQTS |
| | | QESHAEAAAKAAEAAAKATTTPAPRPPTPAPTIASQPL |
| | | SLRPEACRPAAGGAVHTRGLDFACDIYIWAPLAGTCG |
| | | VLLLSLVITLYCKRGRKKLLYIFKQPFMRPVQTTQEED |
| | | GCSCRFPEEEEGGCELRVKFSRSADAPAYQQGQNQLY |
| | | NELNLGRREEYDVLDKRRGRDPEMGGKPRRKNPQEG |
| | | LYNELQKDKMAEAYSEIGMKGERRRGKGHDGLYQGL |
| | | STATKDTYDALHMQALPPR |
| 90 | GlyTR2^{slCD301(4)} | VHSEMLLRVQQLVQDLKKLTCQVATLNNNASTEGTC |
| | CAR (Extended | CPVNWVEHQDSCYWFSHSGMSWAEAEKYCQLKNAH |
| | CD301CRD- | LVVINSREEQNFVQKYLGSAYTWMGLSDPEGAWKWV |
| | linker- | DGTDYATGFQNWKPGQPDDWQGHGLGGGEDCAHFH |
| | CD301CRD(3)- | PDGRWNDDVCQRPYHWVCEAGLGQTSQESHAEAAA |
| | linker- | KAAEAAAKALTCQVATLNNNASTEGTCCPVNWVEHQ |
| | FVPVFLPAKP- | DSCYWFSHSGMSWAEAEKYCQLKNAHLVVINSREEQ |
| | CD8a H + TM-4- | NFVQKYLGSAYTWMGLSDPEGAWKWVDGTDYATGF |
| | 1BB-CD3z) | QNWKPGQPDDWQGHGLGGGEDCAHFHPDGRWNDDV |
| | | CQRPYHWVCEAGLGQTSQESHAEAAAKAAEAAAKAL |
| | | TCQVATLNNNASTEGTCCPVNWVEHQDSCYWFSHSG |
| | | MSWAEAEKYCQLKNAHLVVINSREEQNFVQKYLGSA |
| | | YTWMGLSDPEGAWKWVDGTDYATGFQNWKPGQPDD |
| | | WQGHGLGGGEDCAHFHPDGRWNDDVCQRPYHWVCE |
| | | AGLGQTSQESHAEAAAKAAEAAAKALTCQVATLNNN |
| | | ASTEGTCCPVNWVEHQDSCYWFSHSGMSWAEAEKYC |
| | | QLKNAHLVVINSREEQNFVQKYLGSAYTWMGLSDPEG |
| | | AWKWVDGTDYATGFQNWKPGQPDDWQGHGLGGGE |
| | | DCAHFHPDGRWNDDVCQRPYHWVCEAGLGQTSQESH |
| | | AEAAAKAAEAAAKAFVPVFLPAKPTTTPAPRPPTPAPT |
| | | IASQPLSLRPEACRPAAGGAVHTRGLDFACDIYIWAPL |
| | | AGTCGVLLLSLVITLYCKRGRKKLLYIFKQPFMRPVQT |
| | | TQEEDGCSCRFPEEEEGGCELRVKFSRSADAPAYQQGQ |
| | | NQLYNELNLGRREEYDVLDKRRGRDPEMGGKPRRKN |
| | | PQEGLYNELQKDKMAEAYSEIGMKGERRRGKGHDGL |
| | | YQGLSTATKDTYDALHMQALPPR |
| 91 | GlyTR2^{CD301} | VHSEMLLRVQQLVQDLKKLTCQVATLNNNASTEGTC |
| | CAR (Extended | CPVNWVEHQDSCYWFSHSGMSWAEAEKYCQLKNAH |
| | CD301CRD- | LVVINSREEQNFVQKYLGSAYTWMGLSDPEGAWKWV |
| | CD28-4-1BB- | DGTDYATGFQNWKPGQPDDWQGHGLGGGEDCAHFH |
| | CD3z) | PDGRWNDDVCQRPYHWVCEAGLGQTSQESHIEVMYP |
| | | PPYLDNEKSNGTIIHVKGKHLCPSPLFPGPSKPFWVLVV |
| | | VGGVLACYSLLVTVAFIIFWVRSKRSRLLHSDYMNMT |
| | | PRRPGPTRKHYQPYAPPRDFAAYRSKRGRKKLLYIFKQ |
| | | PFMRPVQTTQEEDGCSCRFPEEEEGGCELRVKFSRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPR |
| 92 | GlyTR2^{CD301} | VHSEMLLRVQQLVQDLKKLTCQVATLNNNASTEGTC |
| | CAR (Extended | CPVNWVEHQDSCYWFSHSGMSWAEAEKYCQLKNAH |
| | CD301CRD- | LVVINSREEQNFVQKYLGSAYTWMGLSDPEGAWKWV |
| | CD8a H + TM-4- | DGTDYATGFQNWKPGQPDDWQGHGLGGGEDCAHFH |
| | 1BB-CD3z) | PDGRWNDDVCQRPYHWVCEAGLGQTSQESHTTTPAP |
| | | RPPTPAPTIASQPLSLRPEACRPAAGGAVHTRGLDFAC |
| | | DIYIWAPLAGTCGVLLLSLVITLYCKRGRKKLLYIFKQP |
| | | FMRPVQTTQEEDGCSCRFPEEEEGGCELRVKFSRSADA |
| | | PAYQQGQNQLYNELNLGRREEYDVLDKRRGRDPEMG |
| | | GKPRRKNPQEGLYNELQKDKMAEAYSEIGMKGERRR |
| | | GKGHDGLYQGLSTATKDTYDALHMQALPPR |
| 93 | CD301 CAR | CPVNWVEHQDSCYWFSHSGMSWAEAEKYCQLKNAH |
| | (CD301CRD181- | LVVINSREEQNFVQKYLGSAYTWMGLSDPEGAWKWV |
| | 306-CD28-4- | DGTDYATGFQNWKPGQPDDWQGHGLGGGEDCAHFH |
| | 1BB-CD3ζ) | PDGRWNDDVCQRPYHWVCEAIEVMYPPPYLDNEKSN |
| | | GTIIHVKGKHLCPSPLFPGPSKPFWVLVVVGGVLACYS |
| | | LLVTVAFIIFWVRSKRSRLLHSDYMNMTPRRPGPTRKH |
| | | YQPYAPPRDFAAYRSKRGRKKLLYIFKQPFMRPVQTT |
| | | QEEDGCSCRFPEEEEGGCELRVKFSRSADAPAYQQGQ |
| | | NQLYNELNLGRREEYDVLDKRRGRDPEMGGKPRRKN |
| | | PQEGLYNELQKDKMAEAYSEIGMKGERRRGKGHDGL |
| | | YQGLSTATKDTYDALHMQALPPR |
| 94 | CD301 CAR | LTCQVATLNNNASTEGTCCPVNWVEHQDSCYWFSHS |
| | (CD301CRD163- | GMSWAEAEKYCQLKNAHLVVINSREEQNFVQKYLGS |
| | 306-CD28-4- | AYTWMGLSDPEGAWKWVDGTDYATGFQNWKPGQPD |
| | 1BB-CD3ζ) | DWQGHGLGGGEDCAHFHPDGRWNDDVCQRPYHWVC |
| | | EAIEVMYPPPYLDNEKSNGTIIHVKGKHLCPSPLFPGPS |
| | | KPFWVLVVVGGVLACYSLLVTVAFIIFWVRSKRSRLLH |
| | | SDYMNMTPRRPGPTRKHYQPYAPPRDFAAYRSKRGRK |
| | | KLLYIFKQPFMRPVQTTQEEDGCCRFPEEEEGGCELR |
| | | VKFSRSADAPAYQQGQNQLYNELNLGRREEYDVLDK |
| | | RRGRDPEMGGKPRRKNPQEGLYNELQKDKMAEAYSEI |
| | | GMKGERRRGKGHDGLYQGLSTATKDTYDALHMQALP |
| | | PR |
| 95 | CD301 CAR | LTCQVATLNNNASTEGTCCPVNWVEHQDSCYWFSHS |
| | (CD301CRD163- | GMSWAEAEKYCQLKNAHLVVINSREEQNFVQKYLGS |
| | 316-CD28-4- | AYTWMGLSDPEGAWKWVDGTDYATGFQNWKPGQPD |
| | 1BB-CD3ζ) | DWQGHGLGGGEDCAHFHPDGRWNDDVCQRPYHWVC |
| | | EAGLGQTSQESHIEVMYPPPYLDNEKSNGTIIHVKGKH |
| | | LCPSPLFPGPSKPFWVLVVVGGVLACYSLLVTVAFIIFW |
| | | VRSKRSRLLHSDYMNMTPRRPGPTRKHYQPYAPPRDF |
| | | AAYRSKRGRKKLLYIFKQPFMRPVQTTQEEDGCSCRFP |
| | | EEEEGGCELRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPR |
| 96 | CD301 CAR | CPVNWVEHQDSCYWFSHSGMSWAEAEKYCQLKNAH |
| | (CD301CRD181- | LVVINSREEQNFVQKYLGSAYTWMGLSDPEGAWKWV |
| | 316-CD28-4- | DGTDYATGFQNWKPGQPDDWQGHGLGGGEDCAHFH |
| | 1BB-CD3ζ) | PDGRWNDDVCQRPYHWVCEAGLGQTSQESHIEVMYP |
| | | PPYLDNEKSNGTIIHVKGKHLCPSPLFPGPSKPFWVLVV |
| | | VGGVLACYSLLVTVAFIIFWVRSKRSRLLHSDYMNMT |
| | | PRRPGPTRKHYQPYAPPRDFAAYRSKRGRKKLLYIFKQ |
| | | PFMRPVQTTQEEDGCSCRFPEEEEGGCELRVKFSRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPR |
| 97 | CD301(2) CAR | CPVNWVEHQDSCYWFSHSGMSWAEAEKYCQLKNAH |
| | ((CD301CRD181- | LVVINSREEQNFVQKYLGSAYTWMGLSDPEGAWKWV |
| | 316)x2-CD28- | DGTDYATGFQNWKPGQPDDWQGHGLGGGEDCAHFH |
| | 4-1BB-CD3ζ) | PDGRWNDDVCQRPYHWVCEAGLGQTSQESHAEAAA |
| | | KAAEAAAKACPVNWVEHQDSCYWFSHSGMSWAEAE |
| | | KYCQLKNAHLVVINSREEQNFVQKYLGSAYTWMGLS |
| | | DPEGAWKWVDGTDYATGFQNWKPGQPDDWQGHGL |
| | | GGGEDCAHFHPDGRWNDDVCQRPYHWVCEAGLGQT |
| | | SQESHIEVMYPPPYLDNEKSNGTIIHVKGKHLCPSPLFP |
| | | GPSKPFWVLVVVGGVLACYSLLVTVAFIIFWVRSKRSR |
| | | LLHSDYMNMTPRRPGPTRKHYQPYAPPRDFAAYRSKR |
| | | GRKKLLYIFKQPFMRPVQTTQEEDGCSCRFPEEEEGGC |
| | | ELRVKFSRSADAPAYQQGQNQLYNELNLGRREEYDVL |
| | | DKRRGRDPEMGGKPRRKNPQEGLYNELQKDKMAEAY |
| | | SEIGMKGERRRGKGHDGLYQGLSTATKDTYDALHMQ |
| | | ALPPR |
| 98 | CD301(3) | CPVNWVEHQDSCYWFSHSGMSWAEAEKYCQLKNAH |
| | ((CD301CRD181- | LVVINSREEQNFVQKYLGSAYTWMGLSDPEGAWKWV |
| | 316)x3-CD28 | DGTDYATGFQNWKPGQPDDWQGHGLGGGEDCAHFH |
| | 4-1BB-CD3ζ) | PDGRWNDDVCQRPYHWVCEAGLGQTSQESHAEAAA |
| | | KAAEAAAKACPVNWVEHQDSCYWFSHSGMSWAEAE |
| | | KYCQLKNAHLVVINSREEQNFVQKYLGSAYTWMGLS |
| | | DPEGAWKWVDGTDYATGFQNWKPGQPDDWQGHGL |
| | | GGGEDCAHFHPDGRWNDDVCQRPYHWVCEAGLGQT |
| | | SQESHAEAAAKAAEAAAKACPVNWVEHQDSCYWFSH |
| | | SGMSWAEAEKYCQLKNAHLVVINSREEQNFVQKYLG |
| | | SAYTWMGLSDPEGAWKWVDGTDYATGFQNWKPGQP |
| | | DDWQGHGLGGGEDCAHFHPDGRWNDDVCQRPYHW |
| | | VCEAGLGQTSQESHIEVMYPPPYLDNEKSNGTIIHVKG |
| | | KHLCPSPLFPGPSKPFWVLVVVGGVLACYSLLVTVAFII |
| | | FWVRSKRSRLLHSDYMNMTPRRPGPTRKHYQPYAPPR |
| | | DFAAYRSKRGRKKLLYIFKQPFMRPVQTTQEEDGCSC |
| | | RFPEEEEGGCELRVKFSRSADAPAYQQGQNQLYNELN |
| | | LGRREEYDVLDKRRGRDPEMGGKPRRKNPQEGLYNE |
| | | LQKDKMAEAYSEIGMKGERRRGKGHDGLYQGLSTAT |
| | | KDTYDALHMQALPPR |
| 99 | GlyTR2^{slCD301(4)} | CPVNWVEHQDSCYWFSHSGMSWAEAEKYCQLKNAH |
| | CAR | LVVINSREEQNFVQKYLGSAYTWMGLSDPEGAWKWV |
| | (CD301CRD181- | DGTDYATGFQNWKPGQPDDWQGHGLGGGEDCAHFH |
| | 316(4)-linker- | PDGRWNDDVCQRPYHWVCEAGLGQTSQESHAEAAA |
| | CD28-4-1BB- | KAAEAAAKACPVNWVEHQDSCYWFSHSGMSWAEAE |
| | CD3z) | KYCQLKNAHLVVINSREEQNFVQKYLGSAYTWMGLS |
| | | DPEGAWKWVDGTDYATGFQNWKPGQPDDWQGHGL |
| | | GGGEDCAHFHPDGRWNDDVCQRPYHWVCEAGLGQT |
| | | SQESHAEAAAKAAEAAAKACPVNWVEHQDSCYWFSH |
| | | SGMSWAEAEKYCQLKNAHLVVINSREEQNFVQKYLG |
| | | SAYTWMGLSDPEGAWKWVDGTDYATGFQNWKPGQP |
| | | DDWQGHGLGGGEDCAHFHPDGRWNDDVCQRPYHW |
| | | VCEAGLGQTSQESHAEAAAKAAEAAAKACPVNWVEH |
| | | QDSCYWFSHSGMSWAEAEKYCQLKNAHLVVINSREE |
| | | QNFVQKYLGSAYTWMGLSDPEGAWKWVDGTDYATG |
| | | FQNWKPGQPDDWQGHGLGGGEDCAHFHPDGRWNDD |
| | | VCQRPYHWVCEAGLGQTSQESHAEAAAKAAEAAAKA |
| | | IEVMYPPPYLDNEKSNGTIIHVKGKHLCPSPLFPGPSKP |
| | | FWVLVVVGGVLACYSLLVTVAFIIFWVRSKRSRLLHSD |
| | | YMNMTPRRPGPTRKHYQPYAPPRDFAAYRSKRGRKK |
| | | LLYIFKQPFMRPVQTTQEEDGCSCRFPEEEEGGCELRV |
| | | KFSRSADAPAYQQGQNQLYNELNLGRREEYDVLDKR |
| | | RGRDPEMGGKPRRKNPQEGLYNELQKDKMAEAYSEI |
| | | GMKGERRRGKGHDGLYQGLSTATKDTYDALHMQALP |
| | | PR |

### Examples

These examples are provided for illustrative purposes only and not to limit the scope of the claims provided herein.

### Example 1: Improved Targeting of P1,6GlcNAc-Branched N-Glycans in the GlyTR1 Bi-Specific Protein

As virtually all cell surface proteins are glycosylated, TACA target density in cancer cells can be about 100-1000 fold greater than typical protein antigens. Thus, increasing the number of TACA binding domains in GlyTR may drive cancer cells specificity by enhancing binding avidity. distinction to antibodies, where high affinity is used to achieve specificity. High avidity binding is accomplished by the combination of high-density target expression and the presence of multiple carbohydrate-binding domains. The combination of high target density and multiple binding sites should lead to high specificity for high expressing over low expressing cells. Thus, specificity of a multi-valent GlyTR protein for TACAs would not be determined by presence or absence of the target, but rather a threshold density of target expression specifically detected by GlyTRs with multiple TACA binding domains. In this manner, generating multi-valent GlyTR proteins should further improve specificity for high target expressing cancer cells and spare lower-expressing normal tissue. This concept is demonstrated herein using two different GlyTR proteins targeting β1,6GlcNAc-branched N-glycans (GlyTR1) and Tn antigen (GlyTR2).

### Improving the Design of the Original GlyTR1^{LPHAxCD3} Protein

The original GlyTR1^{LPHAxCD3} protein and sequence linked a single L-PHA domain (Phaseolus vulgaris, leukoagglutinin) to a single scFv domain specific to the CD3 protein (OKT3 clone) (FIG. 1A). See e.g., PCT/US2016/030113. L-PHA is a plant lectin with high specificity for β1,6GlcNAc-branched N-glycans. The original GlyTR1^{LPHAxCD3} had modest cancer killing activity (EC₅₀~1 nM). Size exclusion chromatography revealed that GlyTR1^{LPHAxCD3} was predominantly a dimer of ~100 kDa versus 55 kDa predicted (FIG. 1B) and thus contained two L-PHA and two anti-CD3 binding domains. Dimer formation is not unexpected as native L-PHA is a tetramer. The first 5 N-terminal amino acids of the L-PHA protein, which are distant to the carbohydrate binding site, form a β-pleated sheet that induces dimer formation through anti-parallel binding. Deletion of the first five amino acids of the L-PHA domain in GlyTR1^{LPHAxCD3} (FIG. 1A), to block dimerization and generate a monomeric GlyTR with a single L-PHA domain, significantly reduced binding to β1,6GlcNAc branched N-glycans and ability to kill cancer cells relative to dimeric GlyTR1LPHAxCD3 (FIG. 1C-D). Thus, a single TACA binding domain within GlyTR1 proteins showed significantly reduced binding when compared to GlyTR1 proteins comprising two TACA binding domains.

To further improve the activity of the original GlyTR1^{LPHAxCD3} bi-specific protein GlyTR1^{LPHA(2)xCD3} (=GlyTR^{1LPHAxLPHAxCD3}) with two L-PHA domains linked in tandem by three flexible linkers (i.e. (GGGGS)3) (FIG. 1A) were generated. Size exclusion chromatography (SEC) revealed that GlyTR1^{LPHA(2)xCD3} is about 50-70% dimer, with the rest being monomer (~30-40%) or larger multimers (~10-20%) (FIG. 2A). Dimer formation was stable, as re-running the dimer fraction on SEC reveals that >99% remains as a dimer (data not shown). Directly comparing the monomeric (two L-PHA domains) and dimeric (four L-PHA domains) fractions of GlyTR1^{LPHA(2)xCD3} revealed significantly higher binding to β1,6GlcNAc-branched N-glycans in the latter (FIG. 2B), further confirming that increasing the number of TACA binding domains within GlyTR1 proteins led to higher potency.

Indeed, dimeric GlyTR1^{LPHA(2)xCD3} (four L-PHA domains) bound to target cancer cells were significantly better than original dimeric GlyTR1^{LPHAxCD3} (two L-PHA domains), leading to a >3000 fold increase in cancer cell killing activity (FIGS. 2C-D). Moreover, dimeric GlyTR1^{LPHA(2)xCD3} potently triggered human T cell dependent killing of many diverse liquid and solid cancer types with an EC₅₀ as low as <100 femtomolar, including multiple myeloma, T cell leukemia, acute myeloid leukemia, (AML), pancreatic cancer, colon cancer, non-small cell lung cancer, prostate cancer, ovarian cancer and breast cancer, (FIGS. 3A-I). There was little killing without human T cells, confirming that GlyTR1^{LPHA(2)xCD3} induced T cells to kill cancer cells (FIGS. 3A, E-H). Killing was independent of MHC class I as CD8⁺ T cells readily induced killing of breast cancer cells deleted for b2-microglobulin (FIG. 3I). The >3000 fold increase in killing activity resulting from doubling the number of TACA binding domains from 2 to 4 was observed. This >3000 fold increase in killing activity further demonstrated that driving high avidity binding enhanced the specificity of the improved GlyTR1s for cancer cells. In particular, cancer cells that uniquely have high TACA density.

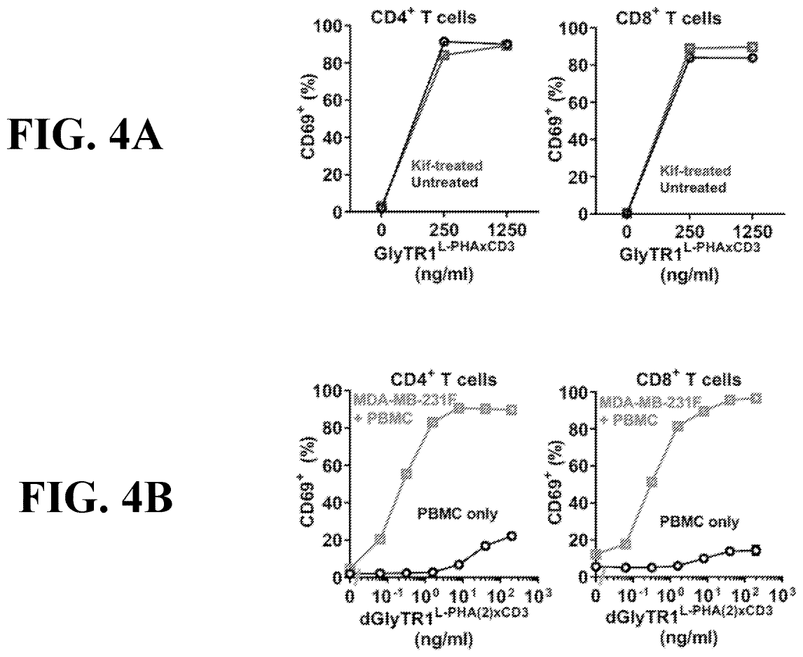

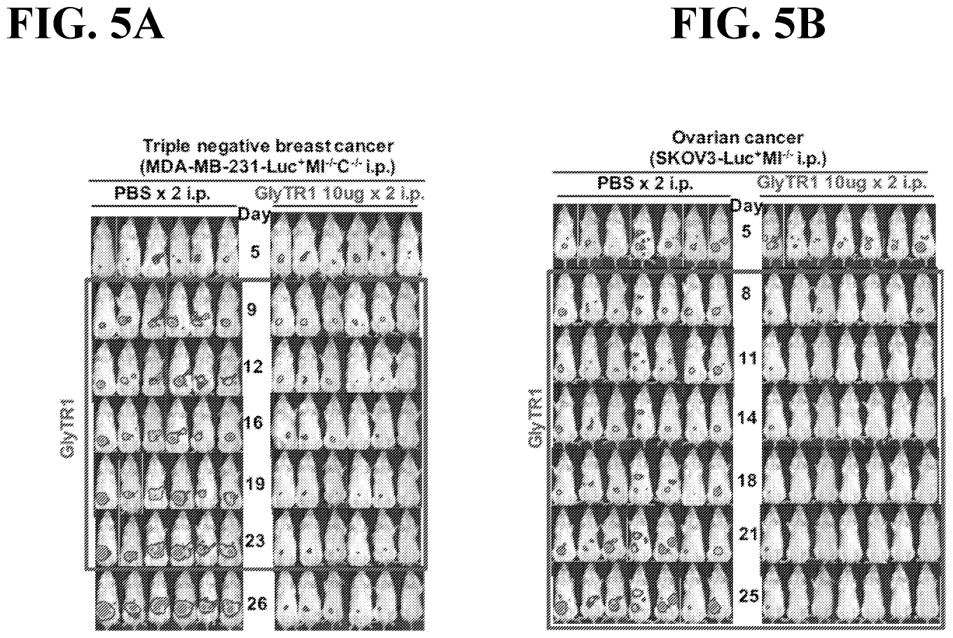

A safety concern with the original dimeric GlyTR1^{LPHAxCD3} bi-specific protein was robust T cell activation in the absence of cancer (FIG. 4A), an unwanted activity that would greatly increase risk of cytokine storm in treated patients. The presence of two CD3 binding domains in the original dimeric GlyTR1^{LPHAxCD3}, which if spatially close would lead to TCR clustering, was likely responsible for robust T cell activation in the absence of cancer. Indeed, decreasing β1,6GlcNAc-branching >85% in T cells with the Golgi inhibitor kifunensine did not reduce the mitogenic activity of original dimeric GlyTR1^{L-PHAxCD3} on T cells (FIG. 4A), confirming that the two CD3 binding domains rather than the two L-PHA domains is the primary driver of its mitogenic activity.

To address this issue, the distance between the two CD3 binding domains in dimeric GlyTR1^{LPHA(2)xCD3} was increased by using three flexible linkers (i.e. (GGGGS)3) to separate the LPHA and CD3 binding domains rather than a single flexible linker (i.e., GGGGS) as in original GlyTR1^{LPHAxCD3}. Further separation of the CD3 binding domains should reduce potential for TCR clustering in the absence of target cancer cells. Indeed, dimeric GlyTR1^{LPHA(2)xCD3} was up to ~3000 fold less effective in activating T cells in the absence than the presence of cancer cells (FIG. 4B). This provides a very large safety window for targeting cancer cells without triggering nonspecific peripheral T cell activation and cytokine release syndrome. Thus, by adding additional L-PHA domains and increasing the distance of CD3 binding domains in dimeric GlyTR1^{LPHA(2)xCD3} cancer cell specificity and killing was increased while also reducing the risk of non-specific T cell activation by up to ~3000 fold. More generally, the data indicate that the number of TACA binding domains and linker length are critical features in designing highly potent and safe GlyTR proteins.

In Vivo Cancer Killing by Dimeric GlyTR1^{LPHA(2)xCD3} (=Dimeric GlyTR1^{LPHAxLPHAxCD3})

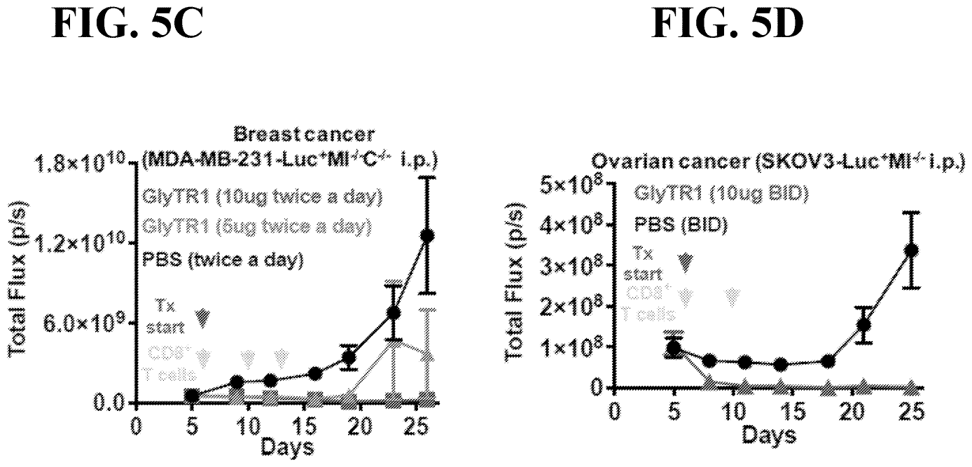

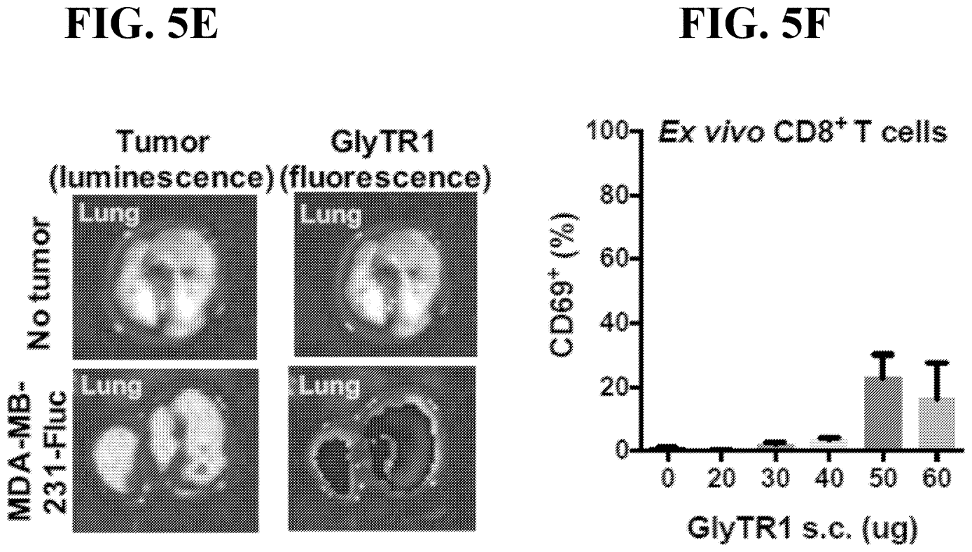

Two cancer cell lines with luciferase to allow imaging in vivo: triple negative breast cancer (MDA-MD-231-Fluc) and ovarian cancer (SKOV3-Fluc) were assessed. To limit allogeneic killing of human cancer cells by non-matched T cells (in the absence of GlyTR1) MHC class I genes (i.e., β2 microglobulin) were deleted in both cell lines (i.e., MDA-MB-231-Fluc-M1⁻ and SKOV3-Fluc-M1⁻). Flow cytometry confirmed loss of HLA ABC class I at the cell surface (data not shown). To assess cancer cell killing in vivo, NSG mice were injected intra-peritoneal (i.p.) with MDA-MB-231-Fluc-M1⁻ or SKOV3-Fluc-M1⁻ cells and once tumor was established after 5 days, mice were injected i.p. with purified CD8⁺ T cells every 3-4 days for 2 or 3 injections, respectively, along with GlyTR1^{LPHAxLPHAxCD3} i.p. twice daily. In both models, GlyTR1^{LPHAxLPHAxCD3} at 10 ug twice daily induced marked tumor regression, with many mice displaying undetectable disease after <1 week of treatment (FIGS. 5A-D). Long term survival curves could not be assessed because NSG mice humanized with PBMC develop GvHD starting ~3-4 weeks, leading to mortality. At lower doses (5 ug and 1.5 ug twice daily), GlyTR1^{LPHAxLPHAxCD3} dose dependently reduced tumor progression but were less effective than the 10 ug dosing (FIGS. 5A-D, data not shown).

Importantly, injection of fluorescently labelled GlyTR1^{LPHAxLPHAxCD3} into NSG mice with or without metastatic MDA-MB-231-Fluc-M1^{-/-} cells (from tail vein injection) demonstrated accumulation of GlyTR1^{LPHAxLPHAxCD3} in lungs with but not without cancer (FIG. 5E), indicating specificity for cancer cells in vivo. Moreover, GlyTR1^{LPHAxLPHAxCD3} did not induce human T cell activation in vivo in non-tumor bearing humanized NSG MI/II-mice at doses up to 40 ug (FIG. 5F), paralleling the in vitro data. Together these data demonstrate that GlyTR1^{LPHAxLPHAxCD3} had potent cancer killing activity in vivo.

Half-Life and In Vivo Distribution of Dimeric GlyTR1^{LPHA(2)xCD3} (=Dimeric GlyTR1^{LPHAxLPHAxCD3})

In C57BL/6 mice, the serum half-life of dimeric GlyTR1^{LPHA(2)xCD3} was ~2.7 hrs (FIG. 6B), which was similar to the FDA approved bi-specific protein blinatumomab (Blincyto). Incubation of dimeric GlyTR1^{LPHA(2)xCD3} in human plasma at 37° C. for up to 21 hrs demonstrated little loss of intact protein (data not shown), indicating that dimeric GlyTR1^{LPHA(2)xCD3} is stable in blood. Tracking distribution of fluorescently tagged dimeric GlyTR1^{LPHA(2)xCD3} in live mice demonstrated marked accumulation in the liver, with much smaller amounts in the spleen >urine/kidney (FIG. 6A, C-E). Thus, GlyTR1^{LPHA(2)xCD3} was rapidly cleared by the liver.

Safety of dimeric G1yTR1^{LPHA(2)xCD3} (=dimeric GlyTR1^{LPHAxLPHAxCD3}) L-PHA and GlyTR^{LPHA(2)xCD3} immunohistochemistry of normal human tissue.

Immuno-histochemistry with L-PHA (the lectin used in GlyTR1) at 250 ng/ml on a normal human tissue microarray ((FDA999u from BioMax, 32 normal human tissues from 3 different individuals per tissue) was compared to staining of metastatic colorectal cancer as a positive control (FIG. 7). Soluble GalNAc and thyroglobulin, which contains β1,6GlcNAc-branched N-glycans, were used as competitive inhibitors and confirmed specificity of binding (data not shown). This analysis revealed mild to moderate but variable L-PHA staining in the brush border of the small bowel, surface epithelial cells of the stomach, exocrine pancreas (acinus, intra-cellular), and kidney cortex (e.g., glomerulus, brush border of proximal tubule) (FIG. 7). The latter was consistent with published data reporting L-PHA binding to glomerular podocytes (bowman's space), brush border of proximal tubule and the thick portion of Henle's loop, with distal tubules being negative (Truong et al., Histochemistry 90: 51-60 (1988)). Higher L-PHA concentrations revealed lower staining in adrenal, parotid duct, colloid (thyroid), testis, prostate, uterus, spleen, CNS white matter and the molecular layer of the cerebellum. However, there is a marked inter-individual difference in the degree of staining. For example, using higher L-PHA concentrations to stain normal adjacent pancreatic tissue microarray containing 60 different individuals, revealed ~40-50% with negative/low vs 50-60% with moderate staining (data not shown).

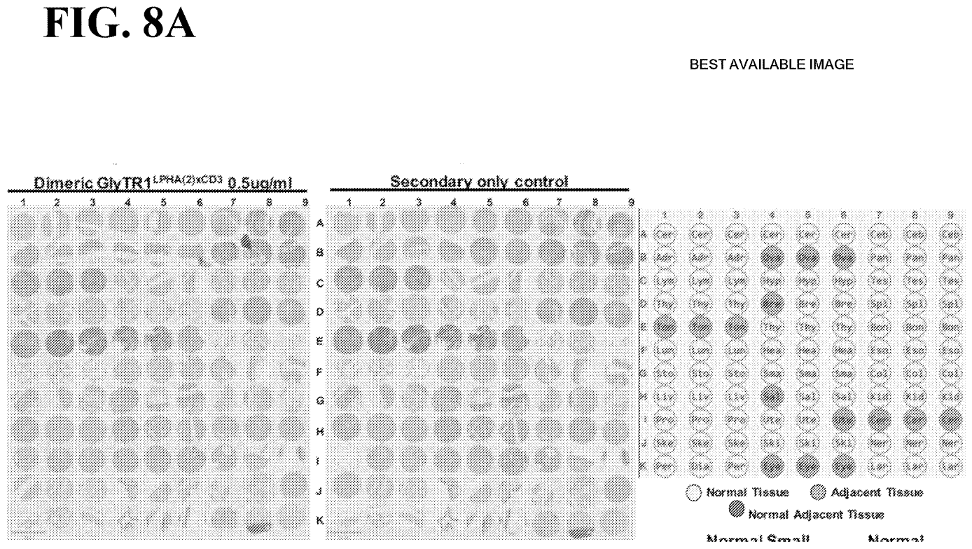

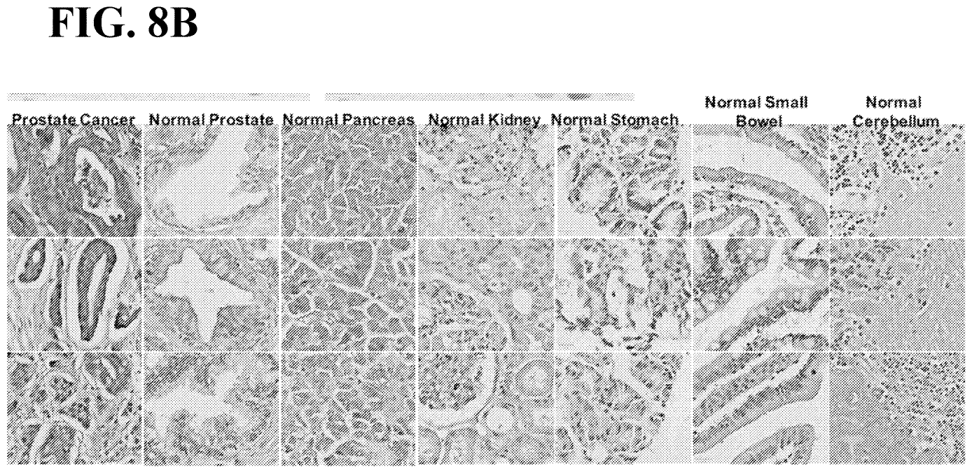

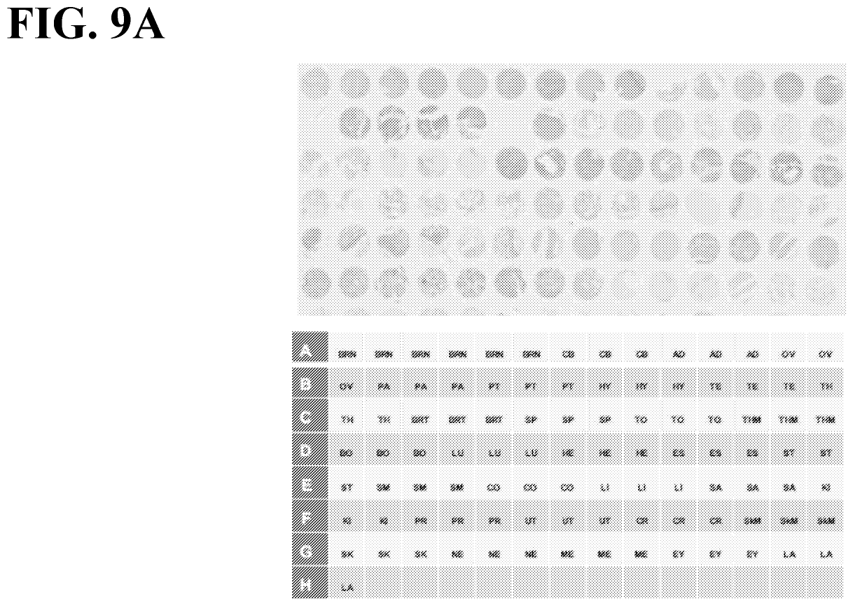

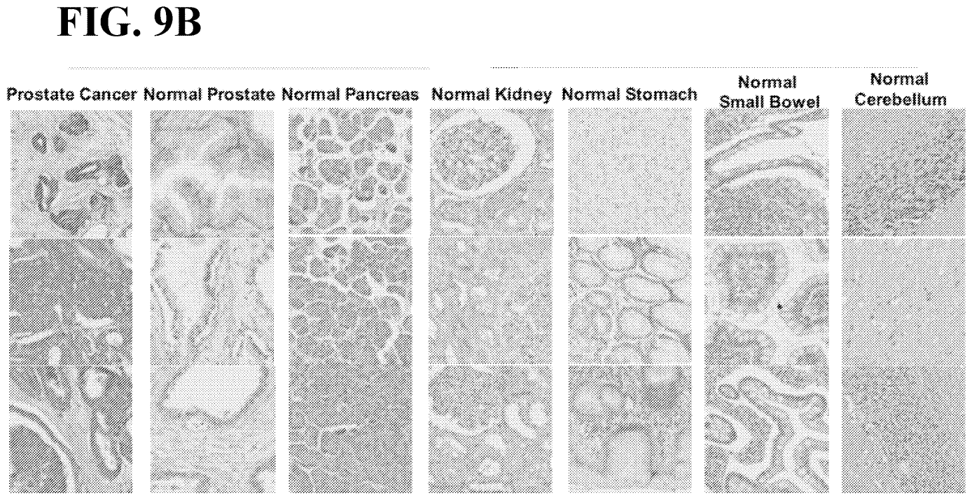

This experiment was repeated using GlyTR1^{LPHA(2)xCD3} to stain FDA999w from BioMax (FIGS. 8A-B) as well as a second independent normal human tissue microarray (FDA999-1 from BioLabs, 32 normal human tissues from 3 different individuals per tissue) (FIGS. 9A-B). Prostate cancer (NBP2-30169, Novus Biologicals) was co-stained as a positive control in both cases. This revealed similar results as L-PHA staining, with low but variable staining of the brush border of the small bowel, surface epithelial cells of the stomach, exocrine pancreas (acinus, intra-cellular), kidney cortex (glomerulus, proximal tubules), prostate and the molecular layer of the cerebellum (FIG. 8-9). Higher concentrations revealed lower and variable staining in adrenal, parotid duct, thyroid colloid, testis, uterus, spleen and CNS white matter.

Dimeric GlyTR1^{LPHA(2)xCD3} does not Trigger Killing of Normal Primary Human Renal Epithelial Cells or Hepatocytes.

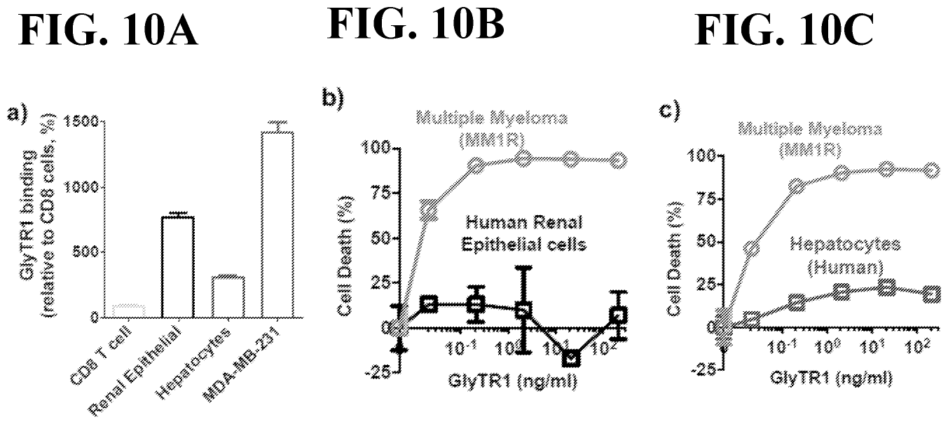

To validate the IHC results, flow cytometry was used to assess binding of dimeric GlyTR1^{LPHA(2)xCD3} to cultured normal primary human renal epithelial cells (ScienCell) and cultured normal primary human hepatocytes (ScienCell); with comparison to normal CD8⁺ T cells and MDA-MB-231 breast cancer cells as controls. Consistent with the IHC results, binding of GlyTR^{LPHA(2)xCD3} to human renal epithelial cells was greater than binding to human hepatocytes but significantly less than binding toMDA-MB-231 breast cancer cells (FIG. 10A). More importantly, GlyTR^{LPHA(2)xCD3} did not induce T cell dependent killing of human renal epithelial cells or human hepatocytes at concentrations that robustly triggered killing of MM1R multiple myeloma cells and (FIGS. 10 B-C). This further exemplifies the critical importance of a threshold level of TACA target density being required to get robust killing of GlyTR1^{LPHA(2)xCD3}

### Lack of T Cell Dependent “On-Target Off-Cancer”” Toxicity of Dimeric GlyTR1^{LPHA(2)xCD3} in Humanized NSG Mice.

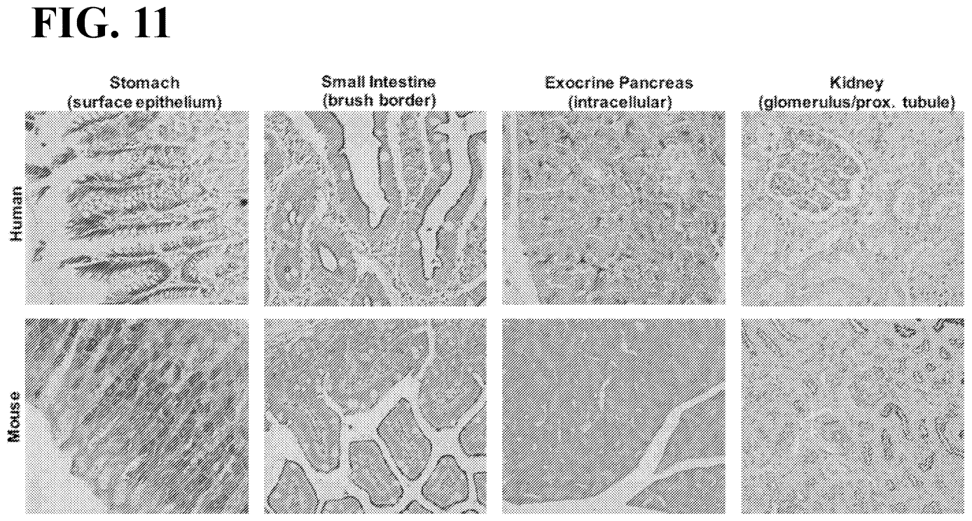

To explore whether mice can provide a model to test “on-target/off-cancer” toxicity of GlyTR1^{LPHA(2)xCD3} in vivo, L-PHA staining of the FDA999u (BioMAx) normal human tissue microarray was compared with a mouse (C57BL6, AMS545 (Pantomics)) tissue microarray containing 22 normal mouse tissues duplicated or triplicated from three C57BL/6J mice. This analysis revealed L-PHA positive staining in mouse surface epithelial cells of the stomach, brush border of the small intestine and kidney (tubules >glomerulus), replicating staining in three of the highest staining organs in normal human tissue (FIG. 11). Lower-level staining in mice was also observed in the molecular layer of the cerebellum and spleen. These results indicated that T cell humanized mice can provide a model to test “on-target/off-cancer” toxicity of dimeric GlyTR1^{LPHA(2)xCD3} for three of the highest positive tissues in humans (i.e. kidney, stomach. small intestine). To minimize xenogeneic graft versus host disease (GvHD) from human T cells as a complicating factor in the toxicity experiment, NSG-MI/II⁻ mice that lack MHC class I and II were utilized. These mice delay, but do not eliminate, development of xenogeneic GvHD due to remaining presence of minor histocompatibility antigens. NSG-MI/II⁻ mice (n=12) were engrafted with 2×10⁷ human PBMCs intravenously on day 0 and day 15, with 25.8% (+/−7.3 std dev.) of blood leukocytes being human (i.e., human CD45 positive) on day 14 (FIG. 12, panel a). Of these hCD45⁺ cells, 94.7% (+/−2.7%) were human CD3⁺ T cells. On day 16, the humanized NSG-M/II⁻ mice were injected subcutaneously twice per day for 12 consecutive days with PBS (n=6) or dimeric GlyTR1^{LPHA(2)xCD3} (n=6), followed by euthanization on day 28 (FIG. 12, panel a). Treatment did not significantly alter weight relative to mock treated mice (FIG. 12, panel b), although one treated mouse had about 15% weight loss at day 18 that recovered with saline injection. One treated mouse developed mild alopecia of the head, but otherwise no other overt clinical toxicity was observed. Clinical laboratory testing of blood on day 28 revealed no treatment induced differences in liver function (AST, ALT, ALP, protein, albumin, total bilirubin), kidney function (BUN, creatinine), electrolytes, glucose, pancreatic function (amylase, precision PSL), thyroid function (total T4, TSH), cholesterol or muscle (CPK) (FIG. 12, panels c-v).

Although the hematopoietic system is not normal in humanized NSG-M/II⁻ mice, which complicates interpretation, dimeric GlyTR1^{LPHA(2)xCD3} treatment also did not alter blood levels of hemoglobin, RBC, hematocrit, WBC, WBC differential or platelets relative to control (FIG. 12, panels w-D). Analysis of the spleen revealed increased size, cellularity and number of hCD45⁺ leukocytes compared to the treated group (FIG. 12, panel E), but no difference in the percentage of total human CD45⁺ leukocytes, CD4⁺ T cells, CD8⁺ T cells, B cells or T regulatory cells (Treg) (FIG. 12, panels F-L). There was also no difference in the T cell activation markers CD69 or CD25 in either CD4⁺ or CD8⁺ T cells, signifying that dimeric GlyTR1^{LPHA(2)xCD3} treatment did not induce T cell activation (FIG. 12, panels K-M). The percentage of PD-1 positive CD4⁺ T and CD8⁺ T cells were very high but did not differ with GlyTR1^{LPHAxLPHAxCD3} treatment (FIG. 12, panels N,O), indicating significant ongoing xenogeneic reactivity that was not further enhanced by GlyTR1^{LPHAxLPHAxCD3}. Consistent with this, serum hIFNγ or hIL-6 levels did not differ between control and dimeric GlyTR1^{LPHA(2)xCD3} treated mice (FIG. 12, panels P,Q).

Due to the lack of MHC, which is required to provide survival signals to T cells via basal T cell receptor signaling, human T cells in NSG-MI/II mice decline over time. For example, a separate experiment showed that by ~6 weeks post-humanization, human leukocyte numbers in blood had declined precipitously from a median of about 30% at 3 weeks to a median of ~6%. Coupled with the lack of up-regulation of serum IFNγ/IL-6 levels or induction of the T cell activation markers CD69 and CD25 by dimeric GlyTR1^{LPHA(2)xCD3}, these data suggest that the larger spleen in treated mice is likely secondary to dimeric GlyTR1^{LPHA(2)xCD3} providing survival signals to T cells rather than direct T cell activation and expansion.

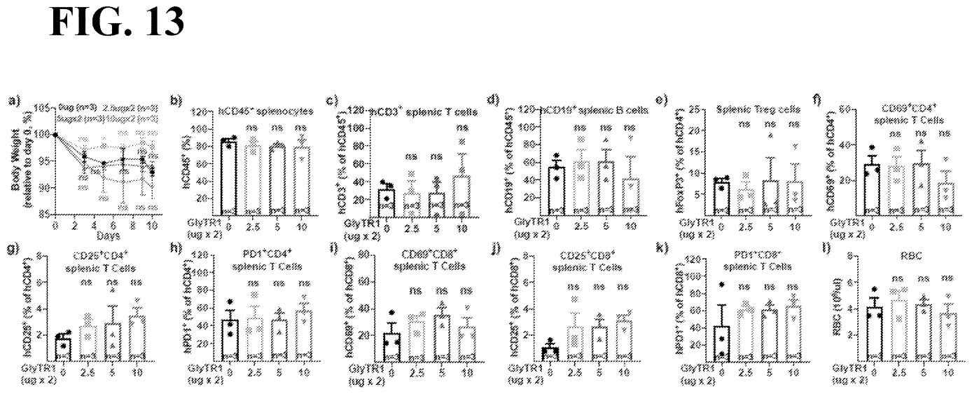

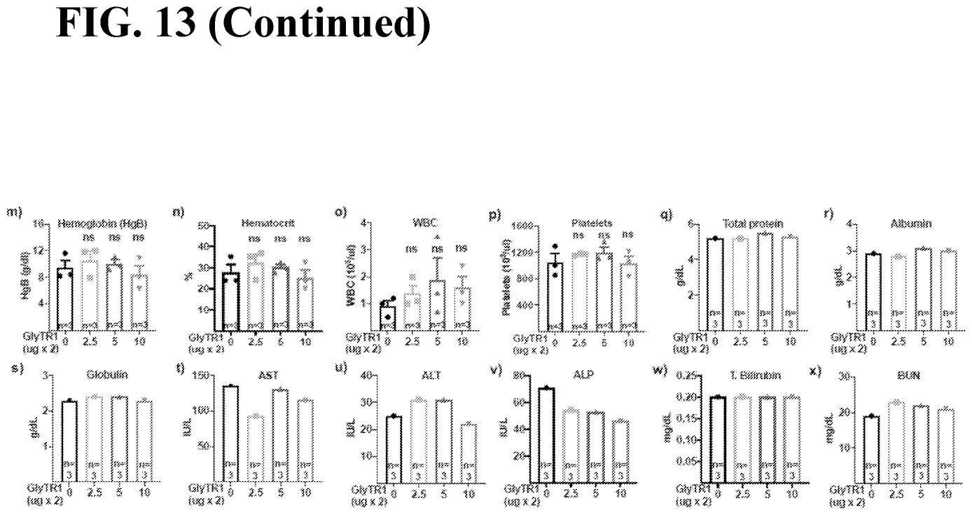

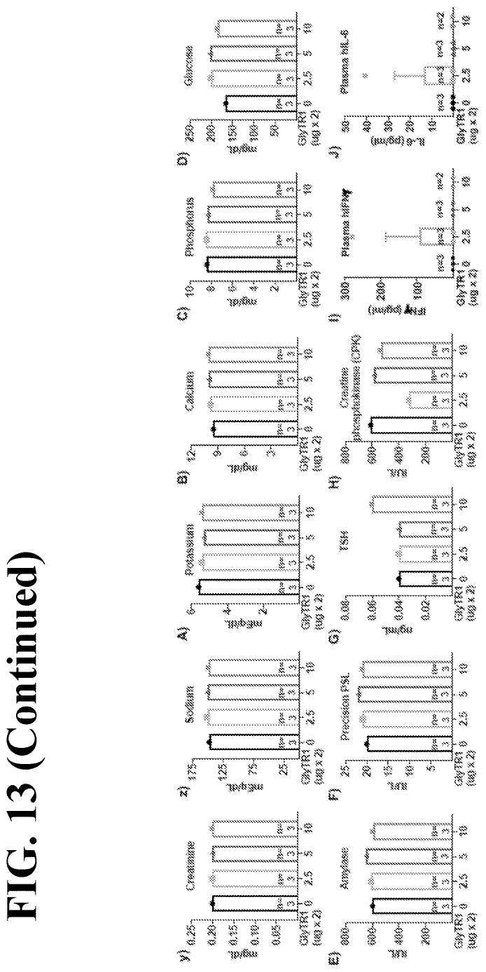

To further examine this possibility and replicate the toxicity results, the experiment was repeated using NSG mice humanized with CD34⁺ hematopoietic stem cells. In these mice, CD34⁺ hematopoietic stem cells (HSC) seed the thymus and bone marrow, leading to multi-lineage hematopoiesis and functional human CD4⁺ and CD8⁺ T cells in blood and lymphoid organs. Since a mouse's MHC is considered self to the human T cells in these mice, there was no issue with lack of survival signal as in the NSG-M/II⁻ mice; however these mice could still develop GvHD starting about 25-30 weeks post engraftment. Dimeric GlyTR1^{LPHA(2)xCD3} treatment (0 ug, 2.5 ug, 5 ug and 10 ug twice daily for 10 days, n=3 per group) of mice 32 weeks post CD34⁺ HSC engraftment did not induce any overt clinical toxicity nor alter weight, spleen size/cellularity, total human splenic CD4⁺ and CD8⁺ T cells, B cells, Treg cells or T cells positive for CD69, CD25 or PD-1 (FIG. 13, panels a-k). Dimeric GlyTR^{LPHA(2)xCD3} treatment also did not alter hemoglobin, RBC, hematocrit, WBC, WBC differential or platelets relative to control (FIG. 13, panels l-p). Pooled blood from the three mice in each treatment group also showed that dimeric GlyTR^{LPHA(2)xCD3} treatment did not alter kidney function (BUN, creatinine), liver function (AST, ALT, ALP, protein, albumin, total bilirubin), electrolytes, pancreatic function (amylase, precision PSL), muscle (CPK) thyroid function (TSH) or cholesterol (FIG. 13, panels q-H). Serum hIFNγ or hIL-6 levels did not differ between control and dimeric GlyTR^{LPHA(2)xCD3} treated mice (FIG. 13, panels I, J).

### SUMMARY

Dimeric GlyTR^{LPHA(2)xCD3} target expression (β1,6 GlcNAc-branched N-glycans) was similar between mice and humans in three of the four highest staining normal organs in humans, namely kidney, stomach and small intestine. Yet at doses that readily triggered robust cancer killing in vivo, dimeric GlyTR^{LPHA(2)xCD3} treatment of humanized mice did not induce 1) “on-target, off cancer” toxicity in major organs, or 2) non-specific T cell activation. The lack of “on-target, off cancer” organ toxicity was consistent with fluorescently tagged dimeric GlyTR1^{LPHA(2)xCD3} not significantly accumulating in mouse tissues with the highest target expression, namely kidney, stomach and small intestine (FIGS. 6 A, C-E). Lack of kidney toxicity was also consistent with the molecular weight of dimeric GlyTR^{LPHA(2)xCD3} (about 182 kDa) being well above the glomerular filtration cut-off of about 70 kDa. This should limit access of dimeric GlyTR^{LPHA(2)xCD3} to the lumen of bowman's space/tubules where target expression is highest, thereby further mitigating potential risk for renal toxicity. Finally, the lack of kidney and liver toxicity in vivo is consistent with the in vitro data that dimeric GlyTR^{LPHA(2)xCD3} did not induce T cell killing of primary human renal epithelial cells or primary human hepatocytes (FIGS. 10 B, C).

### Example 2: Generation and Optimization of the GlyTR2 Bi-Specific Protein Targeting Tn Antigen

Tn antigen. Although not found on the cell surface of normal human tissue, Tn antigens are expressed in ~90% of human carcinomas and many hematopoietic cancers. Indeed, Tn antigens are one of the most specific human cancer associated structures known and promote cell motility, invasiveness and metastasis. The Tn antigen is a single N-acetyl-galactosamine (GalNAc) a-O-linked to serine/threonine in proteins like mucins. A Tn is a biosynthetic precursor of O-glycans that is normally extended with α1,3 linked galactose. The chaperone protein COSMC, a protein required by T-synthase to add galactose to GalNAc, is frequently altered in cancer. Mis-localization of enzymes within the ER/Golgi may also lead to abnormal Tn antigen expression in human cancer. The Tn antigen can be abnormally extended with Sialic Acid to make the sTn antigen; which is also not typically expressed in normal tissue.

Targeting Tn antigen. To generate the first GlyTR protein targeting Tn antigen, human CD301 (CLEC10 A, macrophage galactose lectin) was utilized. CD301 (CLEC10) is a transmembrane lectin expressed in macrophages and dendritic cells that functions as a pattern recognition receptor for non-self antigens and binds to Tn⁺ cancers. See e.g., Nollau et al., J. histochemistry and cytochemistry, 61:199-205 (2013); Lenos et al., Oncotarget 6: 26278-26290 (2015). Detailed binding analysis demonstrated high specificity of human CD301 for small glycans containing GalNAc with exposed 3- and 4-hydroxyl groups, a structure typified by the Tn cancer antigen but not other common glycans. CD301 also strongly binds to three other well-known cancer specific glycan antigens containing 3- and 4-hydroxyl exposed GalNAc, namely sTn38,40 and the gangliosides GD2 and GM236. These three glycan antigens are the only TACAs that have reached Phase III immunotherapy clinical trials, with an anti-GD2 monoclonal antibody being FDA approved for neuroblastoma.

Consistent with being a pattern recognition receptor, CD301 also binds the invertebrate glycan LacdiNAc (GalNAcβ1,4GlcNAc). Mammalian cells generally do not express LacdiNAc, but expression is often induced in many human cancers. The blood group A glycan antigen has a terminal GalNAc residue, however CD301 is expressed in blood group A individuals without inducing toxicity. Indeed, CD301 failed to bind blood group A positive RBC or blood vessels on a tissue microarray (data not shown). Finally, a fully human protein CD301 should be poorly immunogenic. As such, a human CD301 provides high specificity for Tn antigen and three other well-known TACAs.

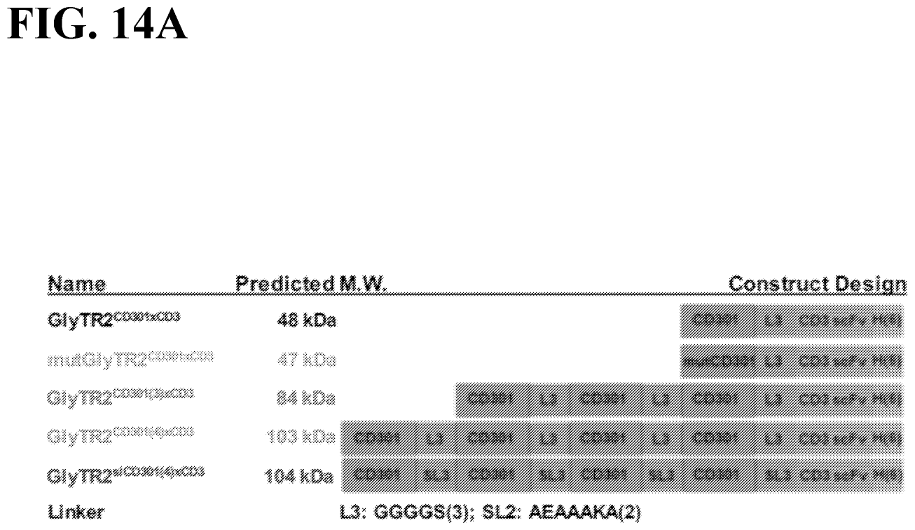

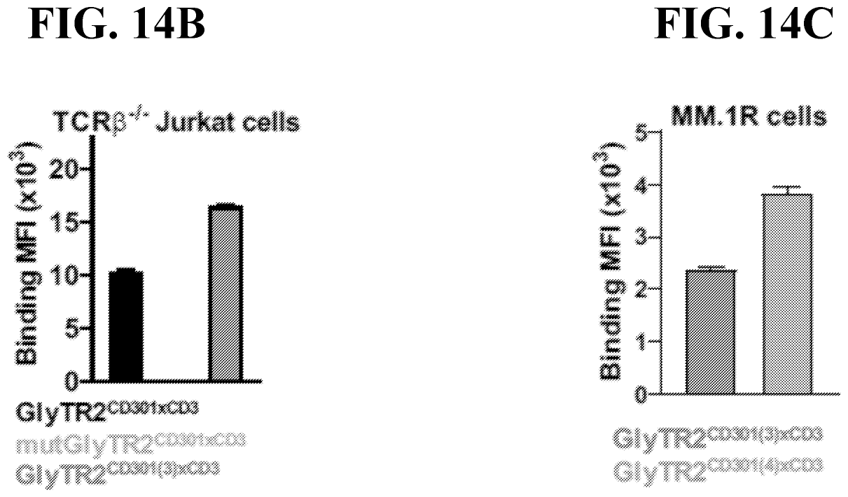

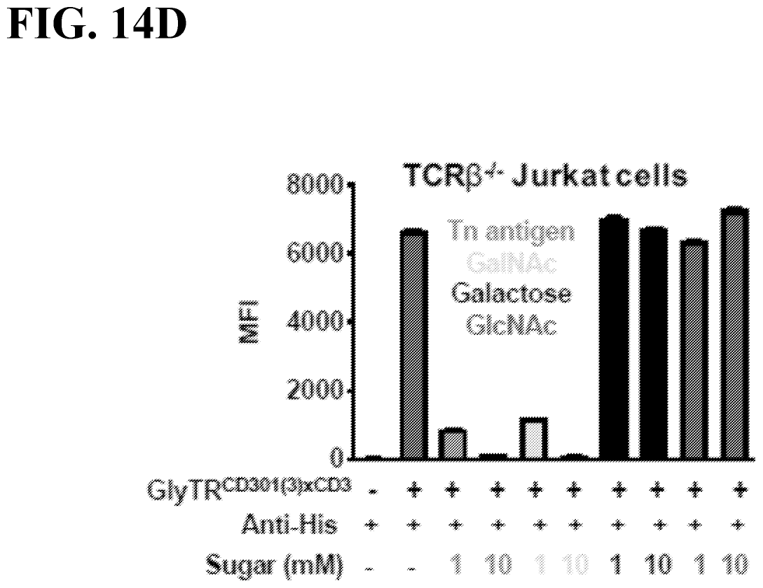

To generate a GlyTR2 bi-specific protein using CD301 to target Tn antigen, the extracellular domain of human CD301 was combined with a scFv domain specific to CD3. See e.g., International Application NO. PCT/US2016/030113. However, this protein was unable to be expressed in CHO cells, presumably because of protein mis-folding. The CD301 extracellular domain consists of a neck region and a single TACA binding domain (e.g., carbohydrate recognition domain (CRD)). The neck region promotes trimerization of CD301. See e.g., Jegouzo et al., Glycobiology 23:853-864 (2013); Napoletano et al., Eur. J. Immunol. 42:936-945 (2012). Therefore, deletion of the neck region should avoid multimerization and may promote folding of GlyTR2 proteins. Indeed, a GlyTR2^{CD301xCD3} containing a single CD301 TACA binding domain (e.g., carbohydrate recognition domain) without most of the neck region was readily expressed and bound Tn^{high} Jurkat-TCRβ^{-/-} leukemic T cells (FIGS. 14 A, D). Jurkat-TCRβ^{-/-} leukemic T cells express maximal levels of Tn antigen due to mutation of the chaperone protein COSMC, a protein required by T-synthase to extend GalNAc with galactose and produce mature O-glycans. Point mutation of 5 amino acids critical for sugar and calcium-binding in the CRD of CD301⁴⁵, namely Gln267Gly, Asp269Gly, Glu280Gly, Asn292Gly andAsp293Gly (NCBI Ref Seq NP_878910.1; SEQ ID NO: 164), abolished binding of mutGlyTR2^{CD301xCD3} to Tn^{high} Jurkat-TCR^{β-/-} leukemic T cells (FIGS. 14 A, B). Oo-Puthinan et al., Biochim. Biophys. Acta 1780: 89-100 (2008). The data on GlyTR1 described above indicated that adding additional TACA binding domains enhanced the binding avidity to TACAs. Consistent with this, GlyTR2^{CD301(3)xCD3} (three CD301 domains) was superior to GlyTR2^{CD301xCD3} (single CD301 domain) at binding to Tn⁺ Jurkat-TCRβ^{-/-} leukemic T cells (FIGS. 14 A, B). Soluble Tn antigen (GalNAcα-Ser) and GalNAc but not related sugars galactose and GlcNAc blocked binding of GlyTR2^{CD301xCD3} to Tn^{high} Jurkat-TCRβ^{-/-} leukemic T cells, confirming specificity of GlyTR2^{CD301(3)xCD3} for Tn antigen (FIG. 14 D). Adding a fourth CD301 domain (i.e., GlyTR2^{CD301(4)xCD3}) further improved binding relative to GlyTR2^{CD301(3)xCD3} with three binding domains (FIGS. 14 A, C).

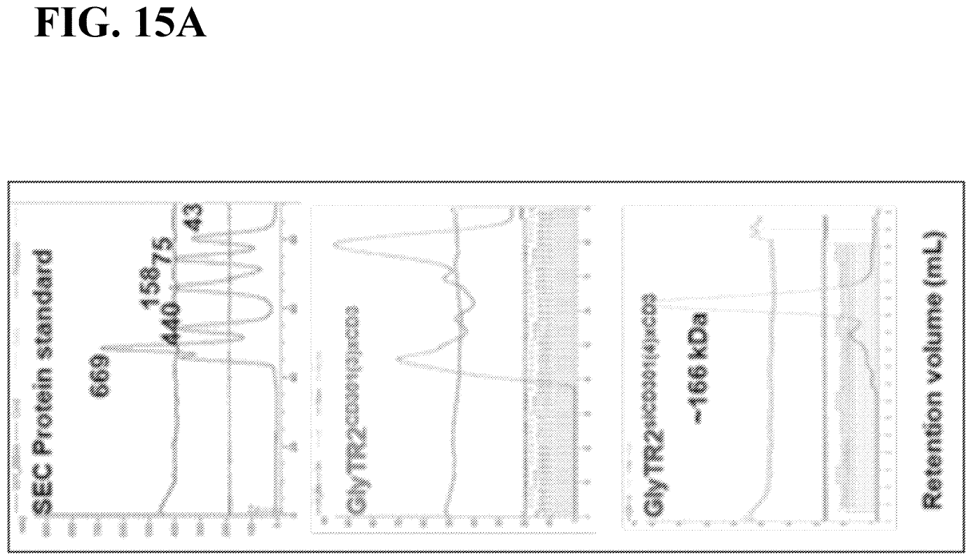

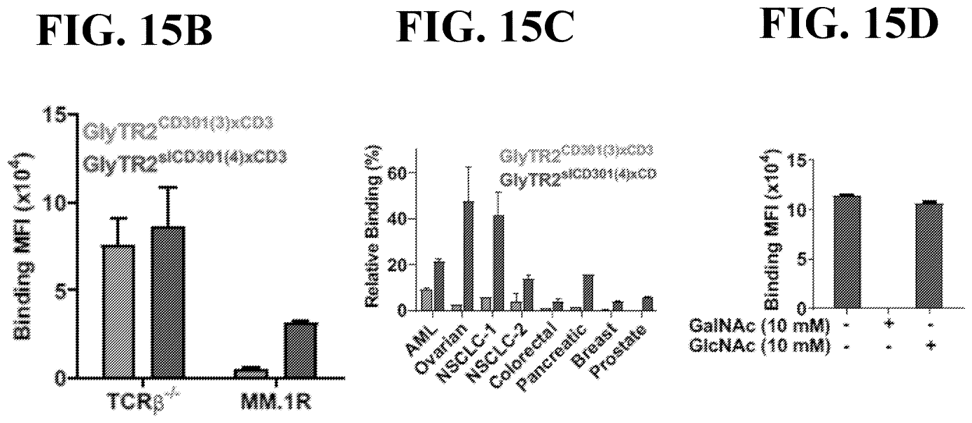

However, size-exclusion chromatography indicated that GlyTR2^{CD301(3)xCD3} was predominantly made up of large multimers (FIG. 15 A), which would negatively impact manufacturing consistency and potentially in vivo activity and safety. Therefore, to reduce potential for multimerization, the flexible linkers (GGGGS(3); SEQ ID NO: 127) separating individual CD301 domains was replaced with stiff linkers (AEAAAKA(2); SEQ ID NO: 131) (GlyTR2^{slCD301(4)xCD3} in FIG. 14 A). Indeed, size exclusion chromatography (SEC) revealed that GlyTR2^{slCD301(4)xCD3} with stiff linkers (AEAAAKA(2); SEQ ID NO: 131) was a monomer (FIG. 15 A). GlyTR2^{slCD301(4)xCD3} (stiff-linkers, four CD301 domains) bound to Tn^{high} Jurkat-TCRβ^{-/-} leukemic T cells similar to GlyTR2^{CD301(3)xCD3} (flexible linkers, three CD301 domains), but bound significantly better to a wide diversity of lower Tn expressing tumor cell lines (FIGS. 15 B, C). GalNAc but not the related sugar GlcNAc readily blocked binding of GlyTR2^{slCD301(4)xCD3} to Tn⁺ MM1R multiple myeloma cells, confirming specificity of binding to Tn antigen (FIG. 15 D). Given these data, GlyTR2^{slCD301(4)xCD3} was selected for further characterization.

### In Vitro and In Vivo Cancer Killing by GlyTR2^{slCD301(4)xCD3}

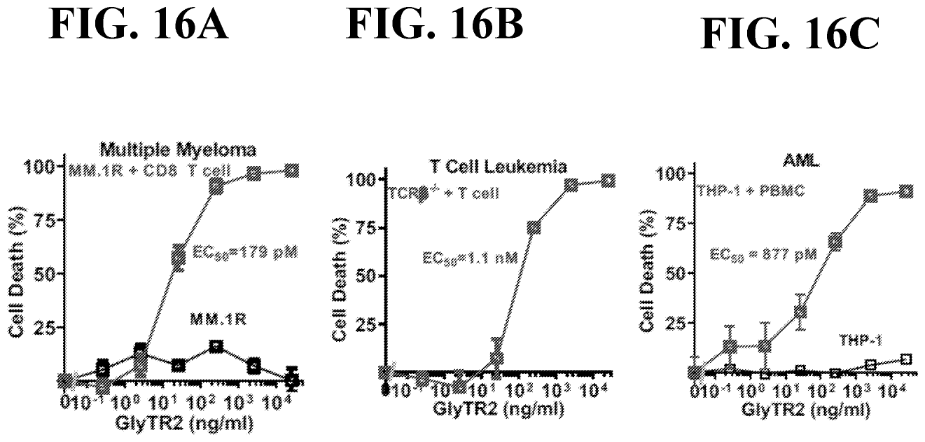

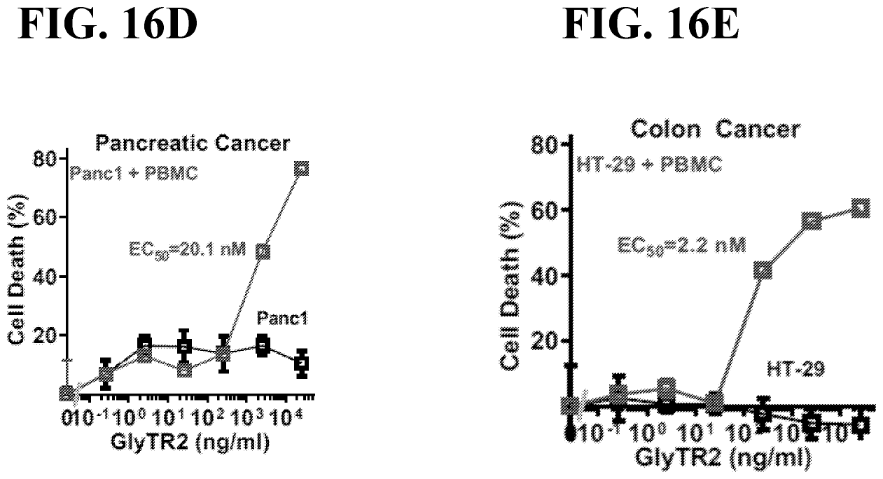

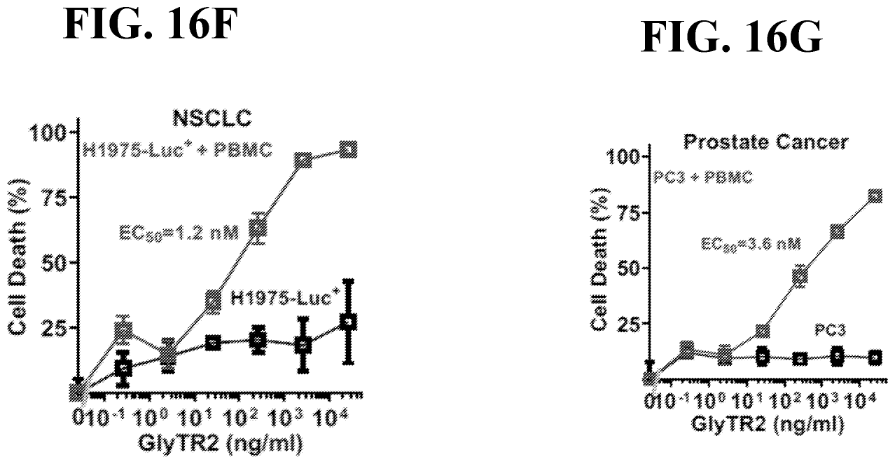

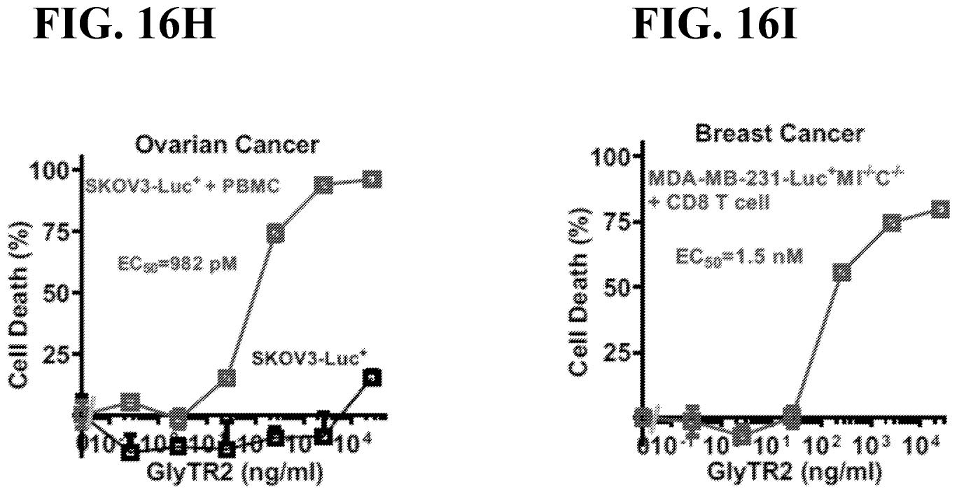

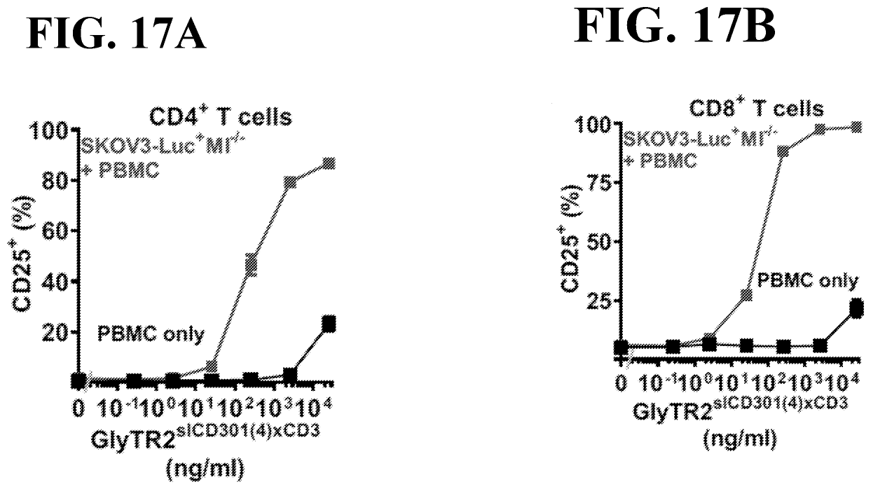

GlyTR2^{slCD301(4)xCD3} dose-dependently triggered T cell mediated killing of diverse Tn⁺ liquid and solid cancers with EC₅₀ in the high pM to low nM range, including multiple myeloma, T cell leukemia, AML, pancreatic cancer, colon cancer, non-small cell lung cancer, prostate cancer, ovarian cancer and breast cancer (FIGS. 16 A-I). There was little killing without PBMCs/T cells, confirming killing by GlyTR2^{slCD301(4)xCD3} requires T cells (FIGS. 16 A, C, D-H). CD8⁺ T cells readily induced killing of breast cancer cells deleted for 02-microglobulin, demonstrating that killing is independent of MHC class I (FIG. 16 I). GlyTR2^{slCD301(4)xCD3} induced robust T cell activation in the presence but not absence of Tn antigen positive cancer cells (FIGS. 17 A, B). Thus, GlyTR2^{slCD301(4)xCD3} should have reduced risk of nonspecific T cell activation and cytokine release syndrome.

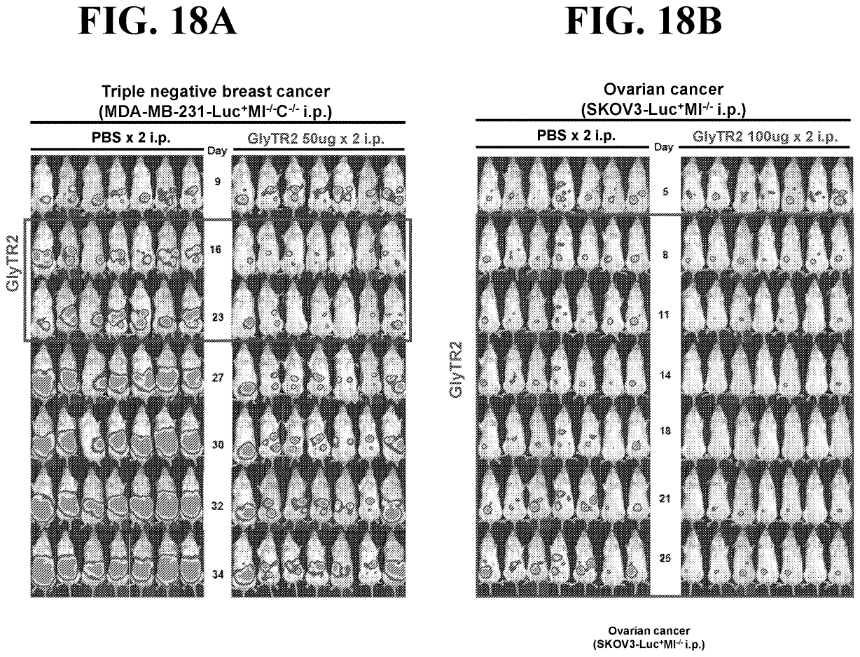

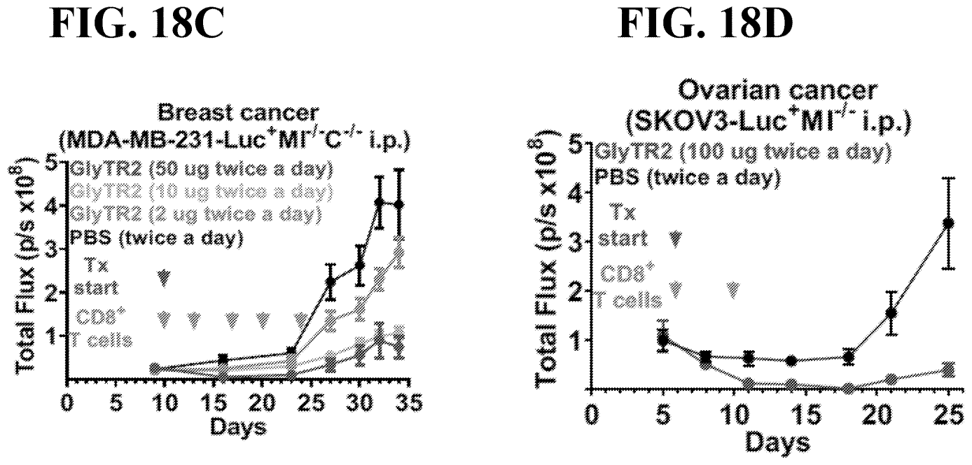

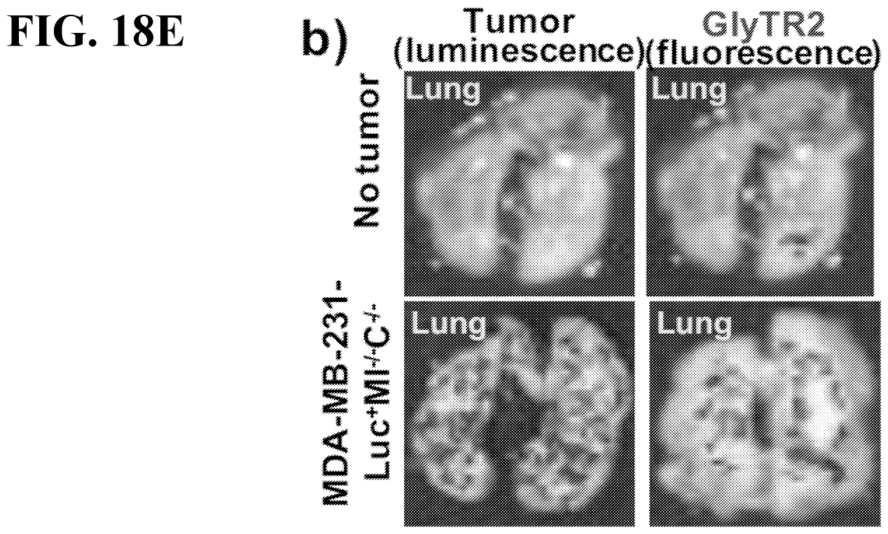

To first assess GlyTR2^{slCD301(4)xCD3} activity in vivo, Tn antigen expression was maximized in MDA-MB-231-Fluc-M1-breast cancer cells by deleting the gene COSMC (i.e. MDA-MB-231-luc⁺MI^{-/-} C^{-/-} cells). GlyTR2^{slCD301(4)xCD3} readily induced killing of these cells by purified CD8⁺ T cells in vitro (FIG. 16 I). In mice with established breast cancer tumors, 15 days of GlyTR2^{slCD301(4)xCD3} treatment dose dependently induced tumor regression in NSG mice humanized with CD8⁺ T cells compared to with CD8⁺ T cells compared to control mice (FIGS. 18 A,C). To confirm in vivo activity in solid cancers with more modest Tn antigen expression, SKOV3 ovarian cancer cells knocked out for MHC class I were utilized. As with breast cancer, GlyTR2^{slCD301(4)xCD3} treatment induced marked ovarian tumor regression in NSG mice humanized with CD8⁺ T cells relative to control mice (FIGS. 18B,D). Injection of fluorescently labelled GlyTR2^{slCD301(4)xCD3} into NSG mice with or without metastatic MDA-MB-231-Fluc-MI^{-/-} C^{-/-} cells (from tail vein injection) demonstrated accumulation of GlyTR2^{slCD301(4)xCD3} in lungs with but not without cancer (FIG. 18 E), indicating specificity for cancer cells in vivo. Together, these data demonstrated that GlyTR^{slCD301(4)xCD3} readily induced killing of Tn⁺ cancers in vivo.

### In Vivo Distribution of GlyTR2slCD301(4)xCD3

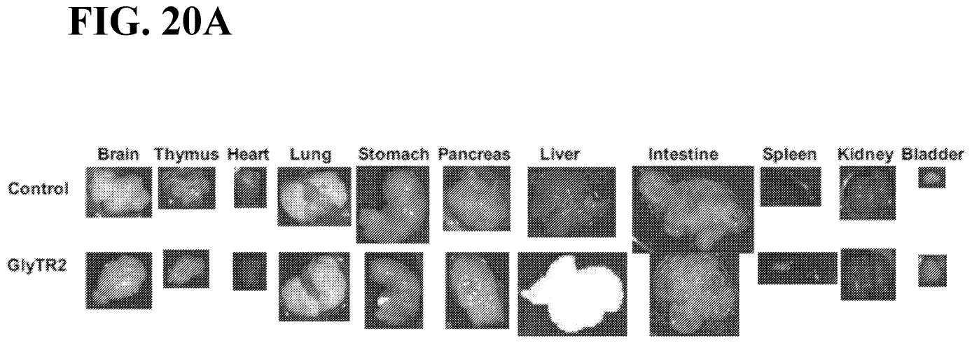

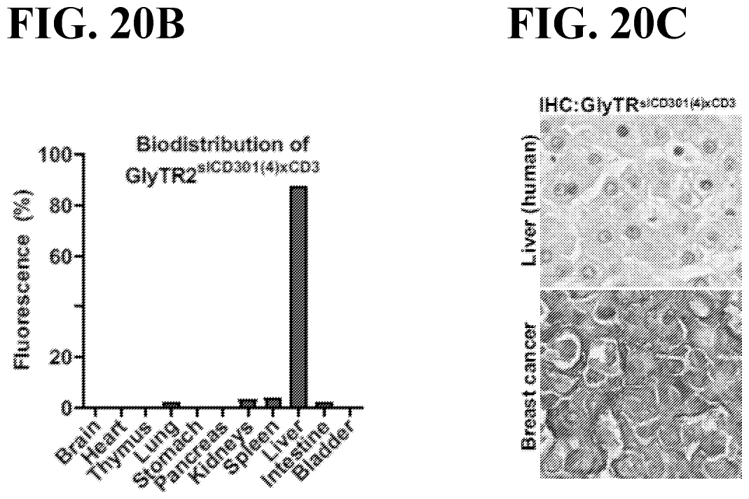

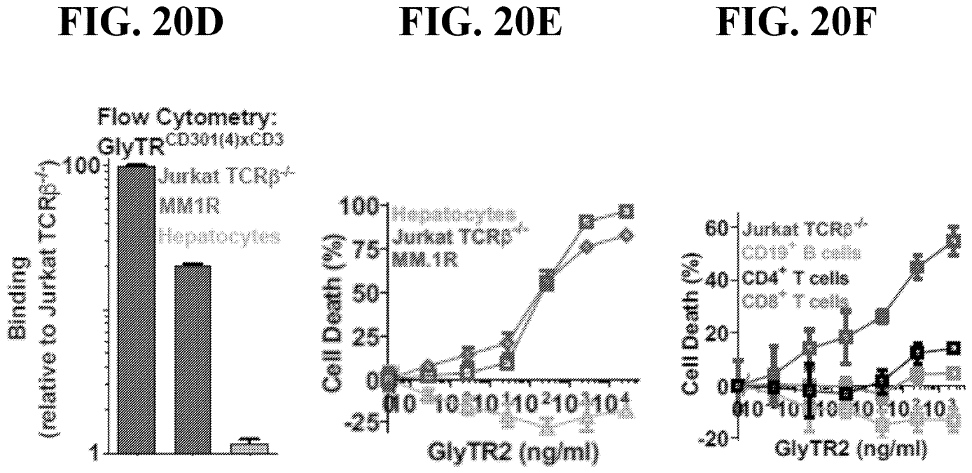

The serum half-life of GlyTR2^{CD301(3)xCD3} was determined to be about 2 hrs (FIG. 19A), similar to the FDA approved bi-specific protein Blincyto and dimeric GlyTR1^{LPHA(2)xCD3}. Incubation of GlyTR2^{CD301(3)xCD3} in human plasma at 37° C. for up to 21 hrs demonstrated little loss of intact protein, indicating that GlyTR2^{CD301(3)xCD3} is stable in blood (FIGS. 19 B,C). Tracking distribution of fluorescently tagged GlyTR2^{slCD301(4)xCD3} in mice demonstrated accumulation in the liver, with minimal amounts in kidney, spleen, lung and intestine (FIGS. 20 A, B), indicating rapid clearance by the liver as observed with GlyTR1^{LPHA(2)xCD3}. In contrast to cancer cells, GlyTR2^{slCD301(4)xCD3} did not significantly bind to human or mouse liver cells (FIGS. 20 C, D and data not shown), indicating that accumulation in the liver is not via binding to GalNAc containing glycans. Rather uptake is via bulk endocytosis, as occurs with other therapeutic proteins. Lack of binding to liver cells should also prevent T cell mediated killing. Indeed, GlyTR2^{slCD3o1(4)xCD3} did not induce T cell dependent killing of human hepatocytes at concentrations that trigger cancer cell killing (FIG. 20 E).

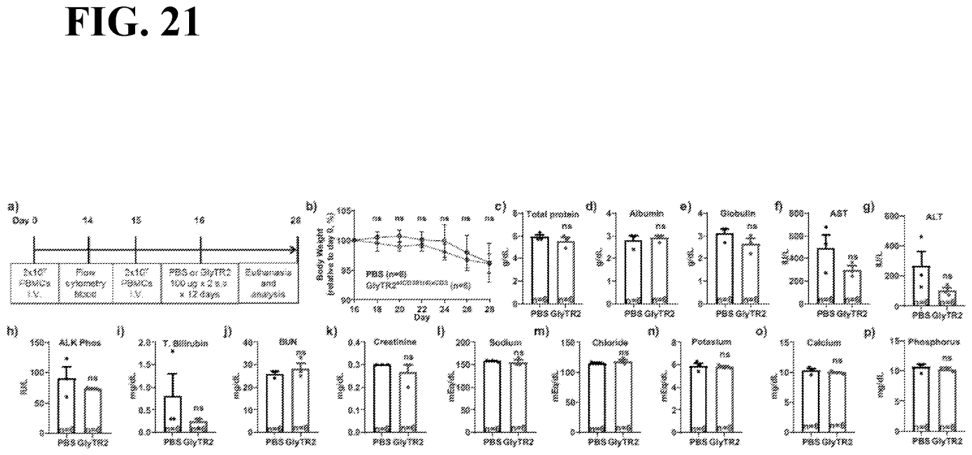

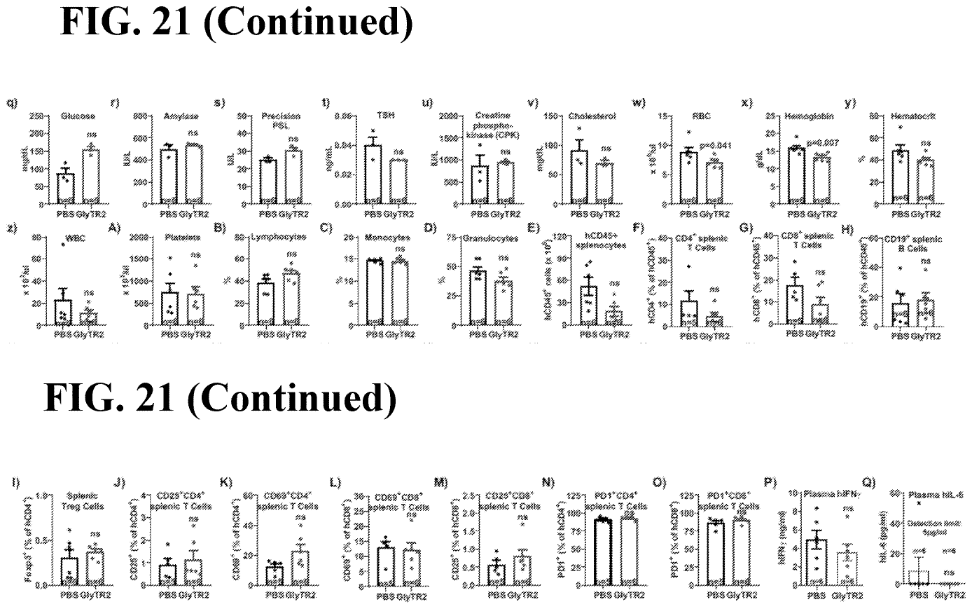

Human renal epithelial cells and normal T cells and B cells were also not killed (FIGS. 20 E, F). Moreover, treatment of PBMC humanized NSGMI/II⁻ mice (i.e., double knockout of MHC class I and II) with the GlyTR2^{slCD301(4)xCD3} @100 ug/day twice daily×12 days, the same dose that maximally killed cancer cells in vivo (FIGS. 18 B, D), demonstrated no significant differences in body weight, liver function (AST,ALT,ALP, bilirubin, protein, albumin), kidney function (urea/creatinine), electrolytes (Na⁺, Cl⁻, K⁺,Ca²⁺), pancreatic function (amylase, precision PSL), thyroid function (total T4, TSH), cholesterol, muscle (CPK), WBC, WBC differential or platelets relative to mock injected mice (FIG. 21, panels a-v, z-D). GlyTR2^{slCD301(4)xCD3} treated mice displayed minimal reductions in hemoglobin/RBC/hematocrit relative to control, but levels remained within the normal range and therefore not clinically significant (FIG. 21, panels w-y). Analysis of the spleen revealed no difference in the number of human CD45⁺ leukocytes or the percentage of CD4⁺ T cells, CD8⁺ T cells, B cells or T regulatory cells (Treg) (FIG. 21, panels E-I). There was also no difference in the T cell activation markers CD69, CD25 or PD-1 in either CD4⁺ or CD8⁺ T cells, signifying that GlyTR2^{slCD301(4)xCD3} treatment did not induce T cell activation (FIG. 21, panels J-O). Serum hIFNγ or hIL-6 levels did not differ between control and dimeric GlyTR1^{LPHA(2)xCD3} treated mice (FIG. 21, panels P, Q)

Lack of major toxicity by GlyTR2^{slCD301(4)xCD3} was consistent with CD301 being normally expressed in dendritic cells (DC) and macrophages; where engagement by Tn antigen triggers DC maturation and robust CD8⁺ T cell responses. As healthy humans lack chronic DC activation, CD301 did not appear to interact with normal human tissue in vivo. The combined data disclose herein, indicated that GlyTR2^{slCD301(4)xCD3} has minimal risk of “on-target, off-cancer” toxicity.

### Example 3: GlyTR Chimeric Antigen Receptor (CAR) T Cells

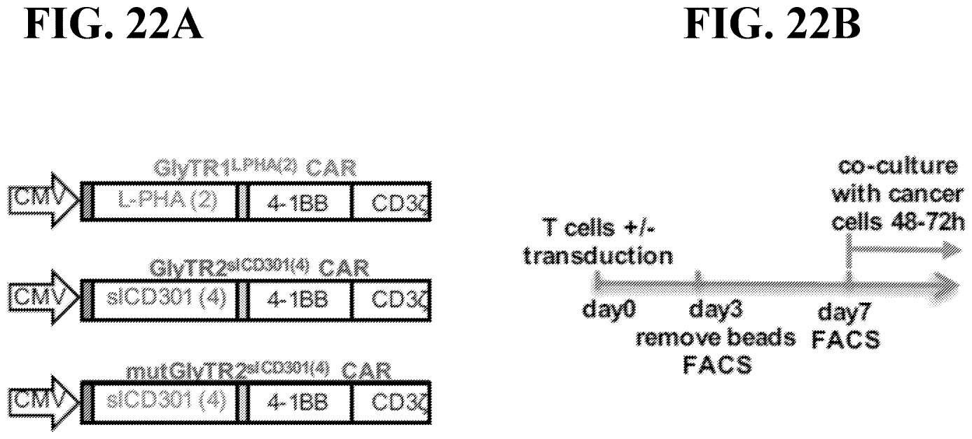

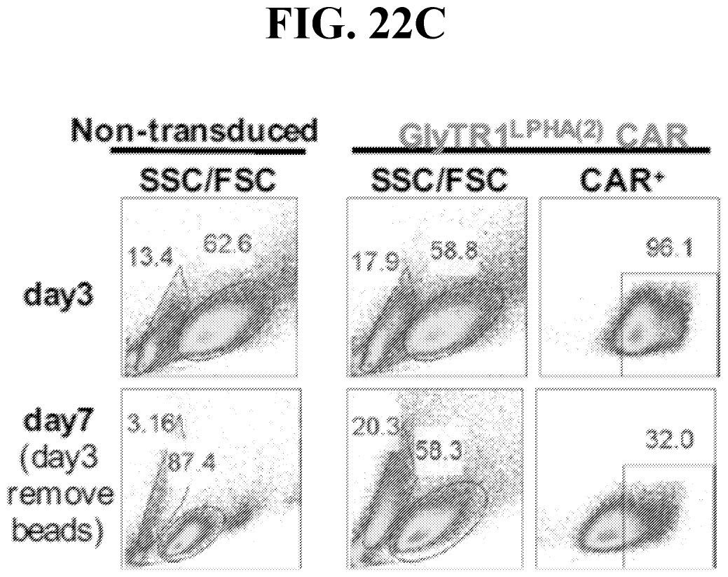

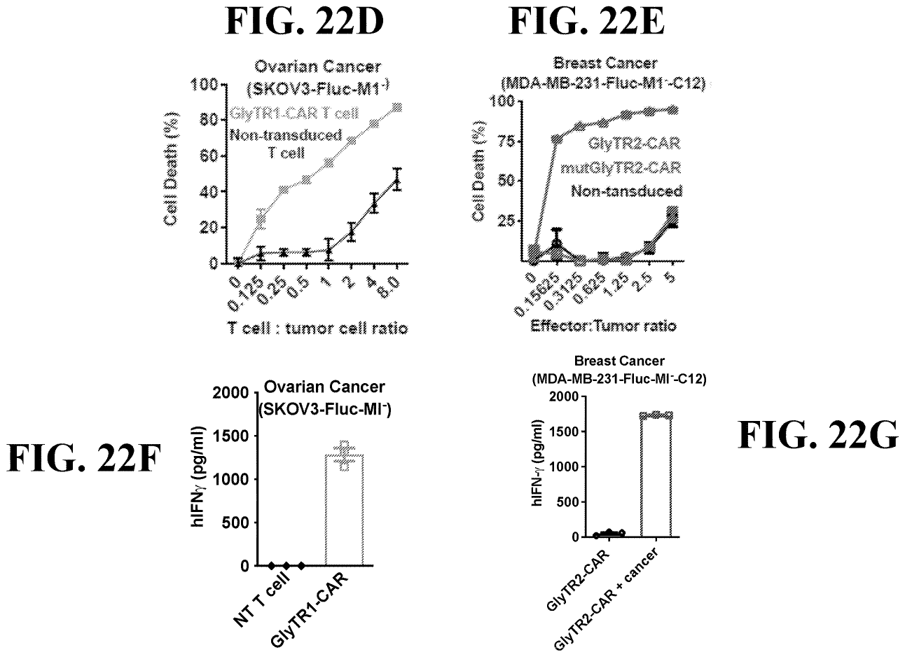

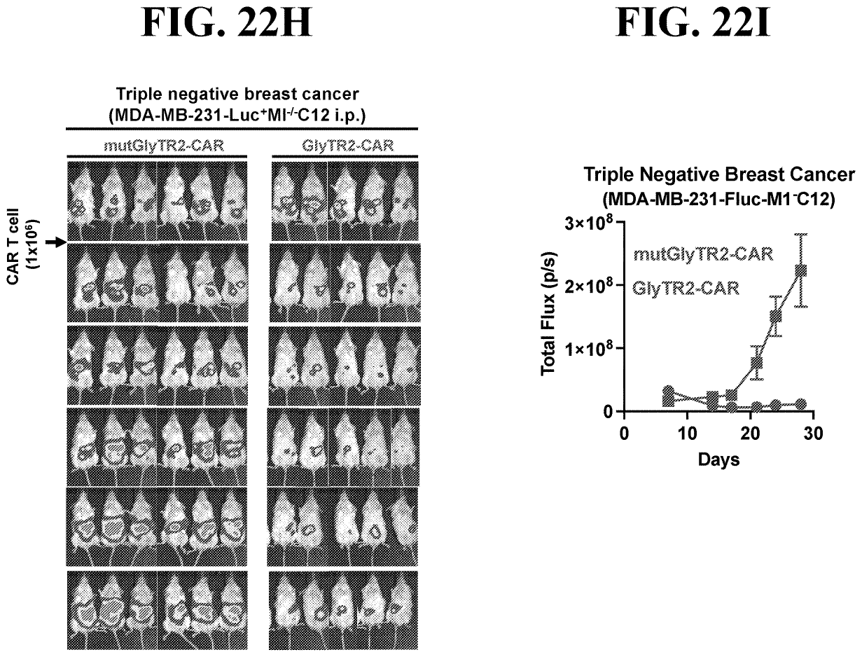

A GlyTR1-CAR and a GlyTR2-CAR were generated by fusing the optimized designs of the GlyTR1^{LPHA(2)xCD3} and GlyTR2^{slCD301(4)xCD3} bi-specific proteins described in examples 1 and 2 above to a CD8 transmembrane domain and 41BB and CD3ζ intracellular signaling domains (FIG. 22 A). To generate GlyTR-CAR T cells, purified T cells were stimulated with Dynabeads and 1-day later transduced with a lentivirus to express the GlyTR-CAR. On day 3, cells were tested by flow cytometry for CAR expression, rested for 4 days by bead removal and then co-cultured with cancer cells at various ratios to assess killing activity (FIG. 22 B). The GlyTR1^{LPHA(2)} and GlyTR2^{slCD301(4)} CAR T cells both readily killed ovarian and breast cancer cells, respectively (FIG. 22 C-E). Non-transduced control T cells induced allogenic killing at high T cell to cancer cell ratios. In addition, the GlyTR1^{LPHA(2)} and GlyTR2^{slCD301(4)} CAR T cells were active as shown by IFNγ production in the presence of cancer cells. (FIG. 22F-G). GlyTR2^{slCD301(4)} CAR T cells also readily induced tumor regression of breast cancer cells in an in vivo mouse model (FIGS. 22H-I).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### REFERENCES

Nunez-Prado, N. et al. The coming of age of engineered multivalent antibodies. Drug discovery today 20, 588-594, doi:10.1016/j.drudis.2015.02.013 (2015).
Loffler, A. et al. A recombinant bi-specific single-chain antibody, CD19×CD3, induces rapid and high lymphoma-directed cytotoxicity by unstimulated T lymphocytes. Blood 95, 2098-2103 (2000).
Scott, A. M., Wolchok, J. D. & Old, L. J. Antibody therapy of cancer. Nature reviews. Cancer 12, 278-287, doi:10.1038/nrc3236 (2012).
Feizi, T. Demonstration by monoclonal antibodies that carbohydrate structures of glycoproteins and glycolipids are onco-developmental antigens. Nature 314, 53-57 (1985).
Kim, Y. J. & Varki, A. Perspectives on the significance of altered glycosylation of glycoproteins in cancer. Glycoconjugate journal 14, 569-576 (1997)
Fernandes, B., Sagman, U., Auger, M., Demetrio, M. & Dennis, J. W. Beta 1-6 branched oligosaccharides as a marker of tumor progression in human breast and colon neoplasia. Cancer research 51, 718-723 (1991).
Lau, K. S. & Dennis, J. W. N-Glycans in cancer progression. Glycobiology 18, 750-760, doi:10.1093/glycob/cwnO71 (2008).
Litynska, A. et al. Comparison of the lectin-binding pattern in different human melanoma cell lines. Melanoma research 11, 205-212 (2001).
Guo, H. B., Randolph, M. & Pierce, M. Inhibition of a specific N-glycosylation activity results in attenuation of breast carcinoma cell invasiveness-related phenotypes: inhibition of epidermal growth factor-induced dephosphorylation of focal adhesion kinase. The Journal of biological chemistry 282, 22150-22162, doi:10.1074/jbc.M611518200 (2007).
Dennis, J. W., Laferte, S., Waghorne, C., Breitman, M. L. & Kerbel, R. S. Beta 1-6 branching of Asn-linked oligosaccharides is directly associated with metastasis. Science 236, 582-585 (1987).
Demetriou, M., Nabi, I. R., Coppolino, M., Dedhar, S. & Dennis, J. W. Reduced contact-inhibition and substratum adhesion in epithelial cells expressing GlcNAc-transferase V. The Journal of cell biology 130, 383-392 (1995).
Granovsky, M. et al. Suppression of tumor growth and metastasis in Mgat5-deficient mice. Nat Med 6, 306-312, doi:10.1038/73163 (2000).
Beheshti Zavareh, R. et al. Suppression of cancer progression by MGAT1 shRNA knockdown. PloS one 7, e43721, doi:10.1371/journal.pone.0043721 (2012).
Dingjan, T. et al. Structural biology of antibody recognition of carbohydrate epitopes and potential uses for targeted cancer immunotherapies. Molecular immunology 67, 75-88, doi:10.1016/j.molimm.2015.02.028 (2015).
Blixt, O. et al. Analysis of Tn antigenicity with a panel of new IgM and IgG1 monoclonal antibodies raised against leukemic cells. Glycobiology 22, 529-542, doi:10.1093/glycob/cwrl78 (2012).
Reusch, U. et al. A tetravalent bi-specific TandAb (CD19/CD3), AFM11, efficiently recruits T cells for the potent lysis of CD19(+) tumor cells. mAbs 7, 584-604, doi:10.1080/19420862.2015.1029216 (2015).
Klinger, M. et al. Immunopharmacologic response of patients with B-lineage acute lymphoblastic leukemia to continuous infusion of T cell-engaging CD19/CD3-bi-specific BiTE antibody blinatumomab. Blood 119, 6226-6233, doi:10.1182/blood-2012-01-400515 (2012).
Spitzer, M. H. et al. Systemic Immunity Is Required for Effective Cancer Immunotherapy. Cell 168, 487-502 e415, doi:10.1016/j.cell.2016.12.022 (2017).
Eyquem, J. et al. Targeting a CAR to the TRAC locus with CRISPR/Cas9 enhances tumour rejection. Nature 543, 113-117, doi:10.1038/nature21405 (2017).
Hamelryck, T. W. et al. The crystallographic structure of phytohemagglutinin-L. J. Biol. Chem. 271, 20479-20485 (1996).
Truong, L. D., Phung, V. T., Yoshikawa, Y. & Mattioli, C. A. Glycoconjugates in normal human kidney. A histochemical study using 13 biotinylated lectins. Histochemistry 90, 51-60, doi:10.1007/BF00495707 (1988).
Shultz, L. D. et al. Human lymphoid and myeloid cell development in NOD/LtSz-scid IL2R gamma null mice engrafted with mobilized human hemopoietic stem cells. J Immunol 174, 6477-6489 (2005).
Sonntag, K. et al. Chronic graft-versus-host-disease in CD34(+)-humanized NSG mice is associated with human susceptibility HLA haplotypes for autoimmune disease. J Autoimmun 62, 55-66, doi:10.1016/j.jaut.2015.06.006 (2015).
Ju, T., Aryal, R. P., Kudelka, M. R., Wang, Y. & Cummings, R. D. The Cosme connection to the Tn antigen in cancer. Cancer biomarkers: section A of Disease markers 14, 63-81, doi:10.3233/CBM-130375 (2014).
Springer, G. F. T and Tn, general carcinoma autoantigens. Science 224, 1198-1206 (1984).
Springer, G. F. Immunoreactive T and Tn epitopes in cancer diagnosis, prognosis, and immunotherapy. Journal of molecular medicine 75, 594-602 (1997).
Kawaguchi, T. Cancer metastasis: characterization and identification of the behavior of metastatic tumor cells and the cell adhesion molecules, including carbohydrates. Current drug targets. Cardiovascular& haematological disorders 5, 39-64 (2005).
Terasawa, K., Furumoto, H., Kamada, M. & Aono, T. Expression of Tn and sialyl-Tn antigens in the neoplastic transformation of uterine cervical epithelial cells. Cancer research 56, 2229-2232 (1996).
Laack, E. et al. Lectin histochemistry of resected adenocarcinoma of the lung: helix pomatia agglutinin binding is an independent prognostic factor. The American journal of pathology 160, 1001-1008, doi:10.1016/S0002-9440(10)64921-8 (2002).
(Konno, A., Hoshino, Y., Terashima, S., Motoki, R. & Kawaguchi, T. Carbohydrate expression profile of colorectal cancer cells is relevant to metastatic pattern and prognosis. Clinical & experimental metastasis 19, 61-70 (2002).
Gill, D. J. et al. Initiation of GalNAc-type O-glycosylation in the endoplasmic reticulum promotes cancer cell invasiveness. Proceedings of the National Academy of Sciences of the United States ofAmerica 110, E3152-3161, doi:10.1073/pnas.1305269110 (2013).
Nguyen, A. T. et al. Organelle Specific O-Glycosylation Drives MMP14 Activation, Tumor Growth, and Metastasis. Cancer cell 32, 639-653 e636, doi:10.1016/j.ccell.2017.10.001 (2017).
Gill, D. J., Chia, J., Senewiratne, J. & Bard, F. Regulation of O-glycosylation through Golgi-to-ER relocation of initiation enzymes. The Journal of cell biology 189, 843-858, doi:10.1083/jcb.201003055 (2010).
Nollau, P. et al. Protein domain histochemistry (PDH): binding of the carbohydrate recognition domain (CRD) of recombinant human glycoreceptor CLEC10A (CD301) to formalin-fixed, paraffin-embedded breast cancer tissues. The journal of histochemistry and cytochemistry: official journal of the Histochemistry Society 61, 199-205, doi:10.1369/0022155412474823 (2013).
Lenos, K. et al. MGL ligand expression is correlated to BRAF mutation and associated with poor survival of stage III colon cancer patients. Oncotarget 6, 26278-26290, doi:10.18632/oncotarget.4495 (2015).
van Vliet, S. J. et al. Carbohydrate profiling reveals a distinctive role for the C-type lectin MGL in the recognition of helminth parasites and tumor antigens by dendritic cells. International immunology 17, 661-669, doi:10.1093/intimm/dxh246 (2005).
Marcelo, F. et al. Delineating binding modes of Gal/GalNAc and structural elements of the molecular recognition of tumor-associated mucin glycopeptides by the human macrophage galactose-type lectin. Chemistry 20, 16147-16155, doi:10.1002/chem.201404566 (2014).
Jegouzo, S. A. et al. Organization of the extracellular portion of the macrophage galactose receptor: a trimeric cluster of simple binding sites for N-acetylgalactosamine. Glycobiology 23, 853-864, doi:10.1093/glycob/cwt022 (2013).
Artigas, G. et al. Glycopeptides as Targets for Dendritic Cells: Exploring MUC1 Glycopeptides Binding Profile toward Macrophage Galactose-Type Lectin (MGL) Orthologs. Journal of medicinal chemistry 60, 9012-9021, doi:10.1021/acs.jmedchem.7b01242 (2017).
Mortezai, N. et al. Tumor-associated Neu5Ac-Tn and Neu5Gc-Tn antigens bind to C-type lectin CLEC10A (CD301, MGL). Glycobiology 23, 844-852, doi:10.1093/glycob/cwt021 (2013).
Hung, J.-T. a. Y., A. L. in Glycosignals in Cancer: Mechanisms ofMalignant Phenotypes (ed K Furukawa, Fukuda, M.) Ch. 11, 197-219 (Springer, 2016).
Hirano, K., Matsuda, A., Shirai, T. & Furukawa, K. Expression of LacdiNAc groups on N-glycans among human tumors is complex. BioMed research international 2014, 981627, doi:10.1155/2014/981627 (2014).
Higashi, N. et al. The macrophage C-type lectin specific for galactose/N-acetylgalactosamine is an endocytic receptor expressed on monocyte-derived immature dendritic cells. The Journal of biological chemistry 277, 20686-20693, doi:10.1074/jbc.M202104200 (2002).
Napoletano, C. et al. Targeting of macrophage galactose-type C-type lectin (MGL) induces DC signaling and activation. Eur J Immunol 42, 936-945, doi:10.1002/eji.201142086 (2012).
Oo-Puthinan, S. et al. The amino acids involved in the distinct carbohydrate specificities between macrophage galactose-type C-type lectins 1 and 2 (CD301a and b) of mice. Biochim Biophys Acta 1780, 89-100, doi:10.1016/j.bbagen.2007.10.017 (2008).
Chen, X., Zaro, J. L. & Shen, W. C. Fusion protein linkers: property, design and functionality. Advanced drug delivery reviews 65, 1357-1369, doi:10.1016/j.addr.2012.09.039 (2013).

1. An isolated nucleic acid molecule encoding:
(i) a bi-specific fusion protein comprising:
(a) an antigen binding domain that selectively binds a tumor-associated carbohydrate antigen (TACA), and
(b) an immune cell recognition domain that specifically binds a receptor on an immune effector cell; or
(i) a chimeric antigen receptor (CAR) comprising: (a) an antigen binding domain that selectively binds a tumor-associated carbohydrate antigen (TACA): (b) a transmembrane domain: (c) a costimulatory signaling region; and (d) an intracellular signaling domain;
wherein the antigen binding domain comprises a TACA-binding domain derived from a lectin; and wherein the antigen binding domain comprises more than one TACA binding domains.

2. (canceled)

3. The isolated nucleic acid molecule of claim 1, wherein the antigen binding domain comprises two, three, four, five, six, seven, eight, nine, ten, or more TACA binding domains.

4. The isolated nucleic acid molecule of claim 1, wherein the more than one TACA binding domains are operably linked by a linker.

5.-8. (canceled)

9. The isolated nucleic acid molecule of claim 4, wherein:
(a) the linker comprises the amino acid sequence selected from the group consisting of SEQ ID NO: 128, SEQ ID NO: 129, SEQ ID NO: 127, SEQ ID NO: 130, SEQ ID NO: 131, and SEQ ID NO: 132; or
(b) the linker is a peptide linker; or
(c) the linker is a glycine-serine linker.

10.-16. (canceled)

17. The isolated nucleic acid molecule of claim 1, wherein the antigen binding domain comprises the amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152.

18.-19. (canceled)

20. The isolated nucleic acid molecule of claim 1, wherein the isolated nucleic acid molecule encodes a bi-specific fusion protein comprising an amino acid sequence selected from SEQ ID NOs: 1-5, 10-34, 39-42, 47-50, 55-58, and 63-66; or an amino acid sequence having at least 90% sequence identity to an amino acid sequence selected from SEQ ID NOs: 1-5, 10-34, 39-42, 47-50, 55-58, and 63-66.

21. The isolated nucleic acid molecule of claim 1, wherein the isolated nucleic acid molecule encodes a bi-specific fusion protein comprising:
(a) an amino acid sequence selected from SEQ ID NO: 3-5, 11-13, 19-21, 28-30, 32-34, 40-42, 48-50, 56-58, or 64-66; or
(b) an amino acid sequence of SEQ ID NOs: 32-34, 40-42, 48-50, 56-58, or 64.

22. (canceled)

23. The isolated nucleic acid molecule of claim 1, wherein:
(a) the bi-specific fusion protein exhibits enhanced binding to Thomsen-nouveau (Tn) antigen expressing tumor cells when compared to a bi-specific fusion protein comprising a flexible linker in the antigen binding domain; and/or
(b) the bi-specific fusion protein exhibits enhanced binding to β1,6GlcNAc-branched N-glycans expressing tumor cells when compared to a bi-specific fusion protein comprising a flexible linker in the antigen binding domain; and
wherein the flexible linker is a glycine-serine linker or a linker comprising an amino acid sequence selected from SEQ ID NO: 124, SEQ ID NO: 128, SEQ ID NO: 129, or SEQ ID NO: 127: or an amino acid sequence having at least 90% sequence identity to an amino acid sequence selected from SEQ ID NO: 124, SEQ ID NO: 128, SEQ ID NO: 129, or SEQ ID NO: 127.

24. (canceled)

25. The isolated nucleic acid molecule of claim 1, wherein the immune effector cell is selected from the group consisting of a T cell, a natural killer (NK) cell, a natural killer T (NKT) cell, a macrophage, a monocyte, a dendritic cell, and a neutrophil.

26.-30. (canceled)

31. The isolated nucleic acid molecule of claim 1, wherein:
(a) the immune cell recognition domain comprises:
(i) an scFv that selectively binds CD3, CD2, CD28, CD25, CD16, NKG2D, NKG2A, CD138, KIR3DL, NKp46, MICA, and CEACAM1;
(ii) the amino acid sequence of SEQ ID NOs: 149, 150 or 151; or
(iii) an amino acid sequence having at least 90% sequence identity to the amino acid sequence of SEQ ID NO: 149, 150, or 151; or
(b) the bi-specific fusion protein is an Fc fusion protein comprising the antigen binding domain that selectively binds a tumor-associated carbohydrate antigen (TACA) and the Fc domain.

32.-44. (canceled)

45. A bi-specific fusion protein that selectively binds a tumor-associated carbohydrate antigen (TACA), wherein the bi-specific fusion protein is encoded by the isolated nucleic acid of claim 1.

46. A bi-specific fusion protein that selectively binds a tumor-associated carbohydrate antigen (TACA) comprising:
(i) an antigen binding domain selected from the group consisting of SEQ ID NOs: 100, 101, 102, 103, 104, 105, 106, 107, 108, 109, 133, 134, 135, 136, 136, 137, 139, 140, 141, 142, 143, 144, 145, 146, and 152; or an amino acid sequence having at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to an amino acid sequence set forth in SEQ ID NOs: 100, 101, 102, 103, 104, 105, 106, 107, 108, 109, 133, 134, 135, 136, 136, 137, 139, 140, 141, 142, 143, 144, 145, 146, or 152; and
(ii) an immune cell recognition domain that specifically binds a receptor on an immune effector cell.

47.-48. (canceled)

49. The bi-specific fusion protein of claim 46, wherein the bi-specific fusion protein comprises:
(a) the amino acid sequence selected from SEQ ID NO: SEQ ID NOs: 1-5, 10-34, 39-42, 47-50, 55-58, and 63-66; or
(b) an amino acid sequence having at least 90% sequence identity to an amino acid sequence selected from SEQ ID NOs: SEQ ID NOs: 1-5, 10-34, 39-42, 47-50, 55-58, and 63-66; or
(c) the amino acid sequence selected from SEQ ID NO: 3-5, 11-13, 19-21, 28-30, 32-34, 40-42, 48-50, 56-58, or 64-66; or
(d) an amino acid sequence having at least 90% sequence identity to an amino acid sequence selected from SEQ ID NO: 3-5, 11-13, 19-21, 28-30, 32-34, 40-42, 48-50, 56-58, or 64-66.

50.-51. (canceled)

52. The bi-specific fusion protein of claim 46, wherein the bi-specific fusion protein that selectively targets_:
(a) a Tn antigen comprises an antigen binding domain having the amino acid sequence selected from SEQ ID NO: 103-109, 142-146, or 152; or an amino acid sequence having at least 90% sequence identity to an amino acid sequence selected from SEQ ID NO: 103-109, 142-146, or 152; or
(b) the bi-specific fusion protein that selectively targets a β1,6GlcNAc-branched N-glycan comprises an antigen binding domain having the amino acid sequence selected from SEQ ID NO: 100-102, or 133-141: or an amino acid sequence having at least 90% sequence identity to an amino acid sequence selected from SEQ ID NO: 100-102, or 133-141.

53. The bi-specific fusion protein of claim 46, wherein the bi-specific fusion protein that selectively targets a Tn antigen comprises:
(a) the amino acid sequence selected from SEQ ID NOs: 26-34, 39-42, 47-50, 55-58, or 63-66; or
(b) an amino acid sequence having at least 90% sequence identity to an amino acid sequence selected from SEQ ID NOs: 26-34, 39-42, 47-50, 55-58, or 63-66;
(c) the amino acid sequence selected from SEQ ID NOs: 28-30, 32-34, 40-42, 48-50, 56-58, or 64-66; or
(d) an amino acid sequence having at least 90% sequence identity to an amino acid sequence selected from SEQ ID NOs: 28-30, 32-34, 40-42, 48-50, 56-58, or 64-66.

54.-69. (canceled)

70. A modified cell comprising the isolated nucleic acid of claim 1.

71. The modified cell of claim 70, wherein the cell is selected from the group consisting of a T cell, a CD4⁺ T cell, a CD8⁺ T cell, a Natural Killer (NK) cell, a cytotoxic T lymphocyte (CTL), and a regulatory T cell.

72.-74. (canceled)

75. A composition comprising the modified cell of claim 70.

76.-77. (canceled)

78. A method of treating cancer in a subject in need thereof, the method comprising administering to the subject a composition of claim 75.

79. (canceled)

80. The method of claim 78, wherein the cancer is selected from the group consisting of a hematological malignancy, a solid tumor, a primary or a metastasizing tumor, a leukemia, a carcinoma, a blastoma, a sarcoma, a leukemia, lymphoid malignancies, a melanoma, and a lymphoma.

81.-89. (canceled)


SEQUENCE LISTING (165 sequences)

SEQ ID NO: 1 - protein, 500 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - protein, 510 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 3 - protein, 777 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 4 - protein, 1044 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 5 - protein, 1311 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 6 - protein, 1109 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKASNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 7 - protein, 1376 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKASNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 8 - protein, 1643 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKASNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 9 - protein, 1910 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKASNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 10 - protein, 507 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 11 - protein, 771 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 12 - protein, 1035 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 13 - protein, 1299 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 14 - protein, 1106 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 15 - protein, 1370 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 16 - protein, 1634 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 17 - protein, 1898 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 18 - protein, 505 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 19 - protein, 767 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 20 - protein, 1029 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 21 - protein, 1291 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 22 - protein, 1104 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 23 - protein, 1366 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 24 - protein, 1628 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 25 - protein, 1890 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 26 - protein, 430 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 27 - protein, 430 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGGPGDWQGHGLGGGGDCAHFHPDGRWGGDVCQRPYHWVCEAGLGQTSQESHGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 28 - protein, 599 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 29 - protein, 768 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 30 - protein, 937 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 31 - protein, 429 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 32 - protein, 597 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 33 - protein, 765 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 34 - protein, 933 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 35 - protein, 1028 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAVHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 36 - protein, 1196 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAVHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 37 - protein, 1364 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAVHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 38 - protein, 1532 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKAVHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 39 - protein, 411 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 40 - protein, 579 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 41 - protein, 747 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 42 - protein, 915 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 43 - protein, 1010 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 44 - protein, 1178 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 45 - protein, 1346 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 46 - protein, 1514 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 47 - protein, 401 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 48 - protein, 559 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 49 - protein, 717 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 50 - protein, 875 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 51 - protein, 1000 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 52 - protein, 1158 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 53 - protein, 1316 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 54 - protein, 1474 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 55 - protein, 393 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 56 - protein, 543 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 57 - protein, 693 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 58 - protein, 843 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 59 - protein, 992 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 60 - protein, 1142 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 61 - protein, 1292 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 62 - protein, 1442 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 63 - protein, 383 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 64 - protein, 523 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 65 - protein, 663 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 66 - protein, 803 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 67 - protein, 982 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 68 - protein, 1122 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 69 - protein, 1262 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 70 - protein, 1402 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAAEAAAKAAEAAAKADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 71 - protein, 490 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 757 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 1024 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 1291 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 543 aa, synthetic construct
GGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 795 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 1062 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 1329 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 485 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 747 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 1009 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 1271 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 487 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 751 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 1015 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 1279 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 793 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILAEAAAKAAEAAAKASNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILAEAAAKAAEAAAKAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 913 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 913 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGGPGDWQGHGLGGGGDCAHFHPDGRWGGDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGGPGDWQGHGLGGGGDCAHFHPDGRWGGDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGGPGDWQGHGLGGGGDCAHFHPDGRWGGDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGGPGDWQGHGLGGGGDCAHFHPDGRWGGDVCQRPYHWVCEAGLGQTSQESHAEAAAKAAEAAAKATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 923 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKALTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKAFVPVFLPAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 433 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 395 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 387 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 405 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 415 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 397 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 547 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 697 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 861 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKACPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHAEAAAKAAEAAAKAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 252 aa, synthetic construct
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 101 - protein, 249 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 102 - protein, 247 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 103 - protein, 172 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESH

SEQ ID NO: 104 - protein, 172 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGGPGDWQGHGLGGGGDCAHFHPDGRWGGDVCQRPYHWVCEAGLGQTSQESH

SEQ ID NO: 105 - protein, 154 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESH

SEQ ID NO: 106 - protein, 154 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGGPGDWQGHGLGGGGDCAHFHPDGRWGGDVCQRPYHWVCEAGLGQTSQESH

SEQ ID NO: 107 - protein, 144 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEA

SEQ ID NO: 108 - protein, 136 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESH

SEQ ID NO: 109 - protein, 126 aa, synthetic construct
CPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEA

SEQ ID NO: 110 - protein, 585 aa, Homo sapiens [sequence omitted]

SEQ ID NO: 111 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 112 - protein, 79 aa, synthetic construct
FVPVFLPAKPTTTPAPRPPTPAPTIASQPLSLRPEACRPAAGGAVHTRGLDFACDIYIWAPLAGTCGVLLLSLVITLYC

SEQ ID NO: 113 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 114 - protein, 42 aa, synthetic construct
KRGRKKLLYIFKQPFMRPVQTTQEEDGCSCRFPEEEEGGCEL

SEQ ID NO: 115 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 14 aa, synthetic construct
  VARIANT 1..14: note = AEAAAKA repeats may vary between 1 to 2. Up to 1 AEAAAKA repeats may be absent
AEAAAKAAEAAAKA

SEQ ID NO: 117 - protein, 7 aa, synthetic construct
AEAAAKA

SEQ ID NO: 118 - protein, 5 aa, synthetic construct
  source 1..5: note = Rigid linker
EAAAK

SEQ ID NO: 119 - protein, 46 aa, synthetic construct
  source 1..46: note = Rigid linker
  VARIANT 2..21: note = EAAAK repeats may vary between 1 upto 4. Up to 3 EAAAK repeats may be absent
  VARIANT 26..45: note = EAAAK repeats may vary between 1 upto 4. Up to 3 EAAAK repeats may be absent
AEAAAKEAAAKEAAAKEAAAKALEAEAAAKEAAAKEAAAKEAAAKA

SEQ ID NO: 120 - protein, 5 aa, synthetic construct
  source 1..5: note = Rigid linker
PAPAP

SEQ ID NO: 121 - skipped

SEQ ID NO: 122 - protein, 4 aa, synthetic construct
  source 1..4: note = Flexible linker
GGGS

SEQ ID NO: 123 - protein, 15 aa, synthetic construct
  source 1..15: note = Flexible linker
  VARIANT 1..15: note = GGGGS repeats may vary between 1 to 3. Up to 2 GGGGS repeats may be absent
GGGGSGGGGSGGGGS

SEQ ID NO: 124 - protein, 13 aa, synthetic construct
  source 1..13: note = Flexible linker
GGGSGGGGSGGGS

SEQ ID NO: 125-126 - skipped (2 entries)

SEQ ID NO: 127 - protein, 15 aa, synthetic construct
  source 1..15: note = Flexible linker
GGGGSGGGGSGGGGS

SEQ ID NO: 128 - protein, 5 aa, synthetic construct
  source 1..5: note = Flexible linker
GGGGS

SEQ ID NO: 129 - protein, 10 aa, synthetic construct
  source 1..10: note = Flexible linker
GGGGSGGGGS

SEQ ID NO: 130 - protein, 7 aa, synthetic construct
  source 1..7: note = Rigid linker
AEAAAKA

SEQ ID NO: 131 - protein, 14 aa, synthetic construct
  source 1..14: note = Rigid linker
AEAAAKAAEAAAKA

SEQ ID NO: 132 - protein, 21 aa, synthetic construct
  source 1..21: note = Rigid linker
AEAAAKAAEAAAKAAEAAAKA

SEQ ID NO: 133 - protein, 519 aa, synthetic construct
  source 1..519: note = L-PHA (2)
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 134 - protein, 786 aa, synthetic construct
  source 1..786: note = L-PHA (3)
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 135 - protein, 1053 aa, synthetic construct
  source 1..1053: note = L-PHA (4)
SNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSSNDIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 136 - protein, 513 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 137 - protein, 777 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 138 - protein, 1041 aa, synthetic construct
IYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSIYFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 139 - protein, 509 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 140 - protein, 771 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 141 - protein, 1033 aa, synthetic construct
FNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKILGGGGSGGGGSGGGGSFNFQRFNETNLILQRDASVSSSGQLRLTNLNGNGEPRVGSLGRAFYSAPIQIWDNTTGTVASFATSFTFNIQVPNNAGPADGLAFALVPVGSQPKDKGGFLGLFDGSNSNFHTVAVEFDTLYNKDWDPTERHIGIDVNSIRSIKTTRWDFVNGENAEVLITYDSSTNLLVASLVYPSQKTSFIVSDTVDLKSVLPEWVSVGFSATTGINKGNVETNDVLSWSFASKLSDGTTSEGLNLANLVLNKIL

SEQ ID NO: 142 - protein, 341 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESH

SEQ ID NO: 143 - protein, 510 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESH

SEQ ID NO: 144 - protein, 323 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESH

SEQ ID NO: 145 - protein, 679 aa, synthetic construct
VHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESH

SEQ ID NO: 146 - protein, 492 aa, synthetic construct
LTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESHGGGGSGGGGSGGGGSLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESH

SEQ ID NO: 147 - protein, 45 aa, synthetic construct
TTTPAPRPPTPAPTIASQPLSLRPEACRPAAGGAVHTRGLDFACD

SEQ ID NO: 148 - protein, 24 aa, synthetic construct
  source 1..24: note = CD8 transmembrane
IYIWAPLAGTCGVLLLSLVITLYC

SEQ ID NO: 149 - protein, 243 aa, synthetic construct
  source 1..243: note = CD3 scFv
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 150 - protein, 119 aa, synthetic construct
  source 1..119: note = CD3 Heavy Chain variable domain
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 151 - protein, 106 aa, synthetic construct
  source 1..106: note = CD3 light chain variable domain
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 152 - protein, 154 aa, synthetic construct; identical to SEQ ID NO: 105
  source 1..154: note = CD301CRD

SEQ ID NO: 153 - protein, 10 aa, synthetic construct
  source 1..10: note = IgG1 hinge/region
DKTHTCPPCP

SEQ ID NO: 154 - protein, 15 aa, synthetic construct
  source 1..15: note = IgG1 hinge/region
EPKSCDKTHTCPPCP

SEQ ID NO: 155 - protein, 12 aa, synthetic construct
  source 1..12: note = IgG2 hinge/region
ERKCCVECPPCP

SEQ ID NO: 156 - protein, 16 aa, synthetic construct
  source 1..16: note = IgG3 hinge/region
ELKTPLDTTHTCPRCP

SEQ ID NO: 157 - protein, 17 aa, synthetic construct
  source 1..17: note = IgG3 hinge/region
ELKTPLGDTTHTCPRCP

SEQ ID NO: 158 - protein, 12 aa, synthetic construct
  source 1..12: note = IgG4 hinge/region
ESKYGPPCPSCP

SEQ ID NO: 159 - skipped

SEQ ID NO: 160 - protein, 48 aa, synthetic construct
  source 1..48: note = Serum albumin binding domain
ASPXAACYSLEUPROXAATRPGLYCYSLEUTRPDXAACLPXAAWGCLW

SEQ ID NO: 161 - protein, 21 aa, synthetic construct
  source 1..21: note = Serum albumin binding domain
QGLIGDICLPRWGCLWGDSVK

SEQ ID NO: 162 - protein, 18 aa, synthetic construct
  source 1..18: note = Serum albumin binding domain
RLIEDICLPRWGCLWEDD

SEQ ID NO: 163 - protein, 14 aa, synthetic construct
  source 1..14: note = Serum albumin binding domain
EDICLPRWGCLWED

SEQ ID NO: 164 - protein, 316 aa, Homo sapiens
  source 1..316: note = "NP_878910.1 C-type lectin domain family 10 member A isoform 1"
MTRTYENFQYLENKVKVQGFKNGPLPLQSLLQRLCSGPCHLLLSLGLGLLLLVIICVVGFQNSKFQRDLVTLRTDFSNFTSNTVAEIQALTSQGSSLEETIASLKAEVEGFKQERQAGVSELQEHTTQKAHLGHCPHCPSVCVPVHSEMLLRVQQLVQDLKKLTCQVATLNNNASTEGTCCPVNWVEHQDSCYWFSHSGMSWAEAEKYCQLKNAHLVVINSREEQNFVQKYLGSAYTWMGLSDPEGAWKWVDGTDYATGFQNWKPGQPDDWQGHGLGGGEDCAHFHPDGRWNDDVCQRPYHWVCEAGLGQTSQESH

SEQ ID NO: 165 - protein, 4 aa, synthetic construct
  source 1..4: note = flexible linker
  VARIANT 1..4: note = GGGS repeats may vary between 1-10
GGGS